(19) United States
(12) Patent Application Publication
(10) Pub. No.: US 2021/0028365 A1
(43) Pub. Date: Jan. 28, 2021

(54) ORGANIC ELECTROLUMINESCENCE DEVICE AND ELECTRONIC APPLIANCE

(71) Applicant: IDEMITSU KOSAN CO.,LTD., Tokyo (JP)

(72) Inventors: Satomi TASAKI, Sodegaura-shi (JP); Yuki NAKANO, Sodegaura-shi (JP); Taro YAMAKI, Sodegaura-shi (JP); Hiroaki ITOI, Sodegaura-shi (JP); Yuichiro KAWAMURA, Sodegaura-shi (JP); Masatoshi SAITO, Sodegaura-shi (JP)

(73) Assignee: IDEMITSU KOSAN CO.,LTD., Tokyo (JP)

(21) Appl. No.: 17/043,247

(22) PCT Filed: Apr. 4, 2019
(86) PCT No.: PCT/JP2019/015035

§ 371 Date: Sep. 29, 2020

(30) Foreign Application Priority Data
Apr. 5, 2018 (JP) 2018-073497
Dec. 5, 2018 (JP) 2018-228510

(51) Int. Cl. H01L 51/00

(52) U.S. Cl. CPC H01L 51/0035; H01L 51/5056; H01L 51/008; H01L 51/0069

(57) ABSTRACT

An organic electroluminescence device comprising: a cathode; an anode; and an organic layer between the cathode and the anode, wherein the organic layer comprises a compound represented by the following formula (1) and a compound represented by the following formula (11).

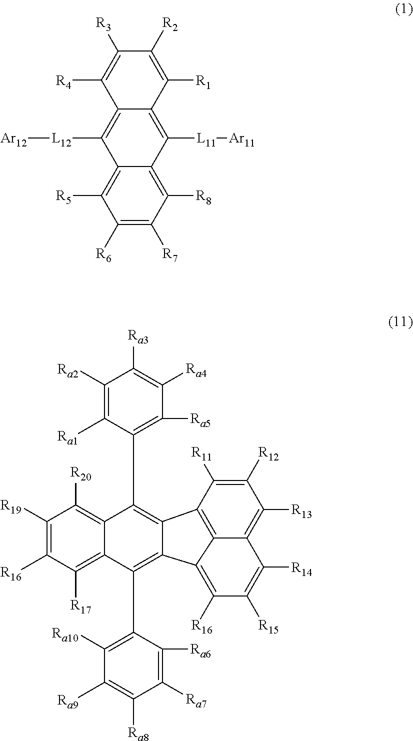

### TECHNICAL FIELD

The invention relates to an organic electroluminescence device and an electronic appliance.

### BACKGROUND ART

When voltage is applied to an organic electroluminescence device (hereinafter, referred to as an organic EL device in several cases), holes and electrons are injected into an emitting layer from an anode and a cathode, respectively. Then, thus injected holes and electrons are recombined in the emitting layer, and excitons are formed therein.

Patent Documents 1 to 3 disclose that a fluoranthene derivative is used as a dopant material for an emitting layer of an organic electroluminescence device.

### RELATED ART DOCUMENTS

### Patent Documents

[Patent Document 1] JP 2015-195348A

[Patent Document 2] JP 2015-164178 A

[Patent Document 3] JP 2013-157552 A

### SUMMARY OF THE INVENTION

It is an object to provide an organic electroluminescence device having a long lifetime or a high efficiency or a low driving voltage.

It is another object of the invention to provide an electronic appliance using an organic electroluminescence device having a long lifetime or a high efficiency or a low driving voltage.

1. According to the invention, the following organic electroluminescence device and electronic appliance are provided.

An organic electroluminescence device comprising: a cathode; an anode; and an organic layer between the cathode and the anode (hereinafter sometimes referred to as an “organic electroluminescence device 1”),

wherein the organic layer comprises a compound represented by the following formula (1) and a compound represented by the following formula (11):

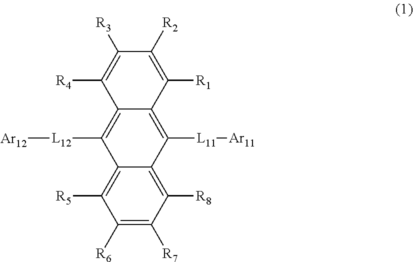

wherein in the formula (1),

one or more among R₁ to R₈ are -L₁₃-Ar₁₃;

L₁₁ to L₁₃ are independently,

a single bond,

a substituted or unsubstituted arylene group including 6 to 50 carbon atoms that form a ring (hereinafter referred to as “ring carbon atoms”), or

a substituted or unsubstituted divalent heterocyclic group including 5 to 50 atoms that form a ring (hereinafter referred to as “ring atoms”);

when two or more L₁₃'s are present, the two or more L₁₃'s may be the same as or different to each other;

Ar₁₁ to Ar₁₃ are independently,

a substituted or unsubstituted aryl group including 6 to 50 ring carbon atoms, or

a substituted or unsubstituted monovalent heterocyclic group including 5 to 50 ring atoms;

when two or more Ar₁₃'s are present, the two or more Ar₁₃'s may be the same as or different to each other;

R₁ to R₈ which are not -L₁₃-Ar₁₃ are independently,

a hydrogen atom,

a substituted or unsubstituted alkyl group including 1 to 50 carbon atoms,

a substituted or unsubstituted alkenyl group including 2 to 50 carbon atoms,

a substituted or unsubstituted alkynyl group including 2 to 50 carbon atoms,

a substituted or unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a halogen atom, a cyano group, a nitro group,

a substituted or unsubstituted aryl group including 6 to 50 ring carbon atoms, or

a substituted or unsubstituted monovalent heterocyclic group including 5 to 50 ring atoms;

R₉₀₁ to R₉₀₇ are independently,

a hydrogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms;

when two or more of each of R₉₀₁ to R₉₀₇ are present, the two or more of each of R₉₀₁ to R₉₀₇ may be the same as or different to each other;

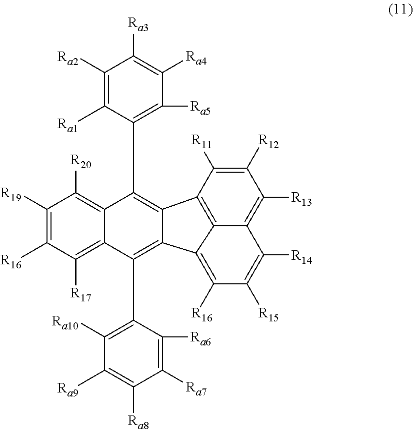

wherein in the formula (11),

any one or more sets among one or more sets of adjacent two or more of R₁₁ to R₂₀, one or more sets of adjacent two or more of Rₐ₁ to Rₐ₅, and one or more sets of adjacent two or more of Rₐ₆ to Rₐ₁₀, form a substituted or unsubstituted, saturated or unsaturated ring including 3 to 30 ring atoms by bonding with each other;

R₁₁ to R₂₀, Rₐ₁ to Rₐ₅ and Rₐ₆ to Rₐ₁₀ which do not form the ring are independently,

a hydrogen atom,

a substituted or unsubstituted alkyl group including 1 to 30 carbon atoms,

a substituted or unsubstituted cycloalkyl group including 3 to 30 ring carbon atoms,

a substituted or unsubstituted alkoxy group including 1 to 30 carbon atoms,

a substituted or unsubstituted alkylthio group including 1 to 30 carbon atoms,

a substituted or unsubstituted amino group,

a substituted or unsubstituted aryl group including 6 to 30 ring carbon atoms,

a substituted or unsubstituted heterocyclic group including 5 to 30 ring atoms.

a substituted or unsubstituted alkenyl group including 2 to 30 carbon atoms,

a substituted or unsubstituted aryloxy group including 6 to 30 ring carbon atoms,

a substituted or unsubstituted arylthio group including 6 to 30 ring carbon atoms,

a substituted or unsubstituted phosphanyl group,

a substituted or unsubstituted phosphoryl group,

a substituted or unsubstituted silyl group,

a substituted or unsubstituted arylcarbonyl group including 6 to 30 ring carbon atoms,

a cyano group, a nitro group, a carboxyl group, or

a halogen atom.

2. An organic electroluminescence device comprising: a cathode; an anode; and an organic layer between the cathode and the anode (hereinafter sometimes referred to as an “organic electroluminescence device 2”),

wherein the organic layer comprises a compound represented by the following formula (1), and

a compound A having a Stokes shift of 20 nm or less and an emission peak wavelength of 440 nm to 465 nm:

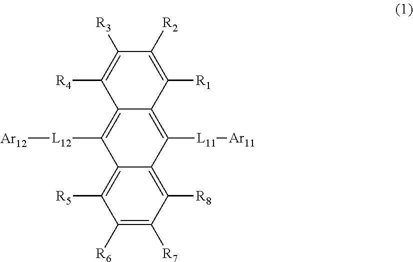

wherein in the formula (1),

one or more among R₁ to R₈ are -L₁₃-Ar₁₃;

L₁₁ to L₁₃ are independently,

a single bond,

a substituted or unsubstituted arylene group including 6 to 50 ring carbon atoms, or

a substituted or unsubstituted divalent heterocyclic group including 5 to 50 ring atoms;

when two or more L₁₃'s are present, the two or more L₁₃'s may be the same as or different to each other;

Ar₁₁ to Ar₁₃ are independently,

a substituted or unsubstituted aryl group including 6 to 50 ring carbon atoms, or

a substituted or unsubstituted monovalent heterocyclic group including 5 to 50 ring atoms;

when two or more Ar₁₃'s are present, the two or more Ar₁₃'s may be the same as or different to each other;

R₁ to R₈ which are not -L₁₃-Ar₁₃ are independently,

a hydrogen atom,

a substituted or unsubstituted alkyl group including 1 to 50 carbon atoms,

a substituted or unsubstituted alkenyl group including 2 to 50 carbon atoms,

a substituted or unsubstituted alkynyl group including 2 to 50 carbon atoms,

a substituted or unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a halogen atom, a cyano group, a nitro group,

a substituted or unsubstituted aryl group including 6 to 50 ring carbon atoms, or

a substituted or unsubstituted monovalent heterocyclic group including 5 to 50 ring atoms;

R₉₀₁ to R₉₀₇ are independently,

a hydrogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms;

when two or more of each of R₉₀₁ to R₉₀₇ are present, the two or more of each of R₉₀₁ to R₉₀₇ may be the same as or different to each other.

3. An electronic appliance, comprising the organic electroluminescence device according to 1 or 2.

According to the invention, it is possible to provide an organic electroluminescence device having a long lifetime or a high efficiency or a low driving voltage.

According to the invention, it is possible to provide an electronic appliance using an organic electroluminescence device having a long lifetime or a high efficiency or a low driving voltage.

### BRIEF DESCRIPTION OF THE DRAWINGS

FIG. 1 shows a schematic configuration of an organic EL device according to the first aspect of the invention.

FIG. 2 shows a schematic configuration of an organic EL device according to the second aspect of the invention.

FIG. 3 shows a schematic configuration of an organic EL device according to the third aspect of the invention.

FIG. 4 shows a schematic configuration of an organic EL device according to the fourth aspect of the invention.

FIG. 5 shows a schematic configuration of an organic EL device according to the fifth aspect of the invention.

### MODE FOR CARRYING OUT THE INVENTION

### Definition

In the specification, a hydrogen atom means an atom including isotopes different in the number of neutrons, namely, a protium, a deuterium and a tritium.

In the specification, a term “ring carbon atoms” represents the number of carbon atoms among atoms forming a subject ring itself of a compound having a structure in which atoms are bonded in a ring form (for example, a monocyclic compound, a fused ring compound, a cross-linked compound, a carbocyclic compound or a heterocyclic compound). When the subject ring is substituted by a substituent, the carbon contained in the substituent is not included in the number of ring carbon atoms. The same shall apply to the “ring carbon atoms” described below, unless otherwise noted. For example, a benzene ring has 6 ring carbon atoms, a naphthalene ring has 10 ring carbon atoms, a pyridine ring has 5 ring carbon atoms, and a furan ring has 4 ring carbon atoms. Further, for example, a 9,9-diphenylfluorenyl group has 13 ring carbon atoms, and a 9,9′-spirobifluorenyl group has 25 ring carbon atoms.

Further, when the benzene ring or the naphthalene ring is substituted by an alkyl group as a substituent, for example, the number of carbon atoms of the alkyl group is not included in the ring carbon atoms.

In the specification, a term “ring atoms” represents the number of atoms forming a subject ring itself of a compound having a structure in which atoms are bonded in a ring form (for example, a monocycle, a fused ring and a ring assembly) (for example, a monocyclic compound, a fused ring compound, a cross-linked compound, a carbocyclic compound or a heterocyclic compound). The term “ring atoms” does not include atoms which do not form the ring (for example, a hydrogen atom which terminates a bond of the atoms forming the ring) or atoms contained in a substituent when the ring is substituted by the substituent. The same shall apply to the “ring atoms” described below, unless otherwise noted. For example, a pyridine ring has 6 ring atoms, a quinazoline ring has 10 ring atoms, and a furan ring has 5 ring atoms. A hydrogen atom bonded with a carbon atom of the pyridine ring or the quinazoline ring or an atom forming the substituent is not included in the number of the ring atoms.

In the specification, a term “XX to YY carbon atoms” in an expression of “substituted or unsubstituted ZZ group including XX to YY carbon atoms” represents the number of carbon atoms when the ZZ group is unsubstituted. The number of carbon atoms of a substituent when the ZZ group is substituted is not included. Here, “YY” is larger than “XX”, and “XX” and “YY” each mean an integer of 1 or more.

In the specification, a term “XX to YY atoms” in an expression of “substituted or unsubstituted ZZ group including XX to YY atoms” represents the number of atoms when the ZZ group is unsubstituted. The number of atoms of a substituent when the group is substituted is not included. Here, “YY” is larger than “XX”, and “XX” and “YY” each mean an integer of 1 or more.

A term “unsubstituted” in the case of “substituted or unsubstituted ZZ group” means that the ZZ group is not substituted by a substituent, and a hydrogen atom is bonded therewith. Alternatively, a term “substituted” in the case of “substituted or unsubstituted ZZ group” means that one or more hydrogen atoms in the ZZ group are substituted by a substituent. Similarly, a term “substituted” in the case of “BB group substituted by an AA group” means that one or more hydrogen atoms in the BB group are substituted by the AA group.

Hereinafter, the substituent described herein will be described.

The number of the ring carbon atoms of the “unsubstituted aryl group” described herein is 6 to 50, preferably 6 to 30, and more preferably 6 to 18, unless otherwise specified.

The number of the ring carbon atoms of the “unsubstituted heterocyclic group” described herein is 5 to 50, preferably 5 to 30, and more preferably 5 to 18, unless otherwise specified.

The number of the carbon atoms of the “unsubstituted alkyl group” described herein is 1 to 50, preferably 1 to 20, and more preferably 1 to 6, unless otherwise specified.

The number of the carbon atoms of the “unsubstituted alkenyl group” described herein is 2 to 50, preferably 2 to 20, and more preferably 2 to 6, unless otherwise specified.

The number of the carbon atoms of the “unsubstituted alkynyl group” described herein is 2 to 50, preferably 2 to 20, and more preferably 2 to 6, unless otherwise specified.

The number of the ring carbon atoms of the “unsubstituted cycloalkyl group” described herein is 3 to 50, preferably 3 to 20, and more preferably 3 to 6, unless otherwise specified.

The number of the ring carbon atoms of the “unsubstituted arylene group” described herein is 6 to 50, preferably 6 to 30, and more preferably 6 to 18, unless otherwise specified.

The number of the ring atoms of the “unsubstituted divalent heterocyclic group” described herein is 5 to 50, preferably 5 to 30, and more preferably 5 to 18, unless otherwise specified.

The number of the carbon atoms of the “unsubstituted alkylene group” described herein is 1 to 50, preferably 1 to 20, and more preferably 1 to 6, unless otherwise specified.

Specific examples (specific example group G1) of the “substituted or unsubstituted aryl group” described herein include an unsubstituted aryl group and a substituted aryl group described below. (Here, a term “unsubstituted aryl group” refers to a case where the “substituted or unsubstituted aryl group” is the “unsubstituted aryl group,” and a term “substituted aryl group” refers to a case where the “substituted or unsubstituted aryl group” is the “substituted aryl group”. Hereinafter, a case of merely “aryl group” includes both the “unsubstituted aryl group” and the “substituted aryl group”.

The “substituted aryl group” refers to a case where the “unsubstituted aryl group” has a substituent, and specific examples thereof include a group in which the “unsubstituted aryl group” has the substituent, and a substituted aryl group described below. It should be noted that examples of the “unsubstituted aryl group” and examples of the “substituted aryl group” listed herein are only one example, and the “substituted aryl group” described herein also includes a group in which a group in which “unsubstituted aryl group” has a substituent further has a substituent, and a group in which “substituted aryl group” further has a substituent, and the like.

An unsubstituted aryl group:
a phenyl group,
a p-biphenyl group,
a m-biphenyl group,
an o-biphenyl group,
a p-terphenyl-4-yl group,
a p-terphenyl-3-yl group,
a p-terphenyl-2-yl group,
a m-terphenyl-4-yl group,
a m-terphenyl-3-yl group,
a m-terphenyl-2-yl group,
an o-terphenyl-4-yl group,
an o-terphenyl-3-yl group,
an o-terphenyl-2-yl group,
a 1-naphthyl group,
a 2-naphthyl group,
an anthryl group,
a benzanthryl group,
a phenanthryl group,
a benzophenanthryl group,
a phenalenyl group,
a pyrenyl group,
a chrysenyl group,
a benzochrysenyl group,
a triphenylenyl group,
a benzotriphenylenyl group,
a tetracenyl group,
a pentacenyl group,
a fluorenyl group,
a 9,9′-spirobifluorenyl group,
a benzofluorenyl group,
a dibenzofluorenyl group,
a fluoranthenyl group,
a benzofluoranthenyl group, and
a perylenyl group.

A substituted aryl group:
an o-tolyl group,
a m-tolyl group,
a p-tolyl group,
a p-xylyl group,
a m-xylyl group,
an o-xylyl group,
a p-isopropyl phenyl group,
a m-isopropyl phenyl group,
an o-isopropyl phenyl group,
a p-t-butylphenyl group,
a m-t-butylphenyl group,
an o-t-butylphenyl group,
a 3,4,5-trimethylphenyl group,
a 9,9-dimethylfluorenyl group,
a 9,9-diphenylfluorenyl group
a 9,9-di(4-methylphenyl)fluorenyl group,
a 9,9-di(4-isopropylphenyl)fluorenyl group,
a 9,9-di(4-t-butylphenyl)fluorenyl group,
a cyanophenyl group,
a triphenylsilylphenyl group,
a trimethylsilylphenyl group,
a phenylnaphthyl group, and
a naphthylphenyl group.

The “heterocyclic group” described herein is a ring group including at least one hetero atom in the ring atom. Specific examples of the hetero atom include a nitrogen atom, an oxygen atom, a sulfur atom, a silicon atom, a phosphorus atom and a boron atom.

The “heterocyclic group” described herein may be a monocyclic group, or a fused ring group.

The “heterocyclic group” described herein may be an aromatic heterocyclic group, or an aliphatic heterocyclic group.

Specific examples (specific example group G2) of the “substituted or unsubstituted heterocyclic group” include an unsubstituted heterocyclic group and a substituted heterocyclic group described below. (Here, the unsubstituted heterocyclic group refers to a case where the “substituted or unsubstituted heterocyclic group” is the “unsubstituted heterocyclic group,” and the substituted heterocyclic group refers to a case where the “substituted or unsubstituted heterocyclic group” is the “substituted heterocyclic group”. Hereinafter, the case of merely “heterocyclic group” includes both the “unsubstituted heterocyclic group” and the “substituted heterocyclic group”.

The “substituted heterocyclic group” refers to a case where the “unsubstituted heterocyclic group” has a substituent, and specific examples thereof include a group in which the “unsubstituted heterocyclic group” has a substituent, and a substituted heterocyclic group described below. It should be noted that examples of the “unsubstituted heterocyclic group” and examples of the “substituted heterocyclic group” listed herein are merely one example, and the “substituted heterocyclic group” described herein also includes a group in which “unsubstituted heterocyclic group” which has a substituent further has a substituent, and a group in which “substituted heterocyclic group” further has a substituent, and the like.

An unsubstituted heterocyclic group including a nitrogen atom:
a pyrrolyl group,
an imidazolyl group,
a pyrazolyl group,
a triazolyl group,
a tetrazolyl group,
an oxazolyl group,
an isoxazolyl group,
an oxadiazolyl group,
a thiazolyl group,
an isothiazolyl group,
a thiadiazolyl group,
a pyridyl group,
a pyridazinyl group,
a pyrimidinyl group,
a pyrazinyl group,
a triazinyl group,
an indolyl group,
an isoindolyl group,
an indolizinyl group,
a quinolizinyl group,
a quinolyl group,
an isoquinolyl group,
a cinnolyl group,
a phthalazinyl group,
a quinazolinyl group,
a quinoxalinyl group,
a benzimidazolyl group,
an indazolyl group,
a phenanthrolinyl group,
a phenanthridinyl group
an acridinyl group,
a phenazinyl group,
a carbazolyl group,
a benzocarbazolyl group,
a morpholino group,
a phenoxazinyl group,
a phenothiazinyl group,
an azacarbazolyl group, and
a diazacarbazolyl group.

An unsubstituted heterocyclic group including an oxygen atom:
a furyl group,
an oxazolyl group,
an isoxazolyl group,
an oxadiazolyl group,
a xanthenyl group,
a benzofuranyl group,
an isobenzofuranyl group,
a dibenzofuranyl group,
a naphthobenzofuranyl group,
a benzoxazolyl group,
a benzisoxazolyl group,
a phenoxazinyl group,
a morpholino group,
a dinaphthofuranyl group,
an azadibenzofuranyl group,
a diazadibenzofuranyl group,
an azanaphthobenzofuranyl group, and
a diazanaphthobenzofuranyl group.

An unsubstituted heterocyclic group including a sulfur atom:
a thienyl group,
a thiazolyl group,
an isothiazolyl group,
a thiadiazolyl group,
a benzothiophenyl group,
an isobenzothiophenyl group,
a dibenzothiophenyl group,
a naphthobenzothiophenyl group,
a benzothiazolyl group,
a benzisothiazolyl group,
a phenothiazinyl group,
a dinaphthothiophenyl group,
an azadibenzothiophenyl group,
a diazadibenzothiophenyl group,
an azanaphthobenzothiophenyl group, and
a diazanaphthobenzothiophenyl group.

A substituted heterocyclic group including a nitrogen atom:
a (9-phenyl)carbazolyl group,
a (9-biphenylyl)carbazolyl group,
a (9-phenyl)phenylcarbazolyl group,
a (9-naphthyl)carbazolyl group,
a diphenylcarbazol-9-yl group,
a phenylcarbazol-9-yl group,
a methylbenzimidazolyl group,
an ethylbenzimidazolyl group,
a phenyltriazinyl group,
a biphenylyltriazinyl group,
a diphenyltriazinyl group,
a phenylquinazolinyl group, and
a biphenylylquinazolinyl group.

A substituted heterocyclic group including an oxygen atom:
a phenyldibenzofuranyl group,
a methyldibenzofuranyl group,
a t-butyldibenzofuranyl group, and
a monovalent residue of spiro[9H-xanthene-9,9′-[9H]fluorene].

A substituted heterocyclic group including a sulfur atom:
a phenyldibenzothiophenyl group,
a methyldibenzothiophenyl group,
a t-butyldibenzothiophenyl group, and
a monovalent residue of spiro[9H-thioxantene-9,9′-[9H]fluorene].

A monovalent group formed from the following unsubstituted heterocyclic ring containing at least one of a nitrogen atom, an oxygen atom and a sulfur atom, and a monovalent group in which a monovalent group formed from the following unsubstituted heterocyclic ring has a substituent:

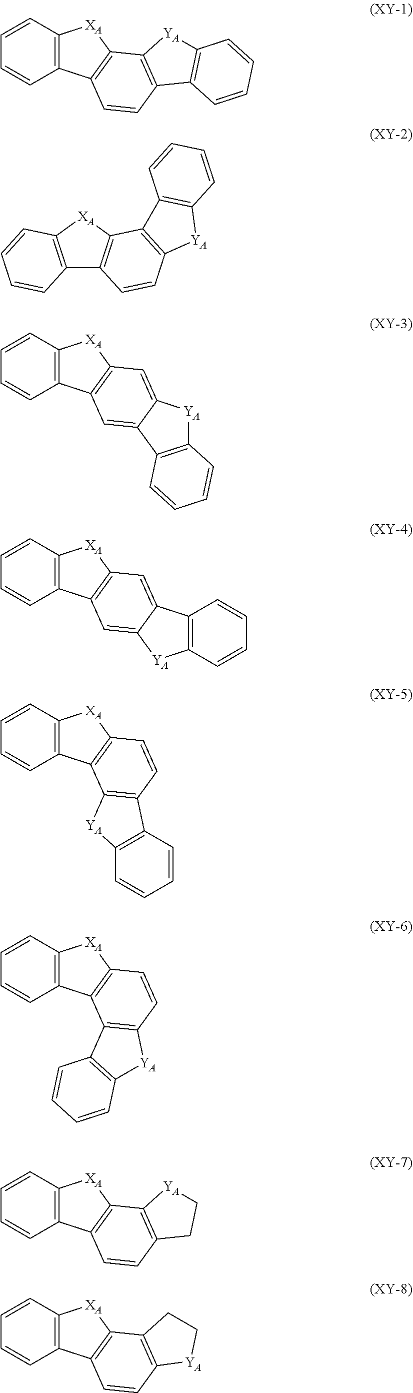

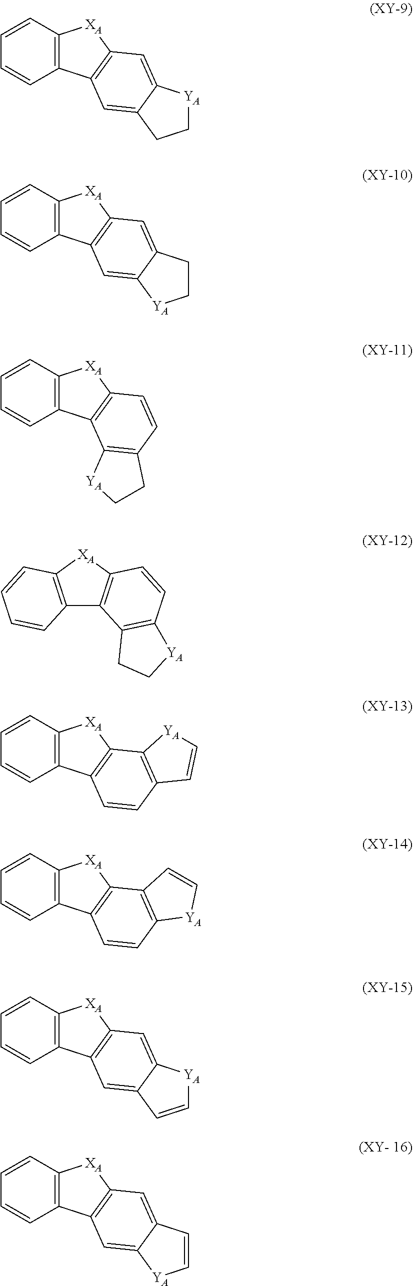

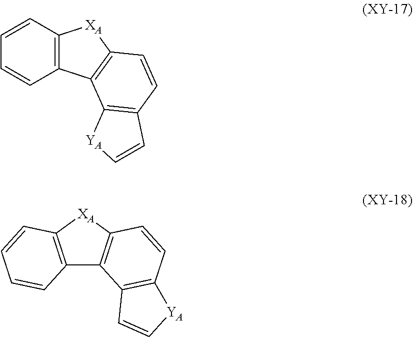

In the formulas (XY-1) to (XY-18), X_{A} and Y_{A} are independently an oxygen atom, a sulfur atom, NH or CH₂. However, at least one of X_{A} and Y_{A} is an oxygen atom, a sulfur atom or NH.

The heterocyclic ring represented by the formulas (XY-1) to (XY-18) becomes a monovalent heterocyclic group including a bond at an arbitrary position.

An expression “the monovalent group formed from the unsubstituted heterocyclic ring represented by the formulas (XY-1) to (XY-18) has a substituent” refers to a case where the hydrogen atom bonded with the carbon atom of a skeleton of the formulas is substituted by a substituent, or a state in which X_{A} or Y_{A} is NH or CH₂, and the hydrogen atom in the NH or CH₂ is replaced with a substituent.

Specific examples (specific example group G3) of the “substituted or unsubstituted alkyl group” include an unsubstituted alkyl group and a substituted alkyl group described below. (Here, the unsubstituted alkyl group refers to a case where the “substituted or unsubstituted alkyl group” is the “unsubstituted alkyl group,” and the substituted alkyl group refers to a case where the “substituted or unsubstituted alkyl group” is the “substituted alkyl group”). Hereinafter, the case of merely “alkyl group” includes both the “unsubstituted alkyl group” and the “substituted alkyl group”.

The “substituted alkyl group” refers to a case where the “unsubstituted alkyl group” has a substituent, and specific examples thereof include a group in which the “unsubstituted alkyl group” has a substituent, and a substituted alkyl group described below. It should be noted that examples of the “unsubstituted alkyl group” and examples of the “substituted alkyl group” listed herein are merely one example, and the “substituted alkyl group” described herein also includes a group in which “unsubstituted alkyl group” has a substituent further has a substituent, a group in which “substituted alkyl group” further has a substituent, and the like.

An unsubstituted alkyl group:
a methyl group,
an ethyl group,
a n-propyl group,
an isopropyl group,
a n-butyl group,
an isobutyl group,
a s-butyl group, and
a t-butyl group.

A substituted alkyl group:
a heptafluoropropyl group (including an isomer),
a pentafluoroethyl group,
a 2,2,2-trifluoroethyl group, and
a trifluoromethyl group.

Specific examples (specific example group G4) of the “substituted or unsubstituted alkenyl group” include an unsubstituted alkenyl group and a substituted alkenyl group described below (Here, the unsubstituted alkenyl group refers to a case where the “substituted or unsubstituted alkenyl group” is the “unsubstituted alkenyl group,” and the substituted alkenyl group refers to a case where the “substituted or unsubstituted alkenyl group” is the “substituted alkenyl group”). Hereinafter, the case of merely “alkenyl group” includes both the “unsubstituted alkenyl group” and the “substituted alkenyl group”.

The “substituted alkenyl group” refers to a case where the “unsubstituted alkenyl group” has a substituent, and specific examples thereof include a group in which the “unsubstituted alkenyl group” has a substituent, and a substituted alkenyl group described below. It should be noted that examples of the “unsubstituted alkenyl group” and examples of the “substituted alkenyl group” listed herein are merely one example, and the “substituted alkenyl group” described herein also includes a group in which “unsubstituted alkenyl group” has a substituent further has a substituent, a group in which “substituted alkenyl group” further has a substituent, and the like.

An unsubstituted alkenyl group and a substituted alkenyl group:
a vinyl group,
an allyl group,
a 1-butenyl group,
a 2-butenyl group,
a 3-butenyl group,
a 1,3-butanedienyl group,
a 1-methylvinyl group,
a 1-methylallyl group,
a 1,1-dimethylallyl group,
a 2-methylallyl group, and
a 1,2-dimethylallyl group.

Specific examples (specific example group G5) of the “substituted or unsubstituted alkynyl group” include an unsubstituted alkynyl group described below. (Here, the unsubstituted alkynyl group refers to a case where the “substituted or unsubstituted alkynyl group” is the “unsubstituted alkynyl group”). Hereinafter, a case of merely “alkynyl group” includes both the “unsubstituted alkynyl group” and the “substituted alkynyl group”.

The “substituted alkynyl group” refers to a case where the “unsubstituted alkynyl group” has a substituent, and specific examples thereof include a group in which the “unsubstituted alkynyl group” described below has a substituent.

An unsubstituted alkynyl group:
an ethynyl group.

Specific examples (specific example group G6) of the “substituted or unsubstituted cycloalkyl group” described herein include an unsubstituted cycloalkyl group and a substituted cycloalkyl group described below. (Here, the unsubstituted cycloalkyl group refers to a case where the “substituted or unsubstituted cycloalkyl group” is the “unsubstituted cycloalkyl group,” and the substituted cycloalkyl group refers to a case where the “substituted or unsubstituted cycloalkyl group” is the “substituted cycloalkyl group”). Hereinafter, a case of merely “cycloalkyl group” includes both the “unsubstituted cycloalkyl group” and the “substituted cycloalkyl group”.

The “substituted cycloalkyl group” refers to a case where the “unsubstituted cycloalkyl group” a the substituent, and specific examples thereof include a group in which the “unsubstituted cycloalkyl group” has a substituent, and a substituted cycloalkyl group described below. It should be noted that examples of the “unsubstituted cycloalkyl group” and examples of the “substituted cycloalkyl group” listed herein are merely one example, and the “substituted cycloalkyl group” described herein also includes a group in which “unsubstituted cycloalkyl group” has a substituent further has a substituent, a group in which “substituted cycloalkyl group” further has a substituent, and the like.

An unsubstituted aliphatic ring group:
a cyclopropyl group,
a cyclobutyl group,
a cyclopentyl group,
a cyclohexyl group,
a 1-adamantyl group,
a 2-adamantyl group,
a 1-norbomyl group, and
a 2-norbomyl group.

A substituted cycloalkyl group:
a 4-methylcyclohexyl group.

Specific examples (specific example group G7) of the group represented by —Si(R₉₀₁)(R₉₀₂)(R₉₀₃) described herein include
Si(G1)(G1)(G1),
Si(G1)(G2)(G2),
Si(G1)(G1)(G2),
Si(G2)(G2)(G2),
Si(G3)(G3)(G3),
Si(G5)(G5)(G5) and
Si(G6)(G6)(G6).

In which,

G1 is the “aryl group” described in the specific example group G1.

G2 is the “heterocyclic group” described in the specific example group G2.

G3 is the “alkyl group” described in the specific example group G3.

G5 is the “alkynyl group” described in the specific example group G5.

G6 is the “cycloalkyl group” described in the specific example group G6.

Specific examples (specific example group G8) of the group represented by —O—(R₉₀₄) described herein include
O(G1),
O(G2),
O(G3) and
O(G6).

In which,

G1 is the “aryl group” described in the specific example group G1.

G2 is the “heterocyclic group” described in the specific example group G2.

G3 is the “alkyl group” described in the specific example group G3.

G6 is the “cycloalkyl group” described in the specific example group G6.

Specific examples (specific example group G9) of the group represented by —S—(R₉₀₅) described herein include
—S(G1),
—S(G2),
—S(G3) and
—S(G6).

In which,

G1 is the “aryl group” described in the specific example group G1.

G2 is the “heterocycle group” described in the specific example group G2.

G3 is the “alkyl group” described in the specific example group G3.

G6 is the “cycloalkyl group” described in the specific example group G6.

Specific examples (specific example group G10) of the group represented by —N(R₉₀₆)(R₉₀₇) described herein include
—N(G1)(G1),
—N(G2)(G2),
—N(G1)(G2),
—N(G3)(G3) and
—N(G6) (G6).

In which,

G1 is the “aryl group” described in the specific example group G1.

G2 is the “heterocycle group” described in the specific example group G2.

G3 is the “alkyl group” described in the specific example group G3.

G6 is the “cycloalkyl group” described in the specific example group G6.

Specific examples (specific example group G11) of the “halogen atom” described herein include a fluorine atom, a chlorine atom, a bromine atom and an iodine atom.

Specific examples of the “alkoxy group” described herein include a group represented by —O(G3), where G3 is the “alkyl group” described in the specific example group G3. The number of carbon atoms of the “unsubstituted alkoxy group” are 1 to 50, preferably 1 to 30, and more preferably 1 to 18, unless otherwise specified.

Specific examples of the “alkylthio group” described herein include a group represented by —S(G3), where G3 is the “alkyl group” described in the specific example group G3. The number of carbon atoms of the “unsubstituted alkylthio group” are 1 to 50, preferably 1 to 30, and more preferably 1 to 18, unless otherwise specified.

Specific examples of the “aryloxy group” described herein include a group represented by —O(G1), where G1 is the “aryl group” described in the specific example group G1. The number of ring carbon atoms of the “unsubstituted aryloxy group” are 6 to 50, preferably 6 to 30, and more preferably 6 to 18, unless otherwise specified.

Specific examples of the “arylthio group” described herein include a group represented by —S(G1), where G1 is the “aryl group” described in the specific example group G1. The number of ring carbon atoms of the “unsubstituted arylthio group” are 6 to 50, preferably 6 to 30, and more preferably 6 to 18, unless otherwise specified.

Specific examples of the “aralkyl group” described herein include a group represented by -(G3)-(G1), where G3 is the “alkyl group” described in the specific example group G3, and G1 is the “aryl group” described in the specific example group G1. Accordingly, the “aralkyl group” is an aspect of the “substituted alkyl group” substituted by the “aryl group”. The number of carbon atoms of the “unsubstituted aralkyl group,” which is the “unsubstituted alkyl group” substituted by the “unsubstituted aryl group,” are 7 to 50, preferably 7 to 30, and more preferably 7 to 18, unless otherwise specified. Specific example of the “aralkyl group” include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-β-naphthylethyl group, a 1-β-naphthylisopropyl group, and a 2-β-naphthylisopropyl group.

The substituted or unsubstituted aryl group described herein is, unless otherwise specified, preferably a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, a 9,9-diphenylfluorenyl group, or the like.

The substituted or unsubstituted heterocyclic group described herein is, unless otherwise specified, preferably a pyridyl group, a pyrimidinyl group, a triazinyl group, a quinolyl group, an isoquinolyl group, a quinazolinyl group, a benzimidazolyl group, a phenanthrolinyl group, a carbazolyl group, a benzocarbazolyl group, an azacarbazolyl group, a diazacarbazolyl group, a dibenzofuranyl group, a naphthobenzofuranyl group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenyl)carbazolyl group, a (9-biphenylyl)carbazolyl group, a (9-phenyl)phenylcarbazolyl group, a diphenylcarbazole-9-yl group, a phenylcarbazol-9-yl group, a phenyltiazinyl group, a biphenylyltriazinyl group, diphenyltriazinyl group, a phenyldibenzofuranyl group, a phenyldibenzothiophenyl group, or the like.

The dibenzofuranyl group and the dibenzothiophenyl group as described above are specifically any group described below, unless otherwise specified.

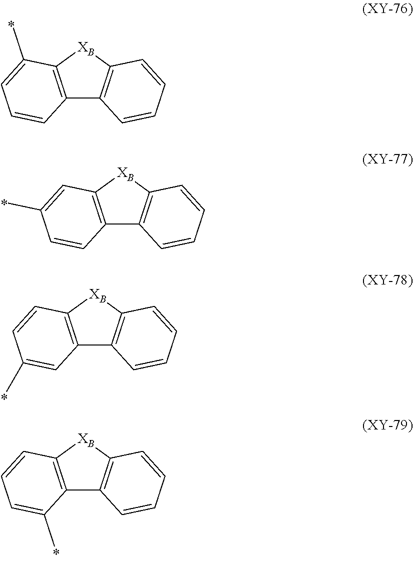

In the formulas (XY-76) to (XY-79), X_{B} is an oxygen atom or a sulfur atom.

The substituted or unsubstituted alkyl group described herein is, unless otherwise specified, preferably a methyl group, an ethyl group, a propyl group, an isopropyl group, a n-butyl group, an isobutyl group, a t-butyl group, or the like.

The “substituted or unsubstituted arylene group” descried herein refers to a group in which the above-described “aryl group” is converted into divalence, unless otherwise specified. Specific examples (specific example group G12) of the “substituted or unsubstituted arylene group” include a group in which the “aryl group” described in the specific example group G1 is converted into divalence.

Specific examples (specific example group G13) of the “substituted or unsubstituted divalent heterocyclic group” include a group in which the “heterocyclic group” described in the specific example group G2 is converted into divalence.

Specific examples (specific example group G14) of the “substituted or unsubstituted alkylene group” include a group in which the “alkyl group” described in the specific example group G3 is converted into divalence.

The substituted or unsubstituted arylene group described herein is any group described below, unless otherwise specified.

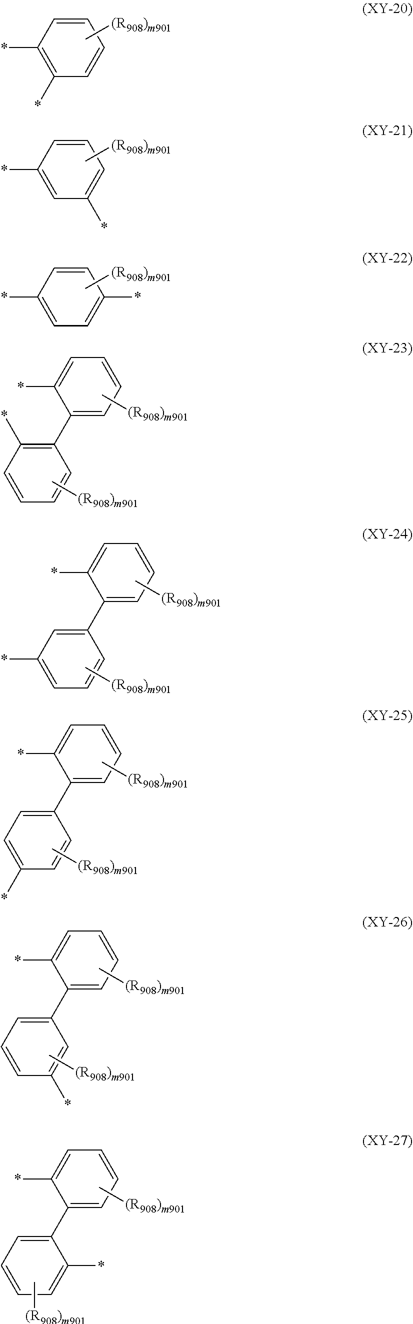

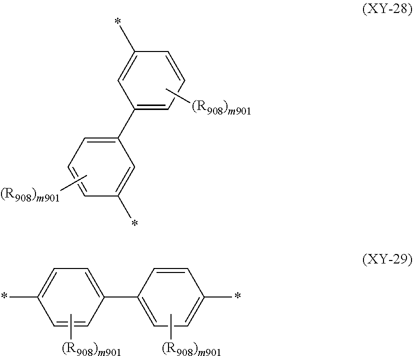

In the formulas (XY-20) to (XY-29), R₉₀₈ is a substituent.

Then, m901 is an integer of 0 to 4, and when m901 is 2 or more, a plurality of R₉₀₈ may be the same with or different from each other.

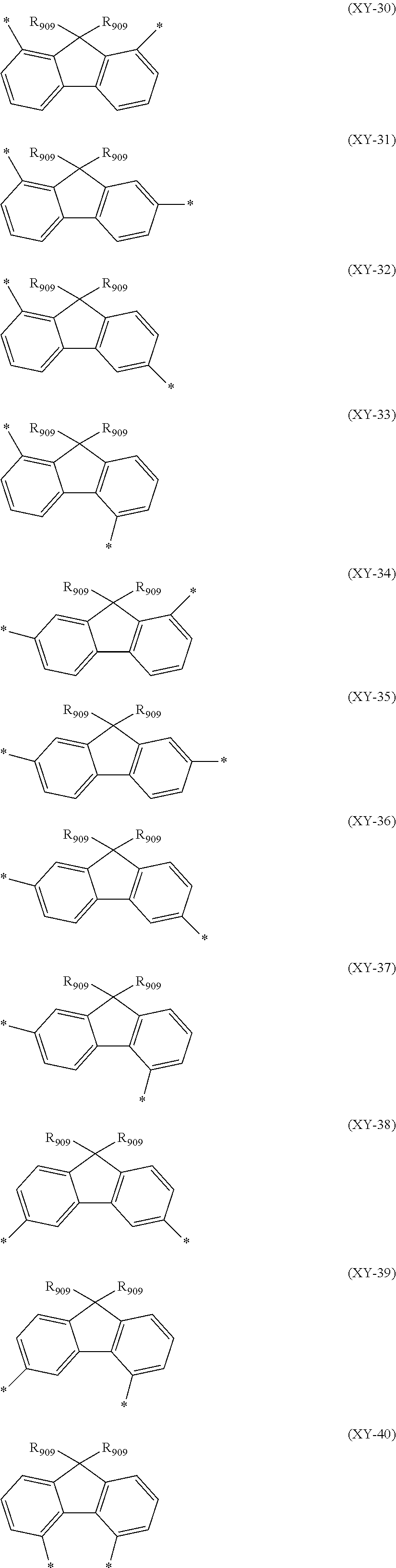

In the formulas (XY-30) to (XY-40), R₉₀₉ is independently a hydrogen atom or a substituent. Two of R₉₀₉ form a ring by bonding with each other through a single bond, or do not form a ring.

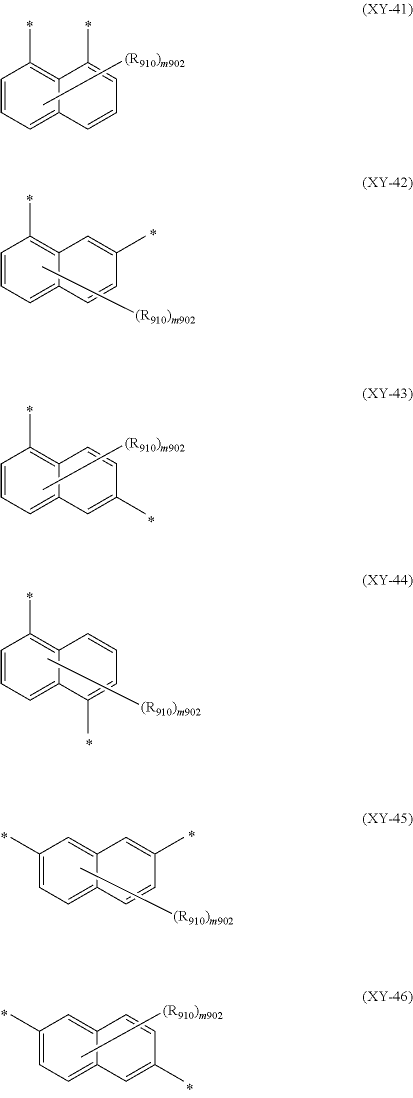

In the formulas (XY-41) to (XY-46), R₉₁₀ is a substituent.

Then, m902 is an integer of 0 to 6. When m902 is 2 or more, a plurality of R₉₁₀ may be the same with or different from each other.

The substituted or unsubstituted divalent heterocyclic group described herein is preferably any group described below, unless otherwise specified.

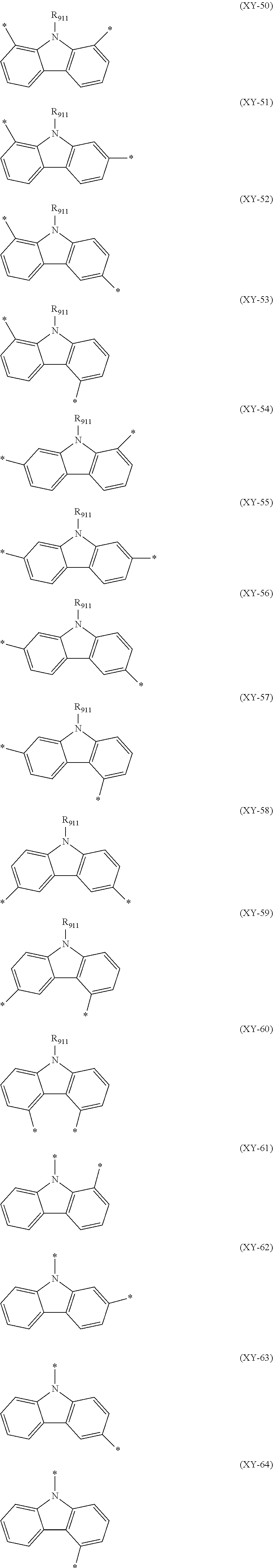

In the formulas (XY-50) to (XY-60), R₉₁₁ is a hydrogen atom or a substituent

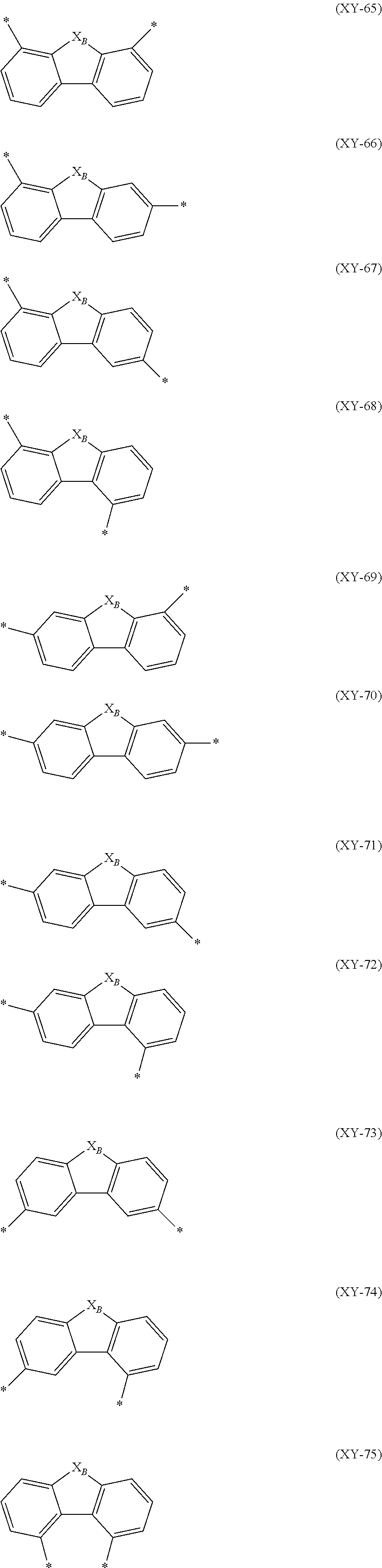

In the formulas (XY-65) to (XY-75), X_{B} is an oxygen atom or a sulfur atom.

Herein, a case where “one or more sets of two or more groups adjacent to each other form a substituted or unsubstituted and saturated or unsaturated ring by bonding with each other” will be described by taking, as an example, a case of an anthracene compound represented by the following formula (XY-80) in which a mother skeleton is an anthracene ring.

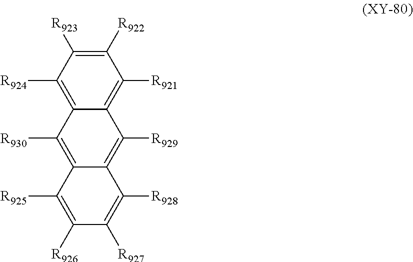

For example, two adjacent to each other into one set when “one or more sets of two or more groups adjacent to each other form the ring by bonding with each other” among R₉₂₁ to R₉₃₀ include R₉₂₁ and R₉₂₂, R₉₂₂ and R₉₂₃, R₉₂₃ and R₉₂₄, R₉₂₄ and R₉₃₀, R₉₃₀ and R₉₂₅, R₉₂₅ and R₉₂₆, R₉₂₆ and R₉₂₇, R₉₂₇ and R₉₂₈, R₉₂₈ and R₉₂₉, and R₉₂₉ and R₉₂₁.

The above-described “one or more sets” means that two or more sets of two groups adjacent to each other may simultaneously form the ring. For example, a case where R₉₂₁ and R₉₂₂ forma ring A by bonding with each other, and simultaneously R₉₂₅ and R₉₂₆ form a ring B by bonding with each other is represented by the following formula (XY-81).

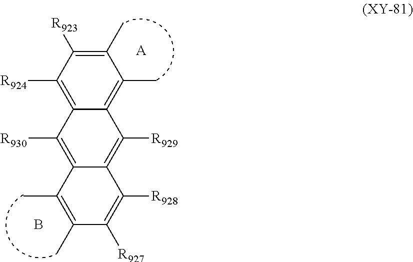

A case where “two or more groups adjacent to each other” form a ring means that, for example, R₉₂₁ and R₉₂₂ form a ring A by bonding with each other, and R₉₂₂ and R₉₂₃ form a ring C by bonding with each other. A case where the ring A and ring C sharing R₉₂₂ are formed, in which the ring A and the ring Care fused to the anthracene mother skeleton by three of R₉₂₁ to R₉₂₃ adjacent to each other, is represented by the following (XY-82).

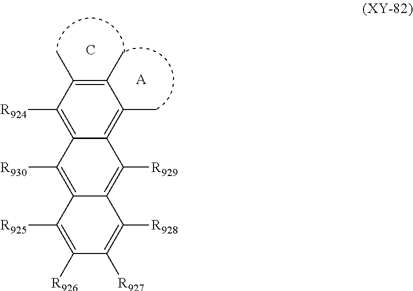

The rings A to C formed in the formulas (XY-81) and (XY-82) are a saturated or unsaturated ring.

A term “unsaturated ring” means an aromatic hydrocarbon ring or an aromatic heterocyclic ring. A term “saturated ring” means an aliphatic hydrocarbon ring or an aliphatic heterocyclic ring.

For example, the ring A formed by R₉₂₁ and R₉₂₂ being bonded with each other, represented by the formula (XY-81), means a ring formed by a carbon atom of the anthracene skeleton bonded with R₉₂₁, a carbon atom of the anthracene skeleton bonded with R₉₂₂, and one or more arbitrary elements. Specific examples include, when the ring A is formed by R₉₂₁ and R₉₂₂, a case where an unsaturated ring is formed of a carbon atom of an anthracene skeleton bonded with R₉₂₁, a carbon atom of the anthracene skeleton bonded with R₉₂₂, and four carbon atoms, in which a ring formed by R₉₂₁ and R₉₂₂ is formed into a benzene ring. Further, when a saturated ring is formed, the ring is formed into a cyclohexane ring.

Here, “arbitrary elements” are preferably a C element, a N element, an O element and a S element. In the arbitrary elements (for example, a case of the C element or the N element), the carbon atoms constituting the anthracene skeleton which do not form the ring may be terminated by a hydrogen atom, or may be substituted by an arbitrary substituent. When the ring contains the arbitrary elements other than the C element, the ring to be formed is a heterocyclic ring.

The number of “one or more arbitrary elements” forming the saturated or unsaturated ring is preferably 2 or more and 15 or less, more preferably 3 or more and 12 or less, and further preferably 3 or more and 5 or less.

When the above-described “saturated or unsaturated ring” has a substituent, the substituent is as described above.

In one embodiment of the specification, the substituent (hereinafter, referred to as an “arbitrary substituent” in several cases) in the case of the “substituted or unsubstituted” is a group selected from the group consisting of

an unsubstituted alkyl group including 1 to 50 carbon atoms,
an unsubstituted alkenyl group including 2 to 50 carbon atoms,
an unsubstituted alkynyl group including 2 to 50 carbon atoms,
an unsubstituted cycloalkyl group including 3 to 50 ring carbon atoms,
—Si(R₉₀₁)(R₉₀₂)(R₉₀₃),

### —O—(R₉₀₄),

### —S—(R₉₀₅)

—N(R₉₀₆)(R₉₀₇)
wherein,
R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and when two or
more of R₉₀₁ to R₉₀₇ exist, two or more of R₉₀₁ to R₉₀₇ may be the same with or different from each other,
a halogen atom, a cyano group, a nitro group,
an unsubstituted aryl group including 6 to 50 ring carbon atoms, and
an unsubstituted monovalent heterocyclic group including 5 to 50 ring atoms.

In one embodiment, the substituent in the case of “substituted or unsubstituted” is a group selected from the group consisting of

an alkyl group including 1 to 50 carbon atoms,
an aryl group including 6 to 50 ring carbon atoms, and
a monovalent heterocyclic group including 5 to 50 ring atoms.

In one embodiment, the substituent in the case of “substituted or unsubstituted” is a group selected from the group consisting of

an alkyl group including 1 to 18 carbon atoms,
an aryl group including 6 to 18 ring carbon atoms, and
a monovalent heterocyclic group including 5 to 18 ring atoms.

Specific examples of each group of the arbitrary substituent described above are as described above.

Herein, unless otherwise specified, the saturated or unsaturated ring (preferably substituted or unsubstituted and saturated or unsaturated five-membered or six-membered ring, more preferably a benzene ring) may be formed by the arbitrary substituents adjacent to each other.

Herein, unless otherwise specified, the arbitrary substituent may further have the substituent. Specific examples of the substituent that the arbitrary substituent further has include to the ones same as the arbitrary substituent described above.

### [Organic Electroluminescence Device 1]

An organic electroluminescence device 1 according to the first aspect of the invention is an organic electroluminescence device including: a cathode; an anode; and an organic layer between the cathode and the anode,

wherein the organic layer contains a compound represented by the following formula (1) and a compound represented by the following formula (11).

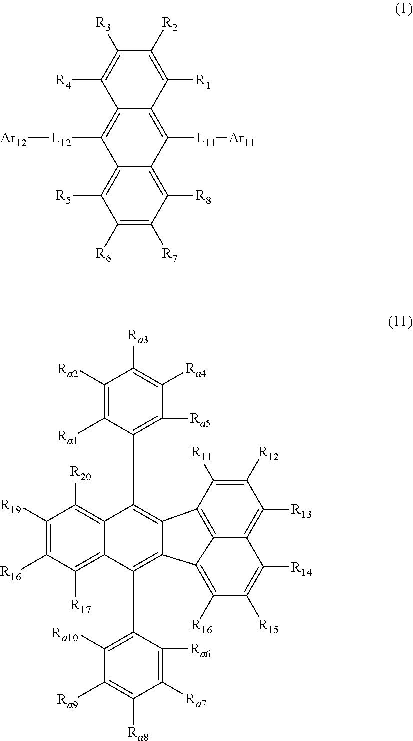

Each substituent in the formulas (1) and (2) will be described later.

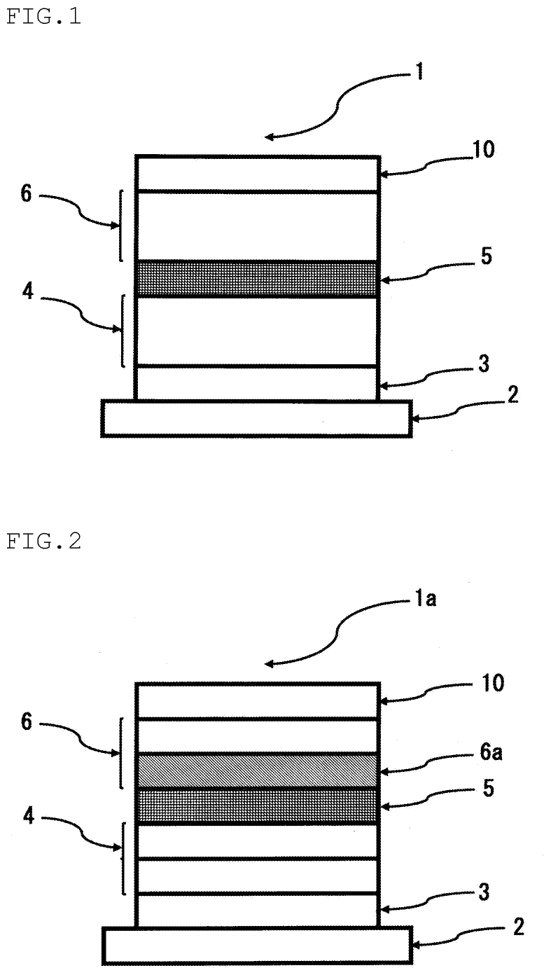

Schematic configuration of the organic EL device of the first aspect will be explained referring to FIG. 1.

The organic EL device 1 of the first aspect includes a substrate 2, an anode 3, an emitting layer 5, a cathode 10, an organic layer 4 between the anode 3 and the emitting layer 5, and an organic layer 6 between the emitting layer 5 and the cathode 10.

The compound represented by the formula (1) and the compound represented by the formula (11) are contained in the organic layers 4 to 6 between the anode 3 and the cathode 10, and are preferably contained in the emitting layer 5.

The compound represented by the formula (1) and the compound represented by the formula (11) contained in the organic layer may each be alone or in combination of two or more.

In the formula (1), one or more among R₁ to R₈ are -L₁-Ar₁. That is, the anthracene compound of the formula (1) has a structure in which three or more groups of -L₁-Ar₁ are substituted (hereinafter, the compound represented by the formula (1) may be referred to as a “tri-substituted anthracene compound (1)” or a “tri-substituted anthracene-based host material (1)”.)

A conventional anthracene-based host material having two substituents corresponding to the group of -L₁-Ar₁ is known (hereinafter, sometimes referred to as a “di-substituted anthracene compound”.)

The inventors have found that when the tri-substituted anthracene compound (1) is used as a host material of an emitting layer and the fluoranthene compound represented by the formula (11) (hereinafter, referred to as a “fluoranthene-based compound (11)” or a “fluoranthene-based dopant material (11)”) is used as a dopant material, the device lifetime is improved.

The organic EL device of the first aspect includes: a cathode; an anode; an organic layer between the cathode and the anode; wherein the organic layer, preferably an emitting layer, contains a compound represented by the formula (1) (hereinafter, sometimes referred to as a “tri-substituted anthracene compound (1)” or a “tri-substituted anthracene host material (1)); and a compound represented by the formula (11), whereby the device lifetime can be improved. In addition, an organic EL device having along device lifetime can be obtained. The inferred reason will be explained below.

Compared with a di-substituted anthracene compound, a tri-substituted anthracene compound (1) has a higher electron mobility and the peripheral materials deteriorate due to excess electrons, so that a sufficient device lifetime cannot be obtained. On the other hand, a compound represented by the formula (11) has strong electron-trapping property, and by combining with a ti-substituted anthracene compound (1), it is considered that the electron mobility can be suppressed and the device lifetime can be improved.

Further, the inventors have also studied a combination with a material constituting a layer directly in contact with the emitting layer containing the tri-substituted anthracene-based host material (1) and the fluoranthene-based dopant material (11). The inventors have found that when a compound represented by the formula (21) (hereinafter, sometimes referred to as an azine-based hole-blocking layer material (21)) or a compound represented by the formula (31) (hereinafter, sometimes referred to as a fluoranthene-based hole-blocking layer material (31)), which will be described later, is used for an hole-blocking layer directly in contact with the emitting layer, a more excellent effect of improving the device lifetime can be obtained.

It has also been found that an organic EL device having a longer lifetime can be obtained by using a compound represented by the formula (41) (hereinafter, sometimes referred to as a “monoamine-based electron-blocking layer material (41)”) in an electron-blocking layer directly in contact with the emitting layer.

The organic EL device of the second aspect of the invention is one embodiment of the organic EL device according to the first aspect, in which a compound represented by the formula (1) and a compound represented by the formula (11) are contained in an emitting layer, the organic layer further includes a hole-blocking layer directly in contact with the emitting layer, the hole-blocking layer contains either or both of a compound represented by the following formula (21) and a compound represented by the following formula (31).

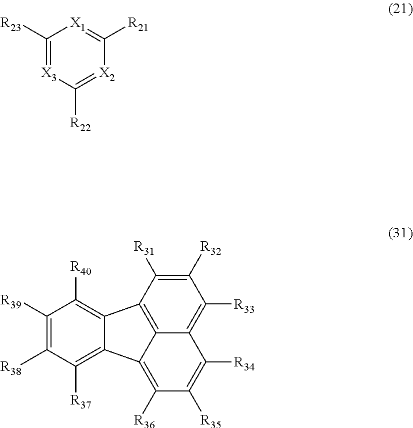

Each substituent in the formulas (21) and (31) will be described later.

Schematic configuration of the organic EL device of the second aspect will be explained referring to FIG. 2.

The organic EL device 1a of the second aspect includes a substrate 2, an anode 3, an emitting layer 5, a cathode 10, an organic layer 4 between the anode 3 and the emitting layer 5, and an organic layer 6 between the emitting layer 5 and the cathode 10, and the organic layers 6 between the emitting layer 5 and the cathode 10 includes a hole-blocking layer 6a directly in contact with the emitting layer 5.

The compound represented by the formula (21) and the compound represented by the formula (31) contained in the hole-blocking layer may each be alone or in combination of two or more.

The organic EL device of the third aspect of the invention is one embodiment of the organic EL device according to the first aspect, in which a compound represented by the formula (1) and a compound represented by the formula (11) are contained in an emitting layer,

the organic layer further includes an electron-blocking layer directly in contact with an emitting layer,

the electron-blocking layer contains either or both of a compound represented by the following formula (41) and a compound represented by the following formula (51).

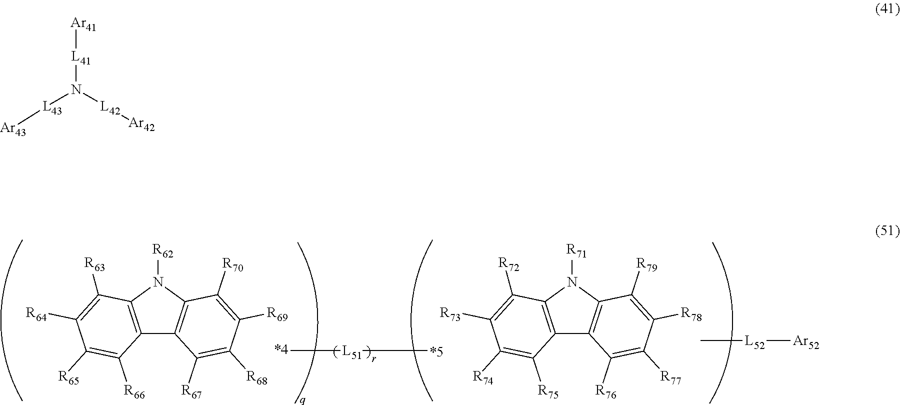

Each substituent in the formulas (41) and (51) will be described later.

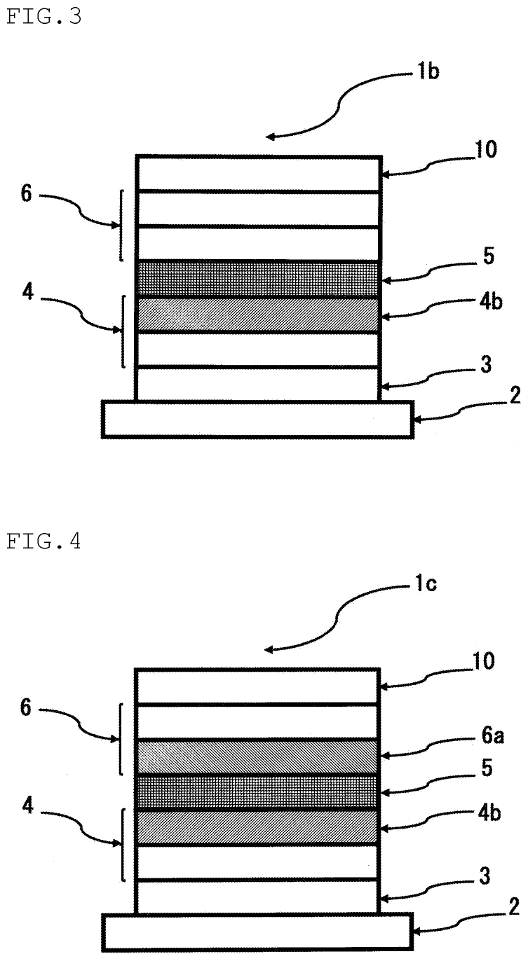

Schematic configuration of the organic EL device of the third aspect will be explained refening to FIG. 3.

The organic EL device 1b of the third aspect includes a substrate 2, an anode 3, an emitting layer 5, a cathode 10, an organic layer 4 between the anode 3 and the emitting layer 5, and an organic layer 6 between the emitting layer 5 and the cathode 10, and the organic layers 4 between the anode 3 and the emitting layer 5 includes an electron-blocking layer 4b directly in contact with the emitting layer 5.

The compound represented by the formula (41) and the compound represented by the formula (51) contained in the electron-blocking layer may each be alone or in combination of two or more.

The organic EL device of the fourth aspect of the invention is one embodiment of the organic EL device according to the first aspect in which a compound represented by the formula (1) and a compound represented by the formula (11) are contained in an emitting layer,

the organic layer further includes a hole-blocking layer directly in contact with the emitting layer,

the hole-blocking layer contains either or both of a compound represented by the following formula (21) and a compound represented by the following formula (31),

the organic layer further includes an electron-blocking layer directly in contact with an emitting layer,

the electron-blocking layer contains either or both of a compound represented by the formula (41) and a compound represented by the formula (51).

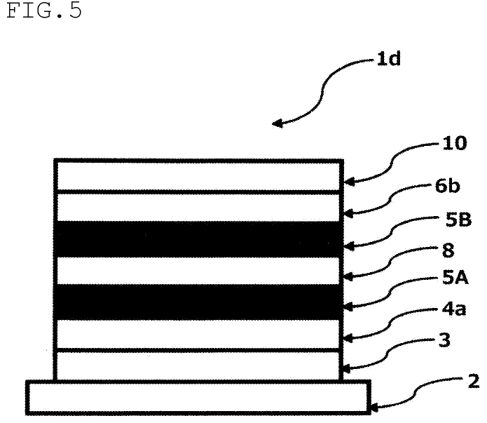

The organic EL device of the fourth aspect of the invention will be explained referring to FIG. 4.

The organic EL device 1c of the fourth aspect is one embodiment of the organic EL devices of the first to third aspects, includes: a substrate 2, an anode 3, an emitting layer 5, a cathode 10, an organic layer 4 between the anode 3 and the emitting layer 5, and an organic layer 6 between the emitting layer 5 and the cathode 10; and the organic layer 6 between the emitting layer 5 and the cathode 10 includes a hole-blocking layer 6a directly in contact with the emitting layer 5, and the organic layer 4 between the anode 3 and the emitting layer 5 includes an electron-blocking layer 4b directly in contact with the emitting layer 5.

The organic layer includes an emitting layer 5, a hole-blocking layer 6a, and an electron-blocking layer 4b, and each layer contains a specific compound, whereby an effect of improving the device lifetime can be obtained.

The organic EL device according to the fifth aspect of the invention has a so-called tandem-type configuration having two or more emitting layers. By having such a tandem-type structure, a white emitting device having a simple structure can be produced.

The organic EL device according to an aspect of the invention may be, for example, a monochromatic emitting device of a fluorescent or phosphorescent type, or a white emitting device of a fluorescent/phosphorescent hybrid type. In addition, it may be a simple type including a single emitting unit or a tandem-type device including a plurality of emitting units.

Here, the term “emitting unit” refers to a minimal unit which includes organic layers, wherein at least one of the organic layers is an emitting layer, and which emits light by recombination of injected holes and electrons.

The “emitting layer” described in the specification is an organic layer having an emitting function. The emitting layer is, for example, a phosphorescent emitting layer, a fluorescent emitting layer, or the like, and may be a single layer or a plurality of layers.

The light-emitting unit may be of a stacked type including a plurality of a phosphorescent emitting layer and a fluorescent emitting layer, and in this case, for example, may include a spacing layer between each emitting layer for preventing excitons generated in the phosphorescent emitting layer from diffusing into the fluorescent emitting layer.

The simple type organic EL device includes, for example, a device configuration such as anode/emitting unit/cathode.

Typical layer configurations of the emitting unit are shown below. The layers in parentheses are optional layers.

(a) (hole-injecting layer/hole-transporting layer/fluorescent emitting layer (/electron-transporting layer/electron-injecting layer)
(b) (hole-injecting layer/hole-transporting layer/phosphorescent emitting layer (/electron-transporting layer/electron-injecting layer)
(c) (hole-injecting layer/hole-transporting layer/first fluorescent emitting layer/second fluorescent emitting layer (/electron-transporting layer/electron-injecting layer)
(d) (hole-injecting layer/hole-transporting layer/first phosphorescence emitting layer/second phosphorescence emitting layer/(electron-transporting layer/electron-injecting layer)
(e) (hole-injecting layer/hole-transporting layer/phosphorescence emitting layer/spacing layer/fluorescence emitting layer (/electron-transporting layer/electron-injecting layer)
(f) (hole-injecting layer/hole-transporting layer/first phosphorescent emitting layer/second phosphorescent emitting layer/spacing layer/fluorescent emitting layer (/electron-transporting layer/electron-injecting layer)
(g) (hole-injecting layer/hole-transporting layer/first phosphorescent layer/spacing layer/second phosphorescent emitting layer/spacing layer/fluorescent emitting layer (/electron-transporting layer/electron-injecting layer)
(h) (hole-injecting layer/hole-transporting layer/phosphorescent emitting layer/spacing layer/first fluorescent emitting layer/second fluorescent emitting layer (/electron-transporting layer/electron-injecting layer)
(i) (hole-injecting layer/hole-transporting layer/electron-blocking layer/fluorescent emitting layer (/electron-transporting layer/electron-injecting layer)
(j) (hole-injecting layer/hole-transporting layer/electron-blocking layer/phosphorescent emitting layer (/electron-transporting layer/electron-injecting layer)
(k) (hole-injecting layer/hole-transporting layer/exciton-blocking layer/fluorescent emitting layer (/electron-transporting layer/electron-injecting layer)
(l) (hole-injecting layer/hole-transporting layer/exciton-blocking layer/phosphorescent emitting layer (/electron-transporting layer/electron-injecting layer)
(m) (hole-injecting layer/first hole-transporting layer/second hole-transporting layer/fluorescent emitting layer (/electron-transporting layer/electron-injecting layer)
(n) (hole-injecting layer/first hole-transporting layer/second hole-transporting layer/fluorescent emitting layer (/first electron-transporting layer/second electron-transporting layer/electron-injecting layer)
(o) (hole-injecting layer/first hole-transporting layer/second hole-transporting layer/phosphorescent emitting layer (/electron-transporting layer/electron-injecting layer)
(p) (hole-injecting layer/first hole-transporting layer/second hole-transporting layer/phosphorescent emitting layer (/first electron-transporting layer/second electron-transporting layer/electron-injecting layer)
(q) (hole-injecting layer/hole-transporting layer/fluorescent emitting layer/hole-blocking layer (/electron-transporting layer/electron-injecting layer)
(r) (hole-injecting layer) hole-transporting layer/phosphorescent emitting layer/hole-blocking layer (/electron-transporting layer/electron-injecting layer)
(s) (hole-injecting layer/hole-transporting layer/fluorescent emitting layer/exciton-blocking layer (/electron-transporting layer/electron-injecting layer)
(t) (hole-injecting layer/hole-transporting layer/phosphorescent emitting layer/exciton-blocking layer (/electron-transporting layer/electron-injecting layer)

However, the layer configuration of the organic EL device according to one aspect of the invention is not limited thereto. For example, when the organic EL device has a hole-injecting layer and a hole-transporting layer, it is preferred that a hole-injecting layer be provided between the hole-transporting layer and the anode. Further, when the organic EL device has an electron-injecting layer and an electron-transporting layer, it is preferred that an electron-injecting layer be provided between the electron-transporting layer and the cathode. Further, each of the hole-injecting layer, the hole-transporting layer, the electron-transporting layer and the electron-injecting layer may be formed of a single layer or be formed of a plurality of layers.

The plurality of phosphorescent emitting layers, and a set of the phosphorescent emitting layer and the fluorescent emitting layer may be emitting layers that emit mutually different colors. For example, the emitting unit (f) may include a hole-transporting layer/first phosphorescent layer (red light emission)/second phosphorescent emitting layer (green light emission)/spacing layer/fluorescent emitting layer (blue light emission)/electron-transporting layer.

An electron-blocking layer may be provided between each light emitting layer and the hole-transporting layer or the spacing layer. Further, a hole-blocking layer may be provided between each emitting layer and the electron-transporting layer. By providing the electron-blocking layer or the hole-blocking layer, it is possible to confine electrons or holes in the emitting layer, thereby to improve the recombination probability of carriers in the emitting layer, and to improve luminous efficiency.

As a representative device configuration of a tandem type organic EL device, for example, a device configuration such as anode/first emitting unit/intermediate layer/second emitting unit/cathode can be given.

The first emitting unit and the second emitting unit are independently selected from the above-mentioned emitting units, for example.

The intermediate layer is also generally referred to as an intermediate electrode, an intermediate conductive layer, a charge-generating layer, an electron withdrawing layer, a connecting layer, a connector layer, or an intermediate insulating layer. The intermediate layer is a layer that supplies electrons to the first emitting unit and holes to the second emitting unit, and can be formed of known materials.

Schematic configuration of one embodiment of an organic EL device according to the fifth aspect of the invention will be explained referring to FIG. 5.

The organic EL device 1d according to the fifth aspect of the invention shown in FIG. 5 includes a substrate 2, an anode 3, a cathode 10, and an organic layer between the anode 3 and the cathode 10. The organic layer includes a first emitting unit 5A, a second emitting unit 5B between the first emitting unit 5A and the cathode 10, an organic layer 4a between the anode 3 and the first emitting unit 5A, and an organic layer 6b between the second emitting unit 5B and the cathode 10. A charge-generating layer 8 is provided between the first emitting unit 5A and the second emitting unit 5B.

### [Organic Electroluminescence Device 2]

The organic electroluminescence device 2 according to another aspect of the invention is a organic electroluminescence device including a cathode, an anode, and an organic layer between the cathode and the anode,

wherein the organic layer contains a compound represented by the formula (1), and

a compound A having a Stokes shift of 20 nm or less and an emission peak wavelength of 440 nm to 465 nm.

Here, “Stokes shift (SS)” is the difference between the maximum wavelength of the absorption spectrum and the maximum wavelength of the fluorescence spectrum, and can be measured by the method described in Examples.

Each substituent in the formula (1), and a compound A will be described later.

The structure of the organic EL devices of the first to fifth aspects is the same in the organic EL device 2, except that the compound A is used in place of the compound represented by the formula (11) in the organic EL device 1.

It has been found that the compound represented by the formula (1) can be applied to an organic EL device with blue fluorescence since energy transfer is more likely to occur and sufficient efficiency can be obtained in the case of being combined with the compound A having a small Stokes shift (SS) and emitting blue light, as compared with the case of being combined with a compound having a large Stokes shift (SS) and emitting blue light. Furthermore, it has been found that an organic EL device with blue fluorescent which can be driven at a lower voltage and has a longer lifetime was obtained as compared with the case where a di-substituted anthracene compound and the compound A are combined.

In one embodiment, the Stokes shift of the compound A is 15 nm or less. The smaller the Stokes shift is, the more the energy transfer efficiency increases.

### [Compound Represented by the Formula (1)]

Next, a compound represented by the formula (1) will be described.

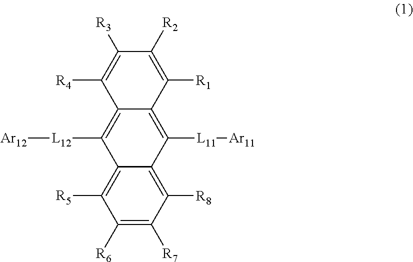

In the formula (1),

one or more among R₁ to R₈ are -L₁₃-Ar₁₃;

L₁₁ to L₁₃ are independently,

a single bond,

a substituted or unsubstituted arylene group including 6 to 50 ring carbon atoms, or

a substituted or unsubstituted divalent heterocyclic group including 5 to 50 ring atoms;

when two or more L₁₃'s are present, the two or more L₁₃'s may be the same as or different to each other;

Ar₁₁ to Ar₁₃ are independently,

a substituted or unsubstituted aryl group including 6 to 50 ring carbon atoms, or

a substituted or unsubstituted monovalent heterocyclic group including 5 to 50 ring atoms;

when two or more Ar₁₃'s are present, the two or more Ar₁₃'s may be the same as or different to each other;

R₁ to R₈ which are not -L₁₃-Ar₁₃ are independently,

a hydrogen atom,

a substituted or unsubstituted alkyl group including 1 to 50 carbon atoms,

a substituted or unsubstituted alkenyl group including 2 to 50 carbon atoms,

a substituted or unsubstituted alkynyl group including 2 to 50 carbon atoms,

a substituted or unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a halogen atom, a cyano group, a nitro group,

a substituted or unsubstituted aryl group including 6 to 50 ring carbon atoms, or

a substituted or unsubstituted monovalent heterocyclic group including 5 to 50 ring atoms;

R₉₀₁ to R₉₀₇ are independently,

a hydrogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms.

When two or more of each of R₉₀₁ to R₉₀₇ are present, the two or more of each of R₉₀₁, to R₉₀₇ may be the same as or different to each other.

Note that -L₁₁-Ar₁, -L₁₂-Ar₁₂, -L₁₃-Ar₁₃ and R₁ to R₈ which are not -L₁₃-Ar₁₃ do not form a ring which fuses to an anthracene ring by bonding with each other.

In one embodiment, L₁₁ to L₁₃ in the formula (1) are independently, a single bond, or a substituted or unsubstituted arylene group including 6 to 50 ring carbon atoms.

In one embodiment, L₁₁ to L₁₃ in the formula (1) are independently, a single bond, or a group selected from the group consisting of:

a substituted or unsubstituted phenylene group,

a substituted or unsubstituted biphenylene group,

a substituted or unsubstituted terphenylene group,

a substituted or unsubstituted quaterphenylene group, and

a substituted or unsubstituted naphthylene group.

In one embodiment, Ar₁ to Ar₁₃ in the formula (1) are independently, a substituted or unsubstituted of aryl group including 6 to 30 ring carbon atoms.

In one embodiment, Ar₁ to Ar₁₃ in the formula (1) are independently a group selected from the group consisting of:

a substituted or unsubstituted phenyl group,

a substituted or unsubstituted naphthyl group,

a substituted or unsubstituted fluorenyl group,

a substituted or unsubstituted 9,9′-spirobifluorenyl group,

a substituted or unsubstituted benzofluorenyl group,

a substituted or unsubstituted phenanthryl group, and

a substituted or unsubstituted benzophenanthryl group.

In one embodiment, one or more among Ar₁₁ to Ar₁₃ in the formula (1) are independently a substituted or unsubstituted monovalent heterocyclic group including 5 to 30 ring atoms.

In one embodiment, the group represented by -L₁₃-Ar₁₃ in the formula (1) is selected from the group consisting of:

a substituted or unsubstituted phenyl group,

a substituted or unsubstituted naphthyl group,

a substituted or unsubstituted biphenyl group,

a substituted or unsubstituted phenanthrenyl group,

a substituted or unsubstituted benzophenanthrenyl group,

a substituted or unsubstituted fluorenyl group,

a substituted or unsubstituted benzofluorenyl group,

a substituted or unsubstituted dibenzofuranyl group,

a substituted or unsubstituted naphthobenzofuranyl group,

a substituted or unsubstituted dibenzothiophenyl group, and

a substituted or unsubstituted carbazolyl group.

In one embodiment, the compound represented by the formula (1) is a compound represented by the following formula (1-1).

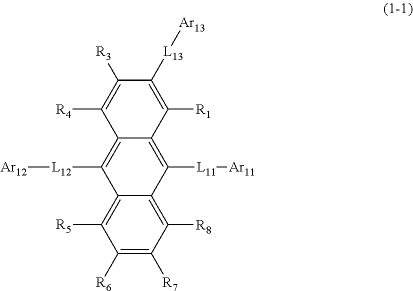

In the formula (1-1), L₁₁ to L₁₃, Ar₁₁ to Ar₁₃, R₁, R₃, R₄ and R₅ to R₈ are as defined in the formula (1).

In one embodiment, the compound represented by the formula (1) is a compound represented by the following formula (1-1H).

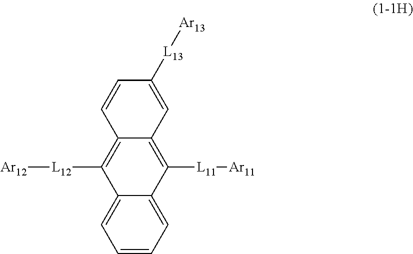

In the formula (1-1H), L₁₁ to L₁₃ and Ar₁₁ to Ar₁₃ are as defined in the formula (1).

In one embodiment, the compound represented by the formula (1) is selected from the group consisting of a compound represented by the following formula (1-2), a compound represented by the following formula (1-3), and a compound represented by the following formula (1-4).

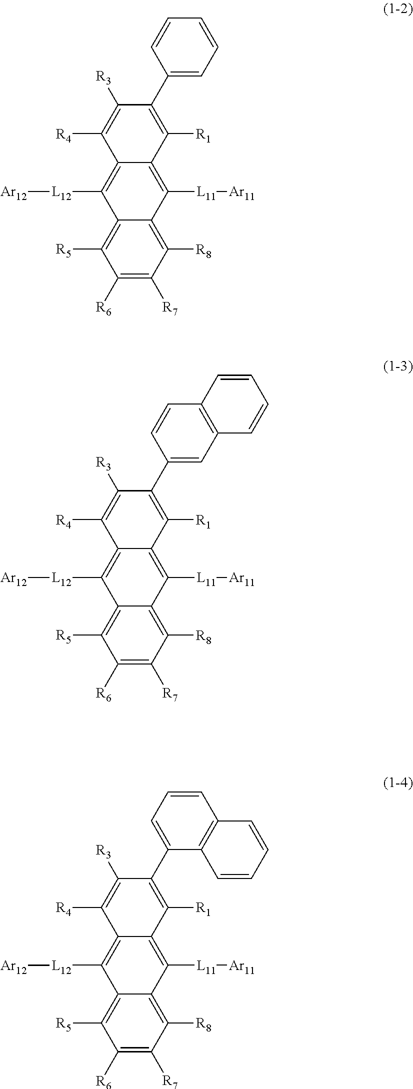

In the formulas (1-2) to (1-4), L₁₁, L₁₂, Ar₁₁, Ar₁₂, R₁, R₃, R₄, and R₅ to R₈ are as defined in the formula (1).

In one embodiment, R₁ to R₈ in the formula (1) which are not-L₁₃-Ar₁₃ are hydrogen atoms.

Details of each substituent in the formula (1) are as described in the section of [Definition] of the specification. Hereinafter, the same applies to each substituent in the formulas (11), (21), (31), (41), and (51).

Specific examples of the compound represented by the formula (1) will be described below, but these are merely examples, and the compound represented by the formula (1) is not limited to the following specific examples.

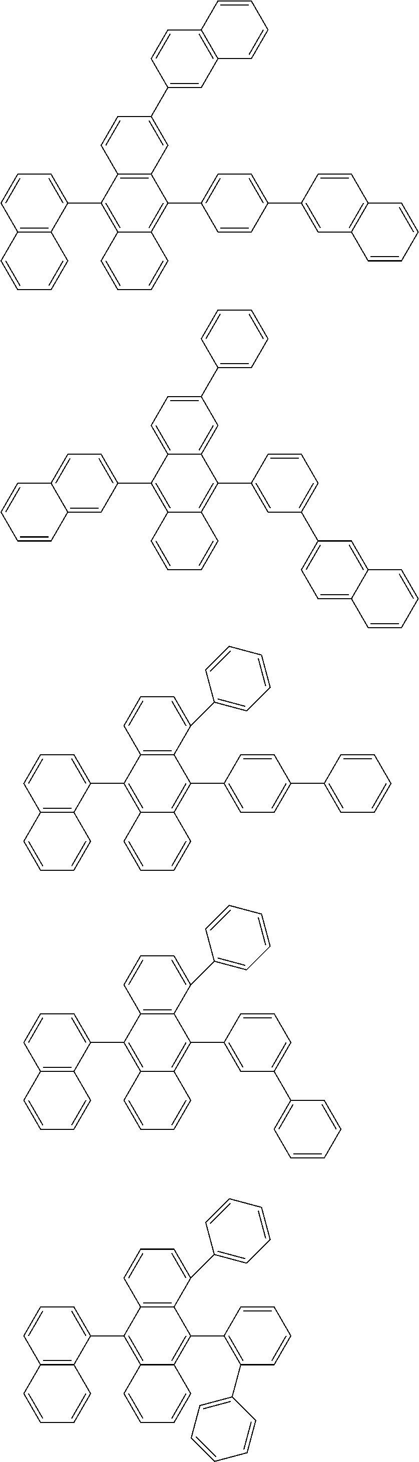

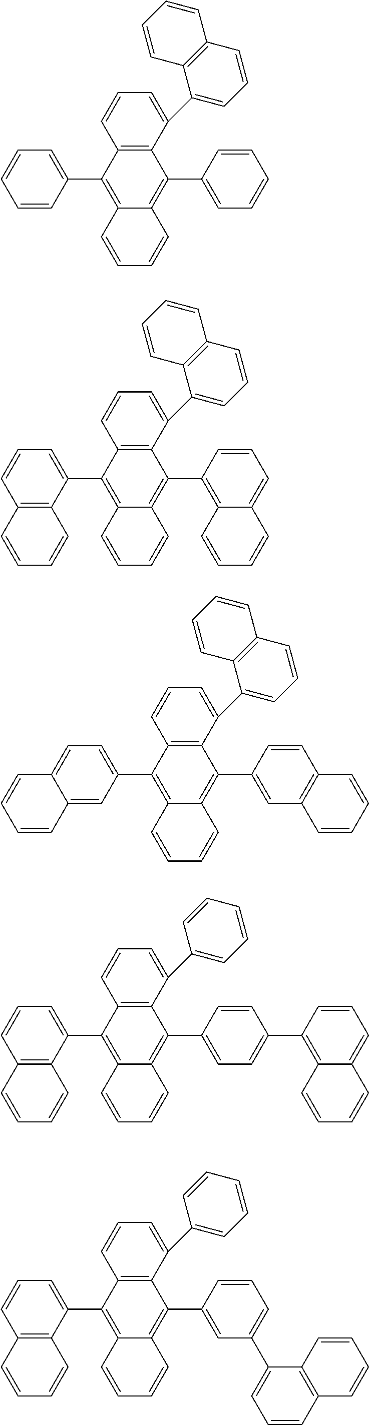

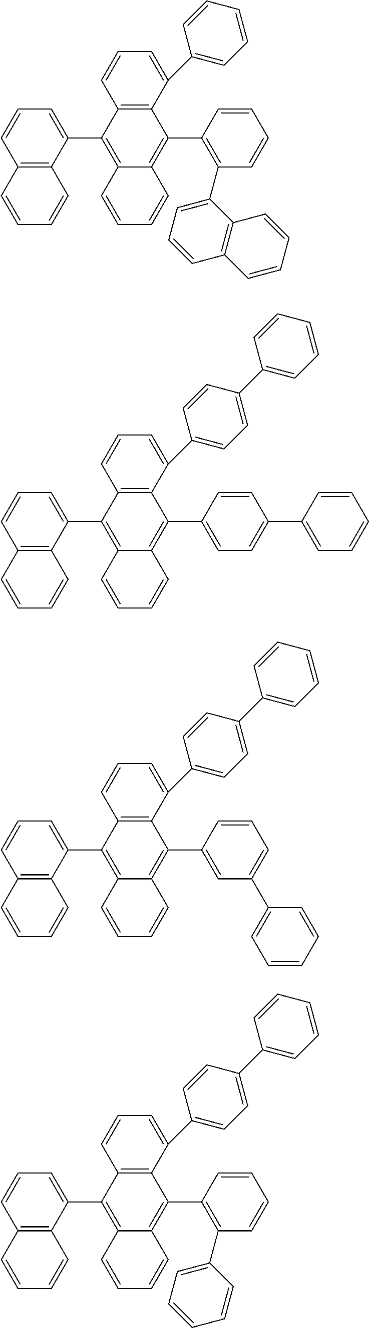

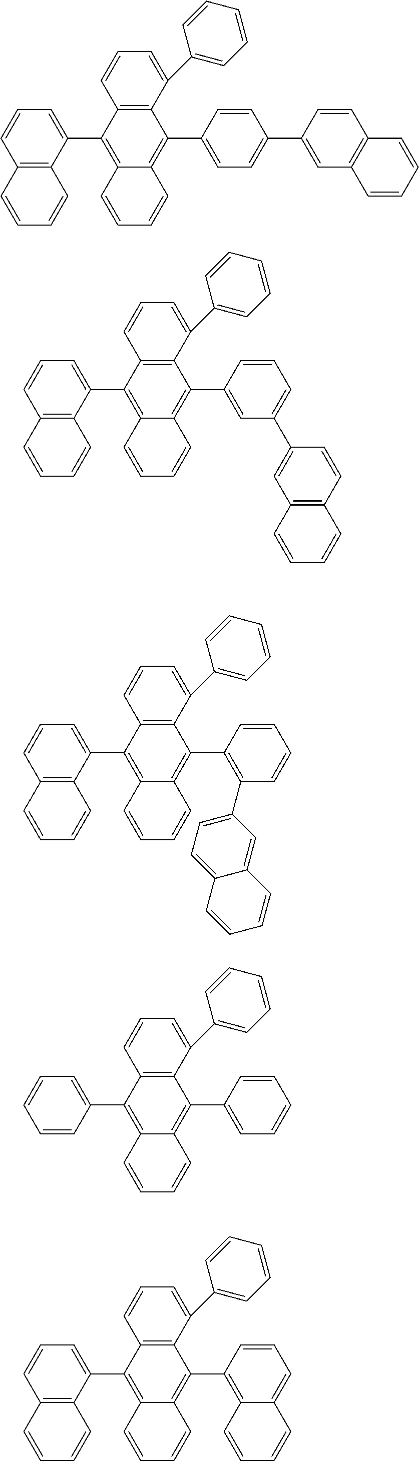

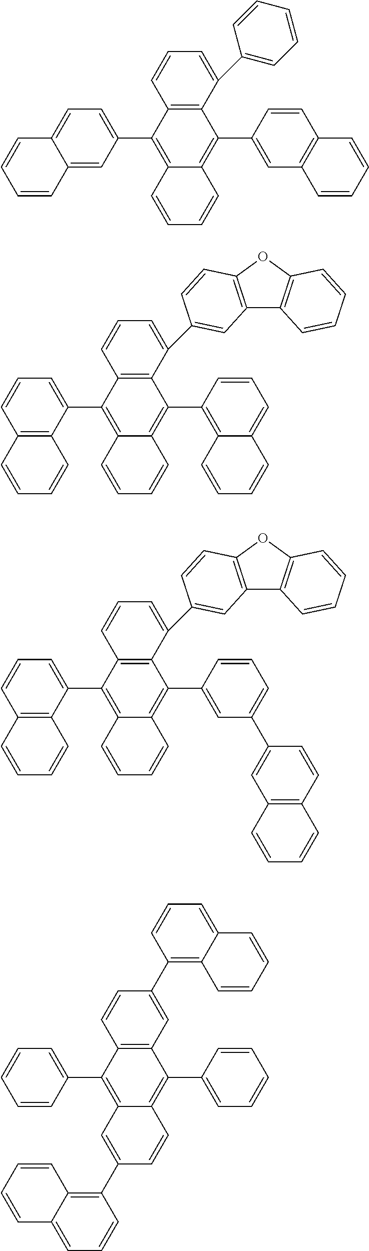

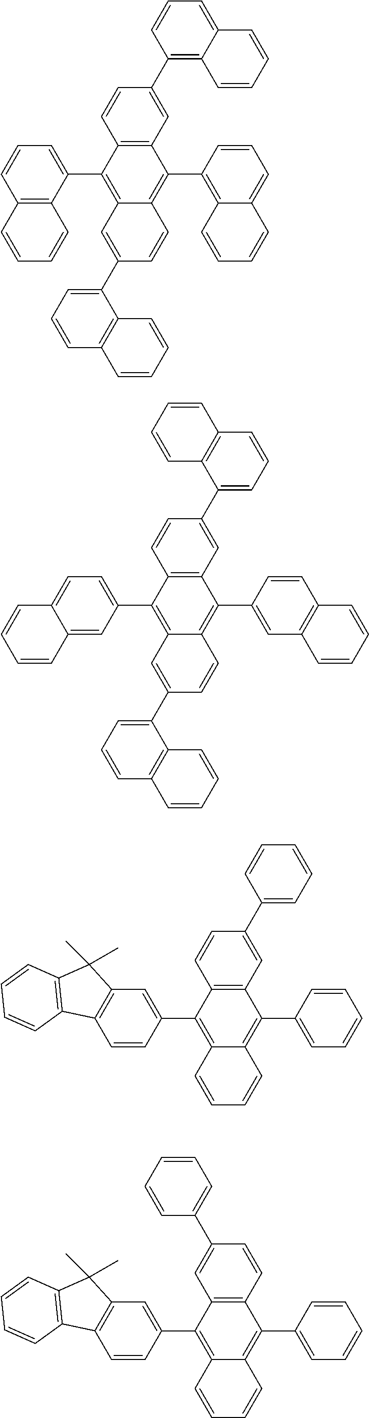

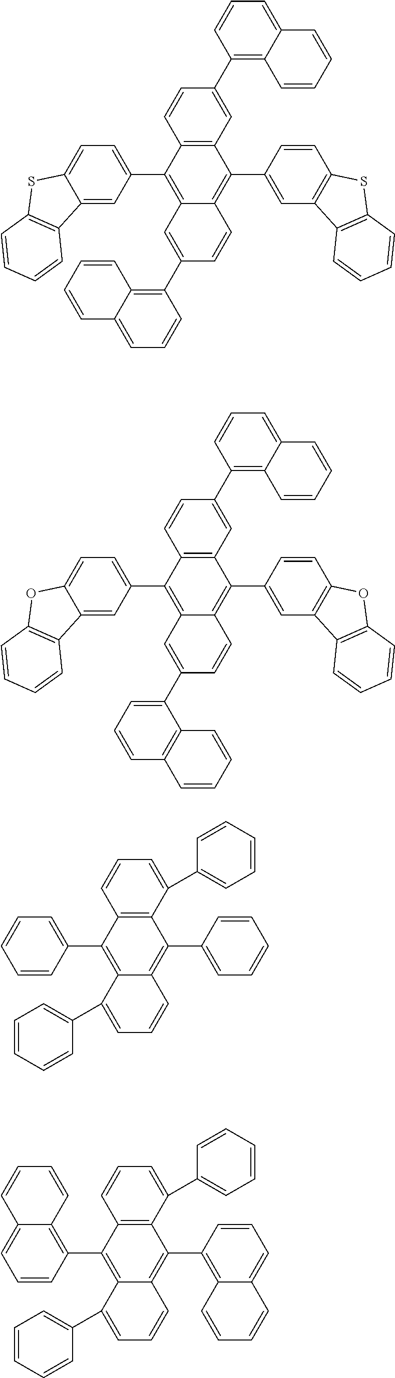

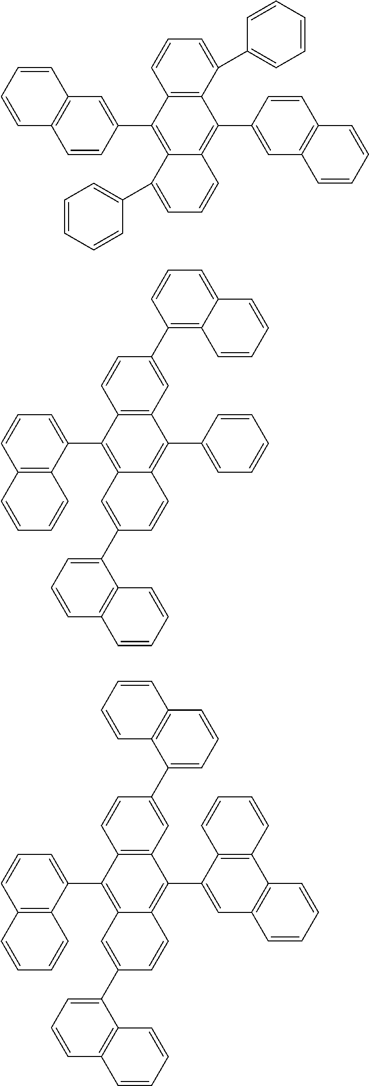

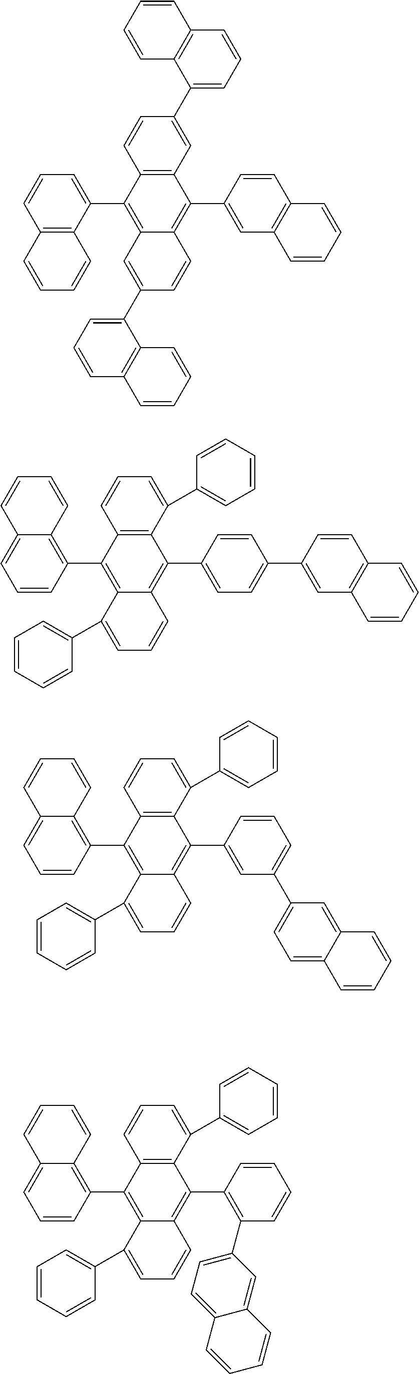

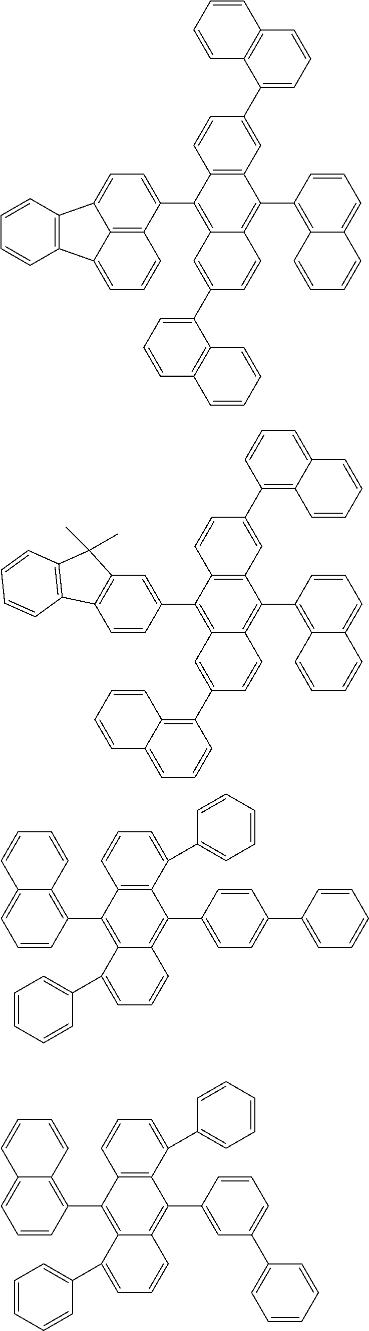

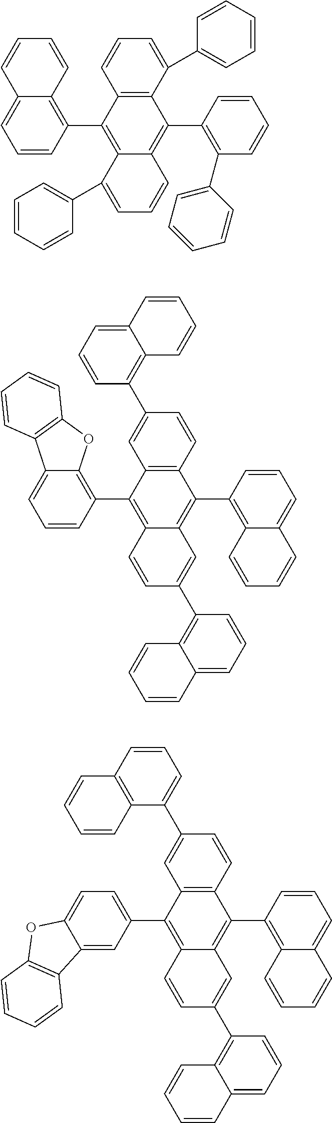

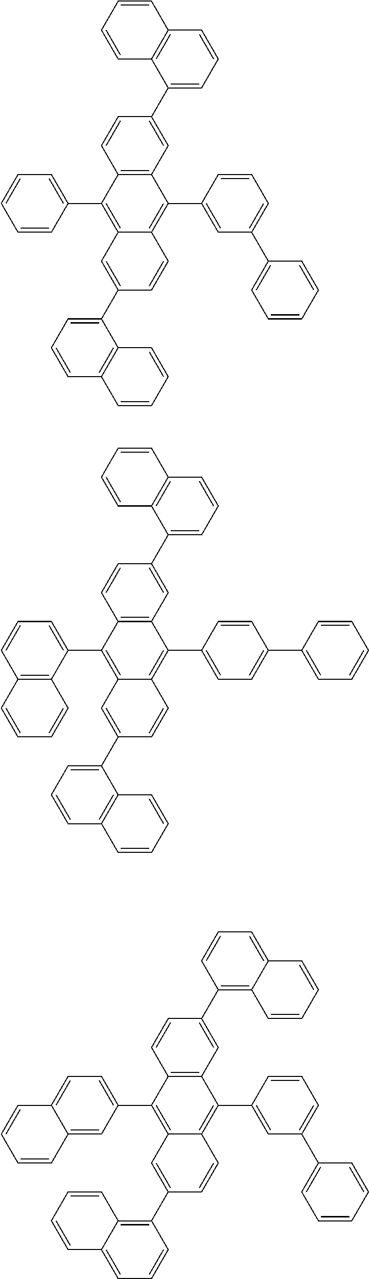

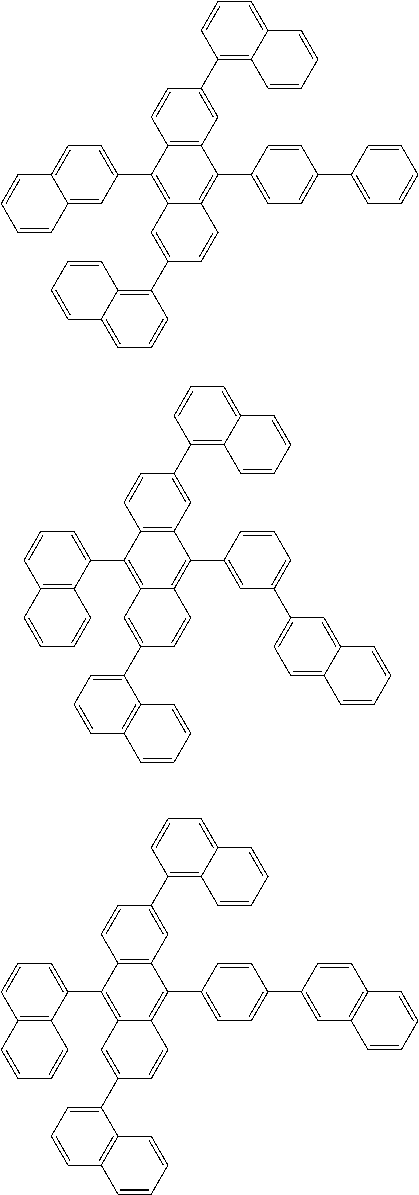

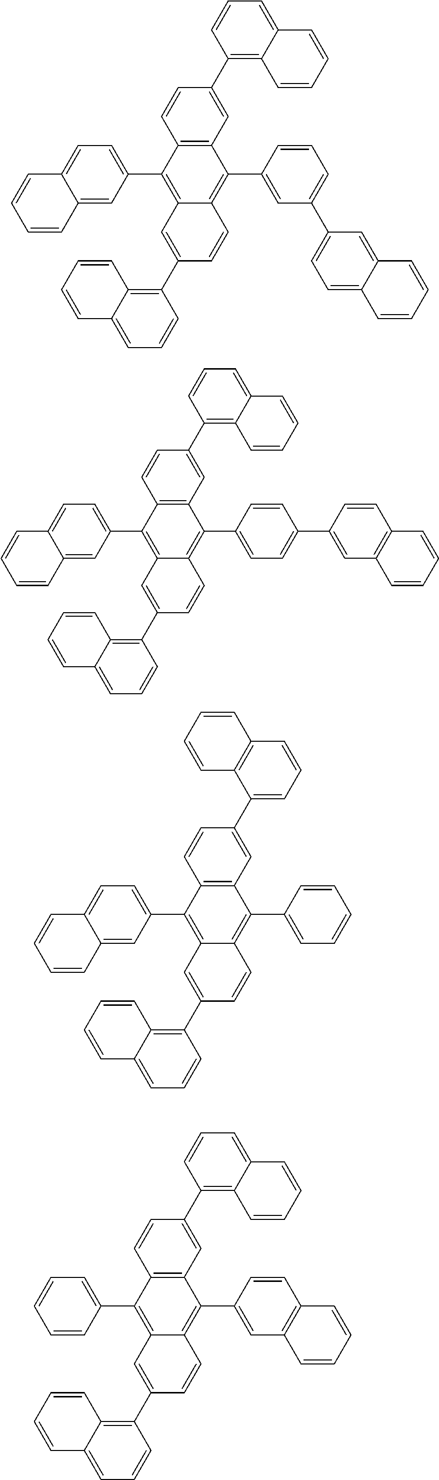

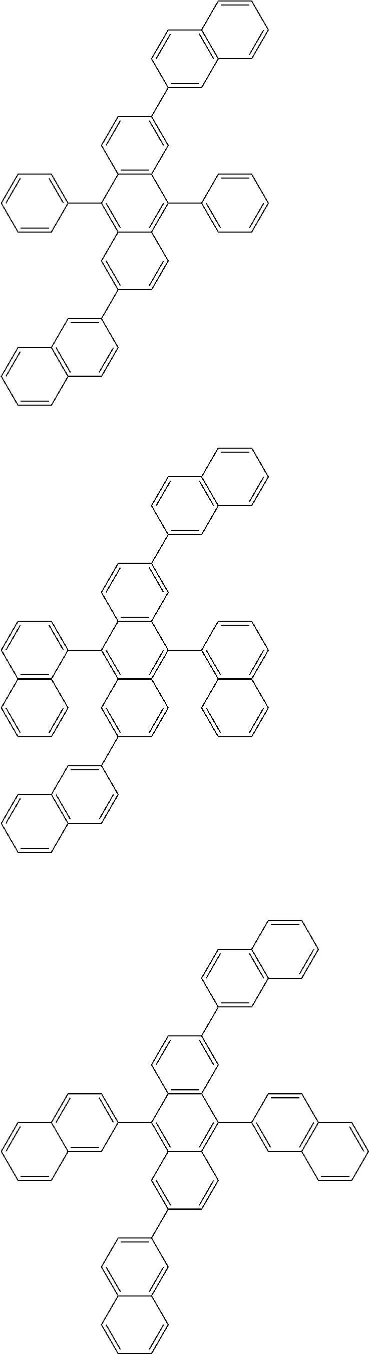

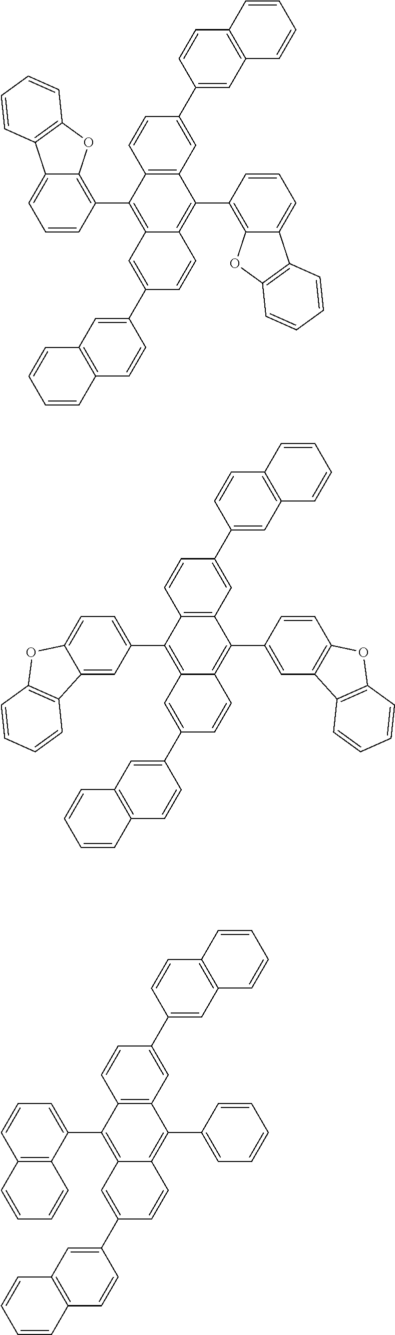

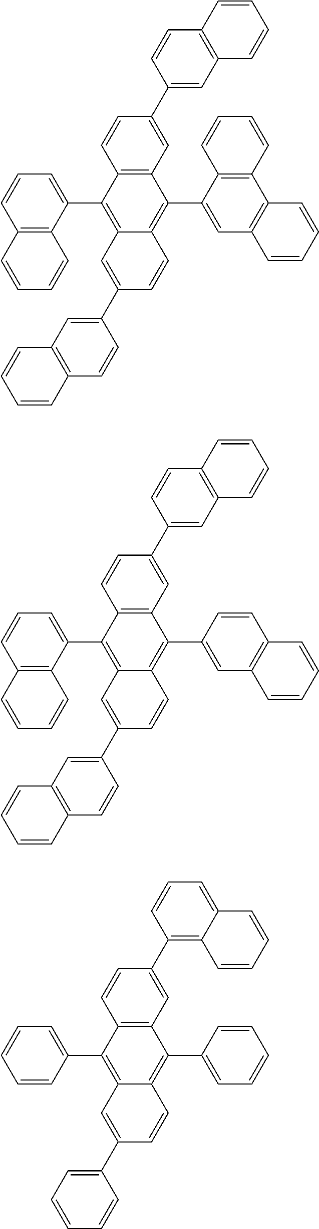

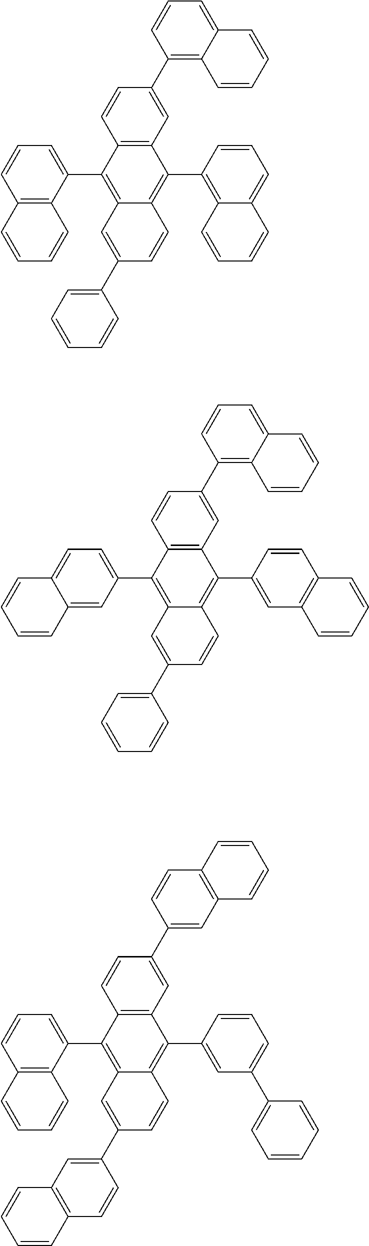

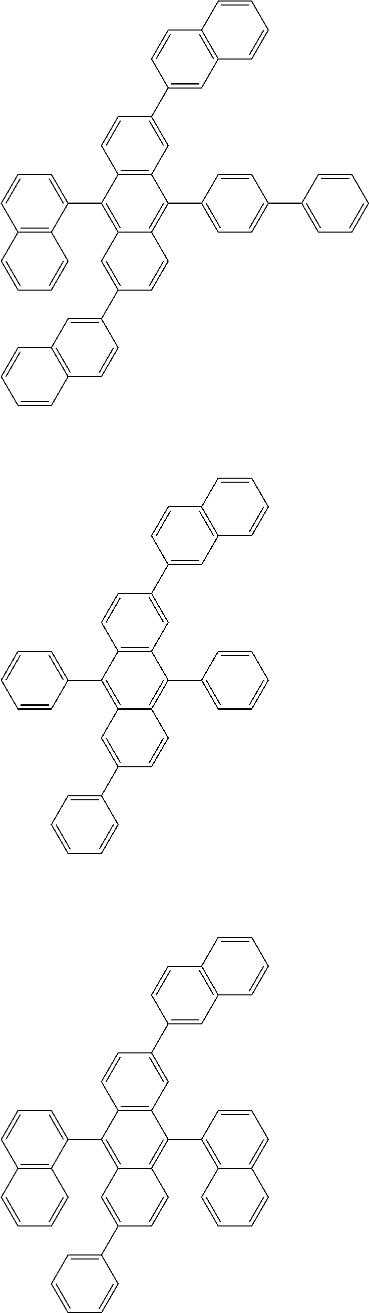

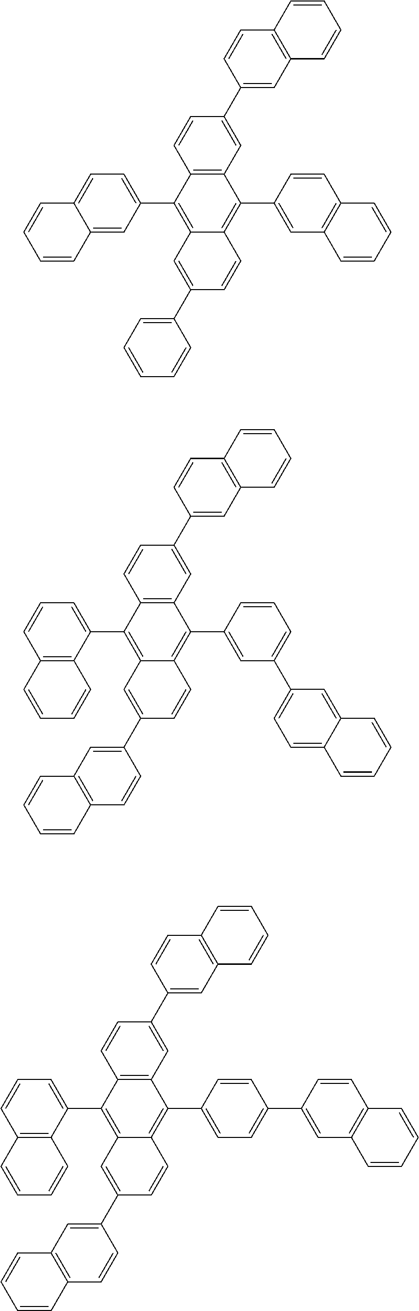

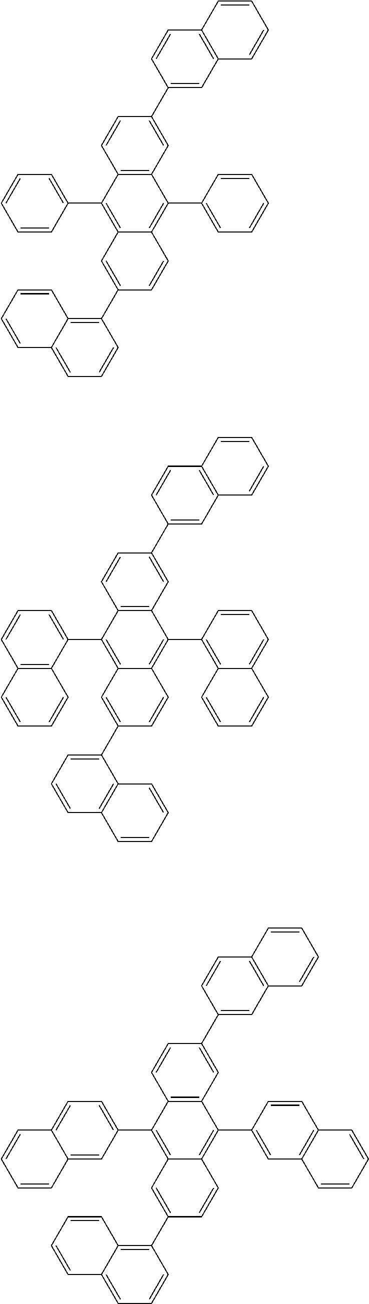

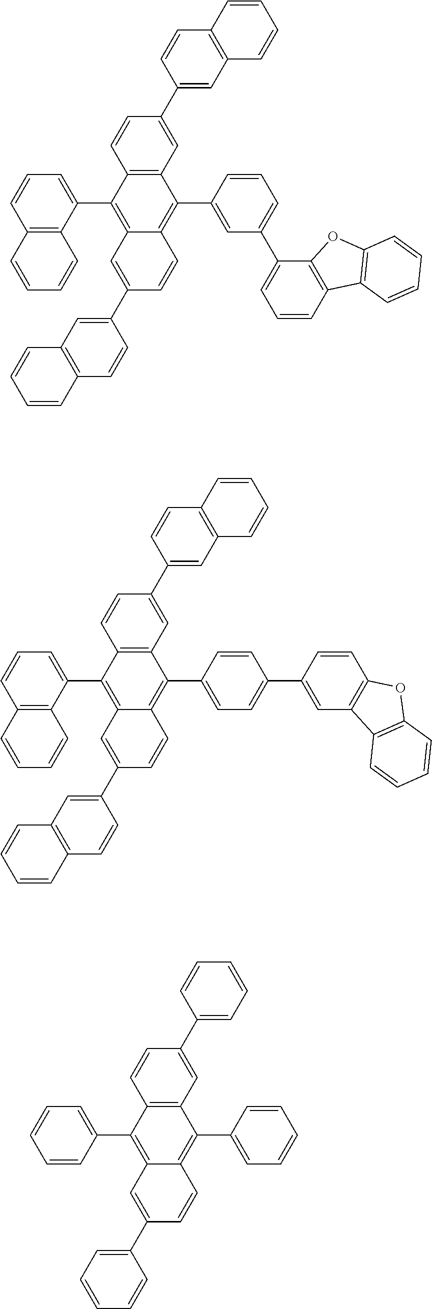

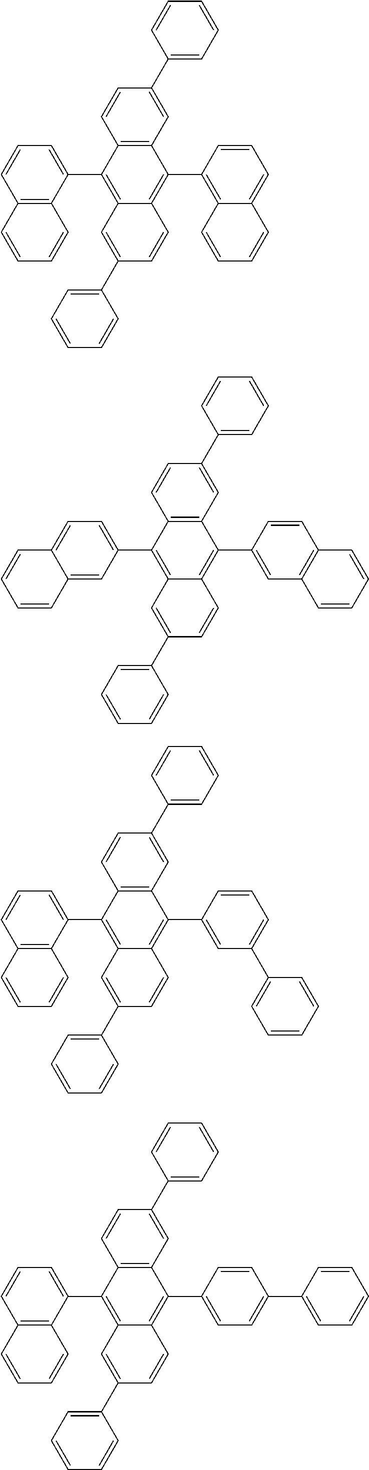

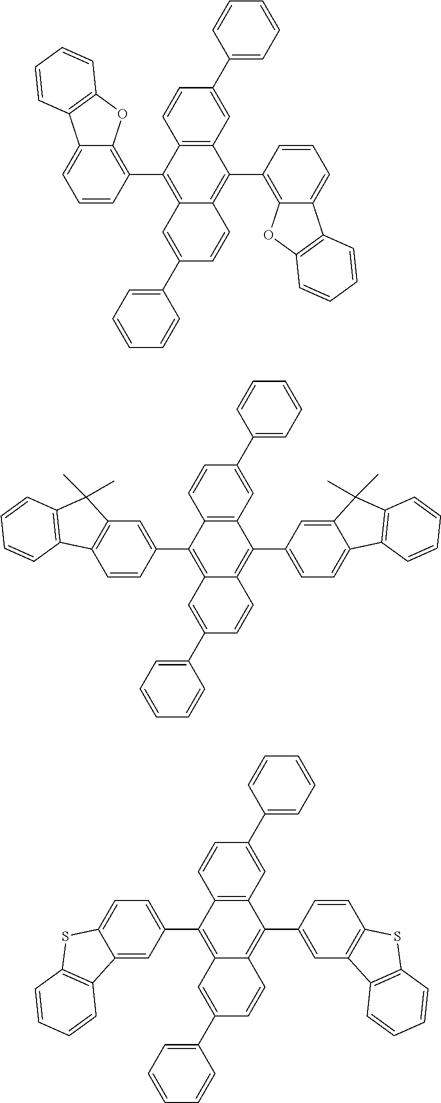

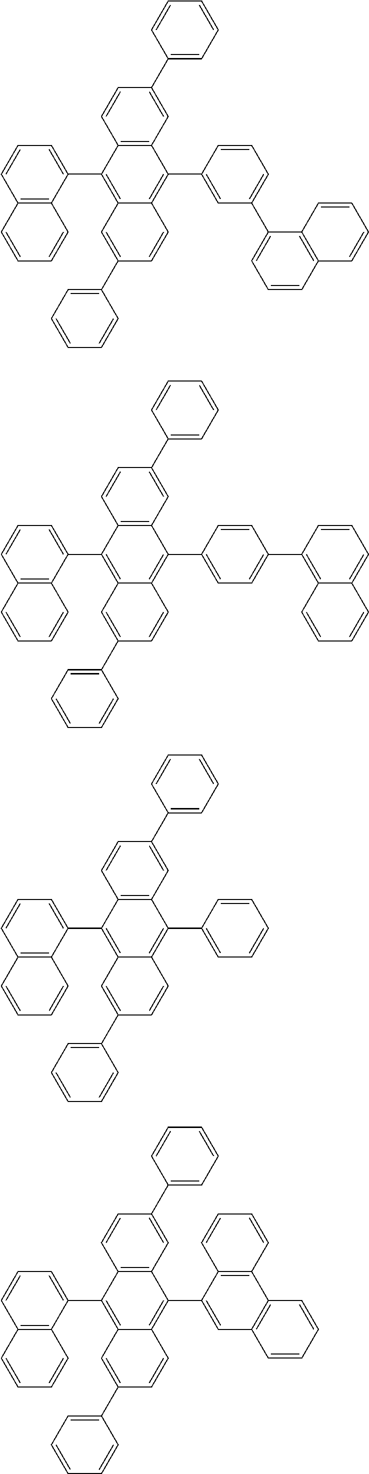

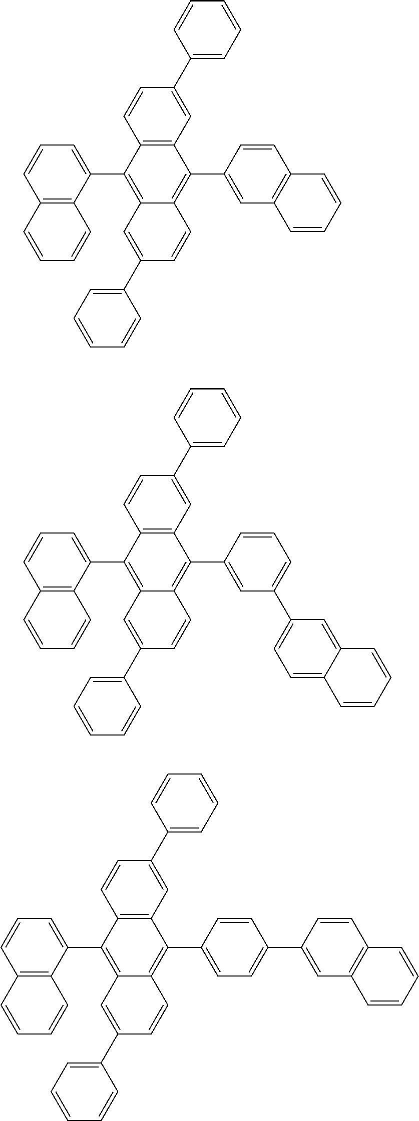

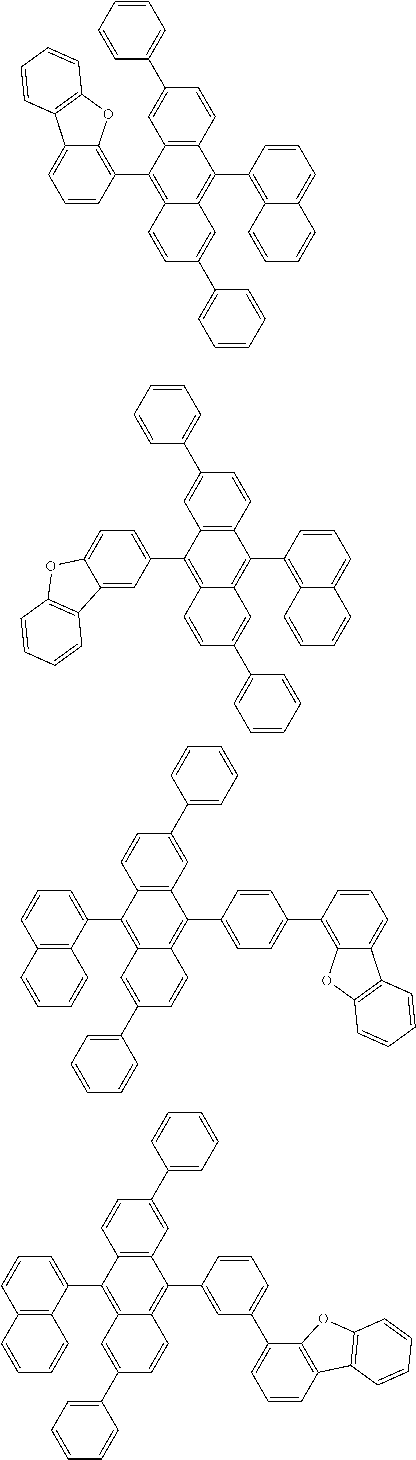

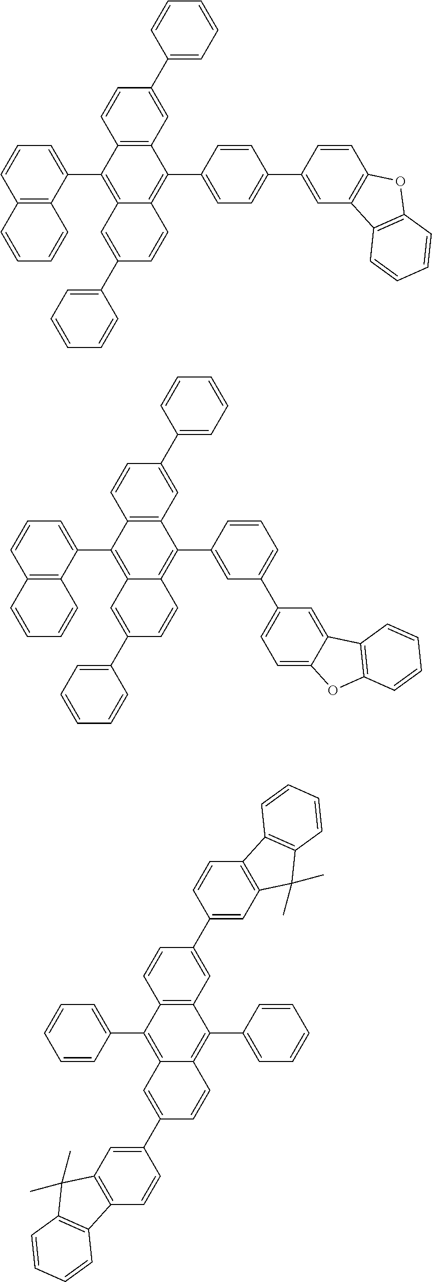

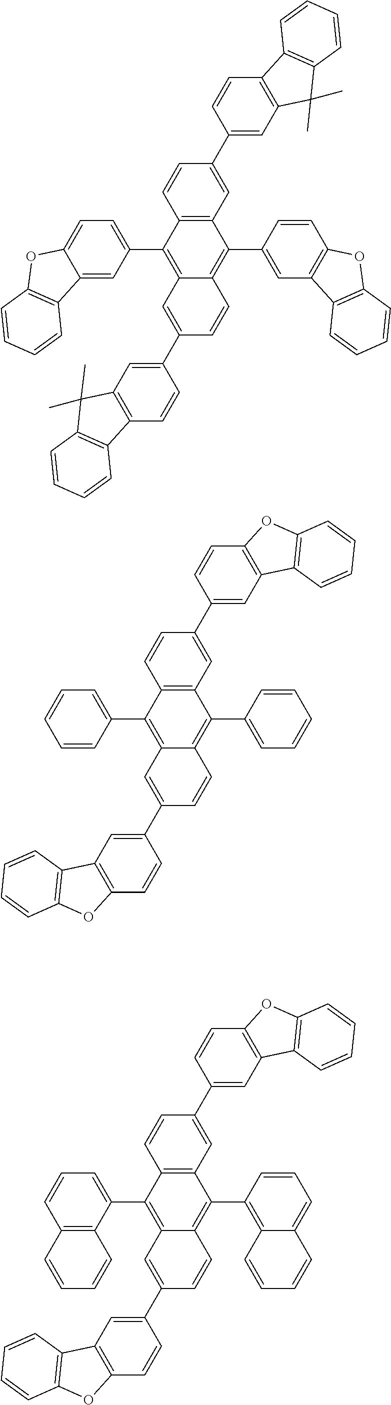 [SMILES: CC1(C)c2ccccc2-c2ccc(-c3ccc4c(-c5ccc6oc7ccccc7c6c5)c5cc(-c6ccc7c(c6)C(C)(C)c6ccccc6-7)ccc5c(-c5ccc6oc7ccccc7c6c5)c4c3)cc21 ; c1ccc(-c2c3ccc(-c4ccc5oc6ccccc6c5c4)cc3c(-c3ccccc3)c3ccc(-c4ccc5oc6ccccc6c5c4)cc23)cc1 ; c1ccc2c(-c3c4ccc(-c5ccc6oc7ccccc7c6c5)cc4c(-c4cccc5ccccc45)c4ccc(-c5ccc6oc7ccccc7c6c5)cc34)cccc2c1]

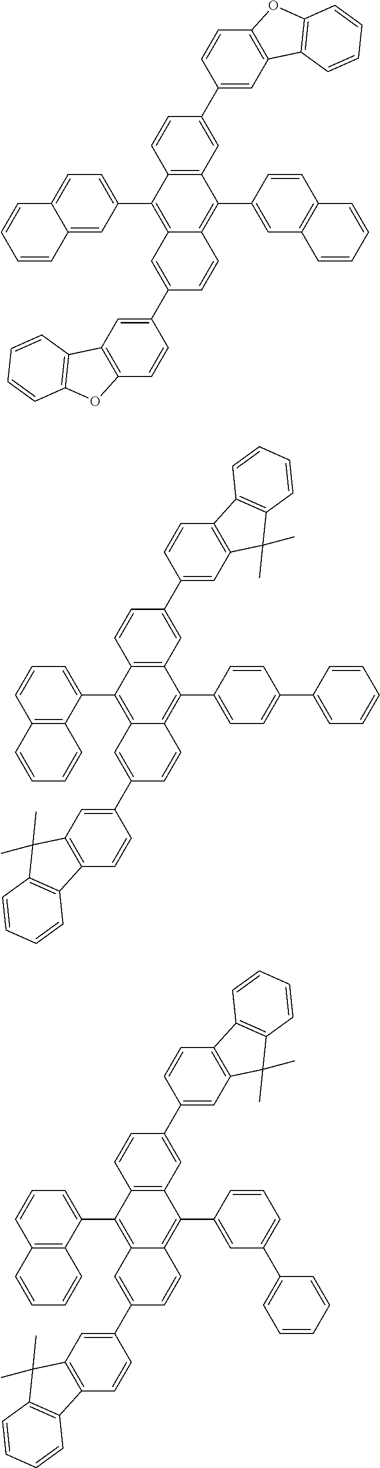

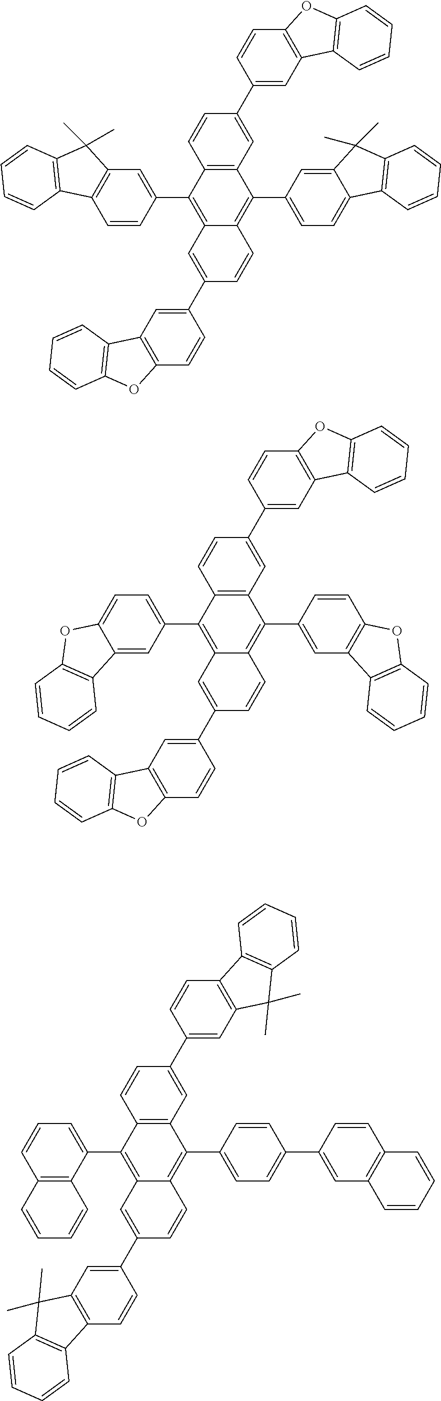

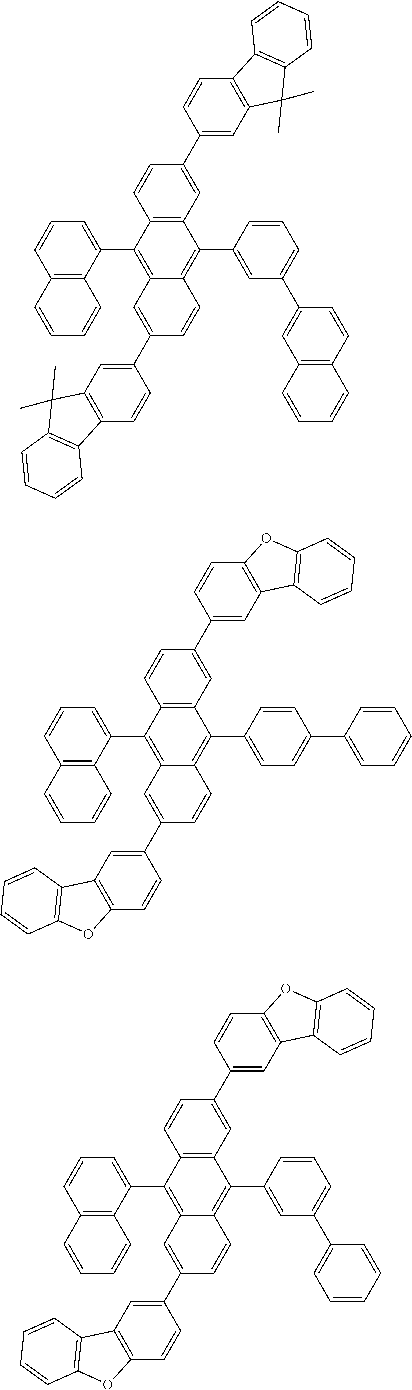

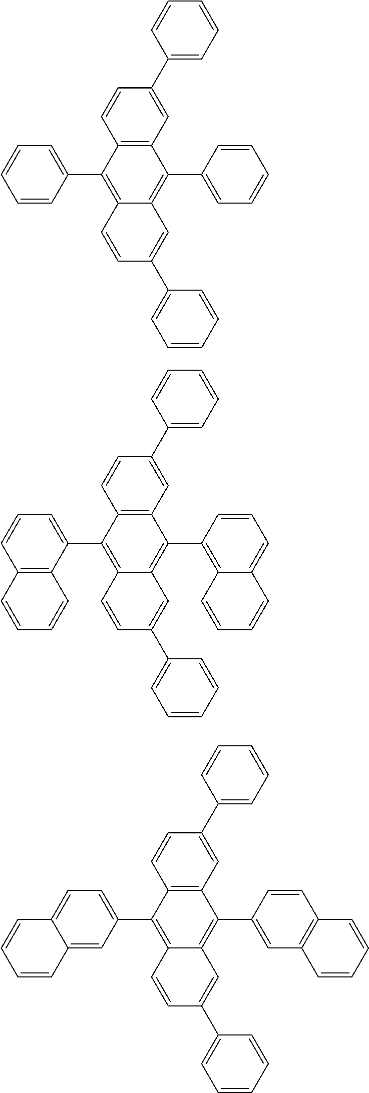

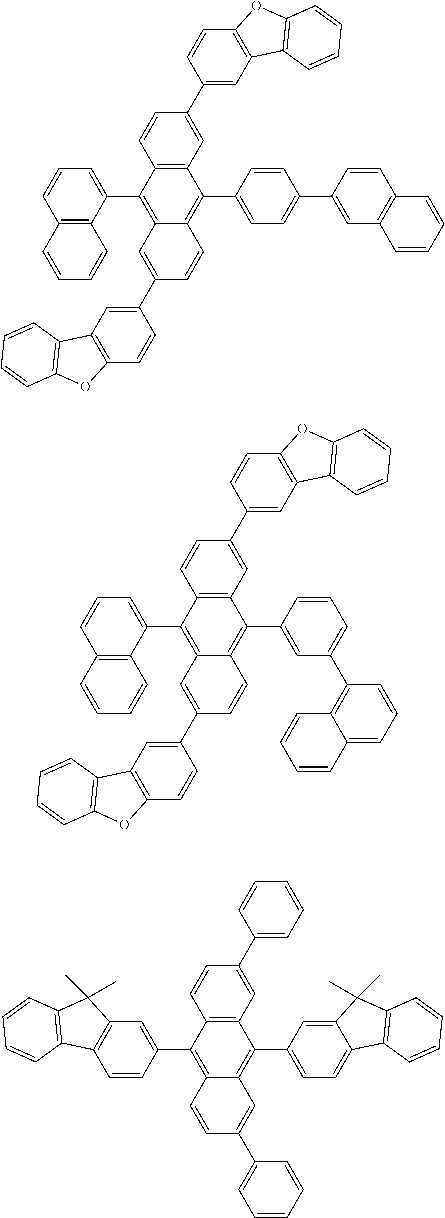

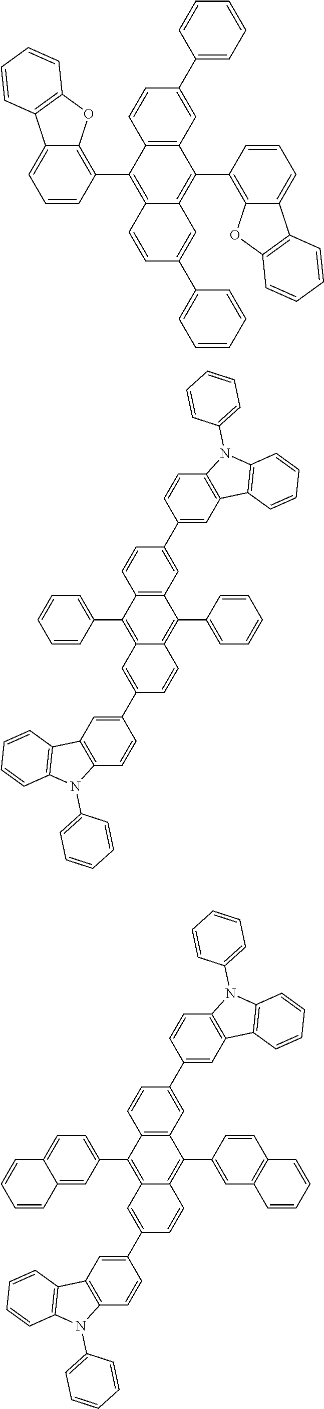

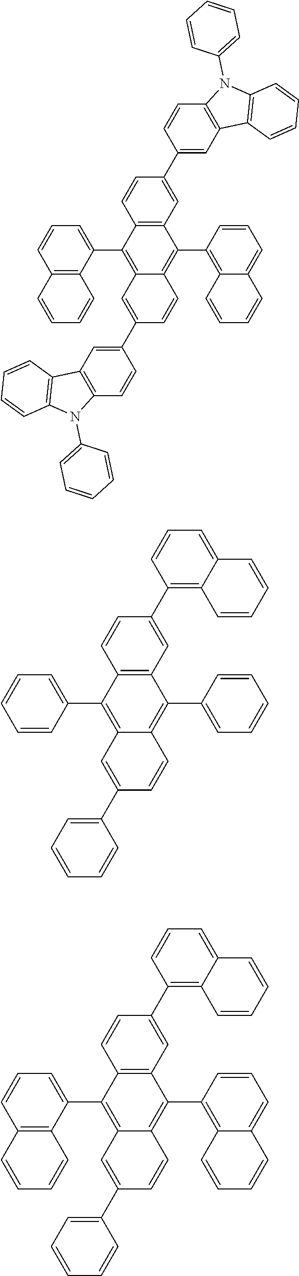

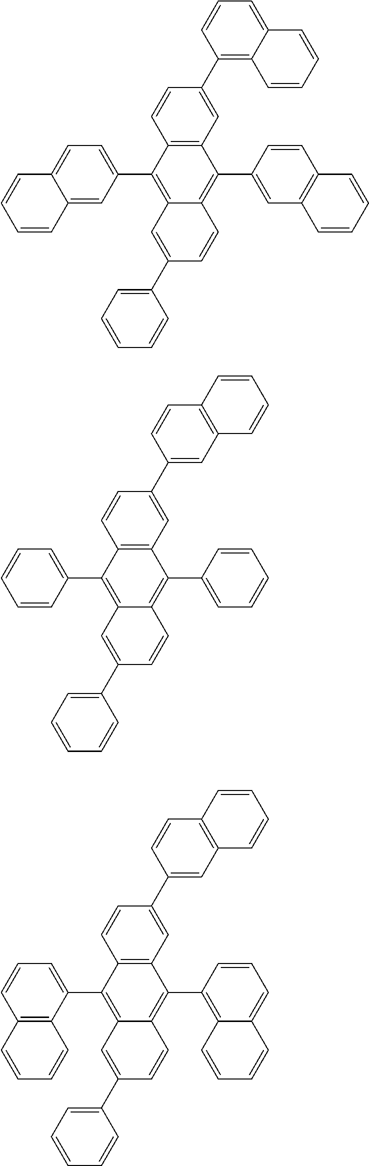

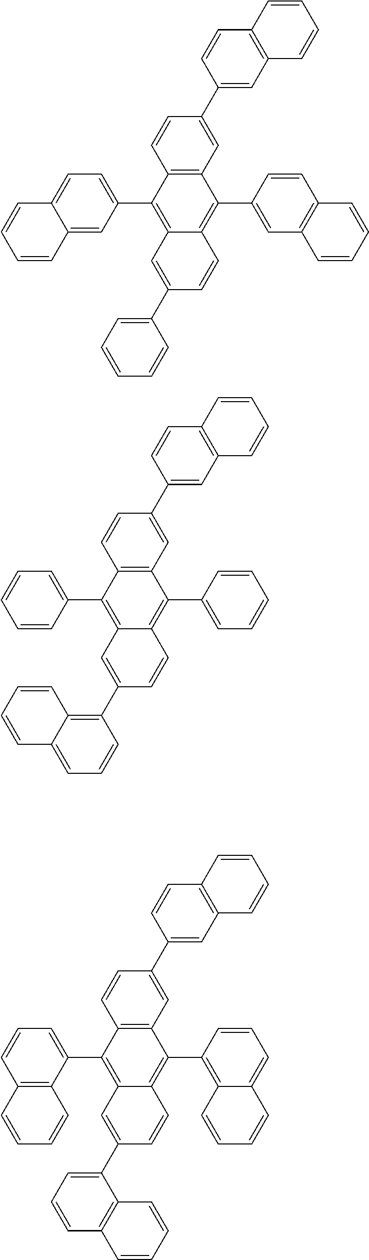

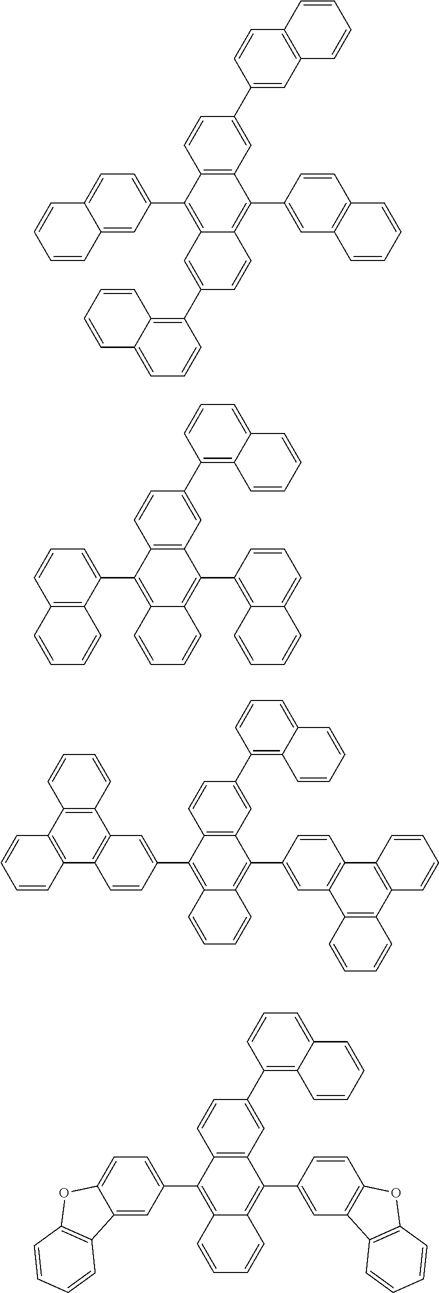

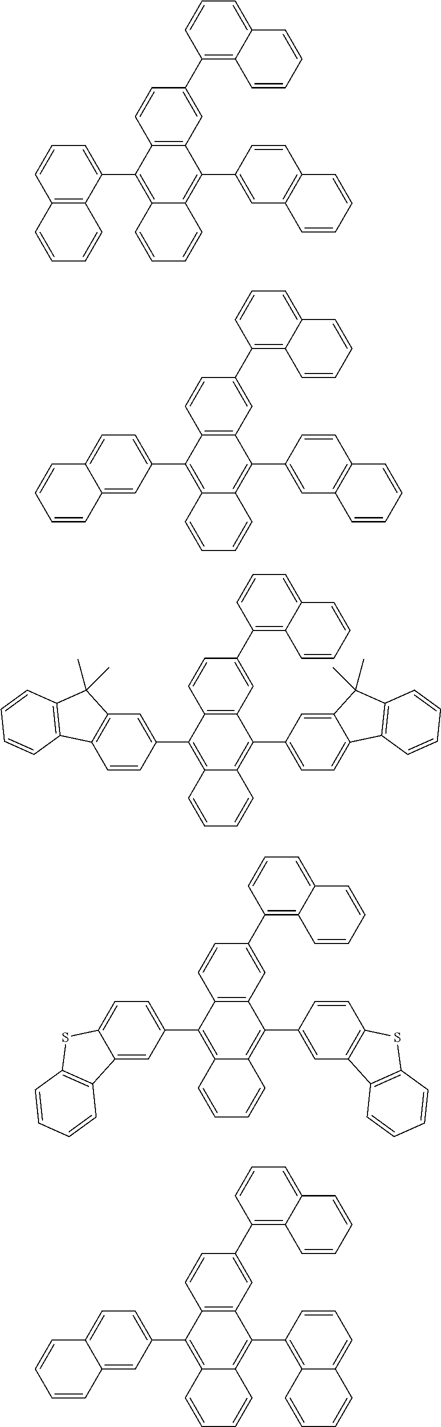

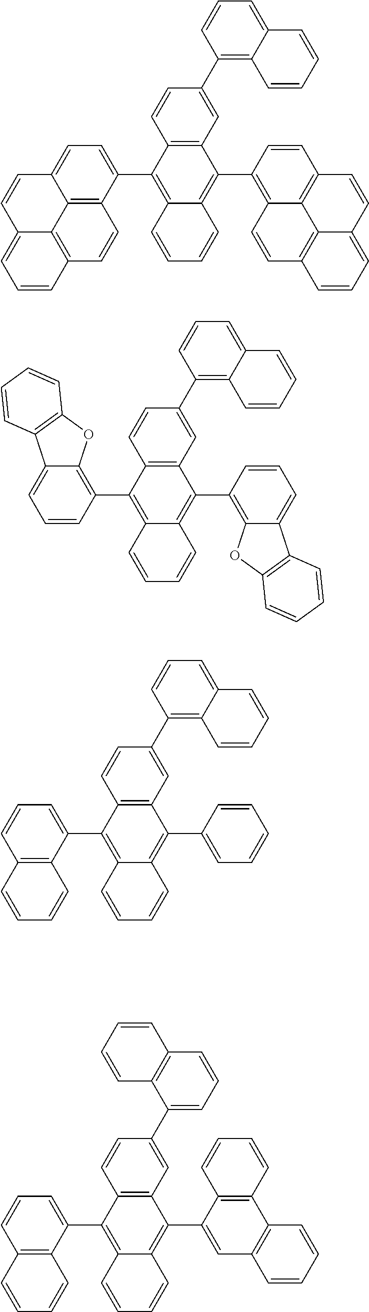

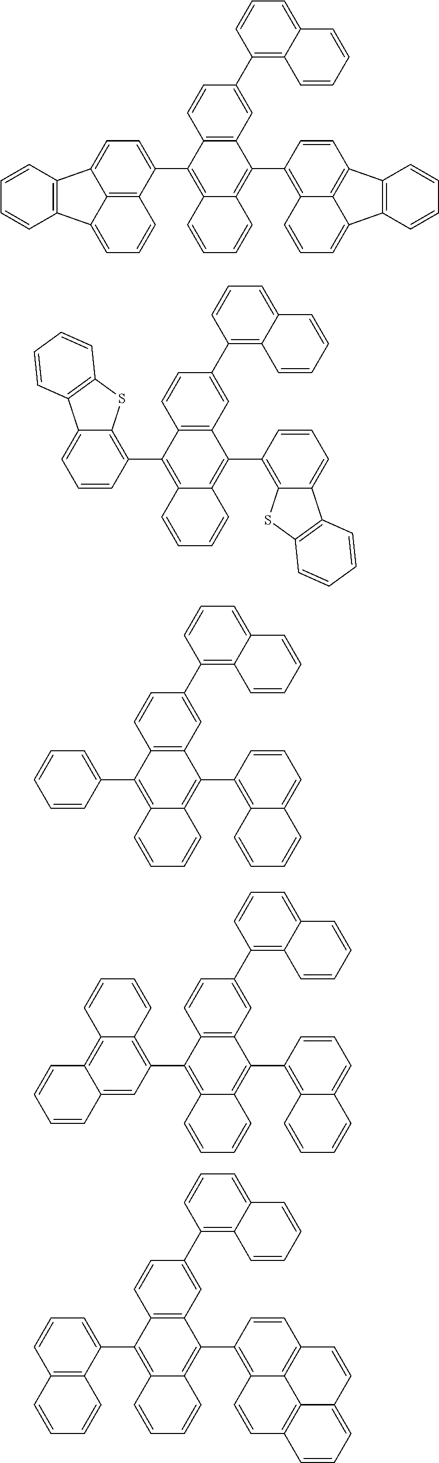

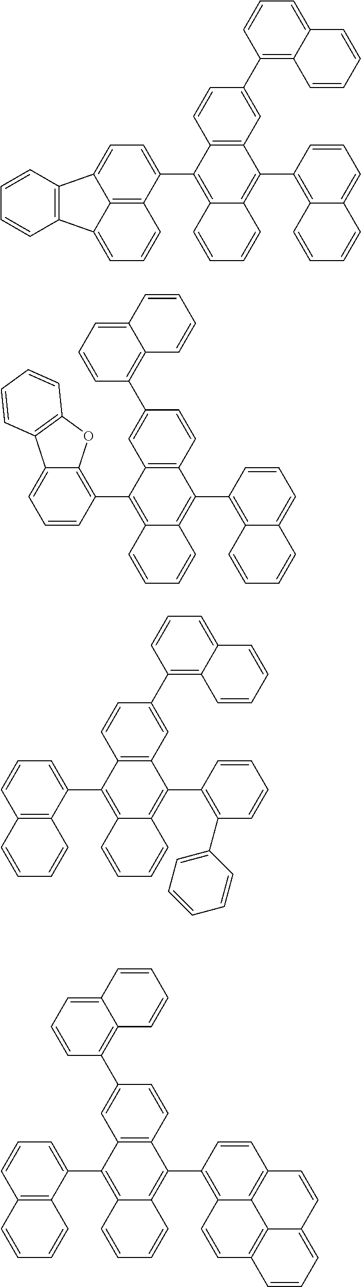

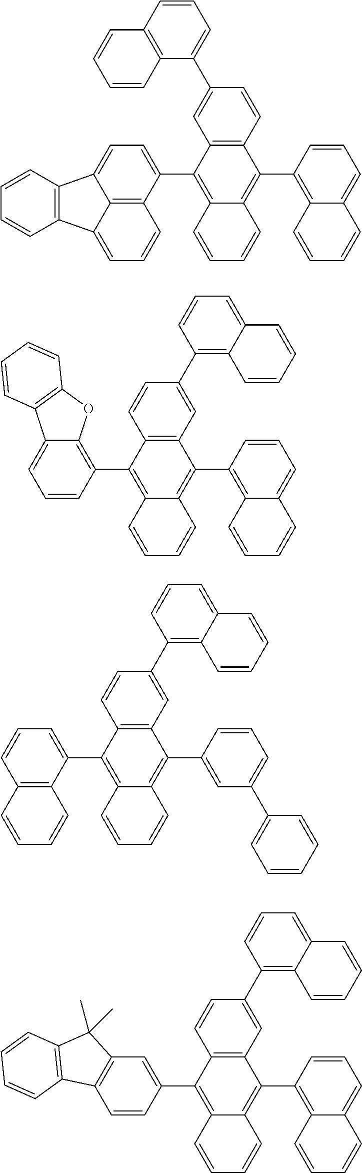

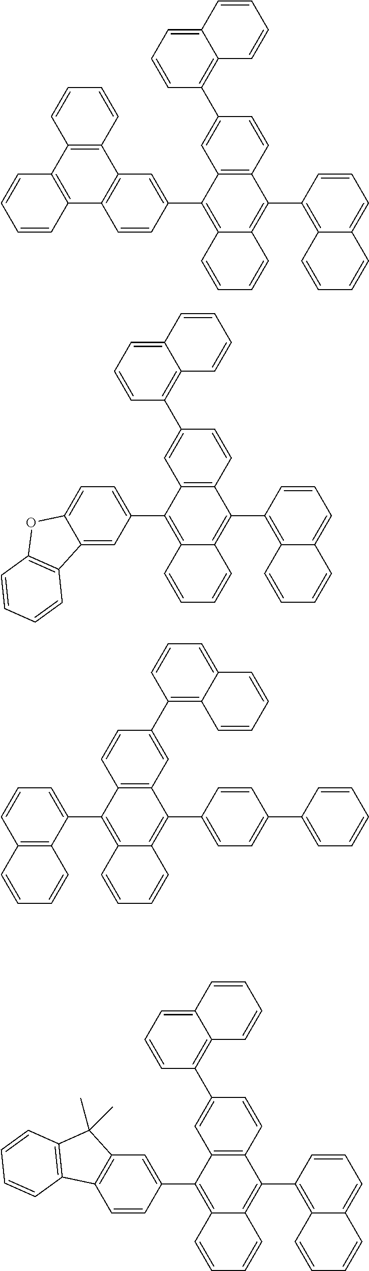

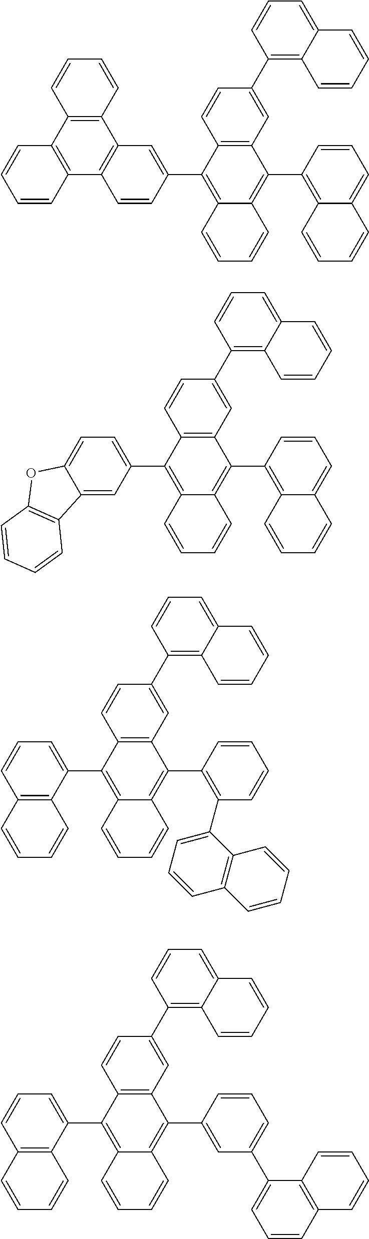

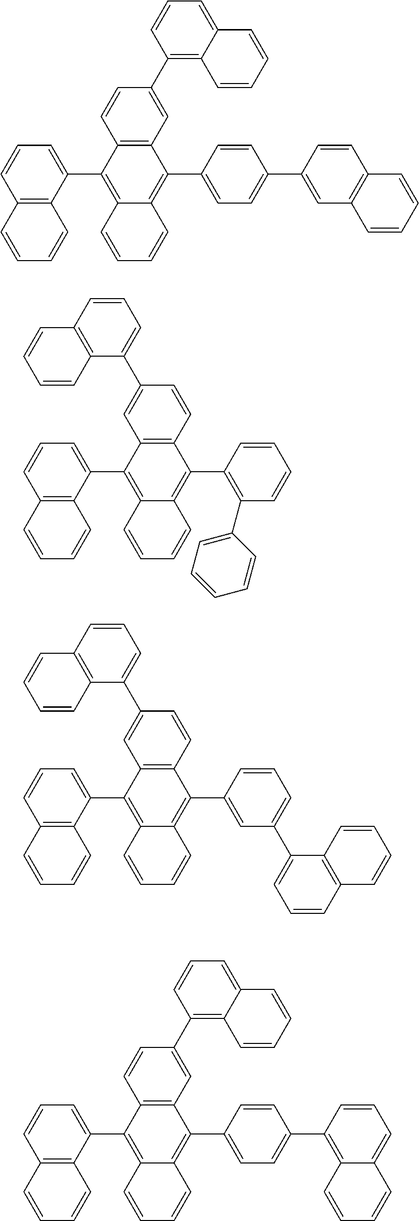

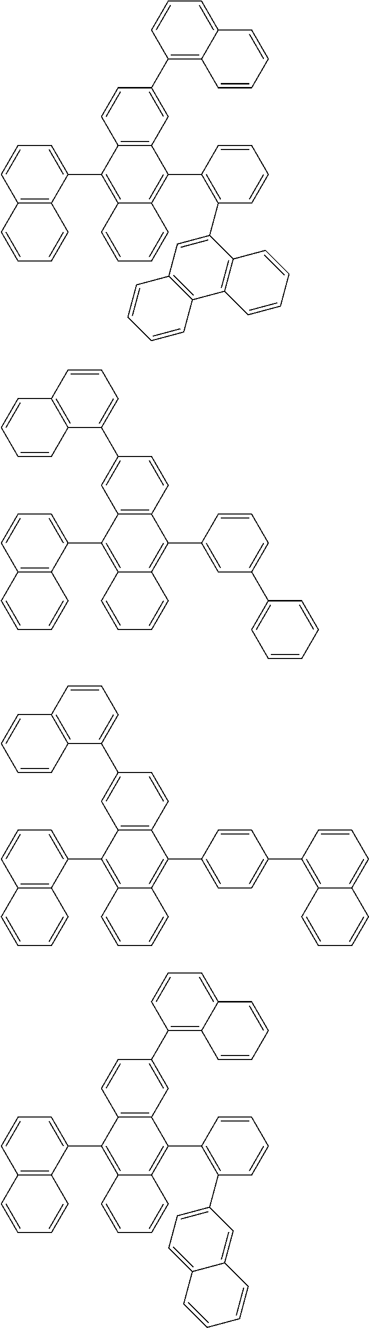

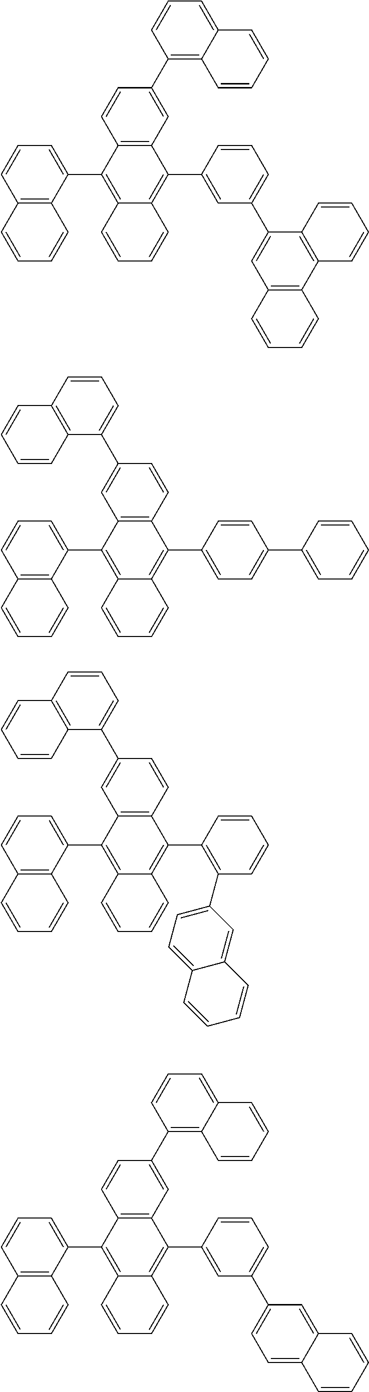

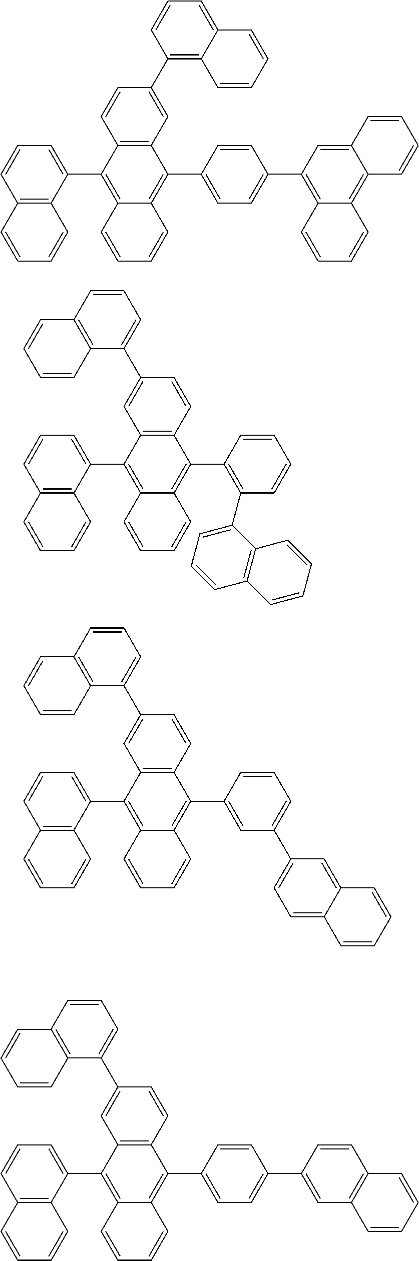

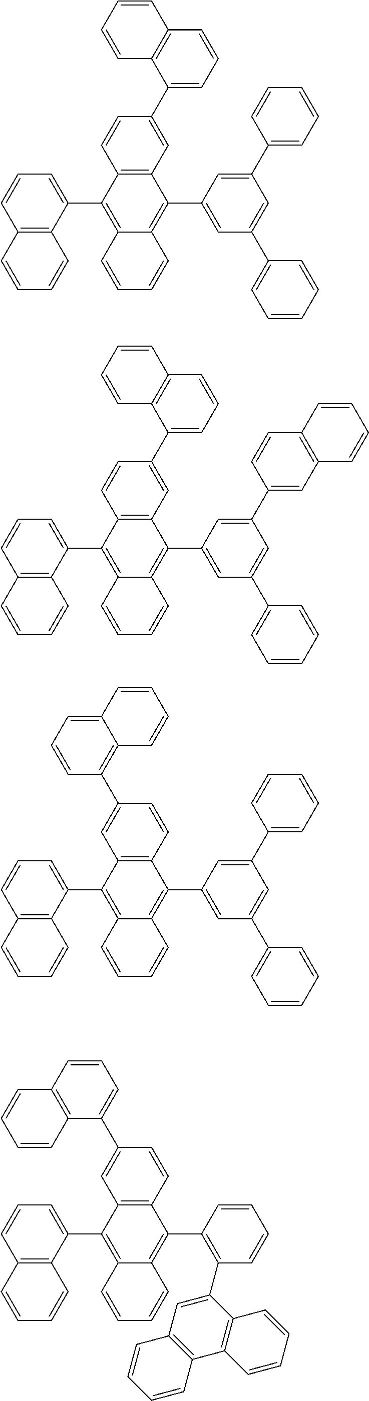

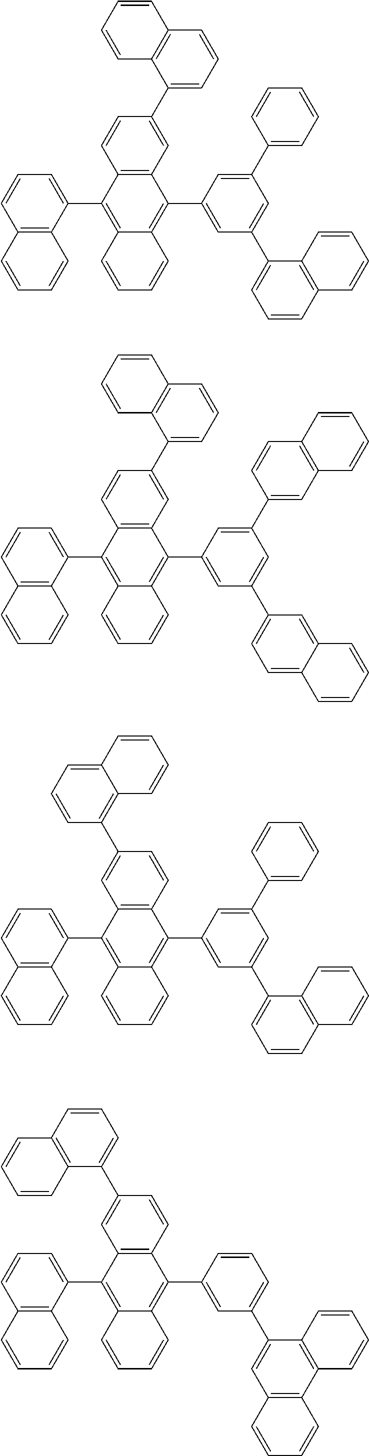

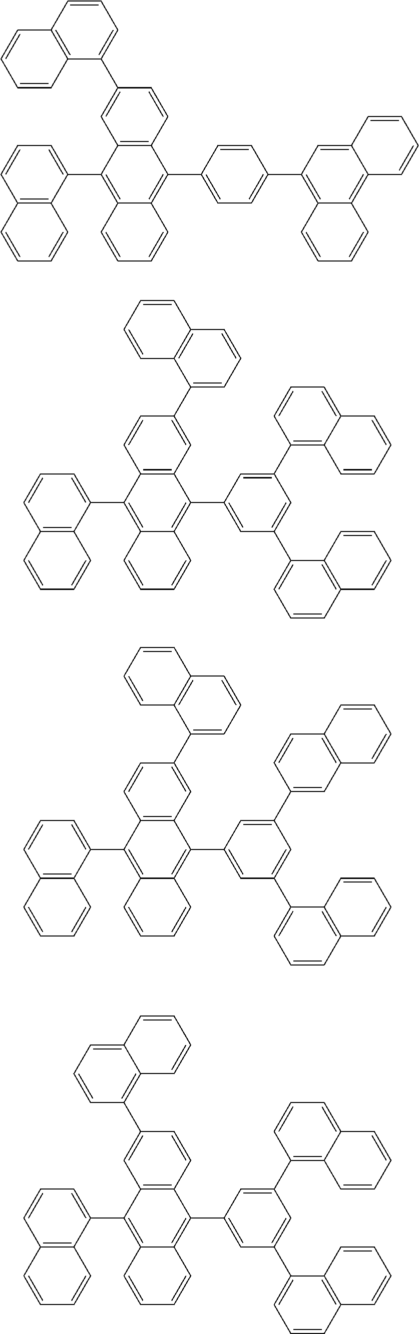

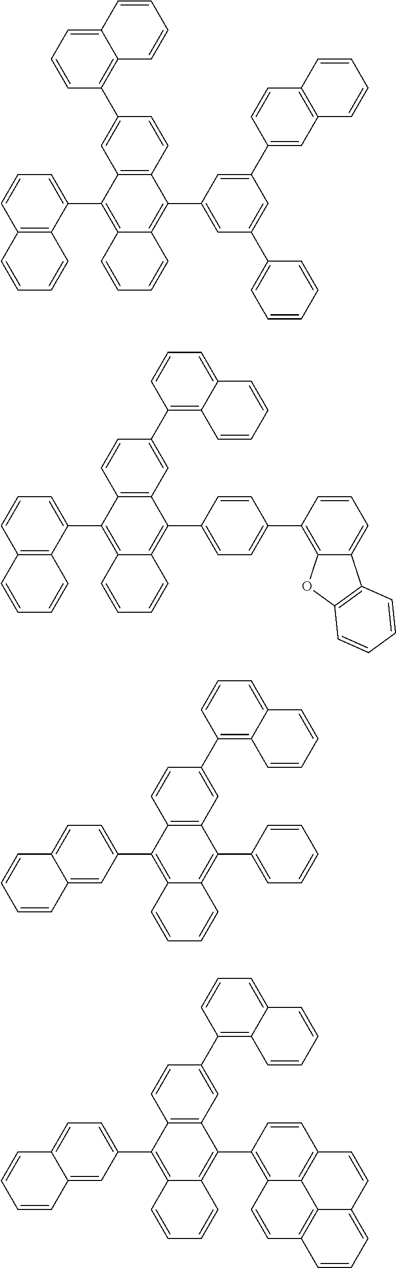

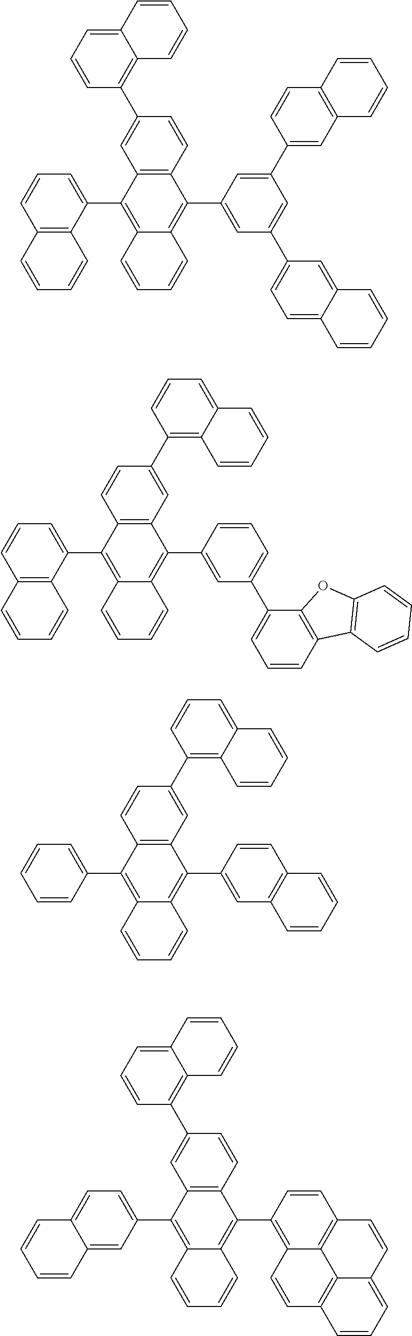

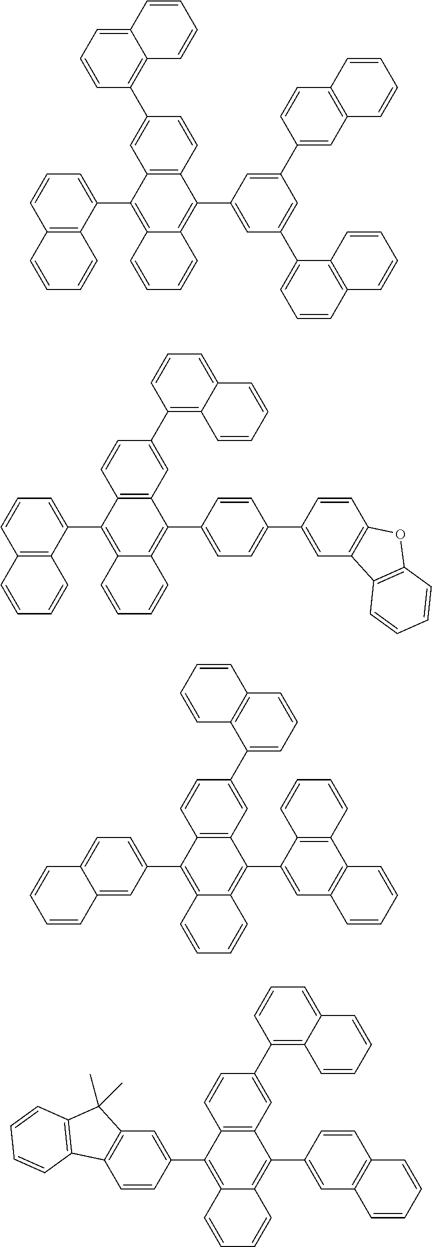

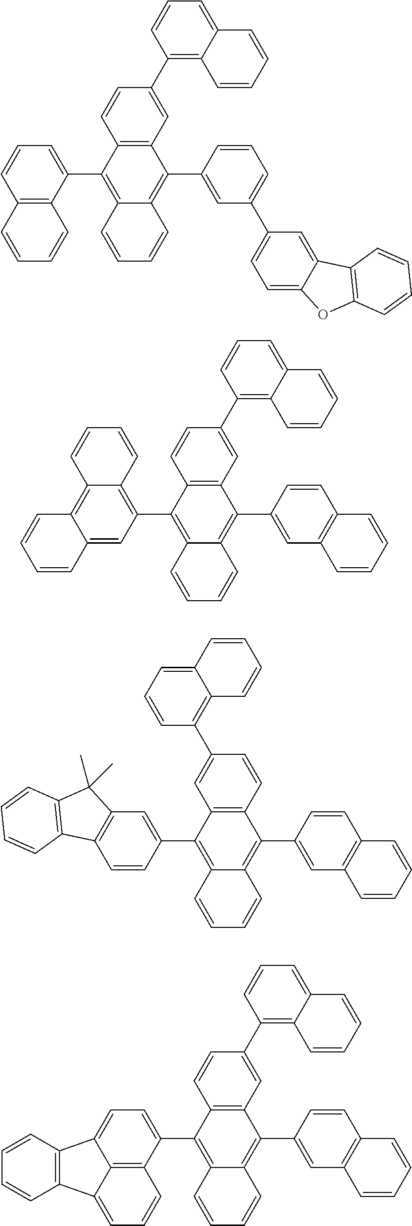

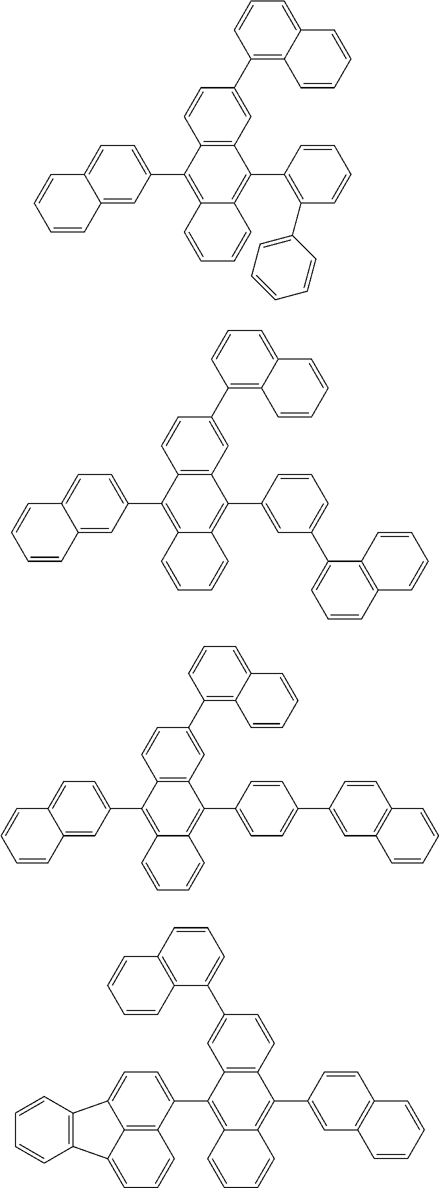

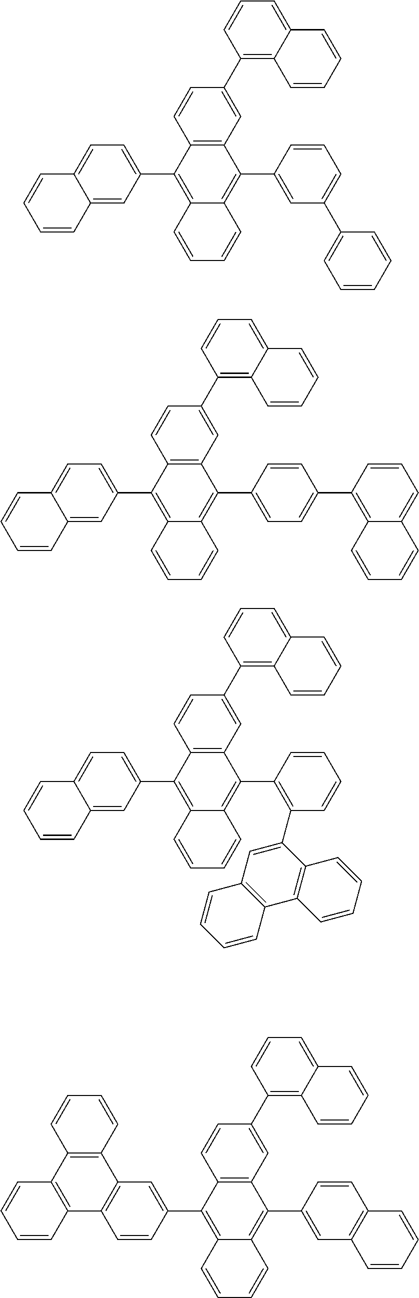

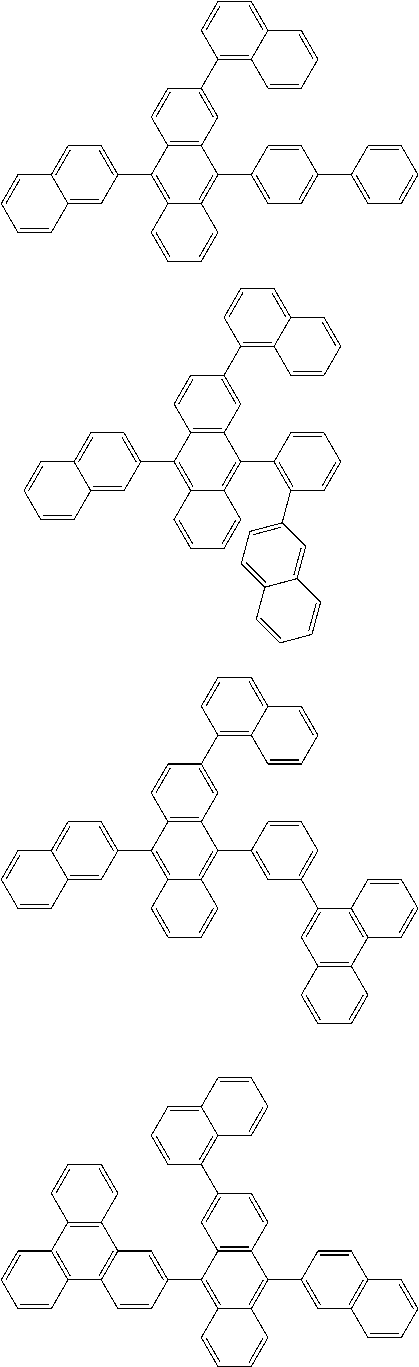

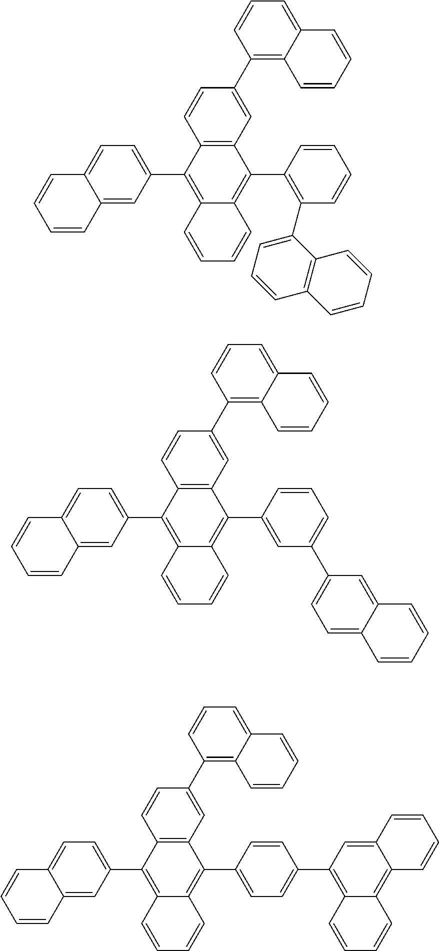

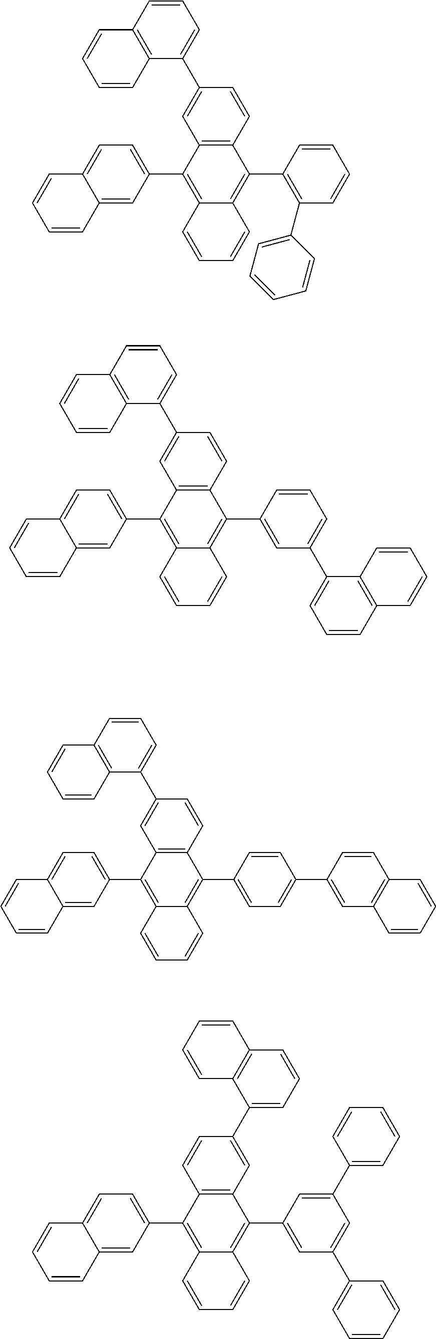

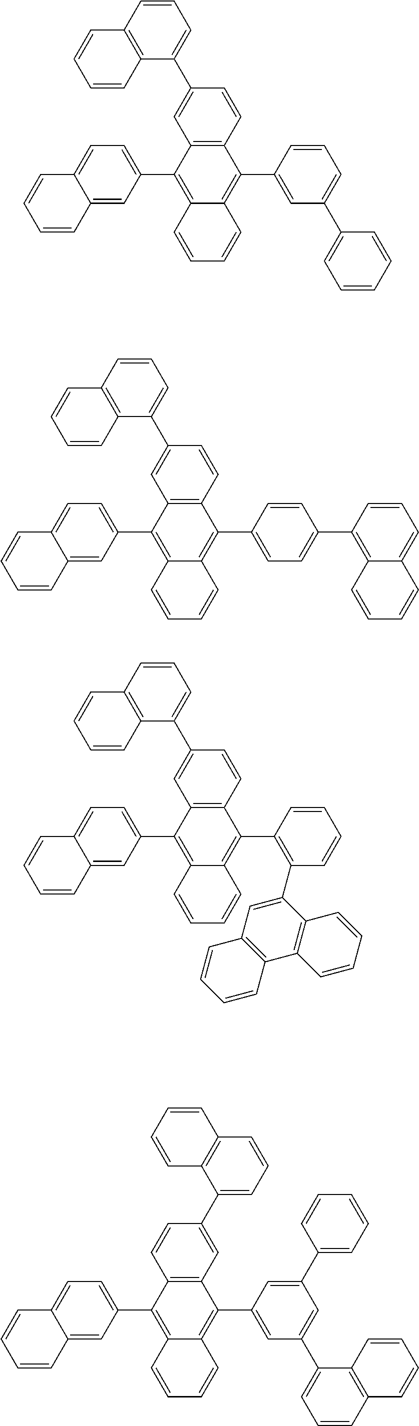

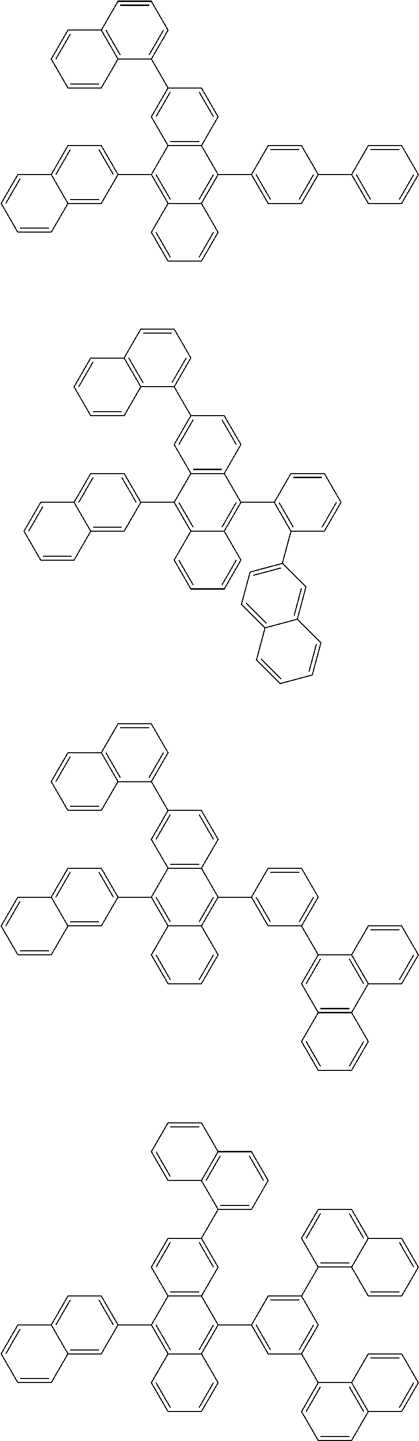

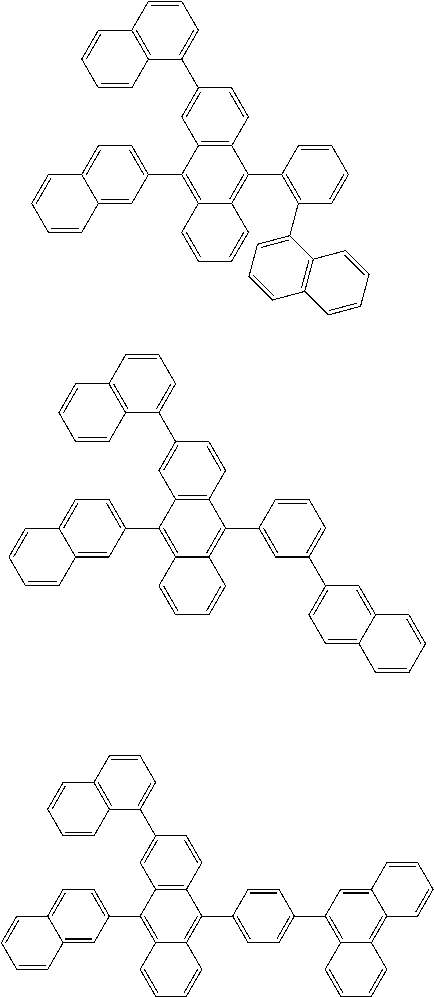

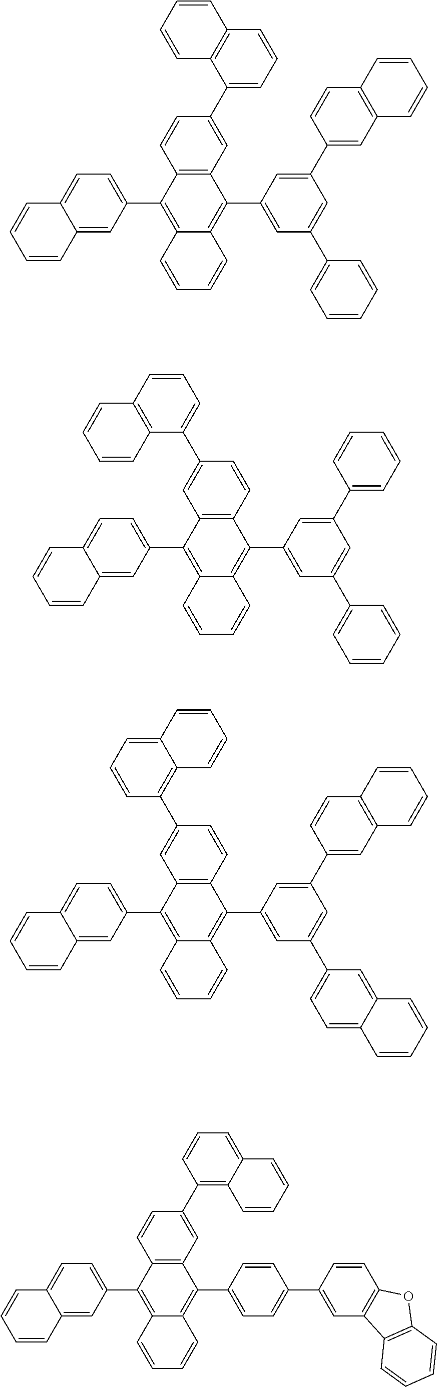

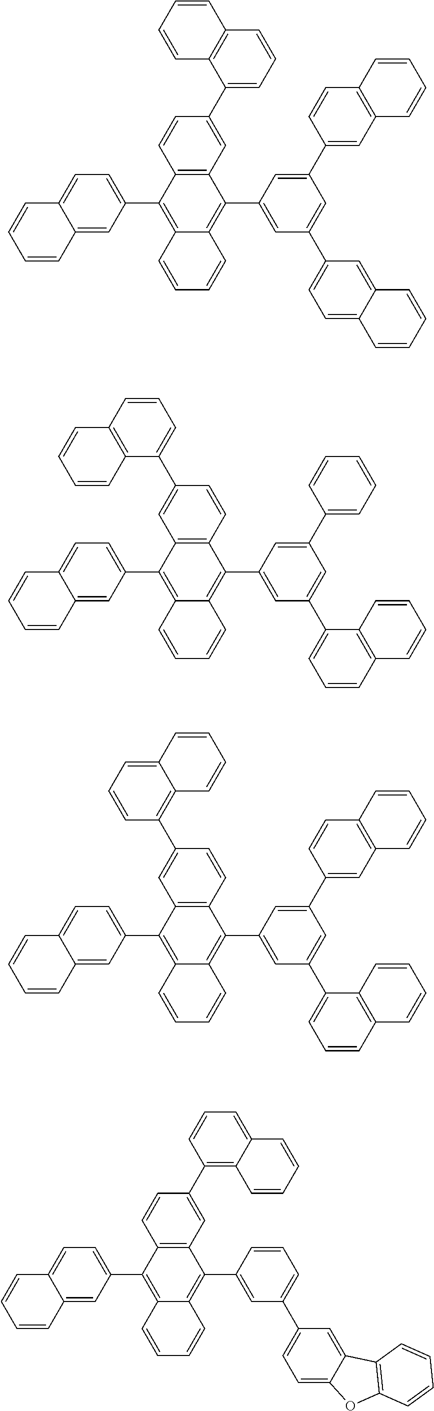

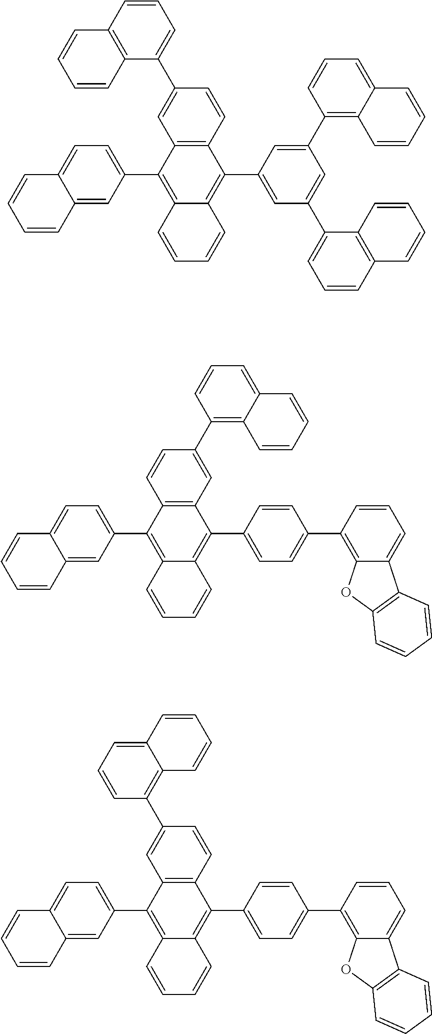

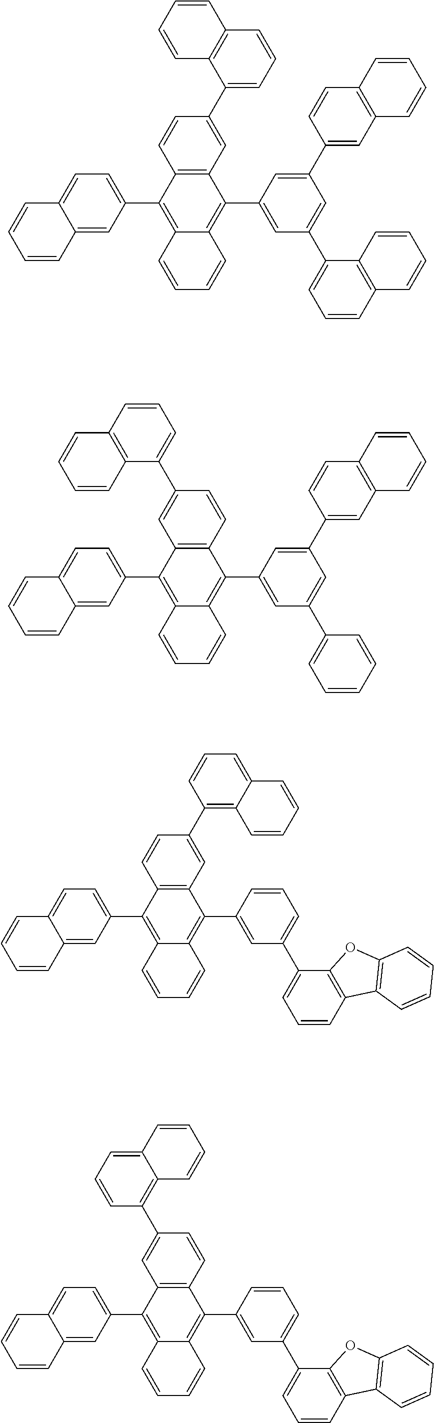

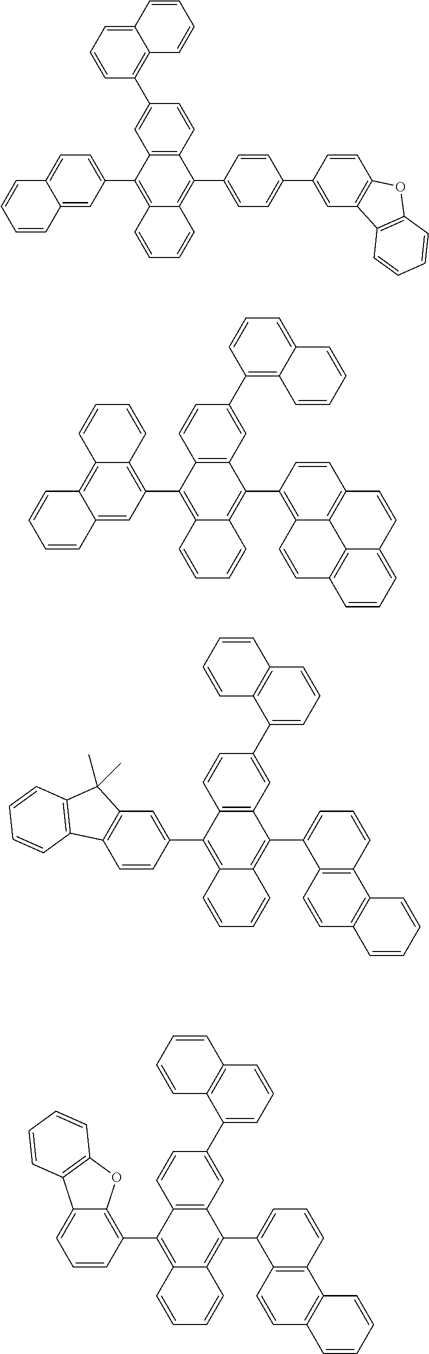

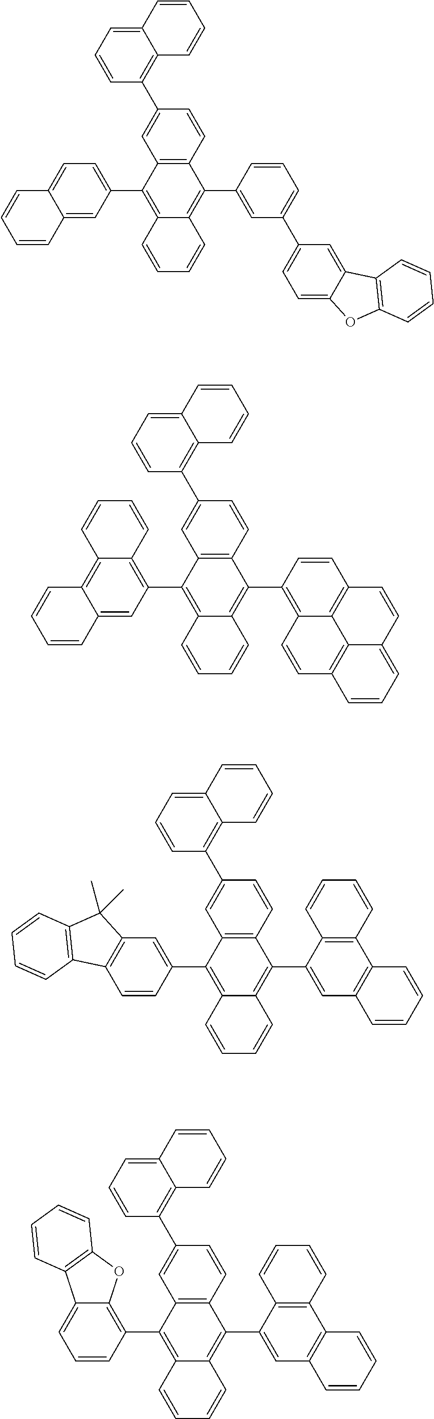

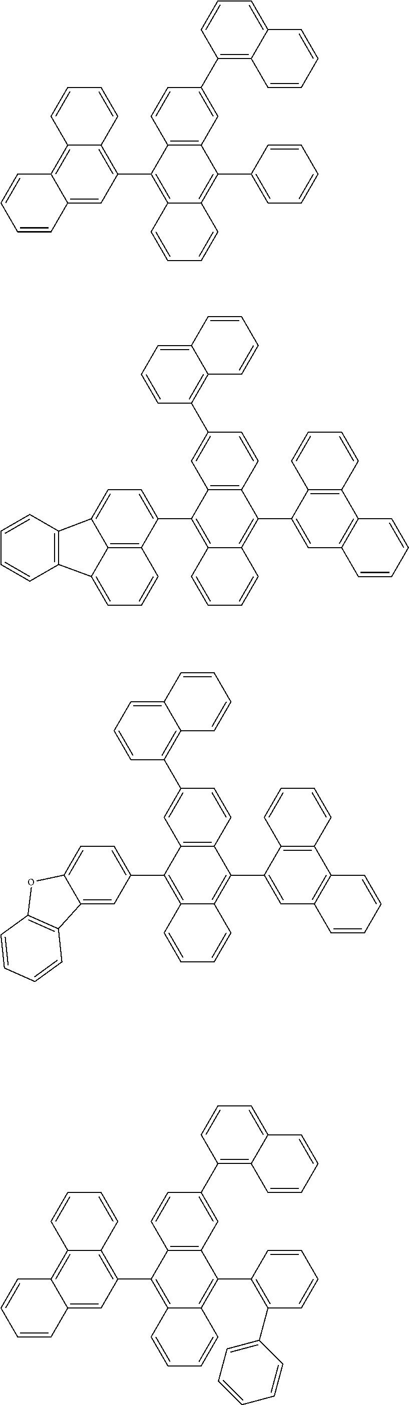

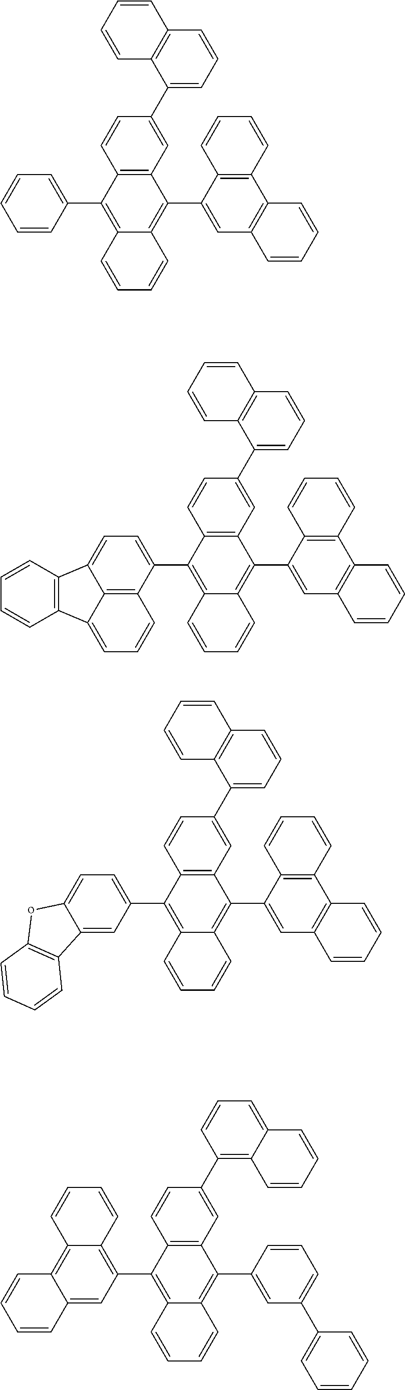

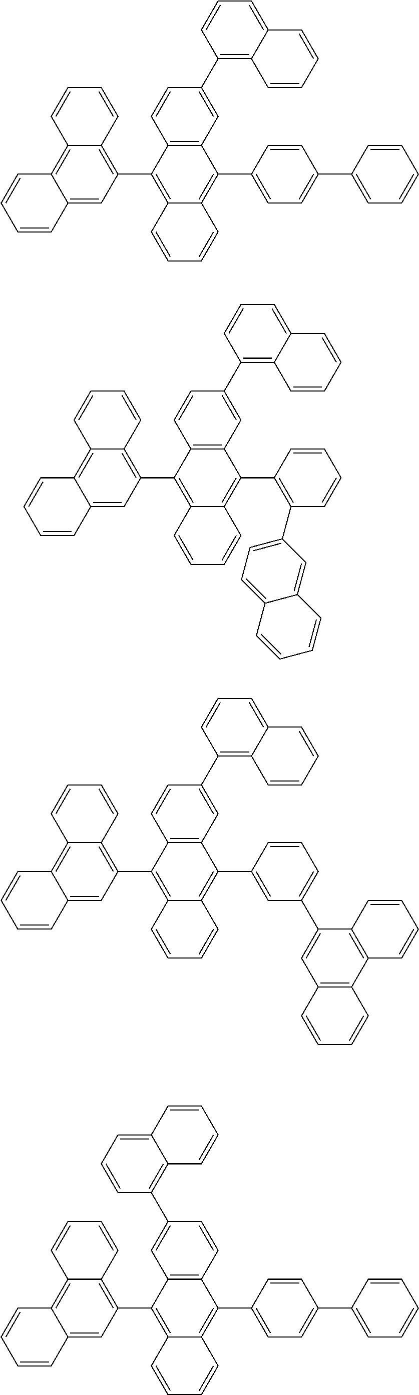

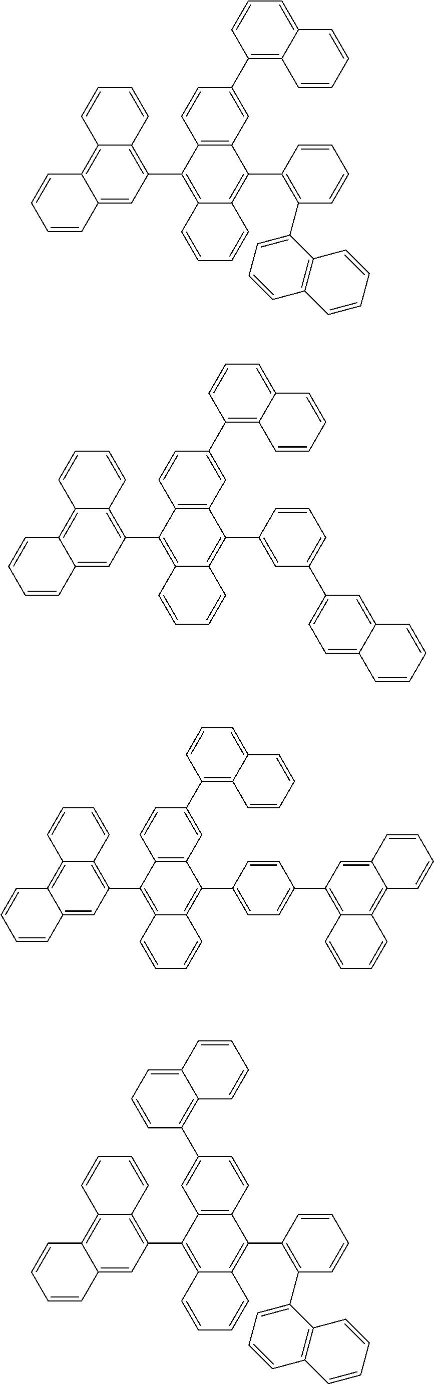

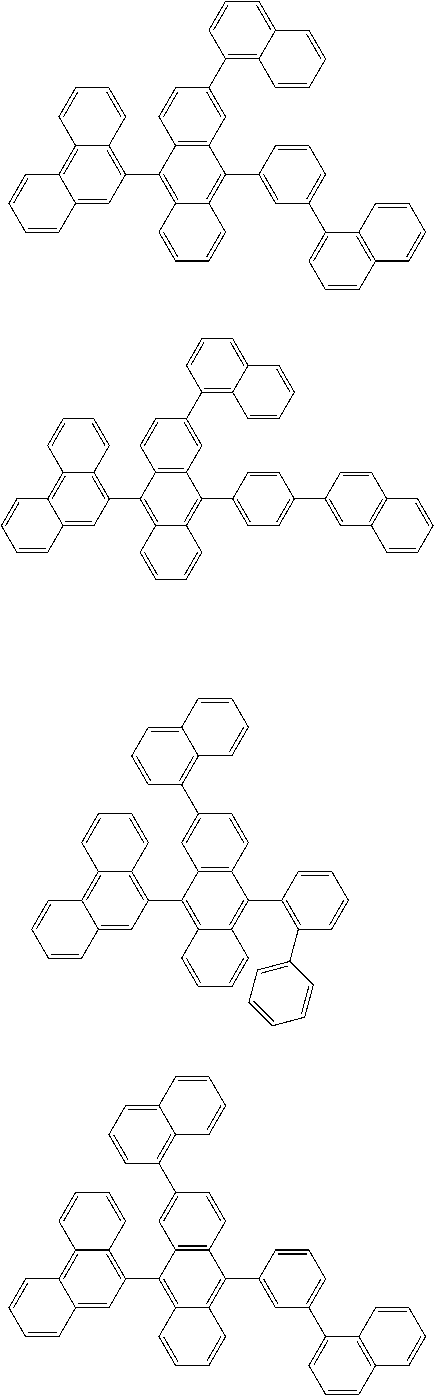

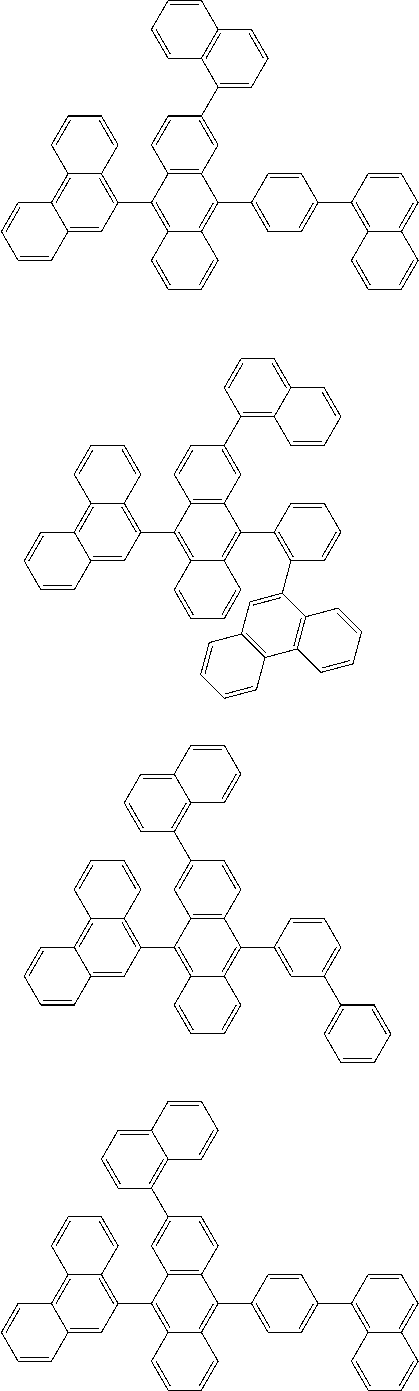

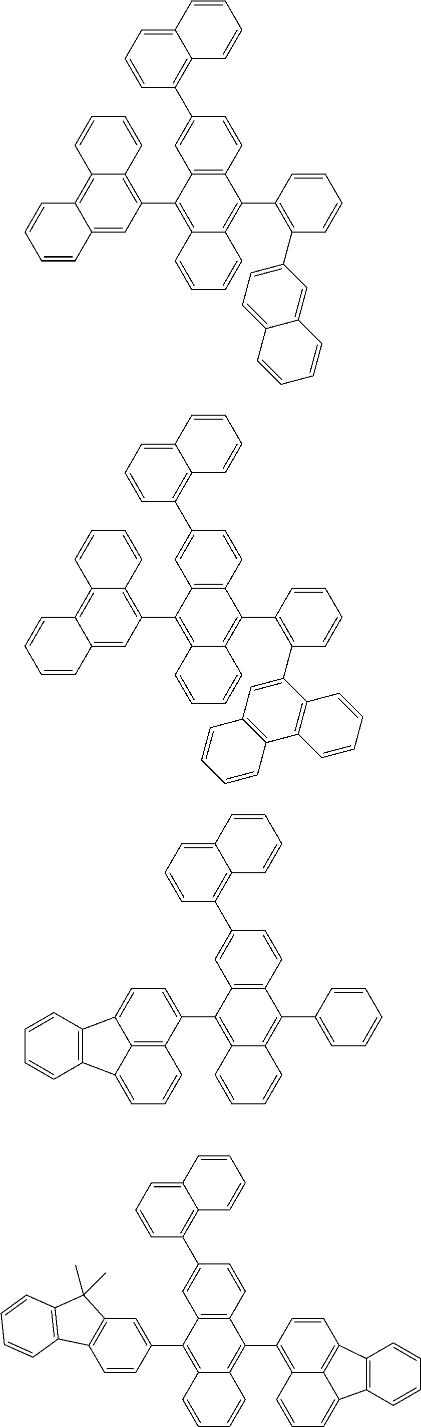

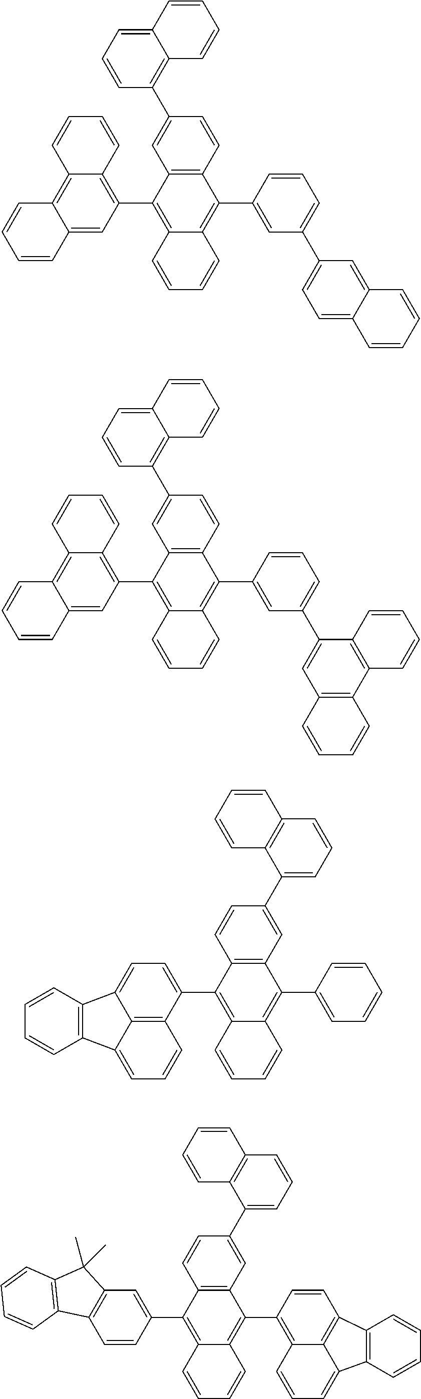

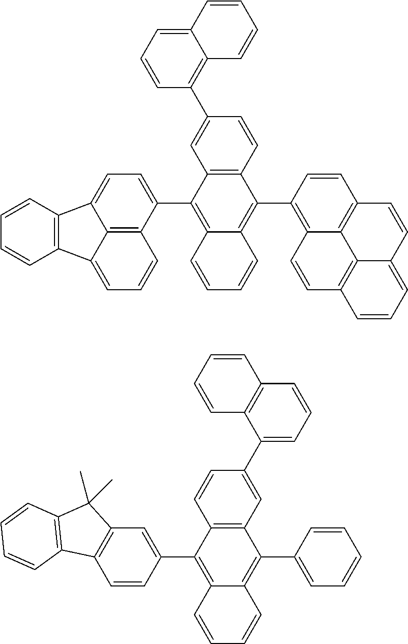

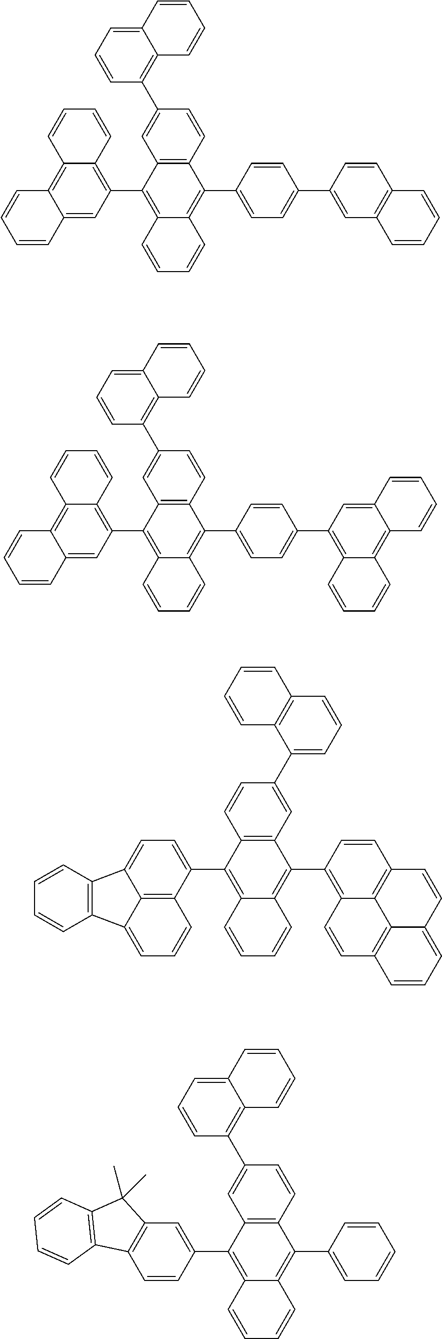

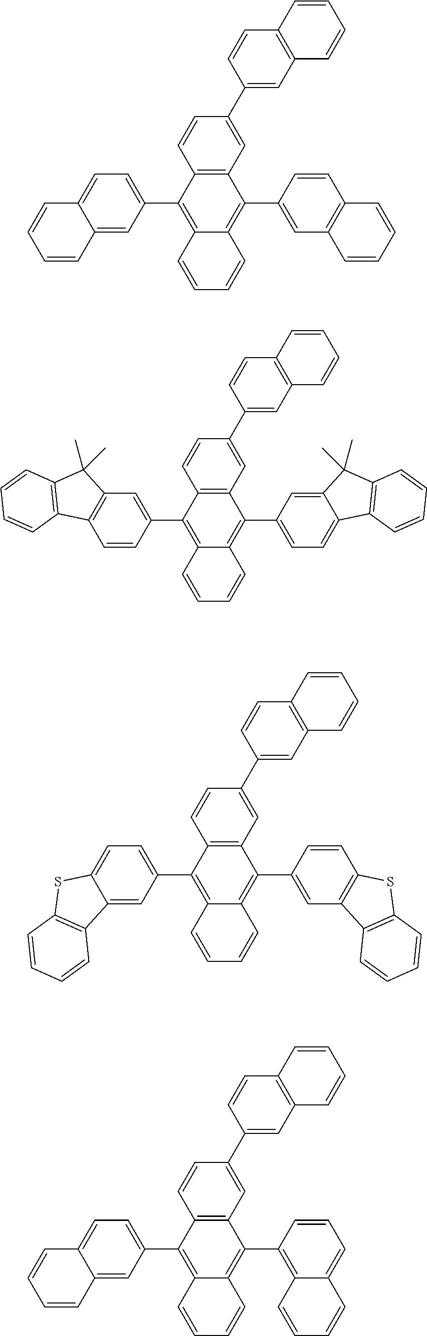

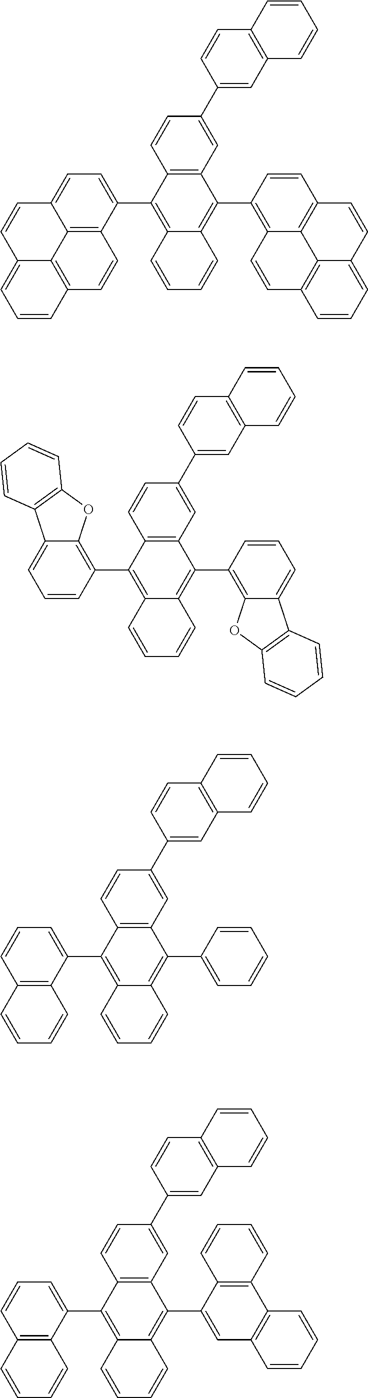

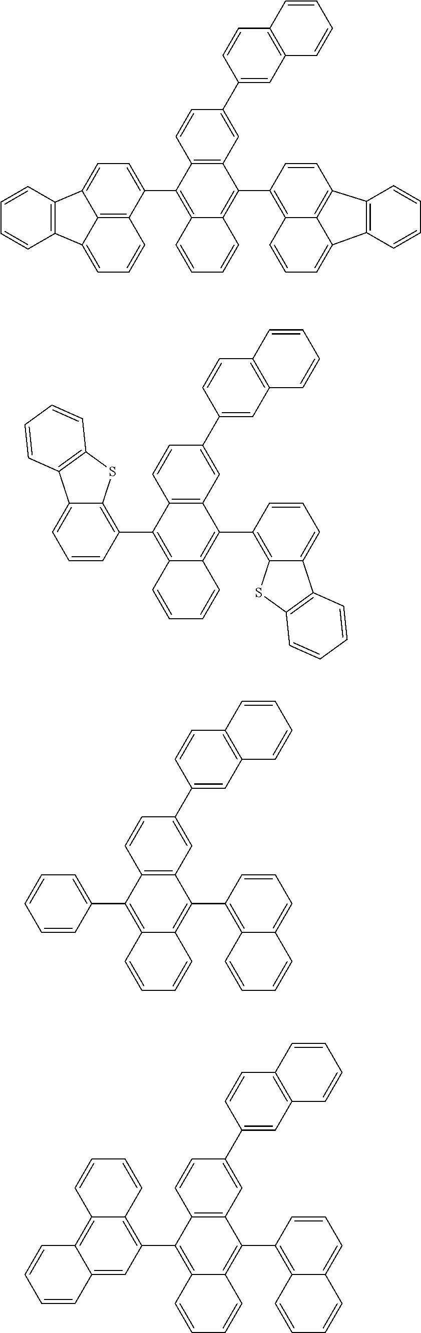

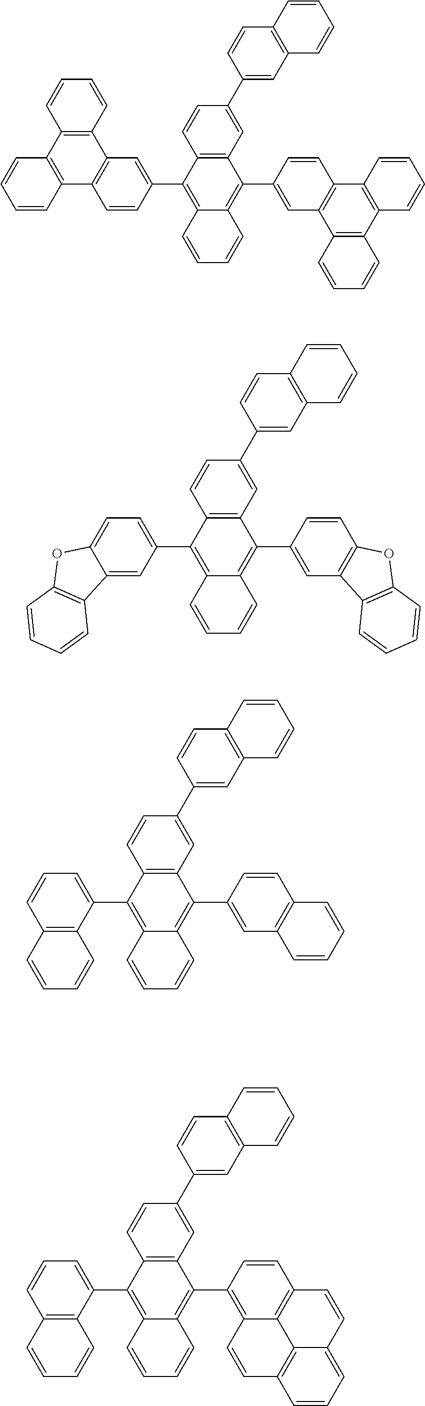

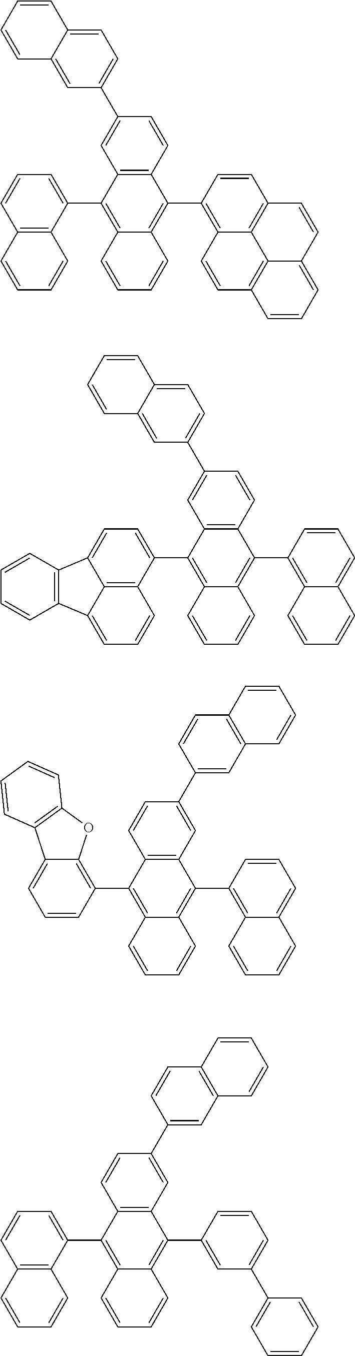

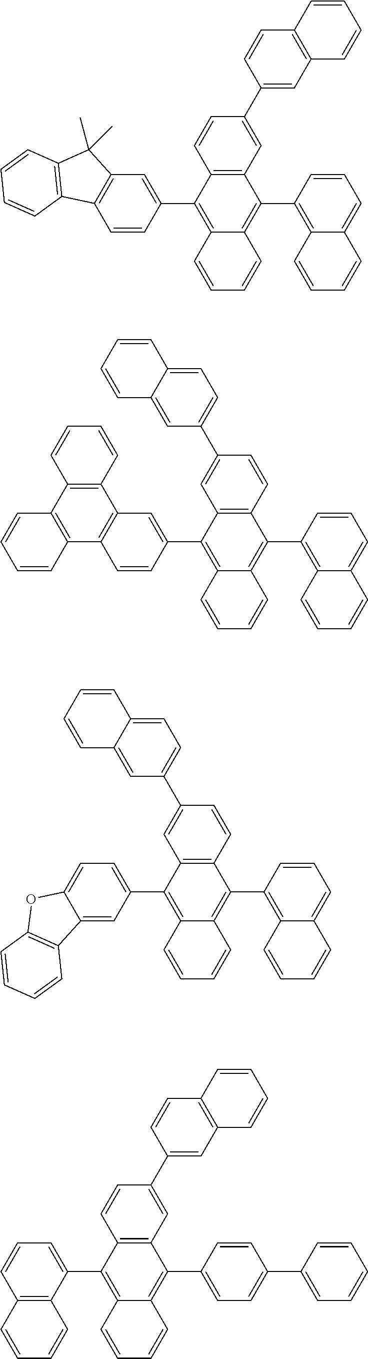

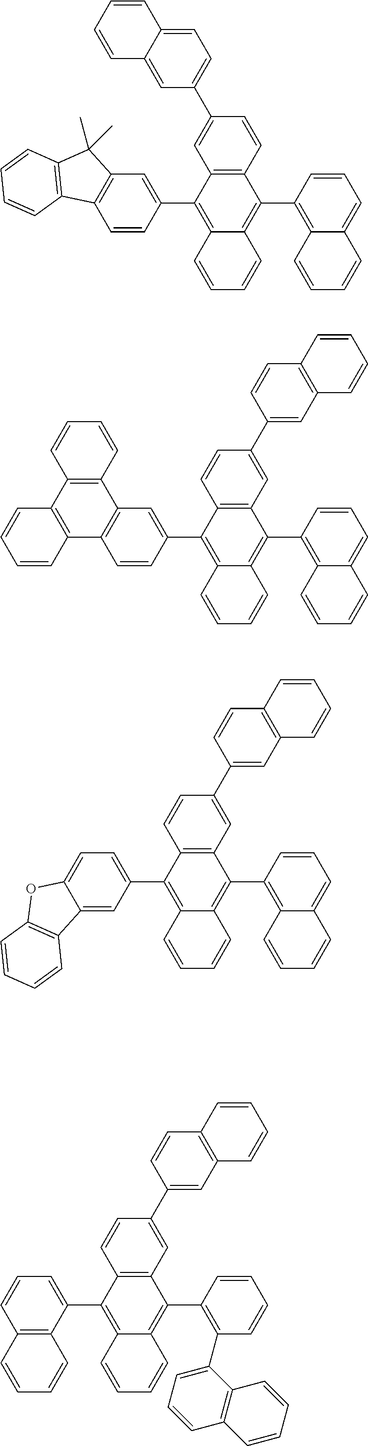

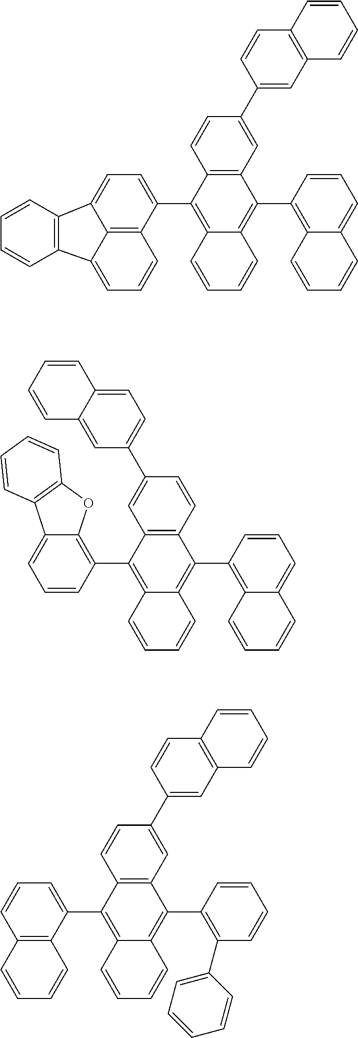

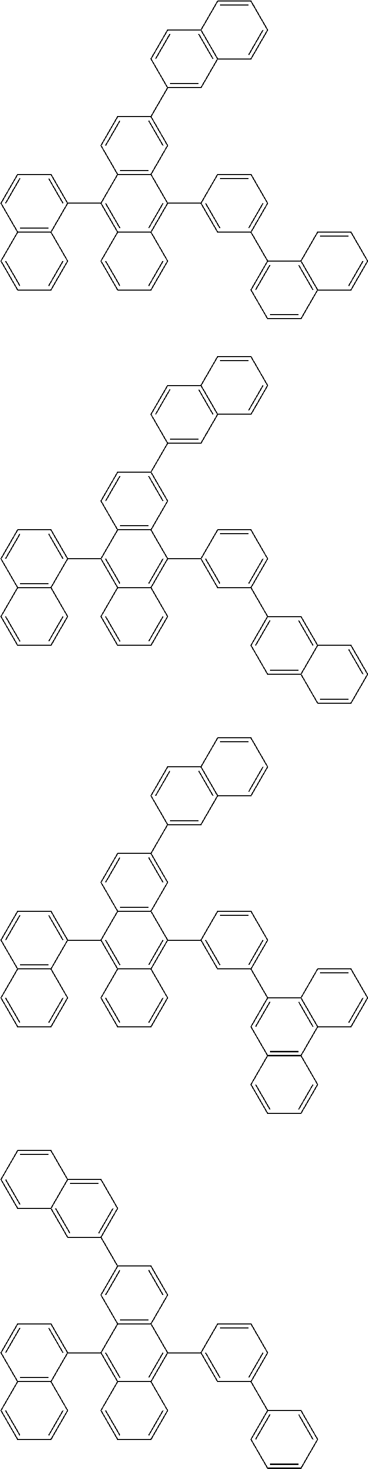

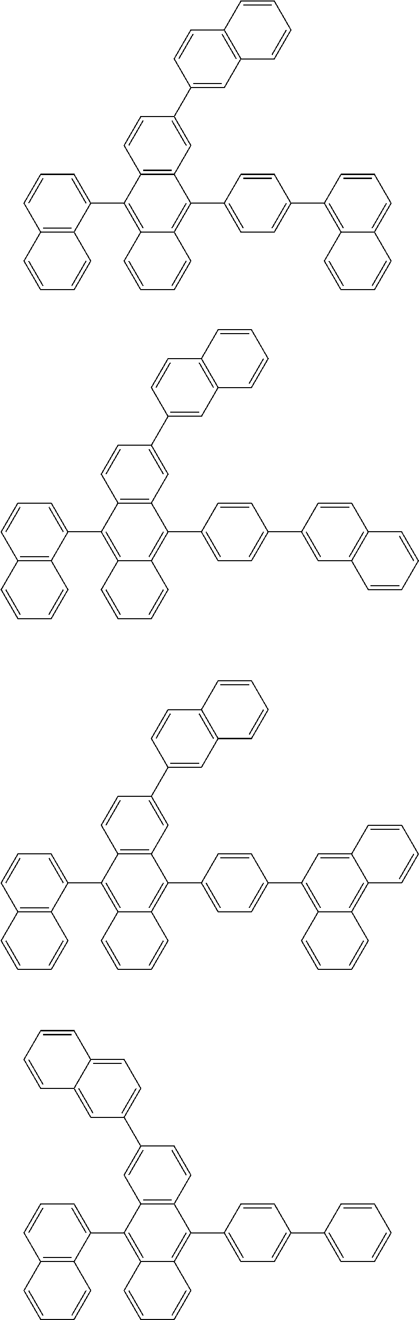

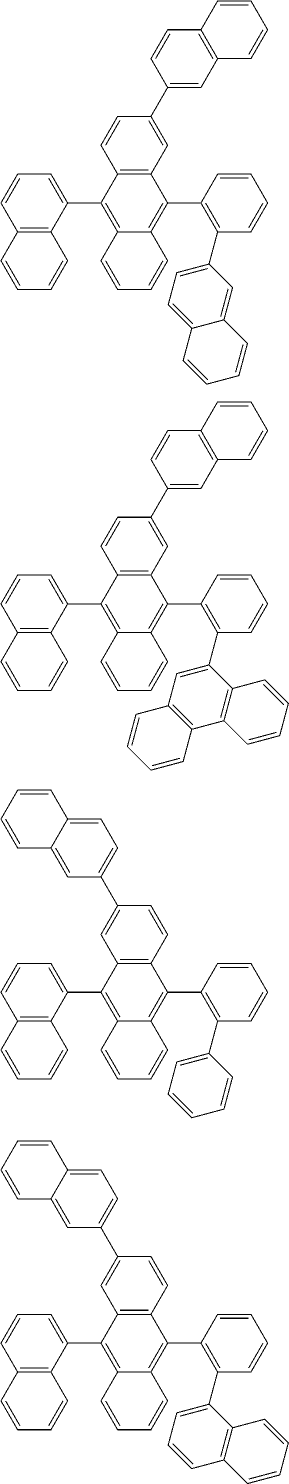

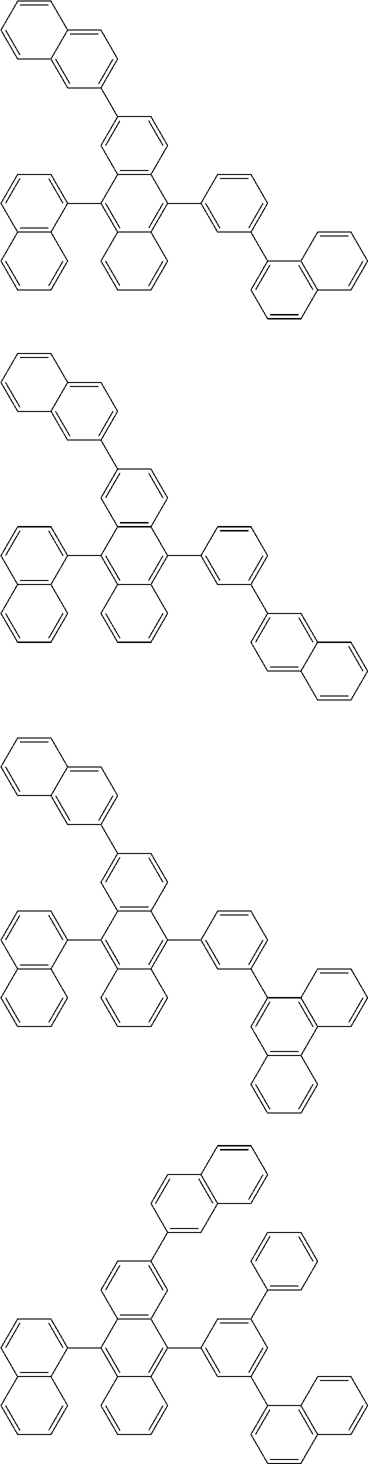

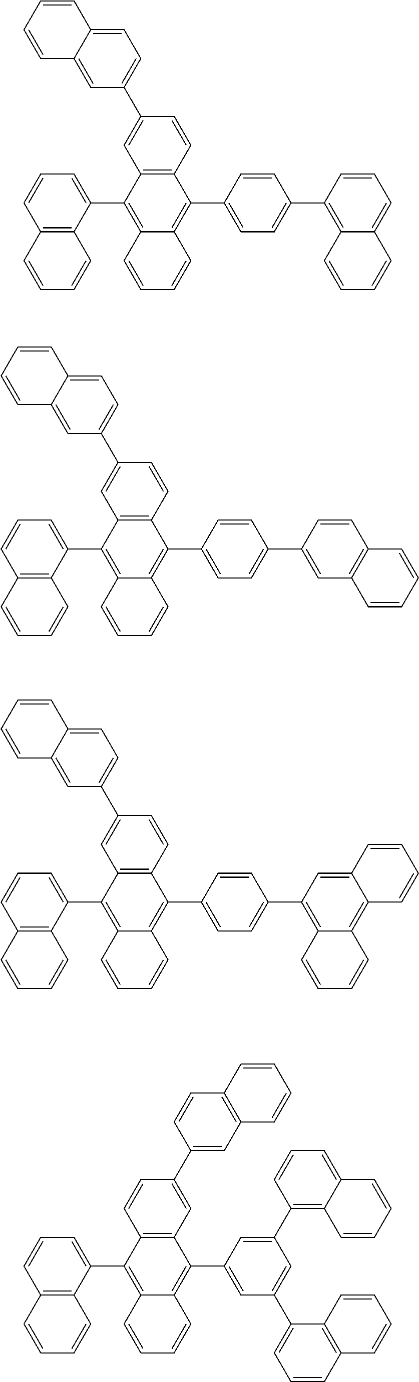

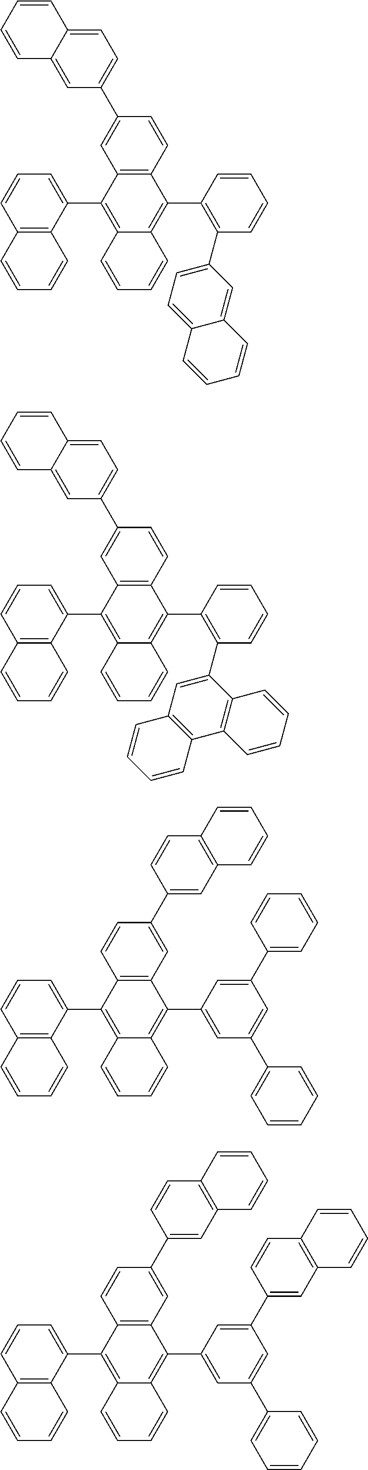

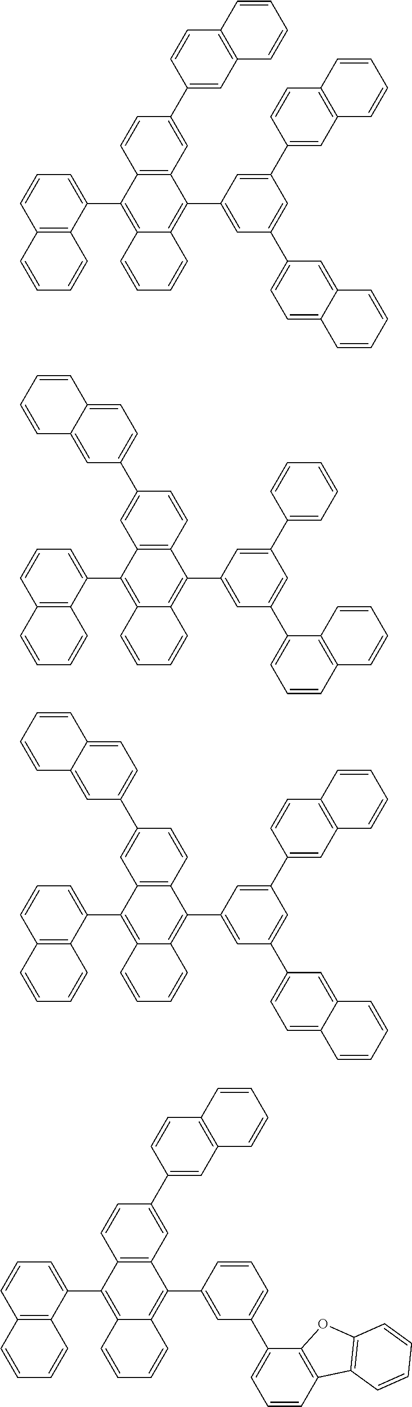

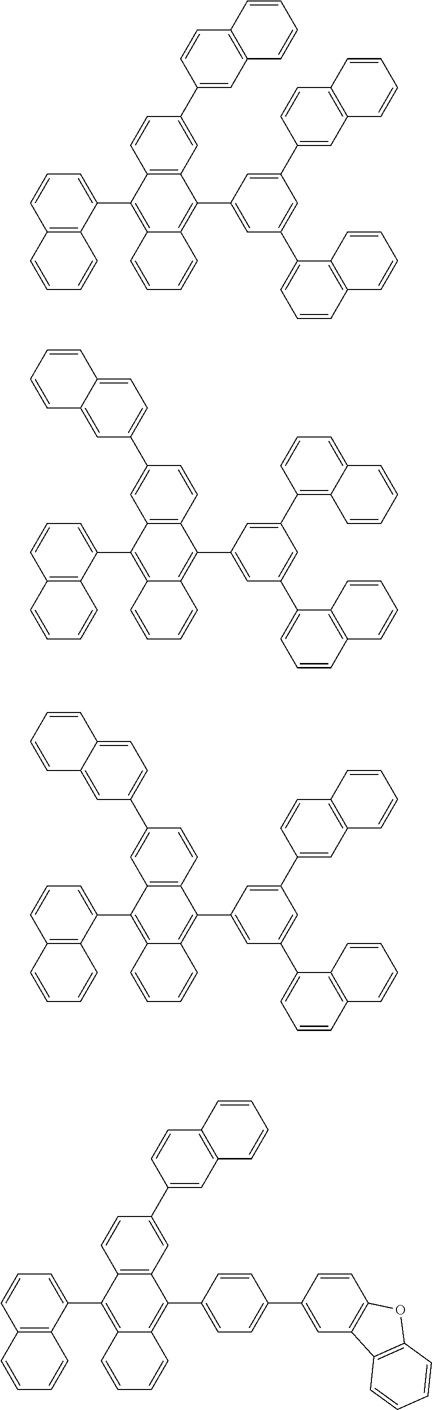

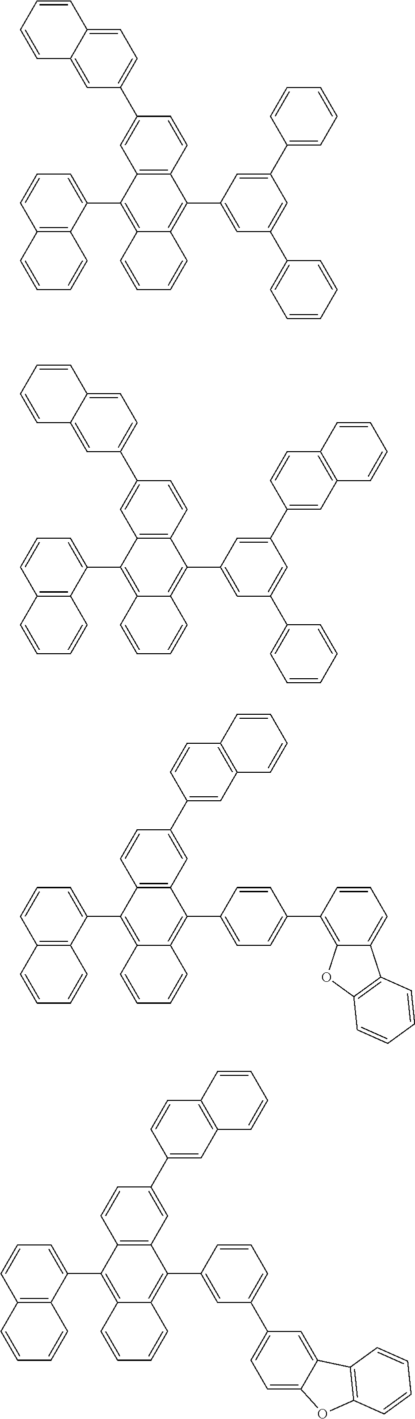

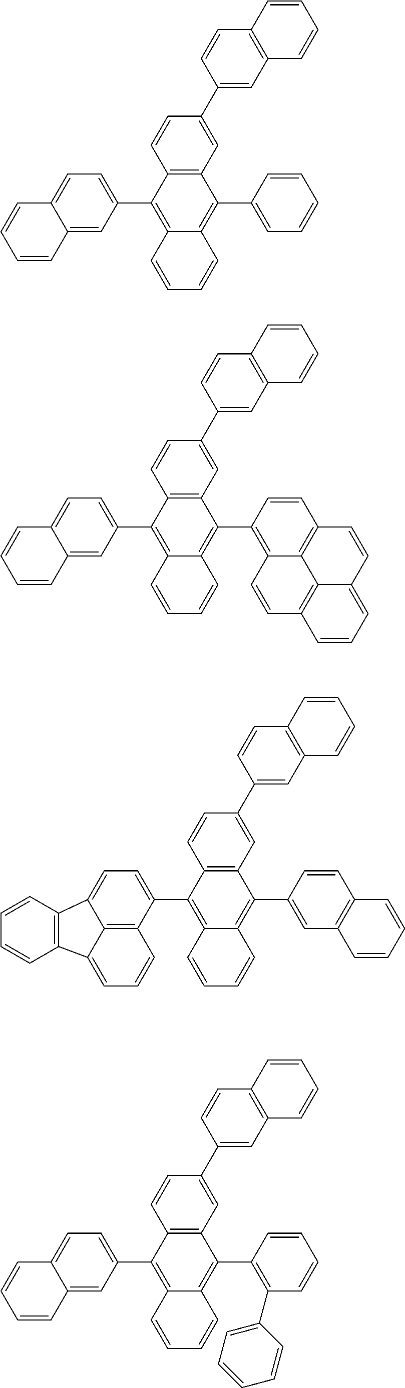

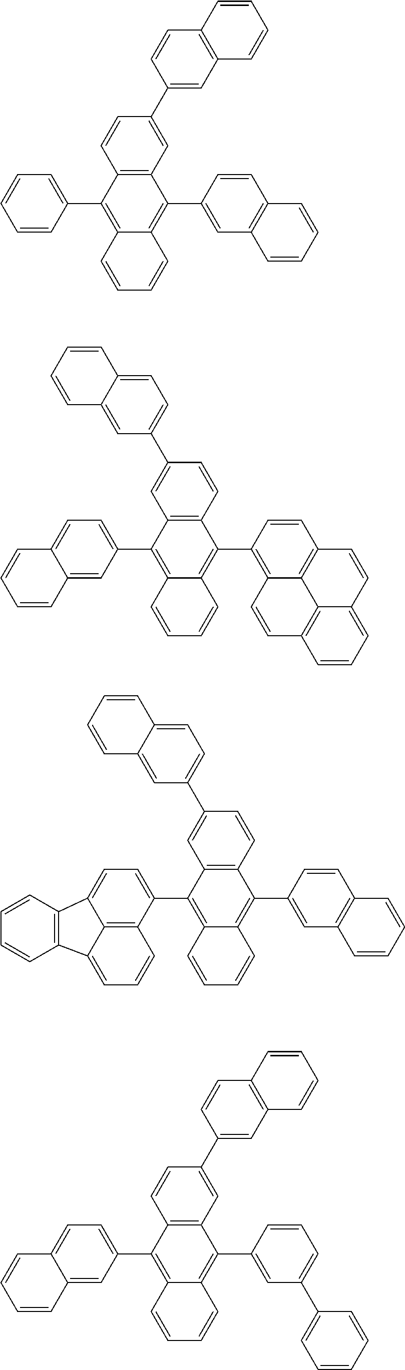

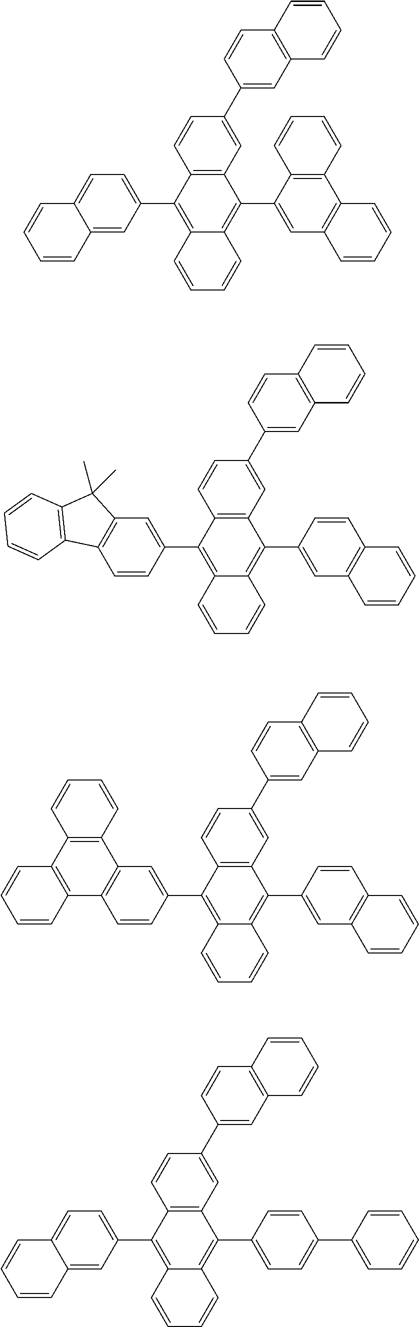

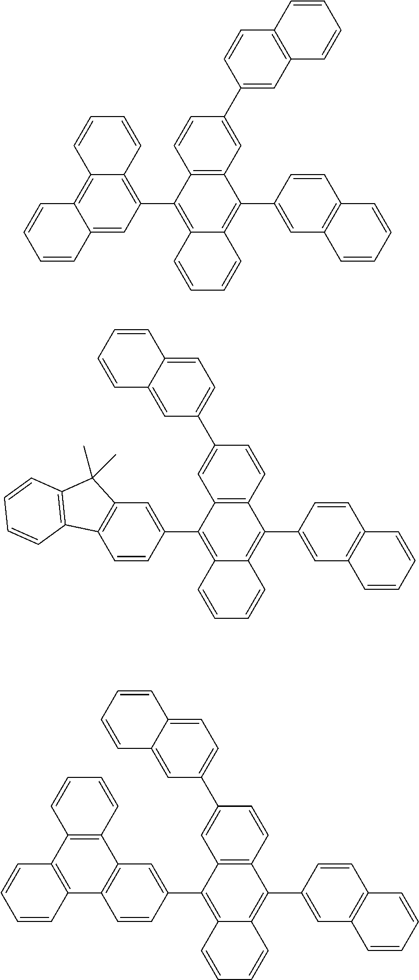

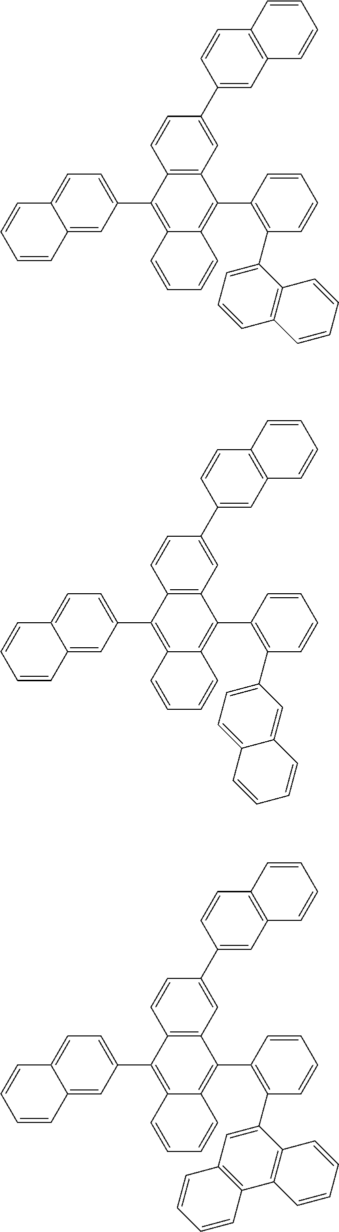

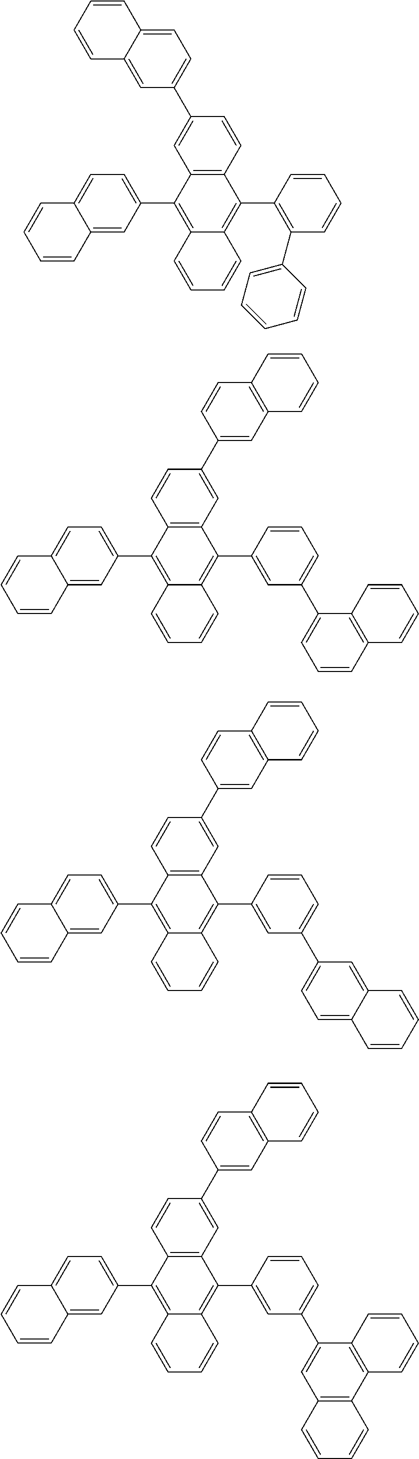

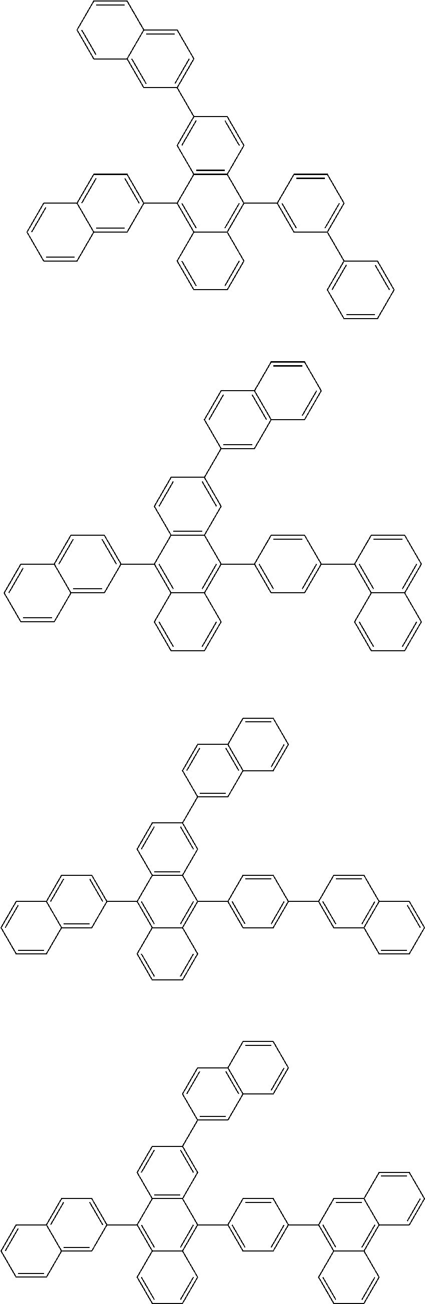

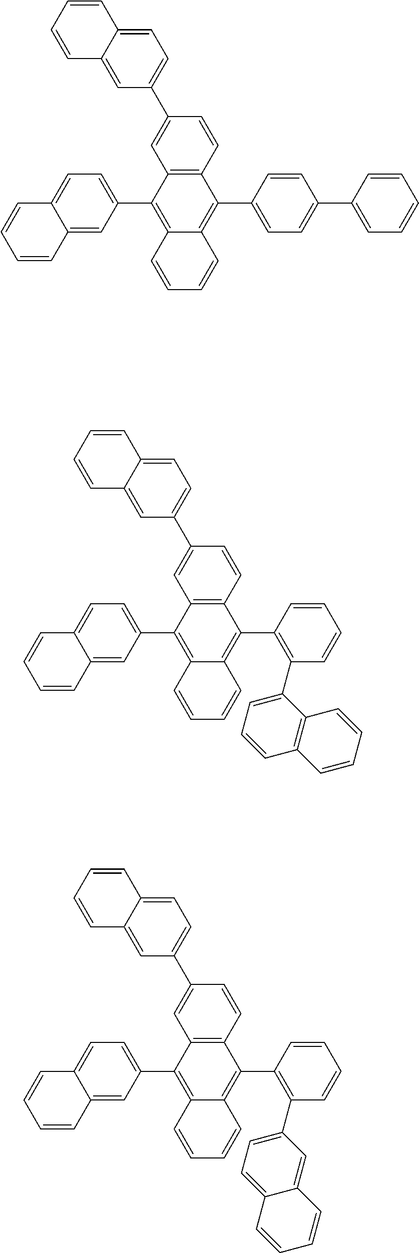

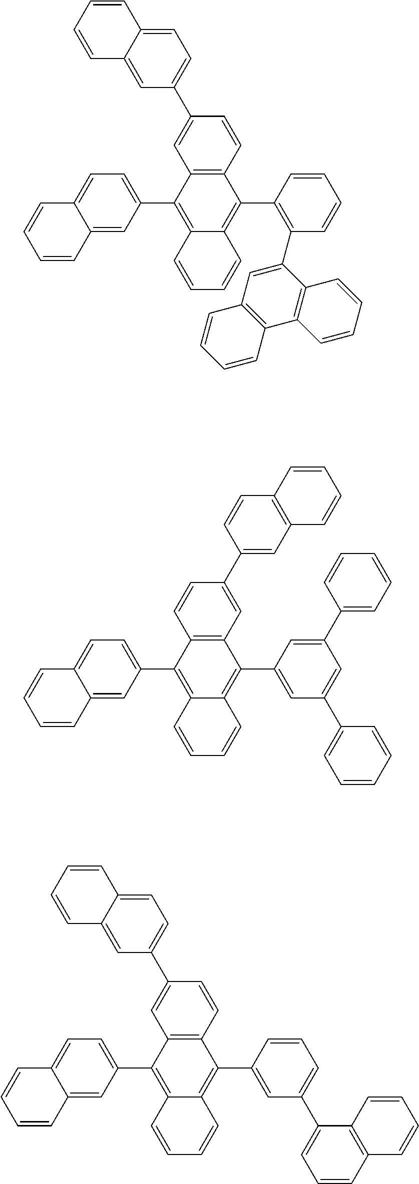

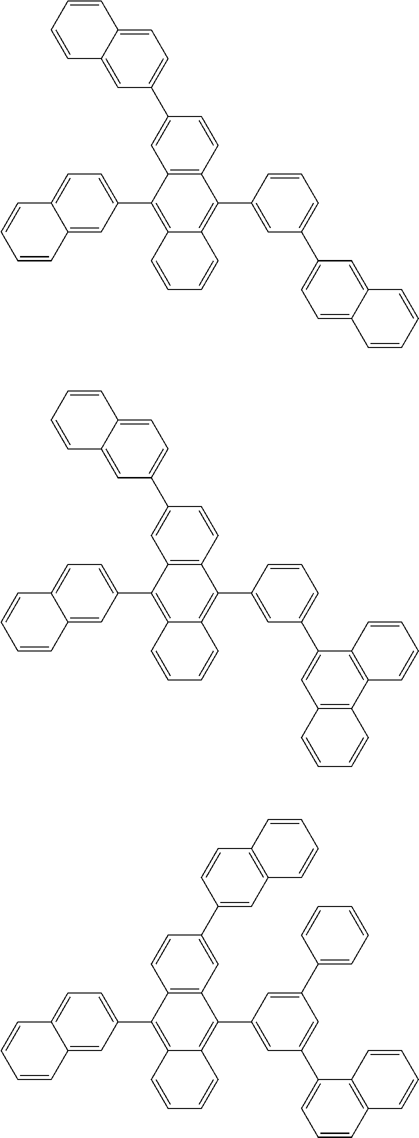

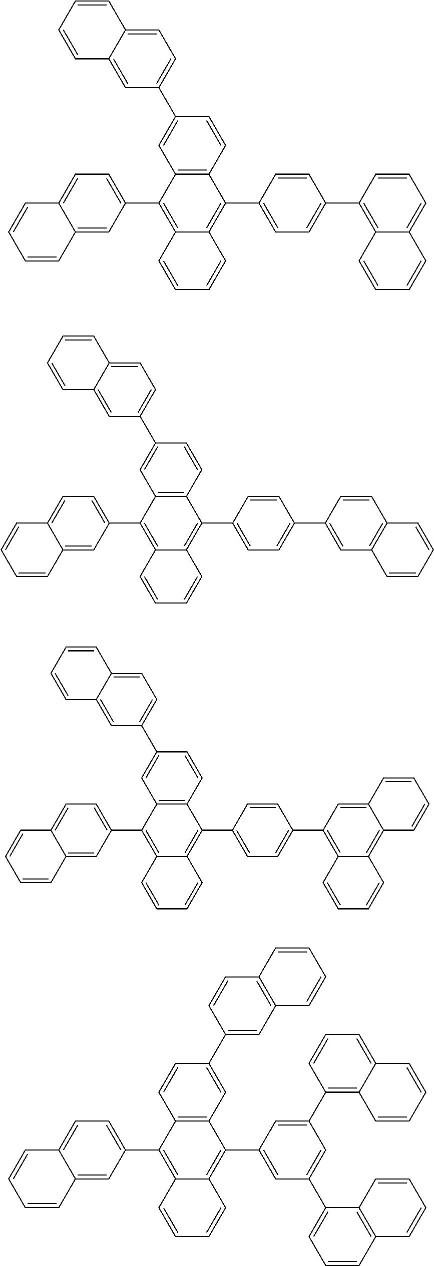

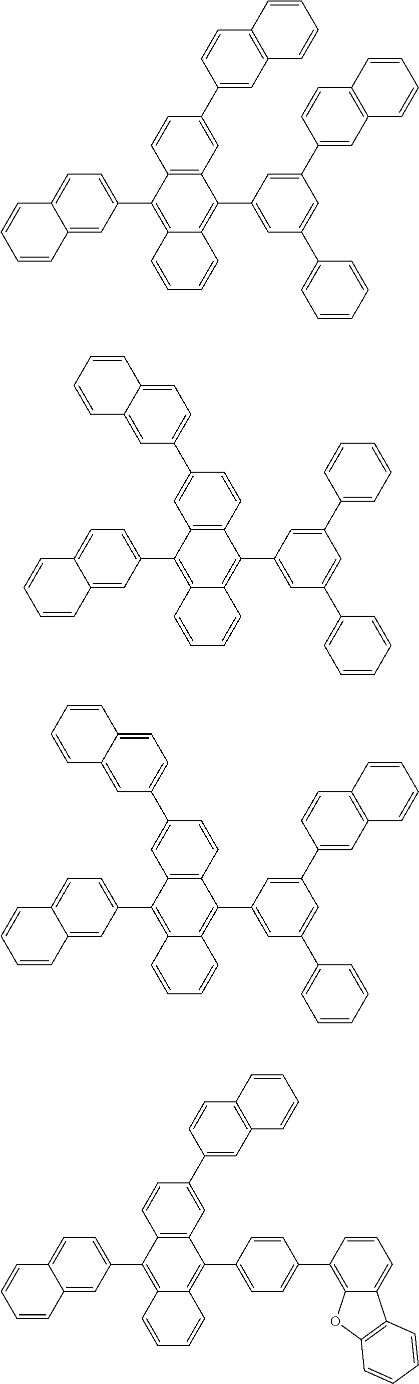

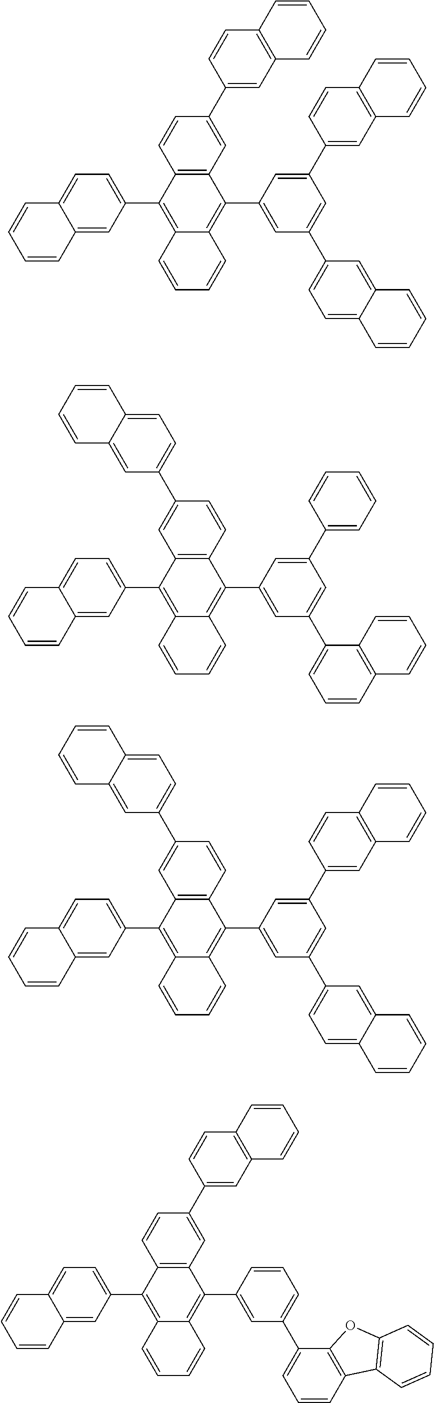

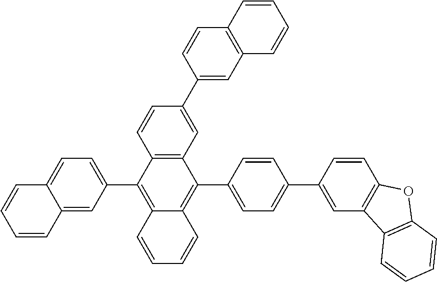 [SMILES: c1ccc2cc(-c3ccc4c(-c5ccc6ccccc6c5)c5ccccc5c(-c5ccc(-c6ccc7oc8ccccc8c7c6)cc5)c4c3)ccc2c1]

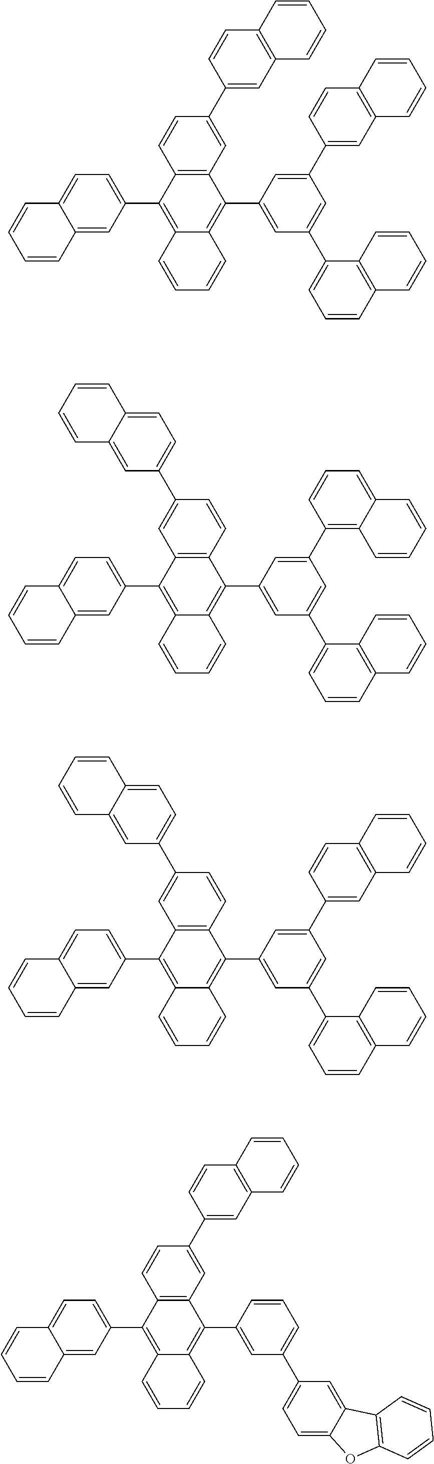

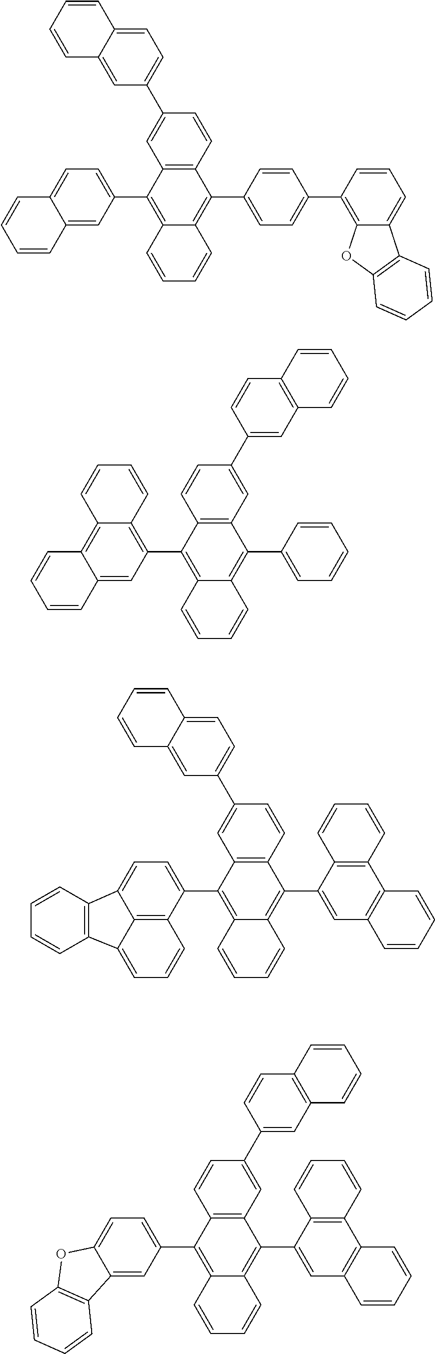

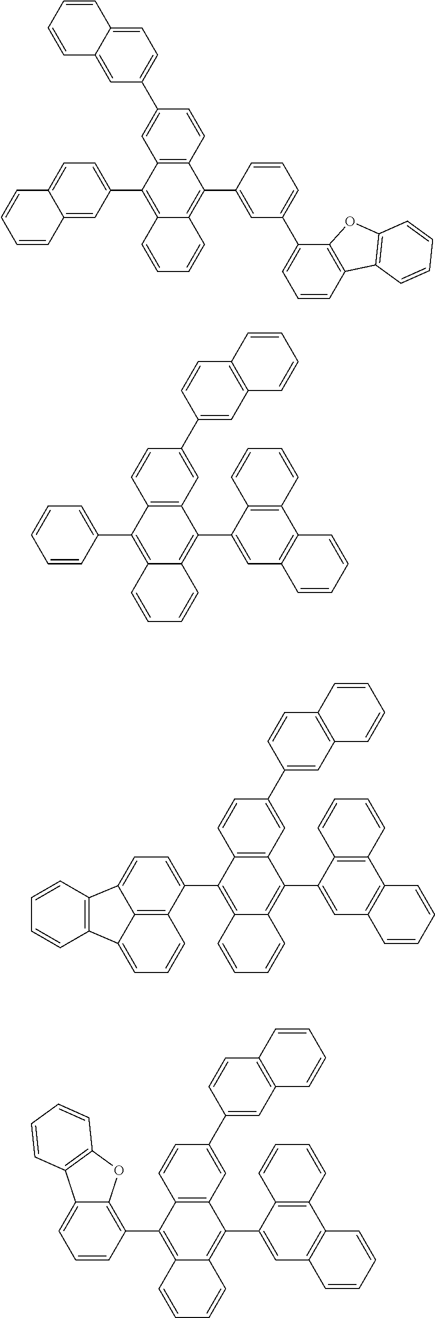

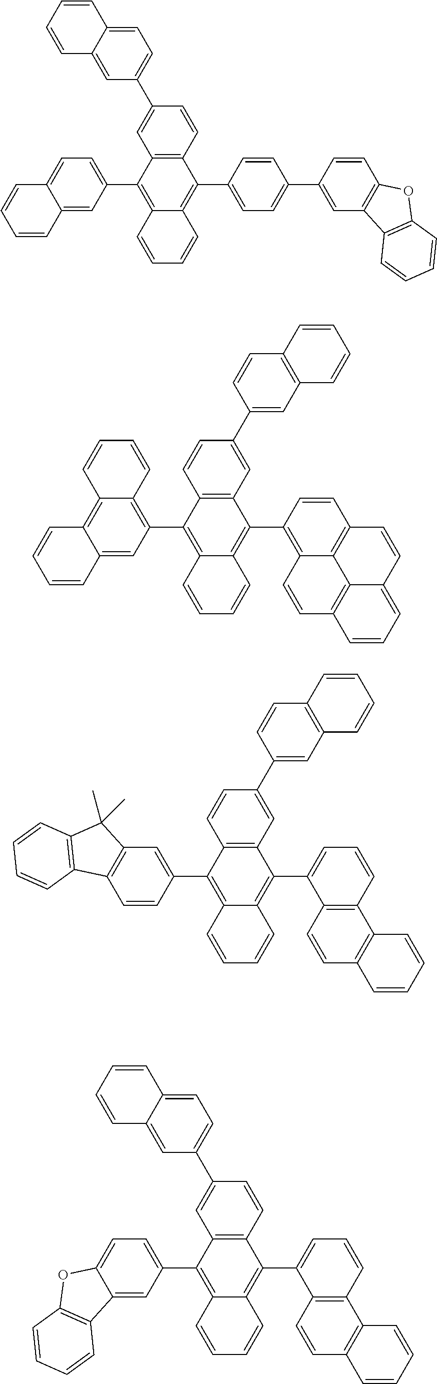

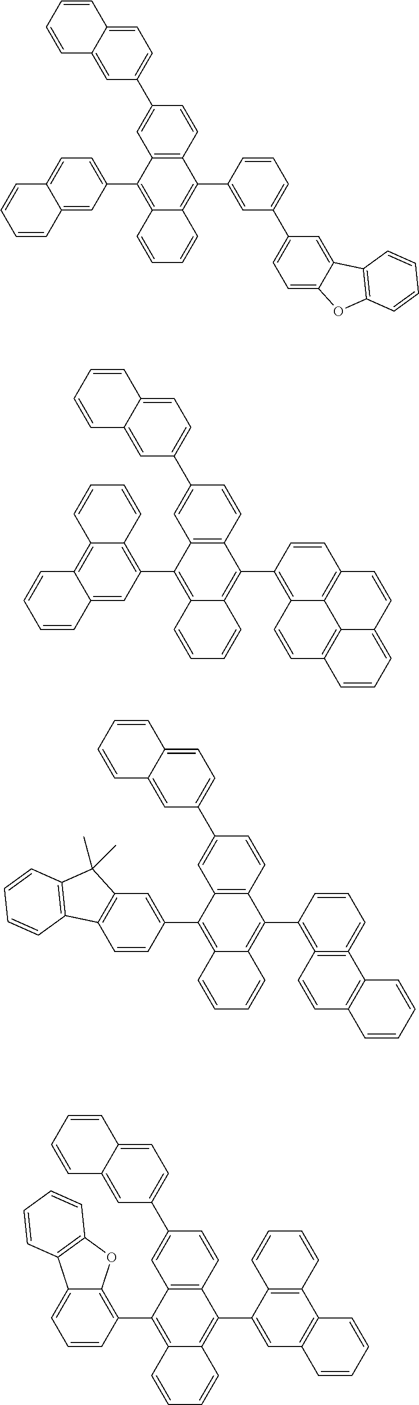

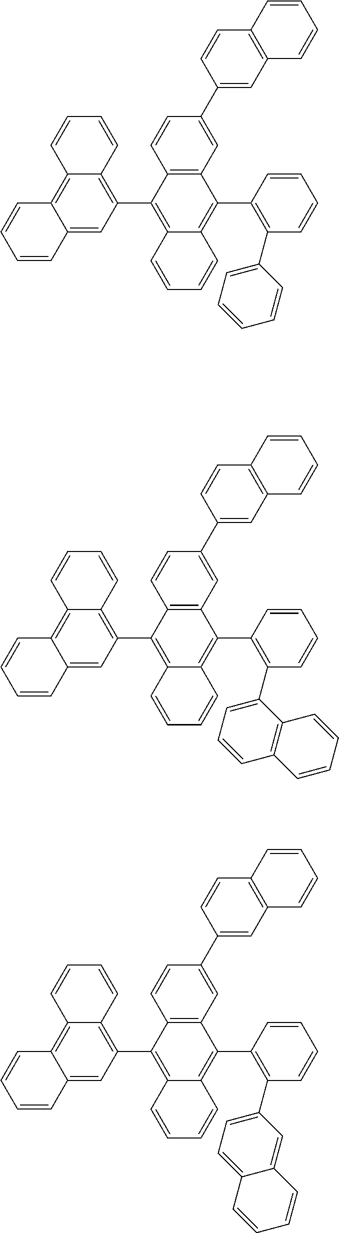

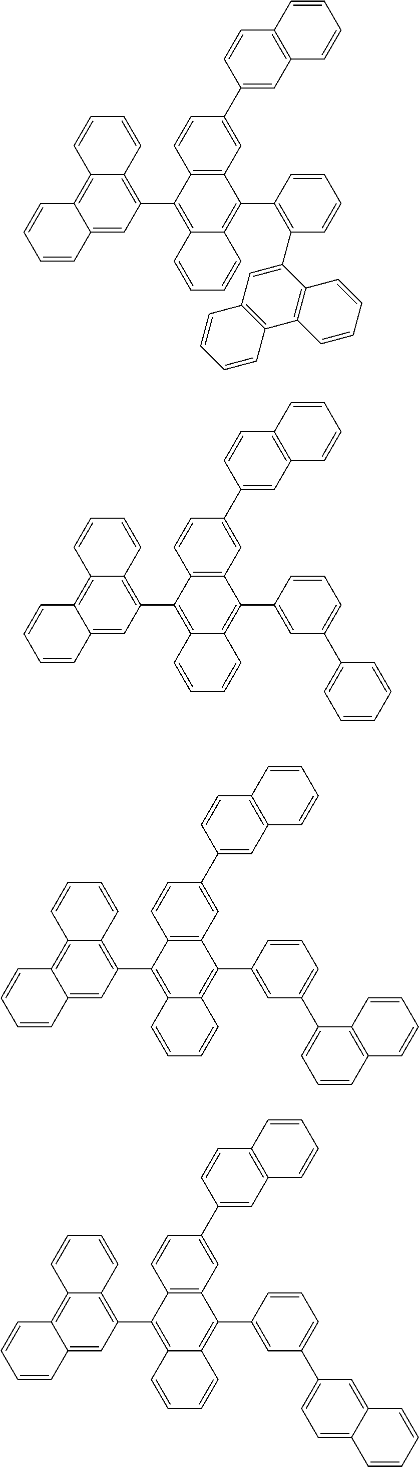

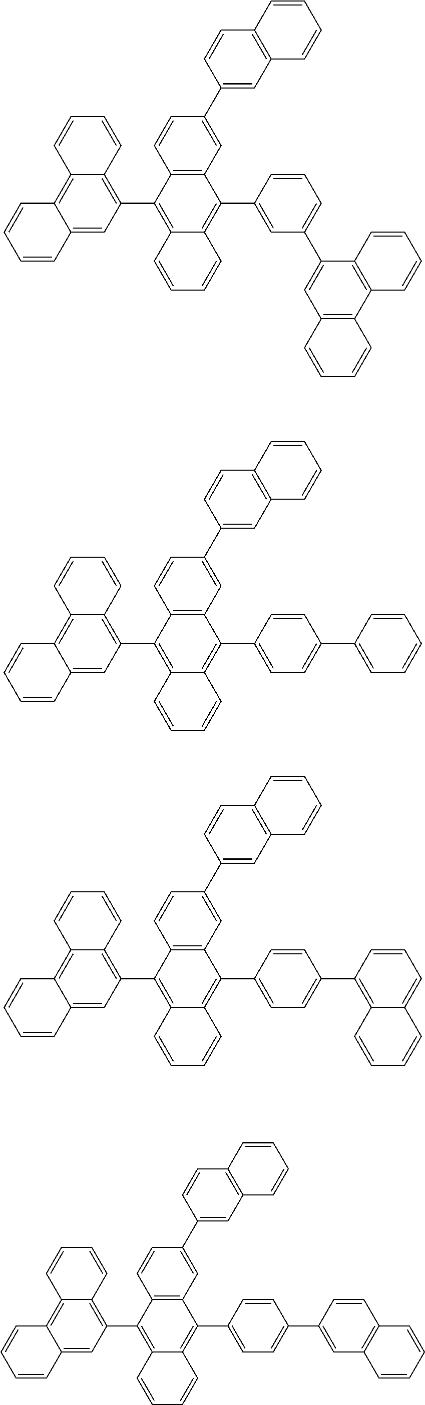

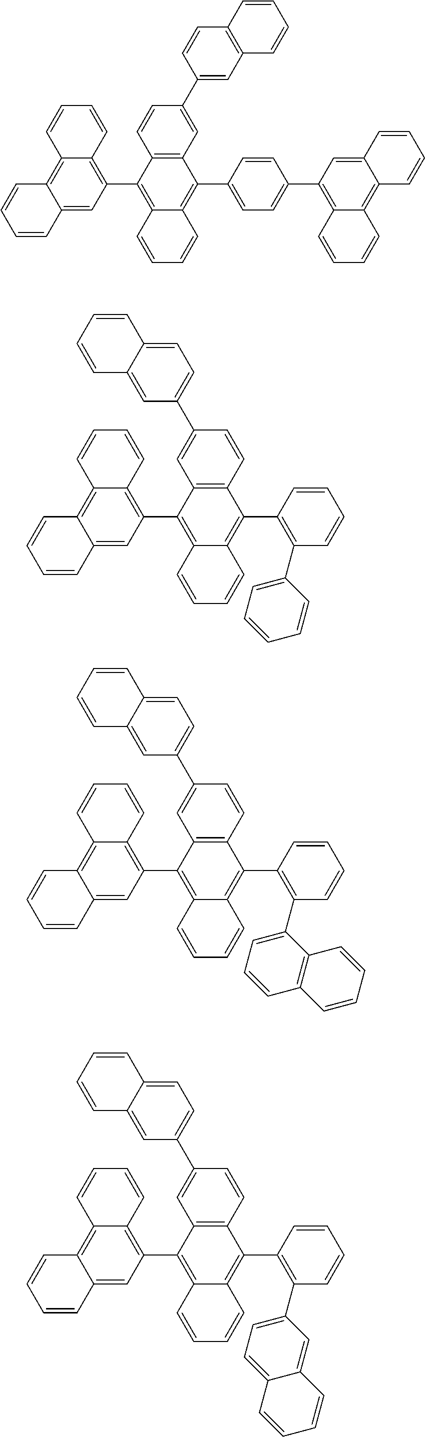

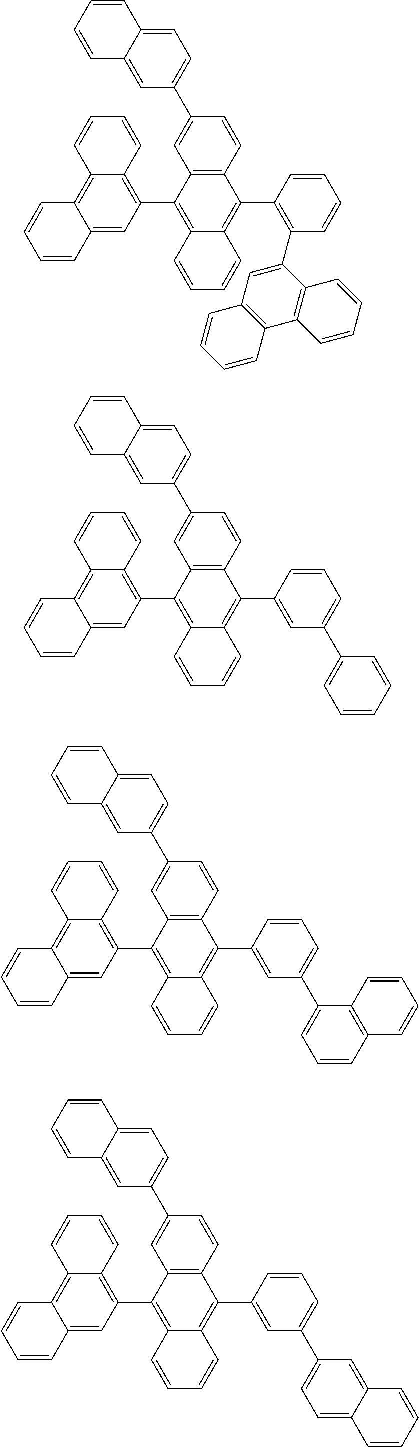

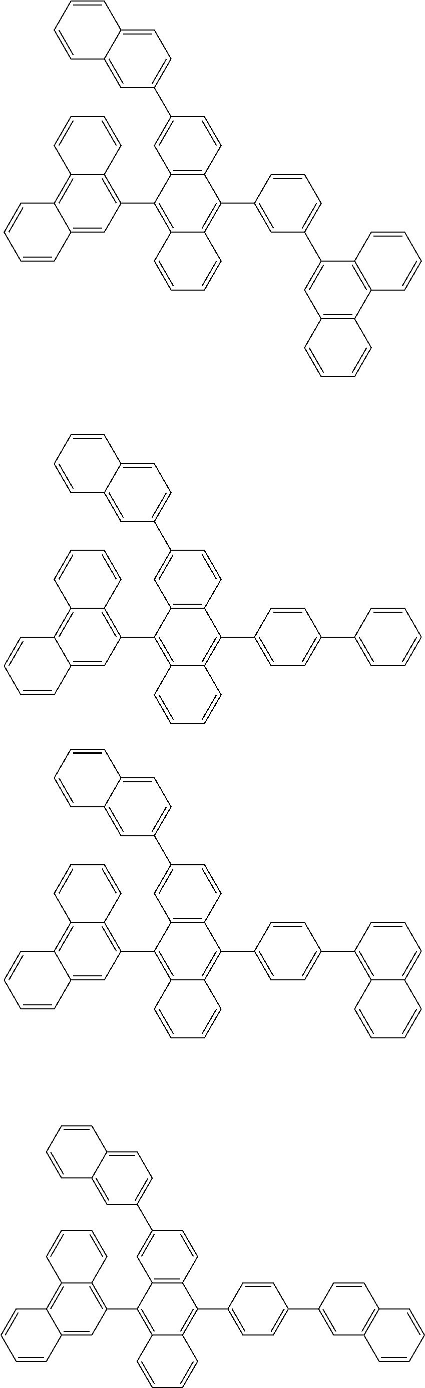

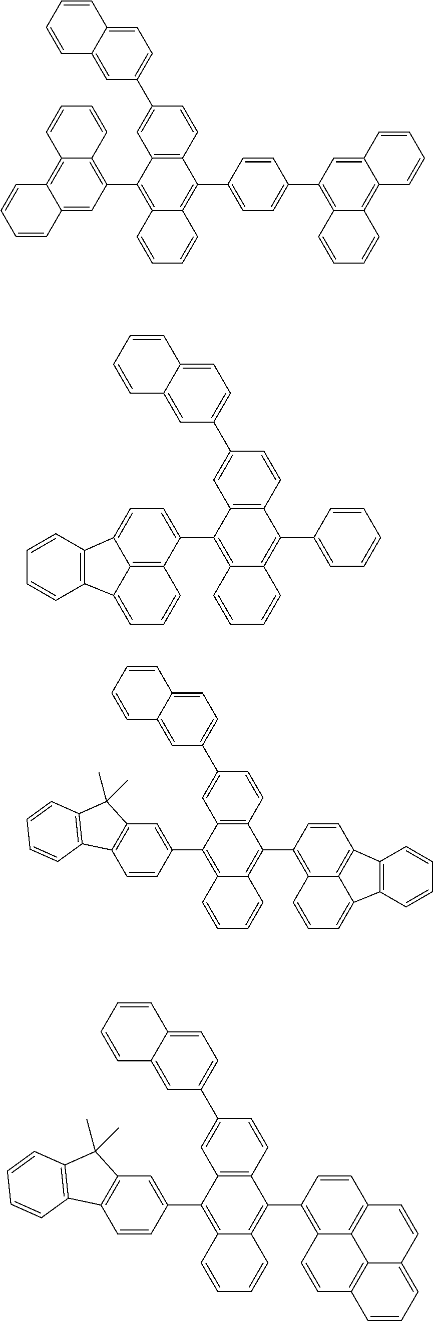

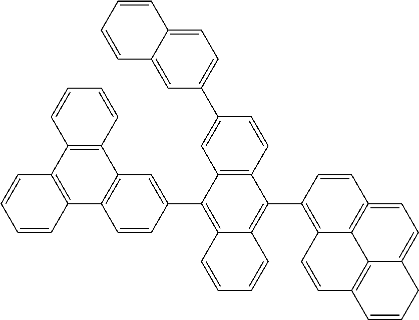

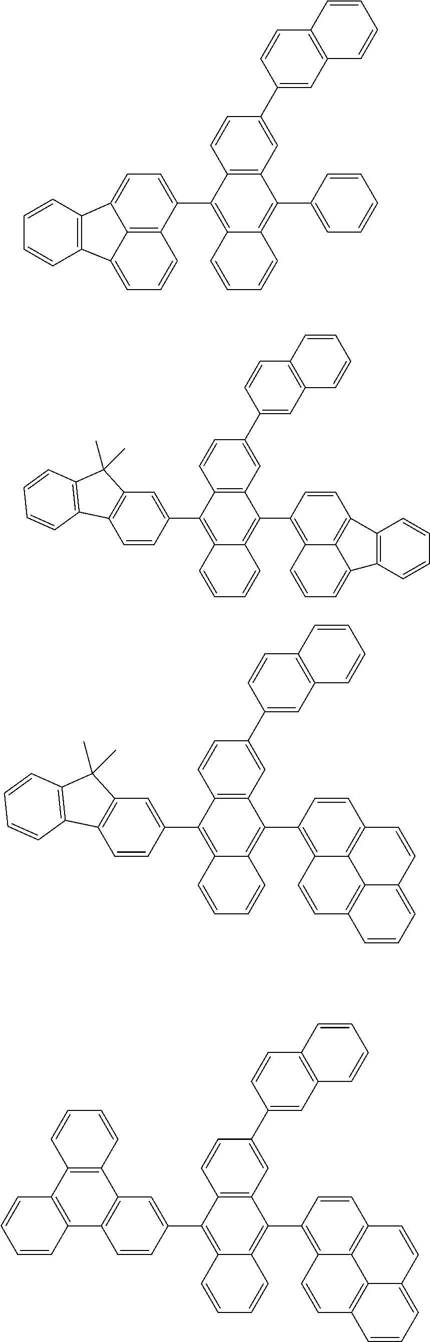

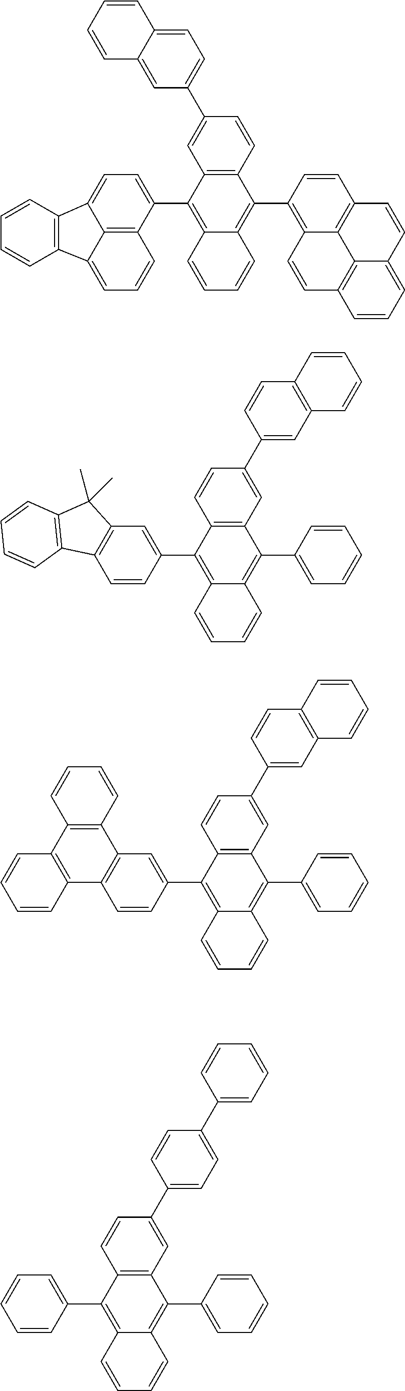

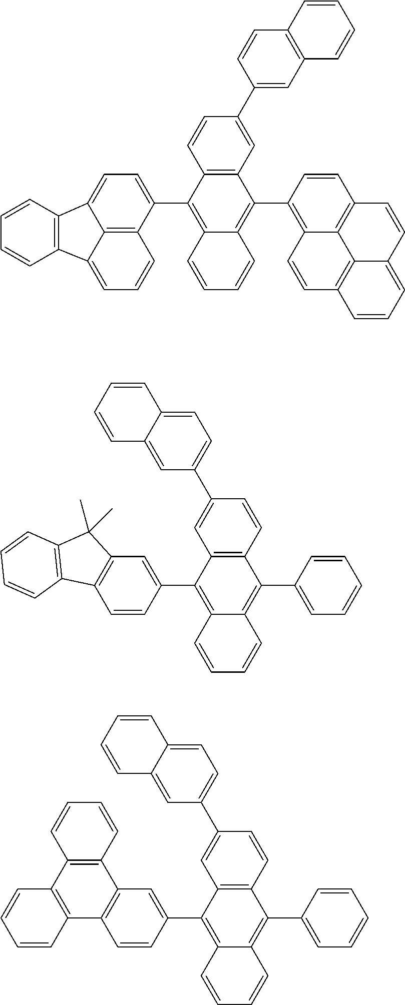

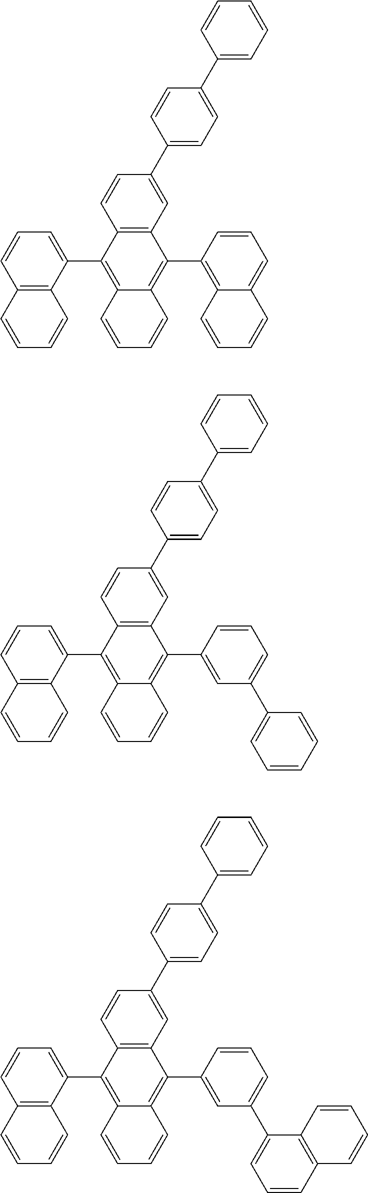

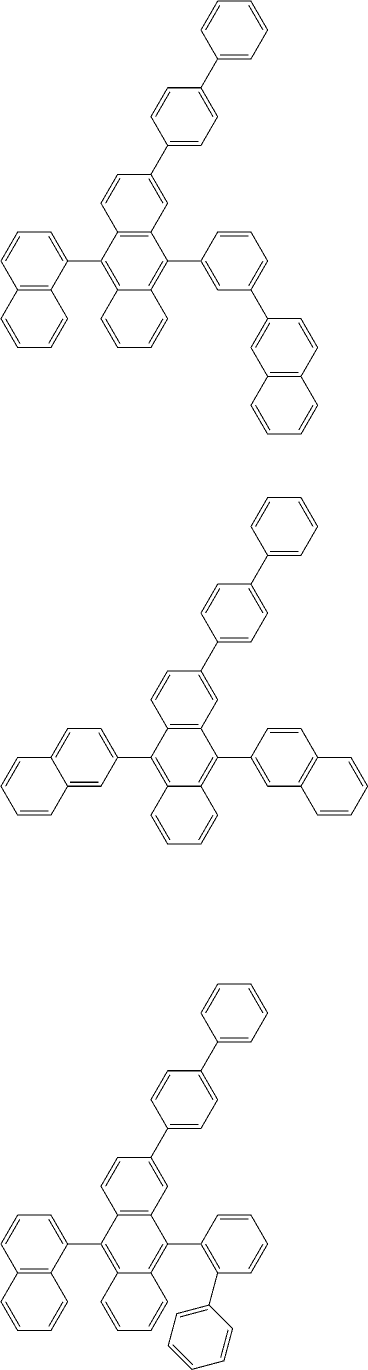

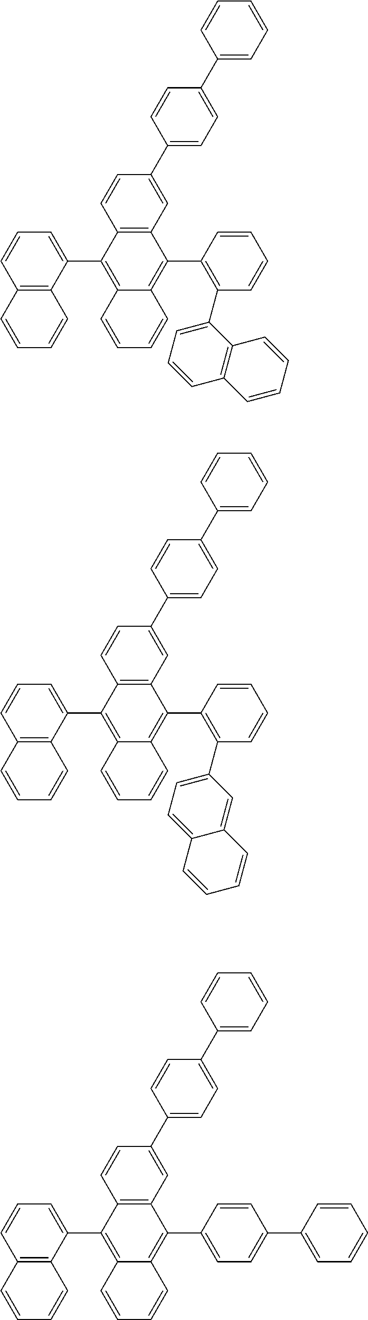

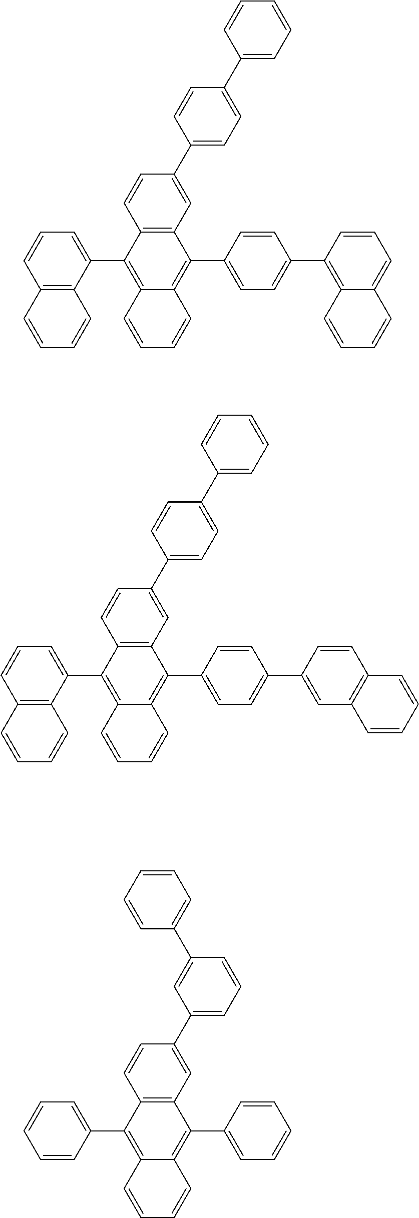

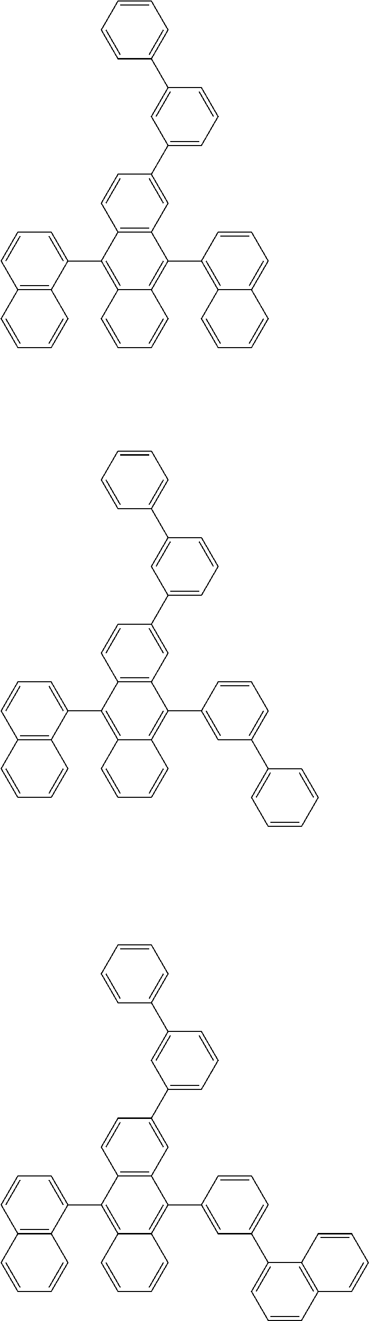

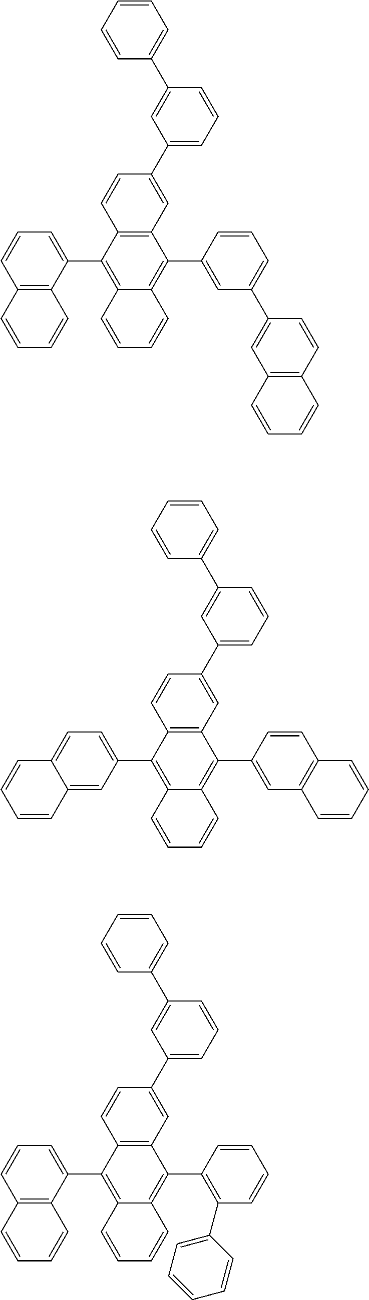

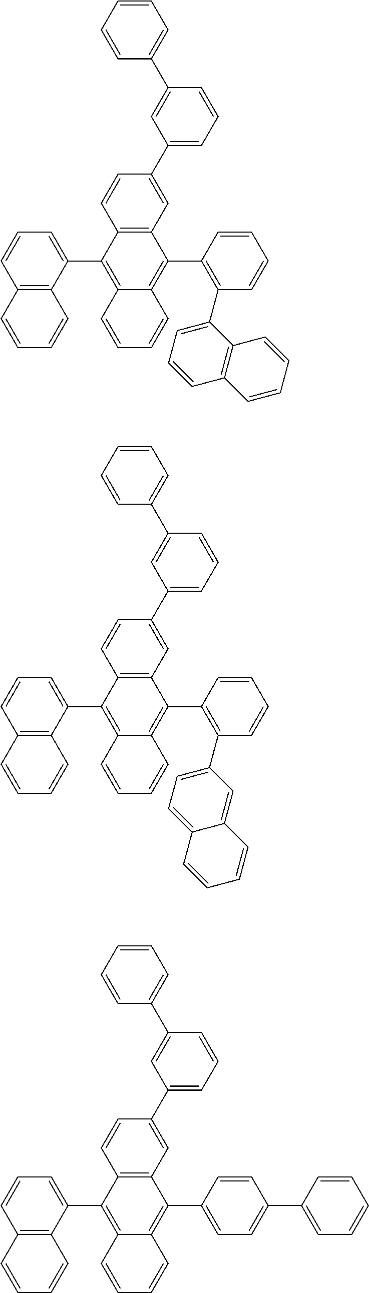

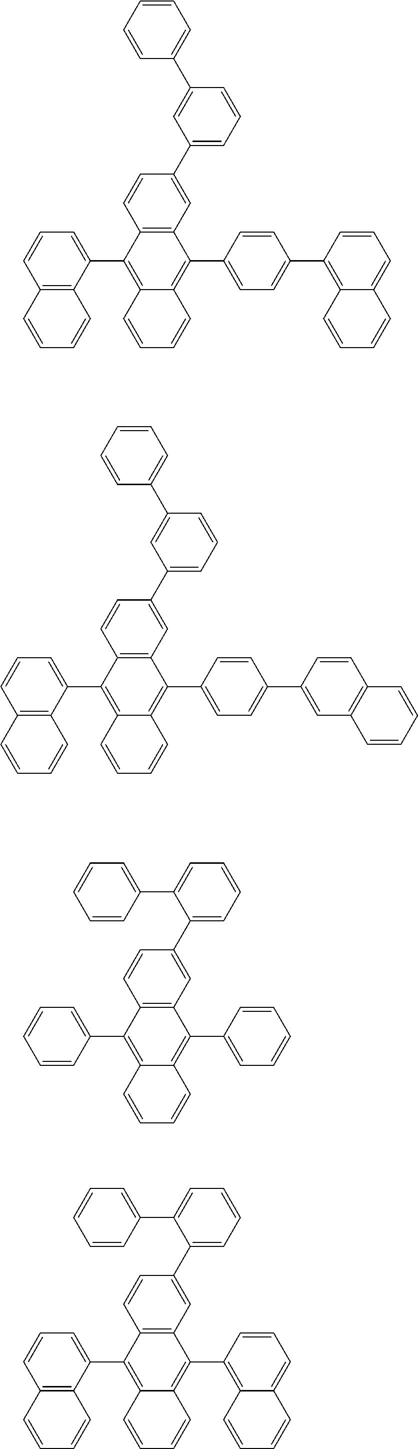

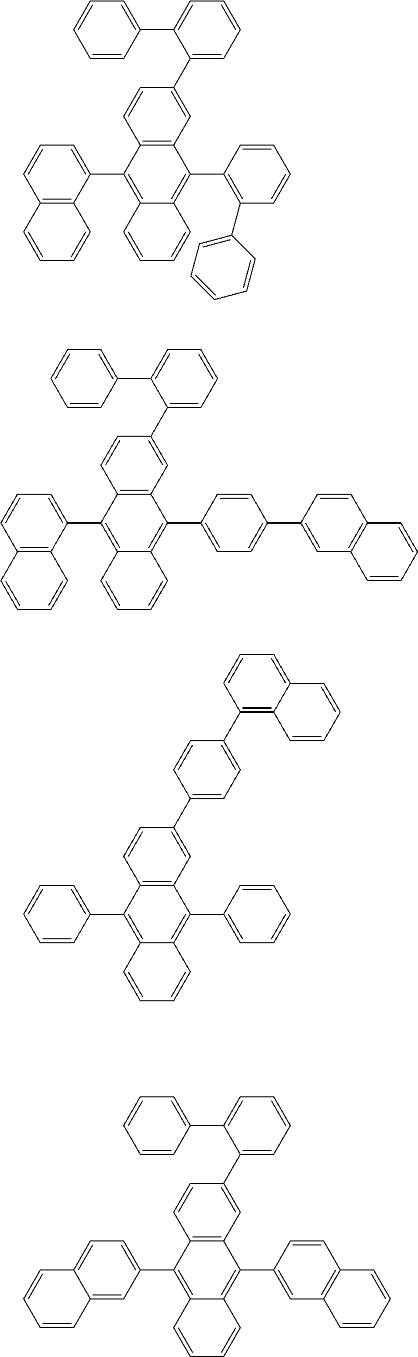

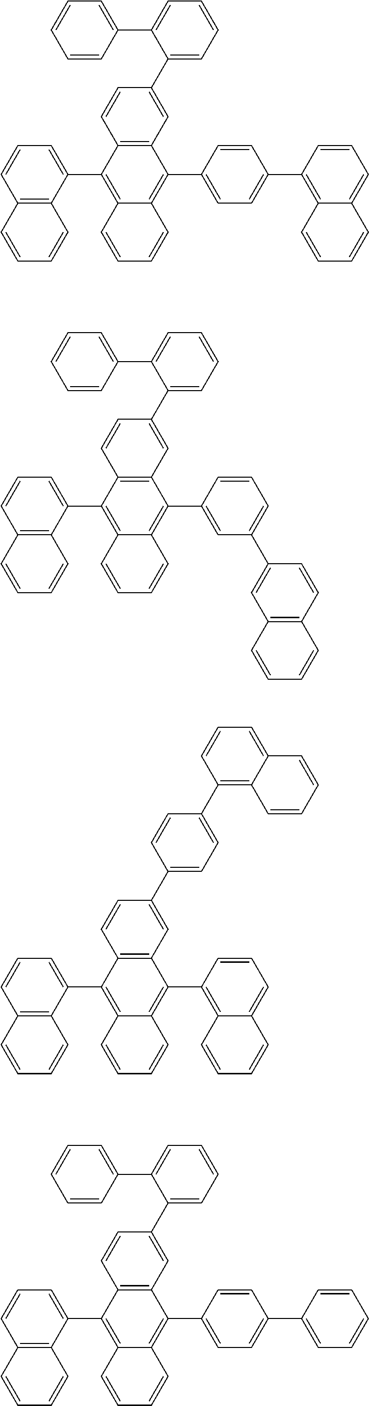

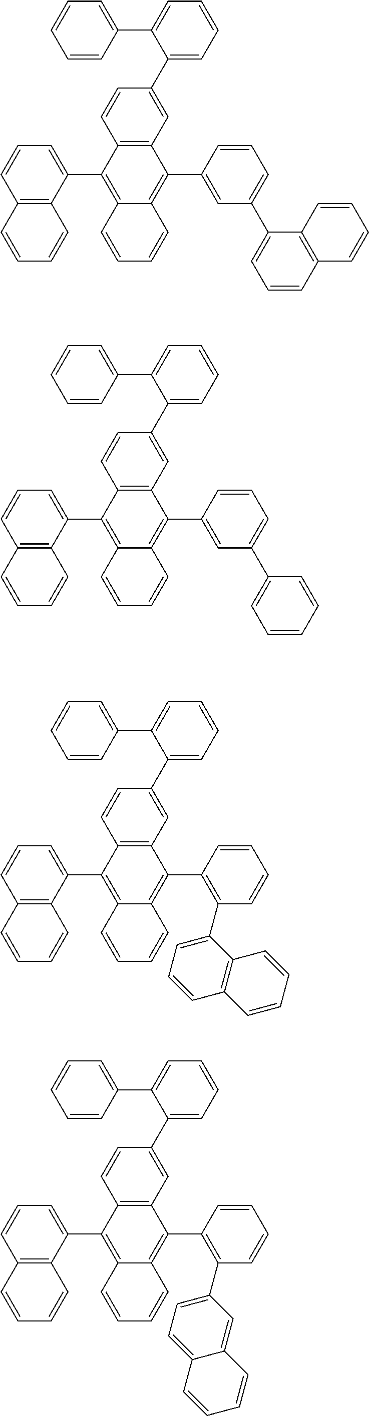

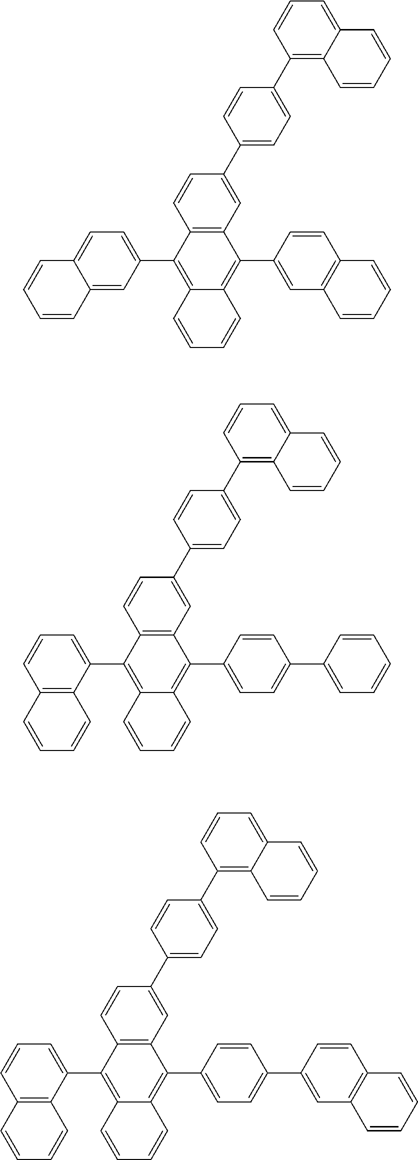

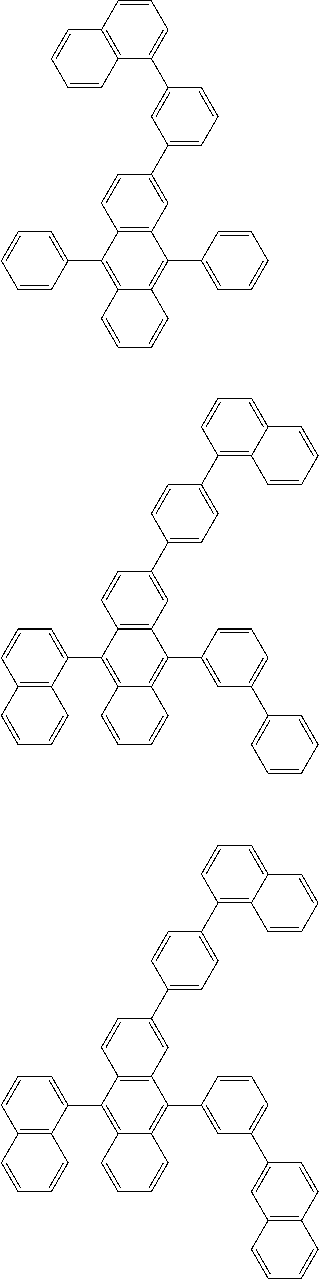

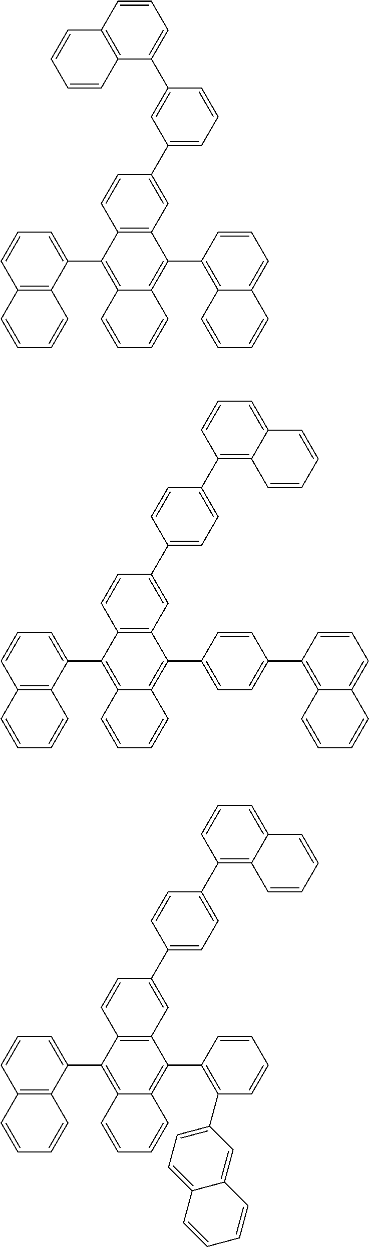

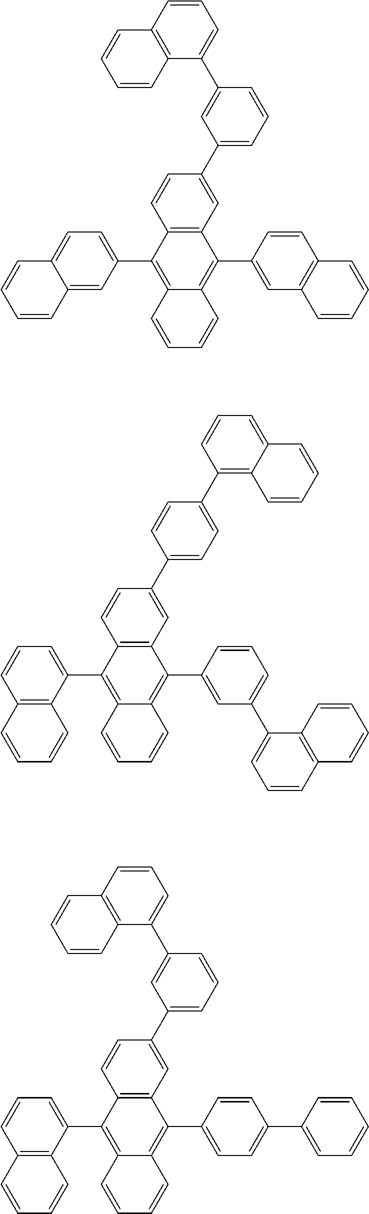

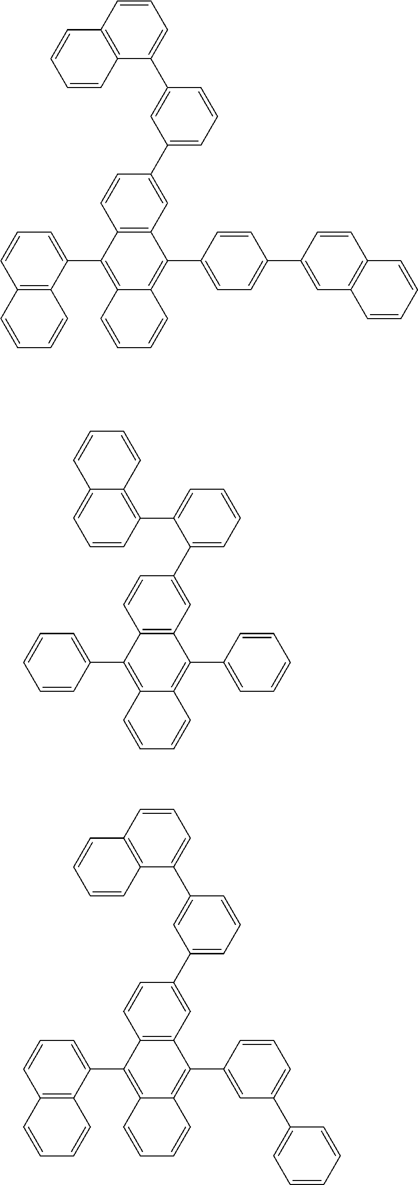

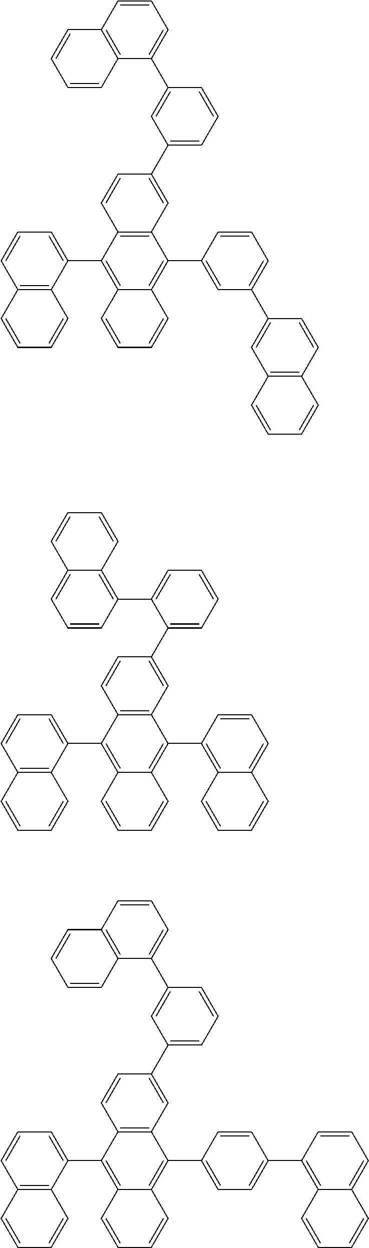

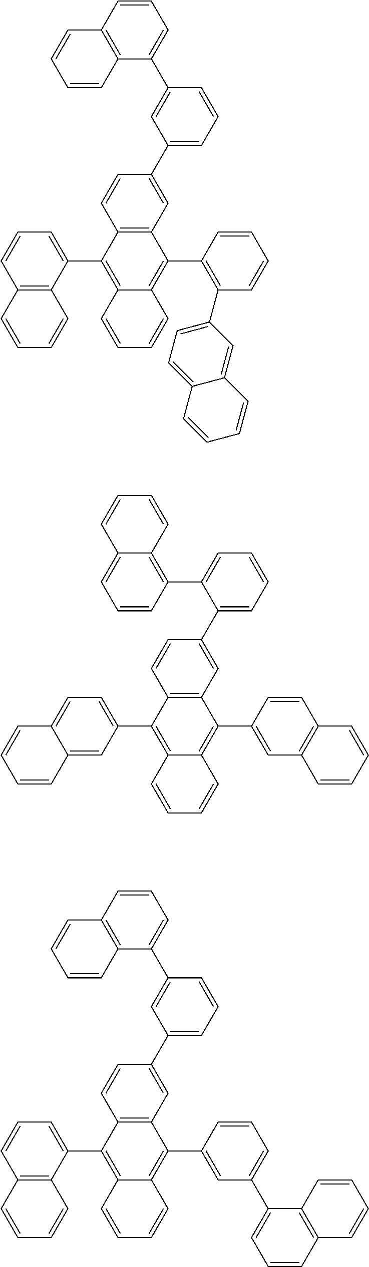

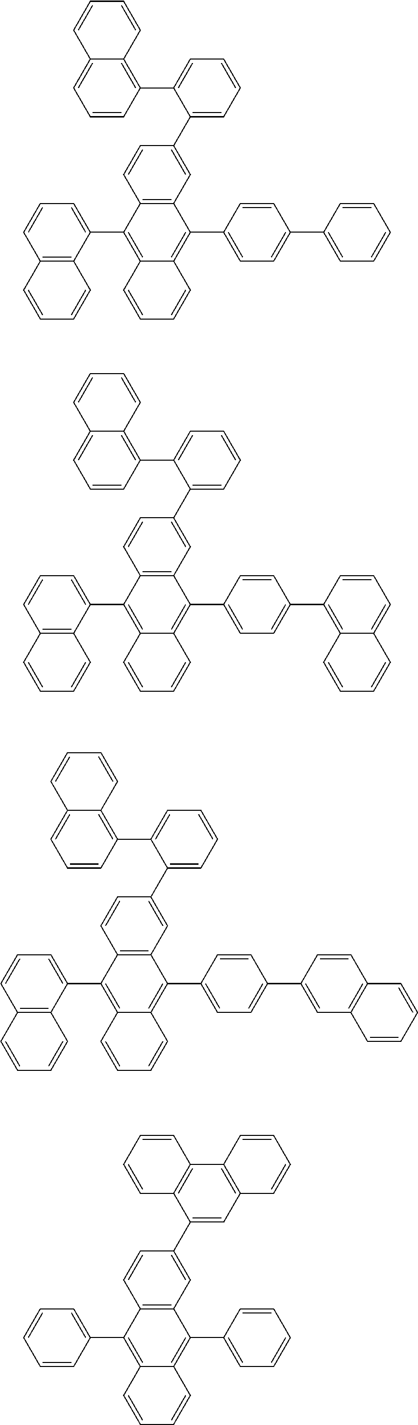

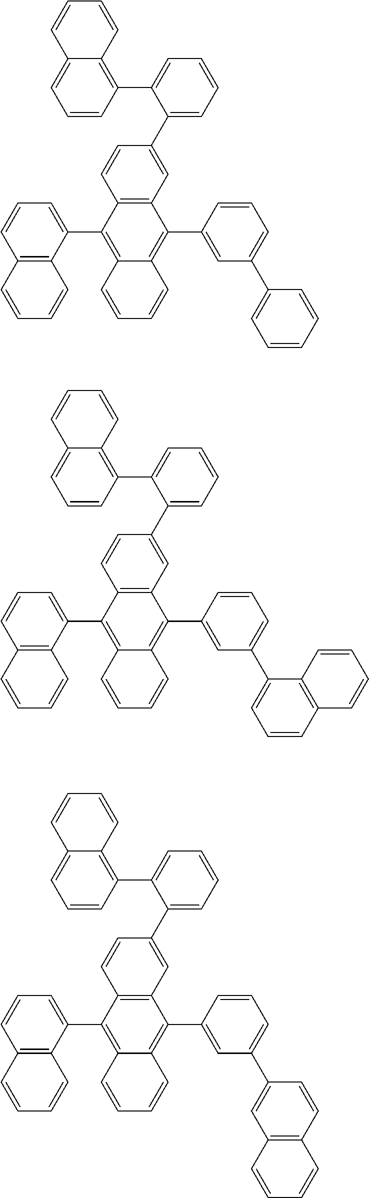

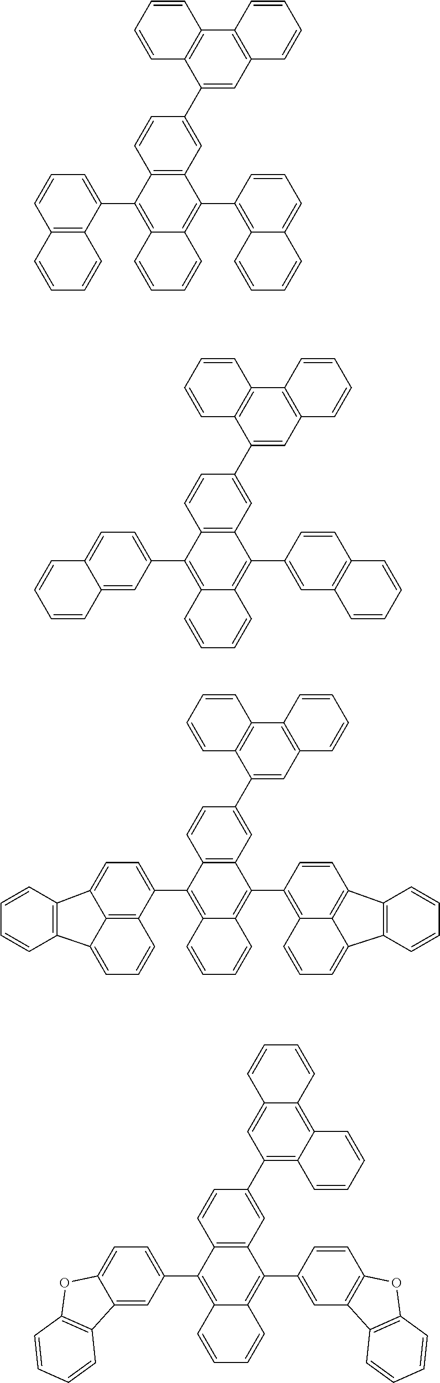

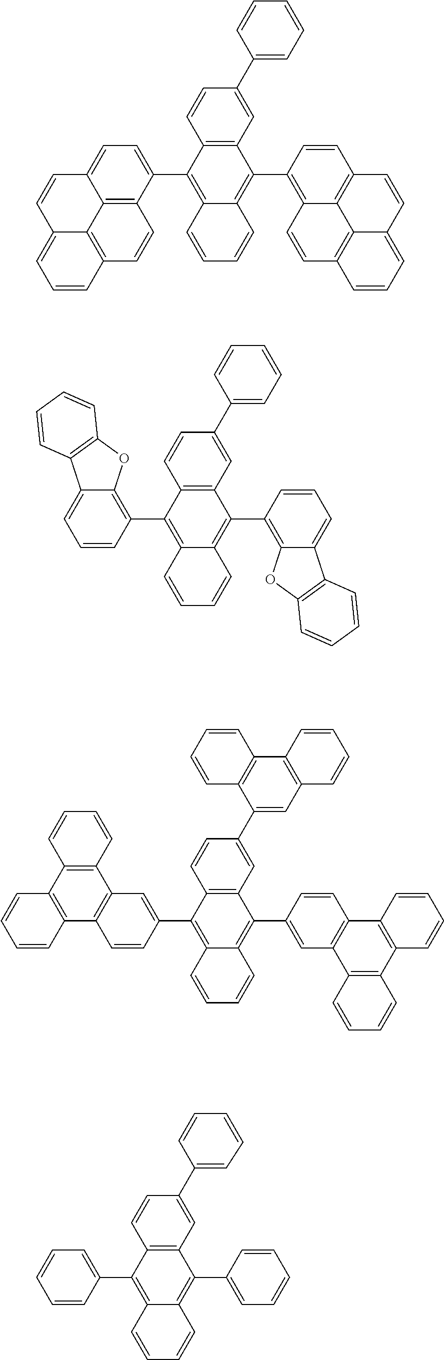

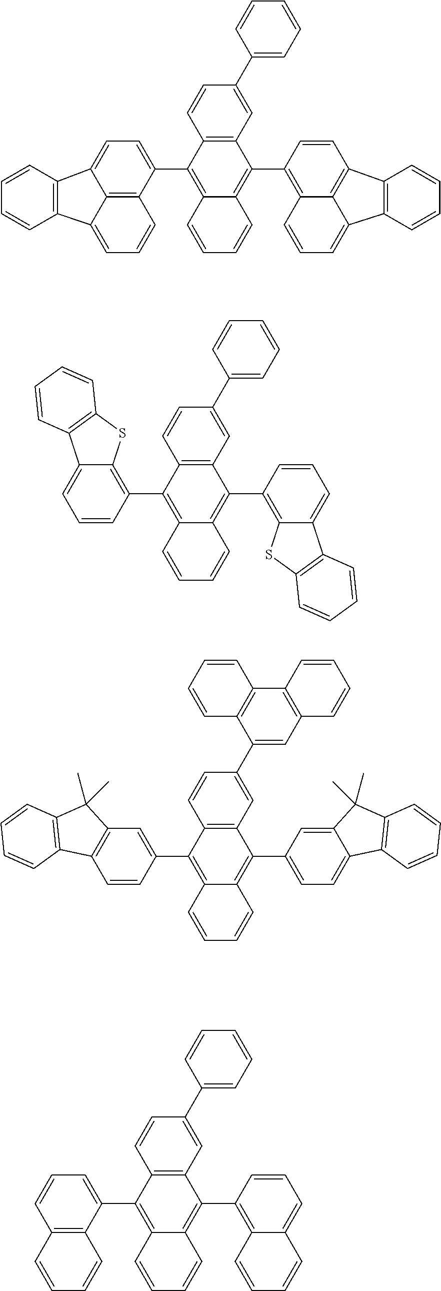

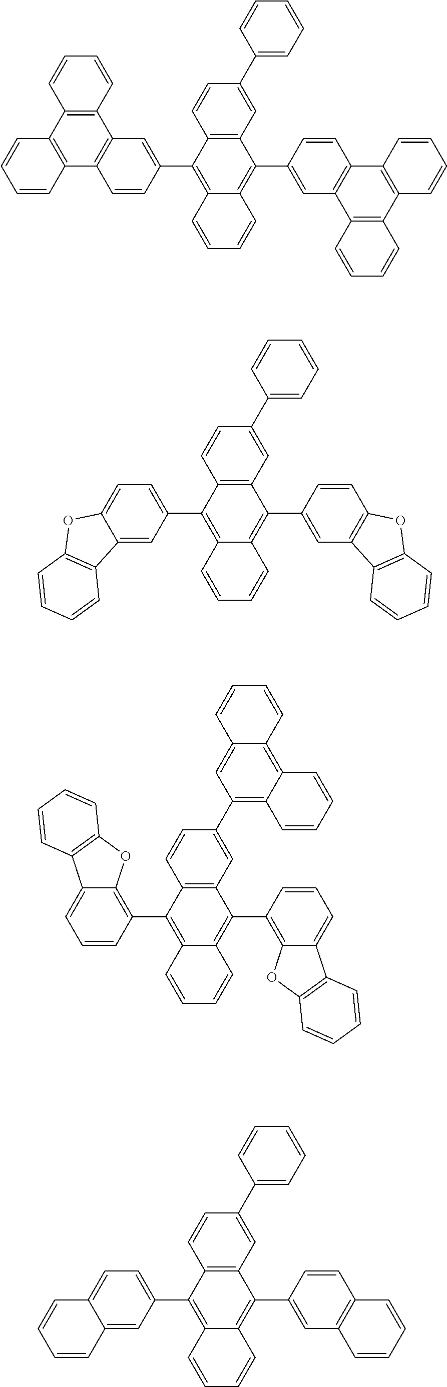

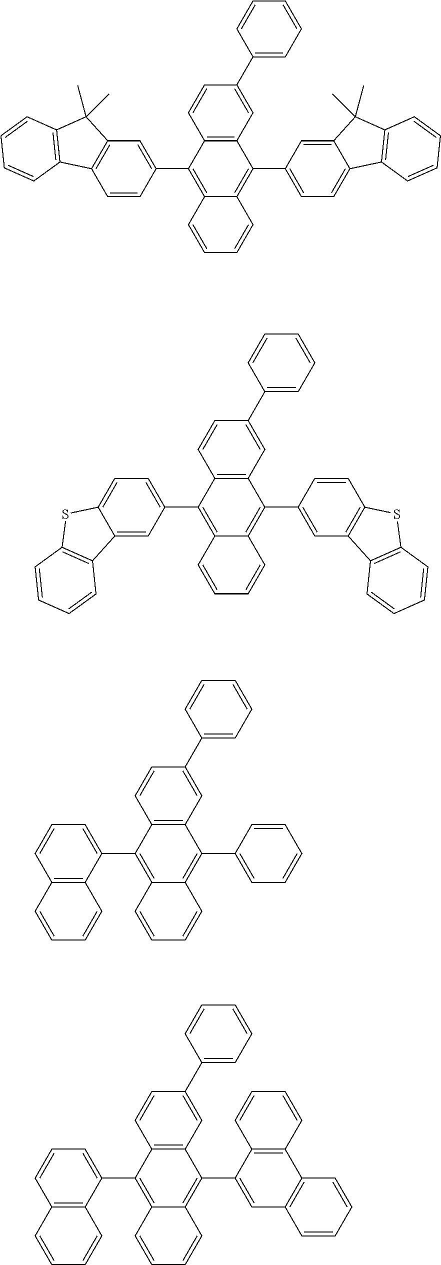

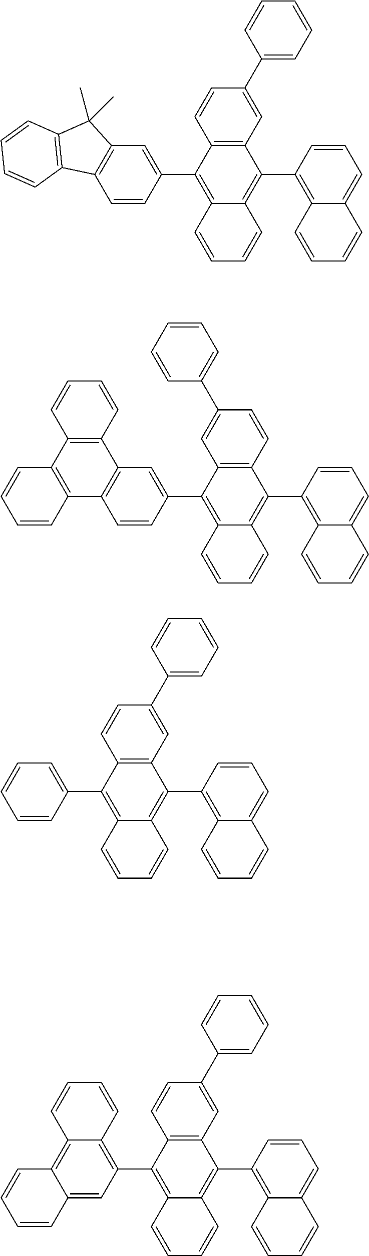

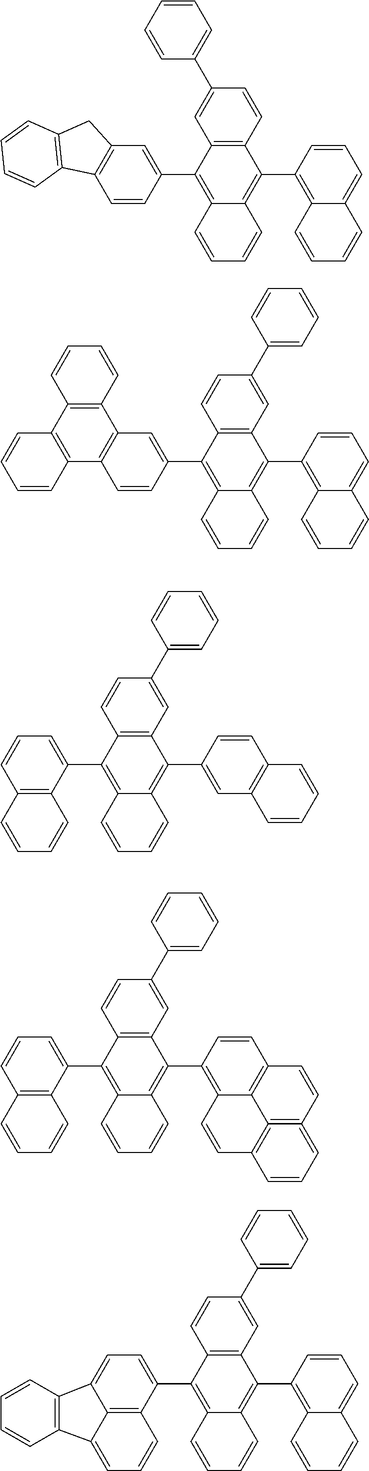

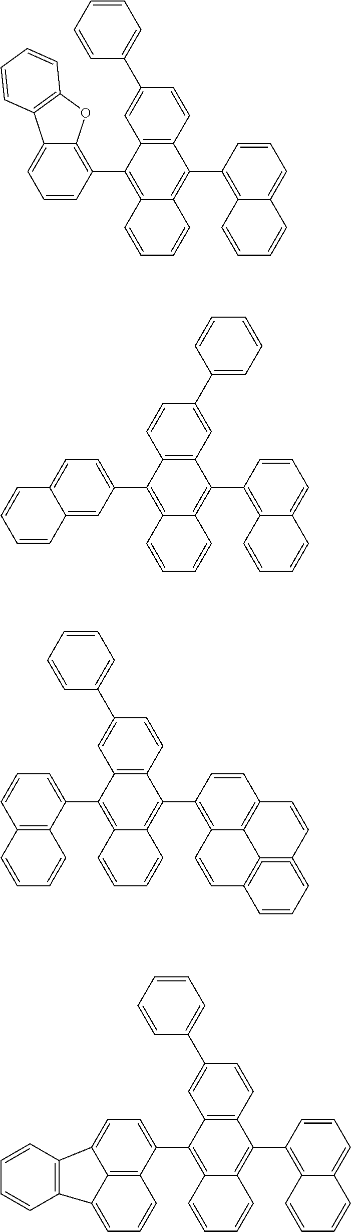

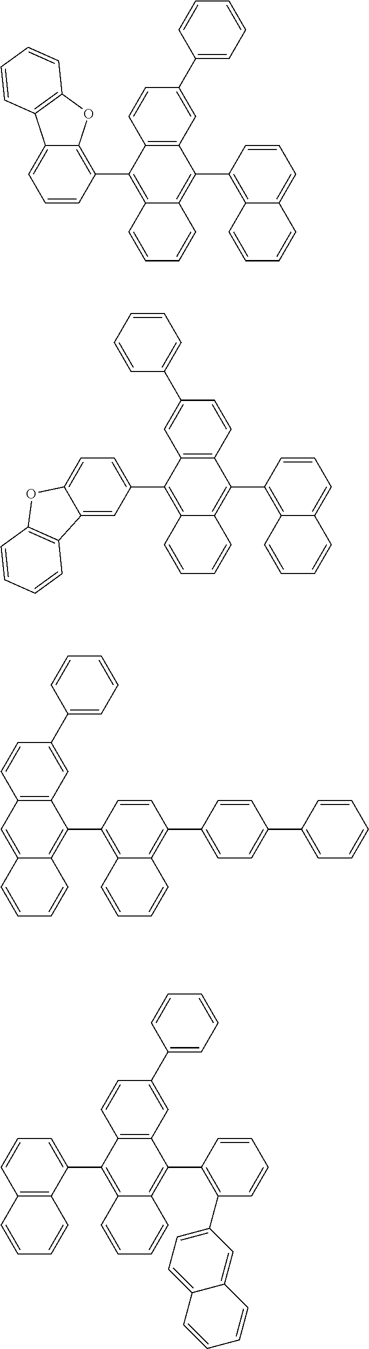

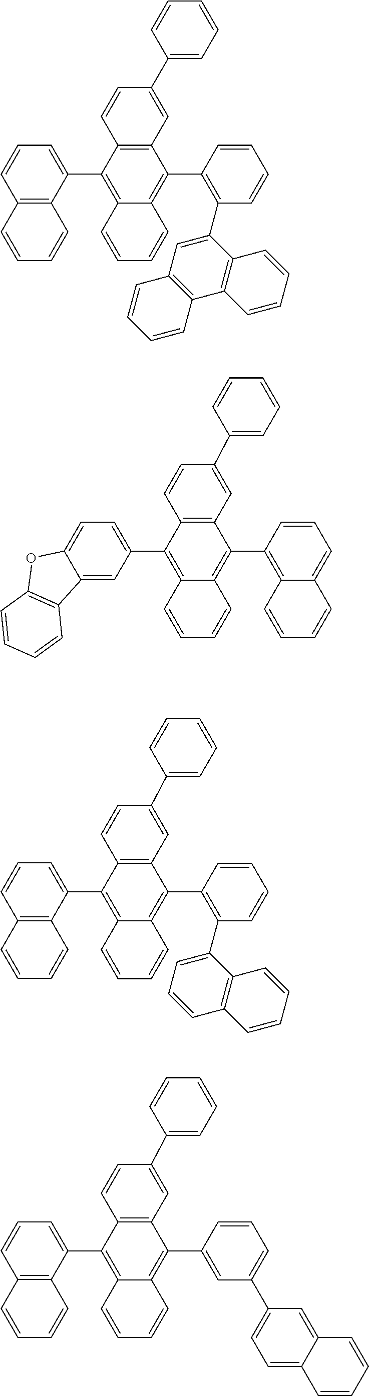

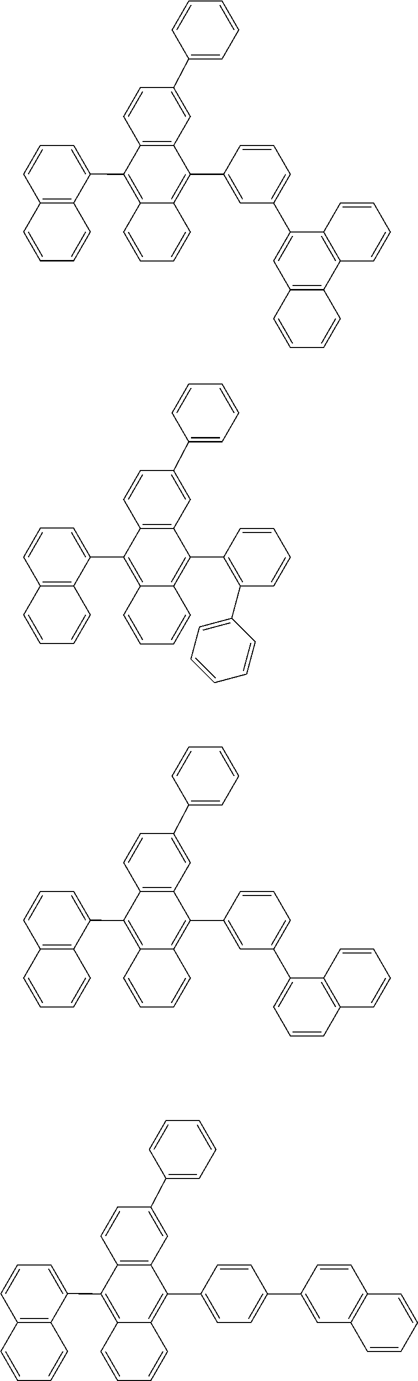

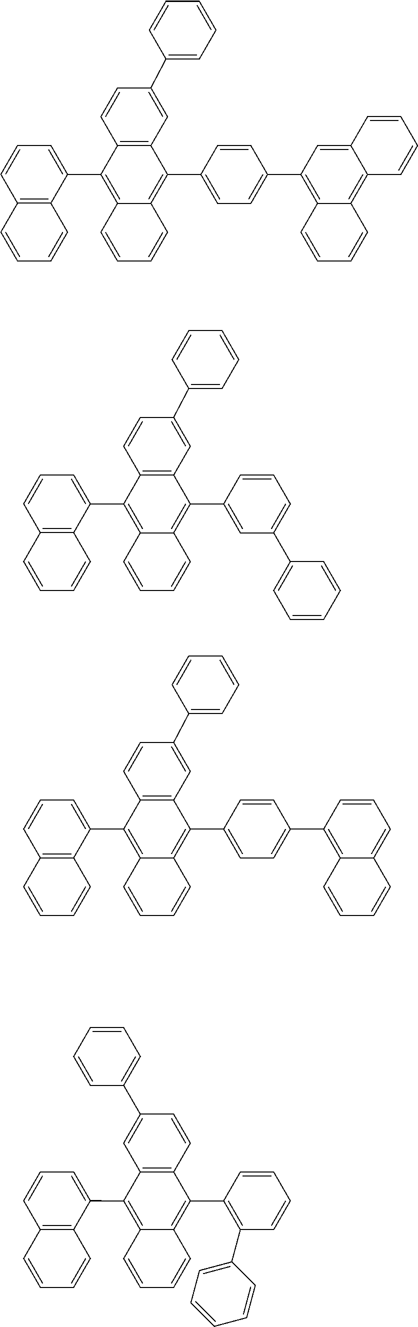

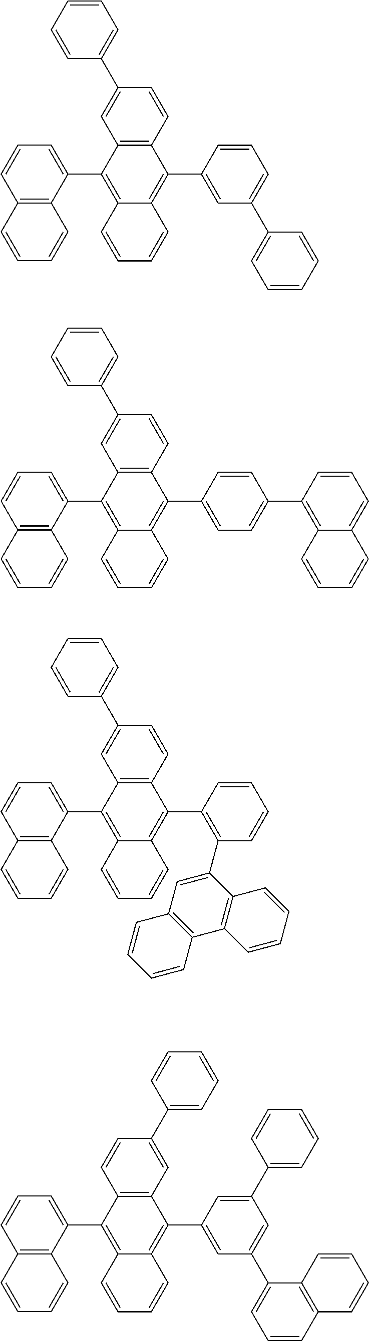

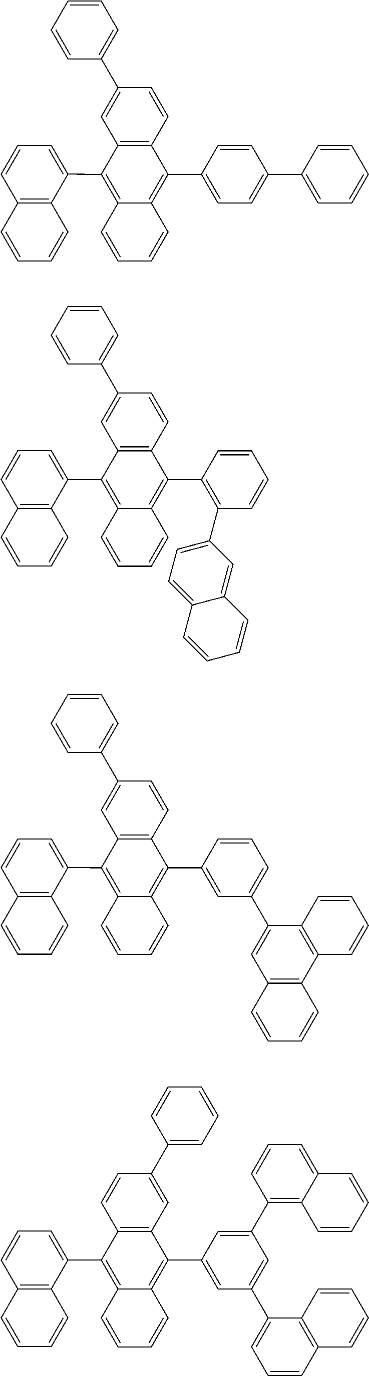

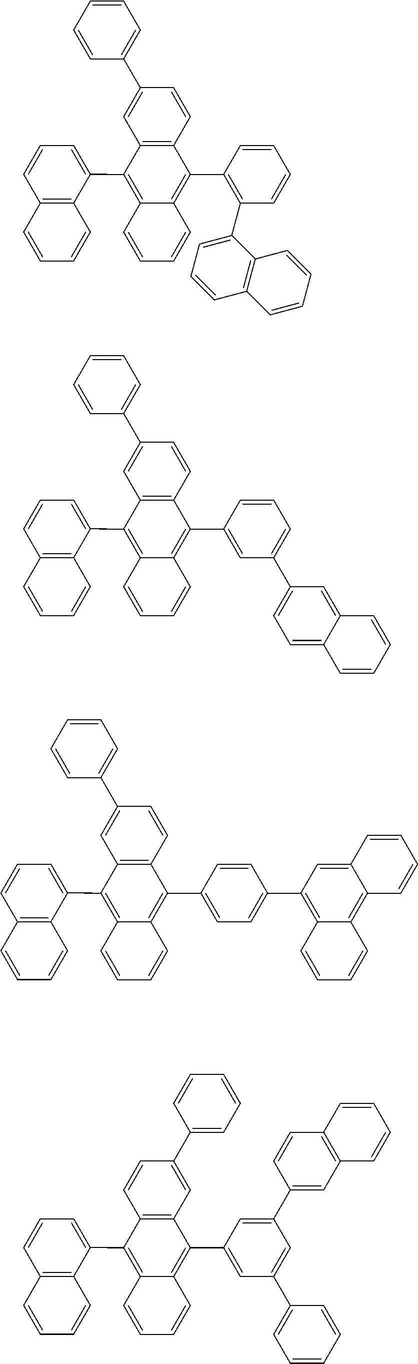

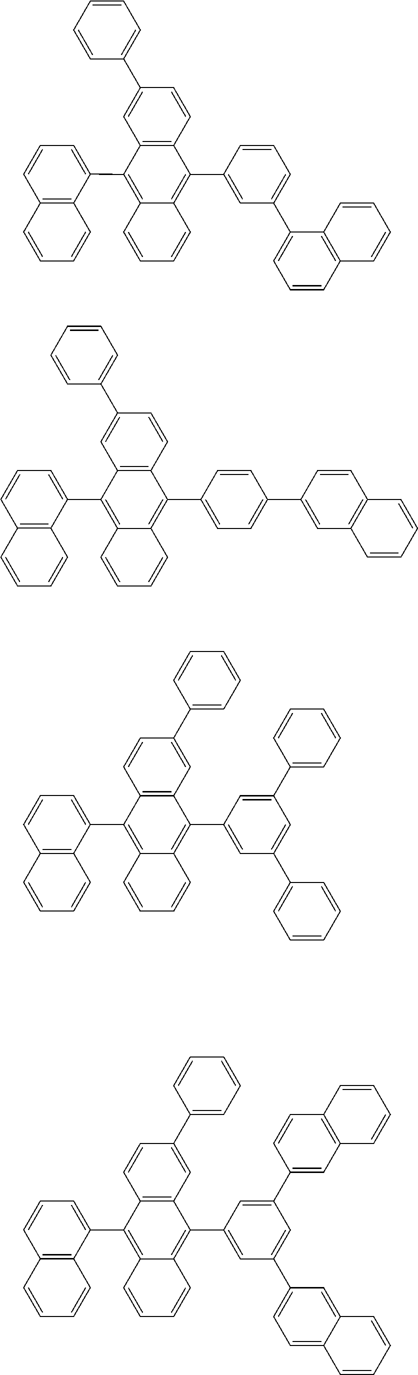

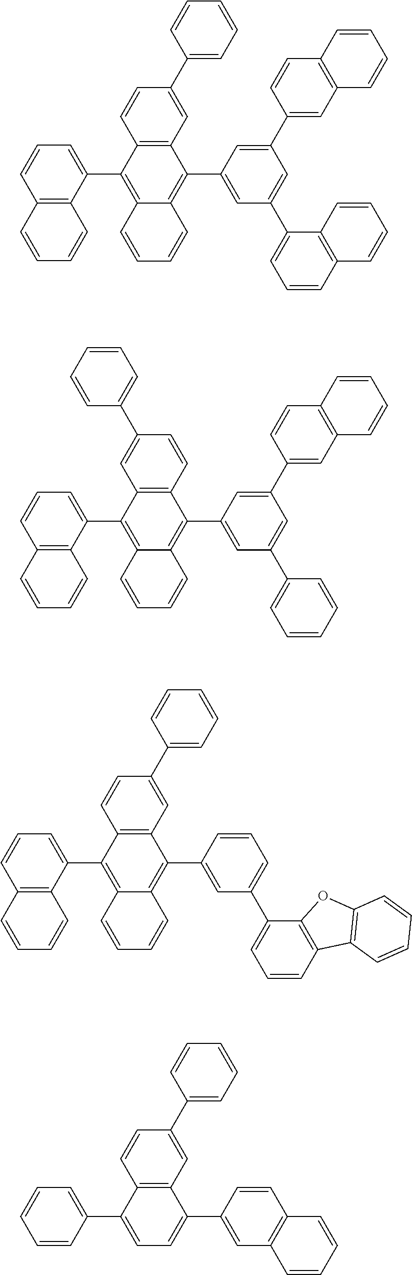

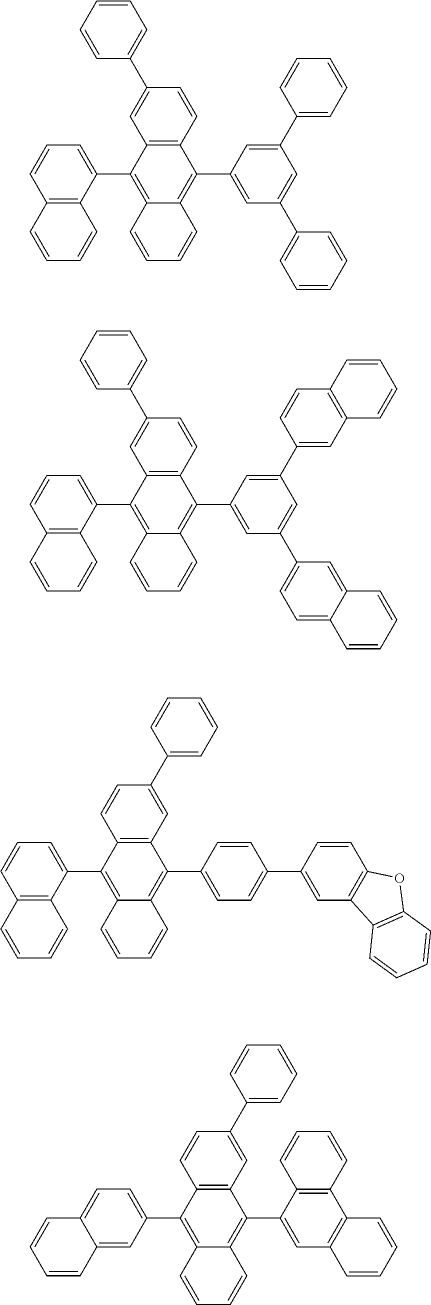

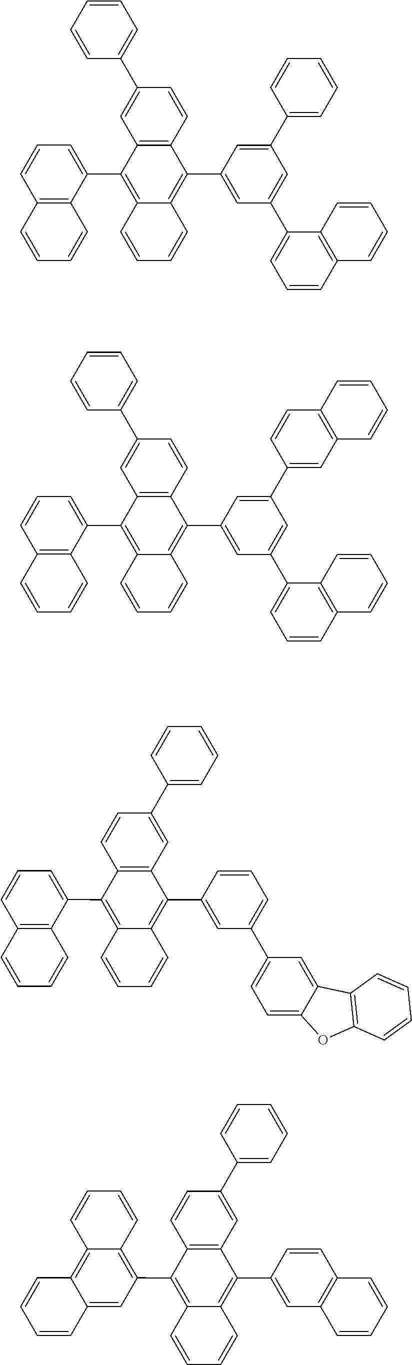

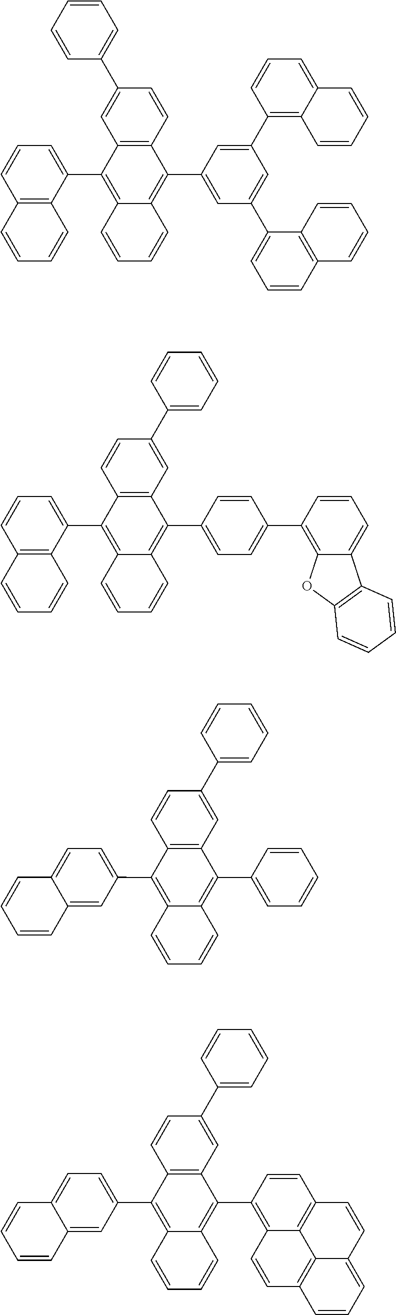

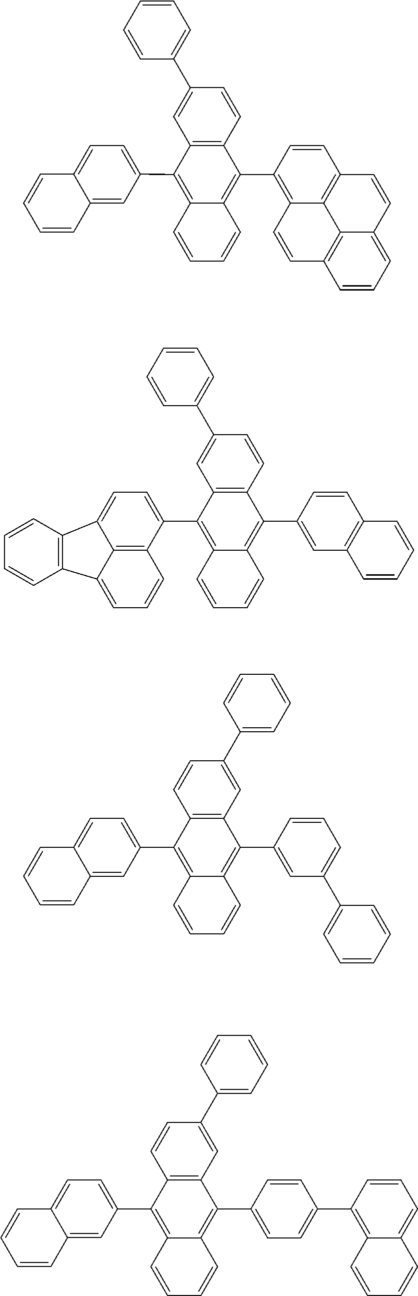

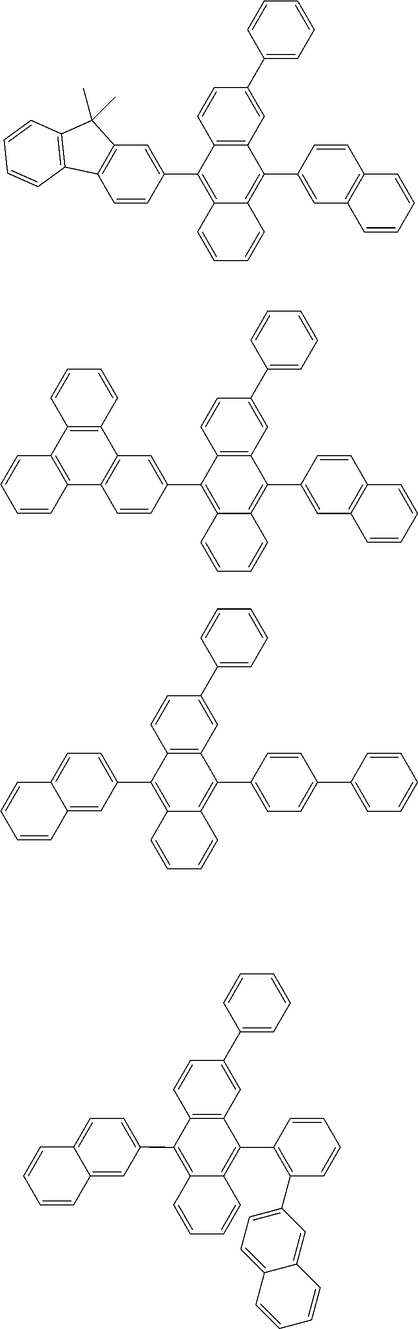

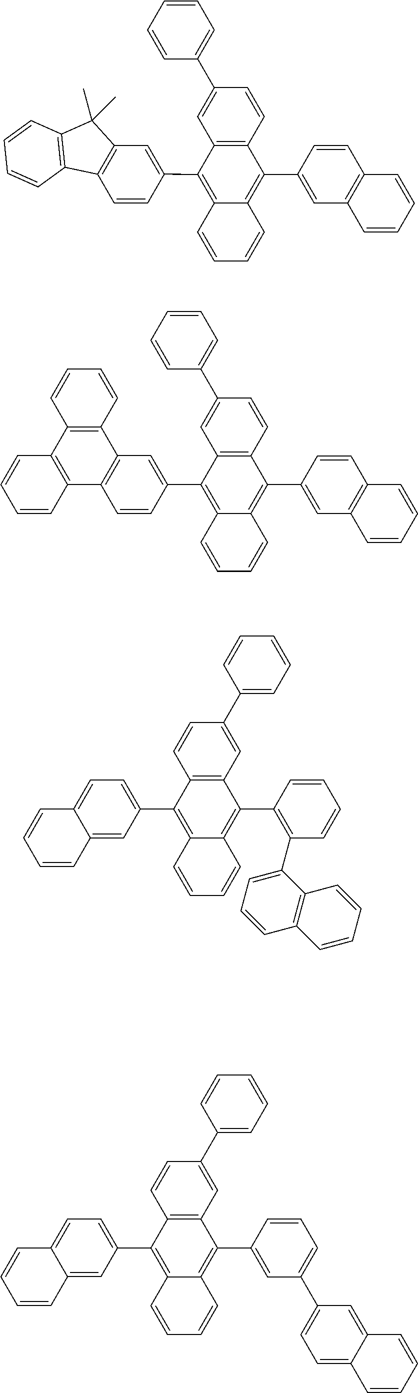

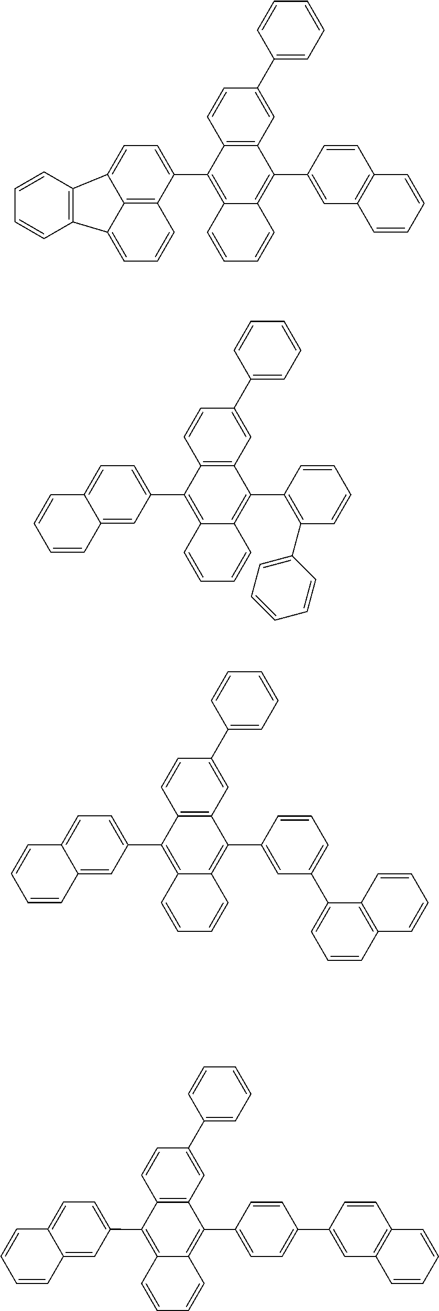

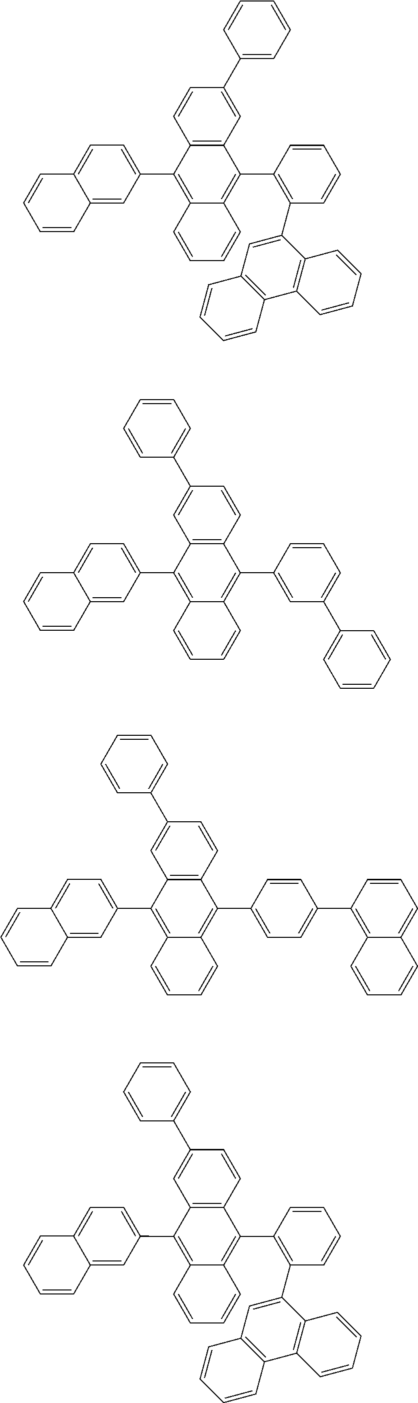

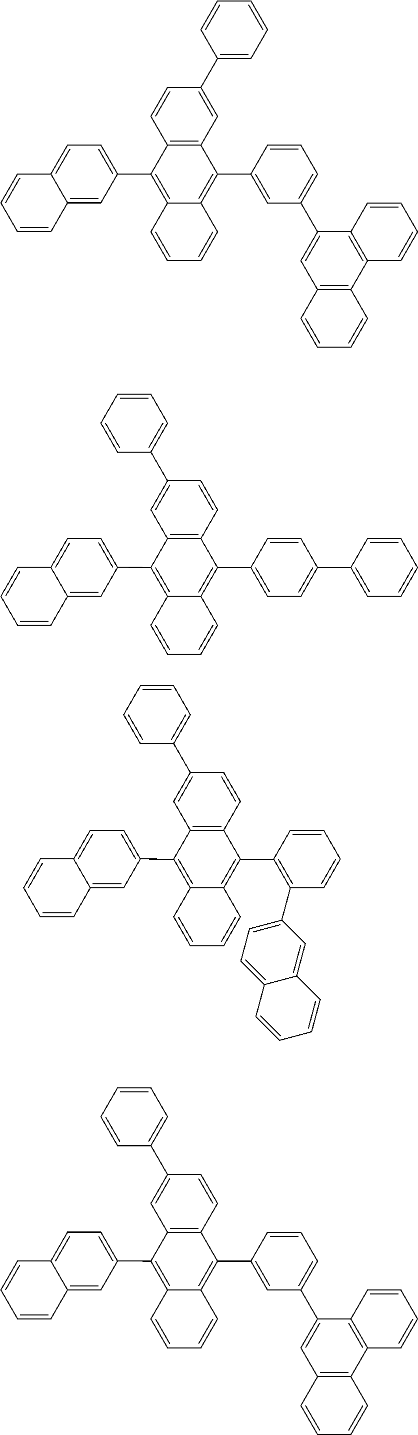

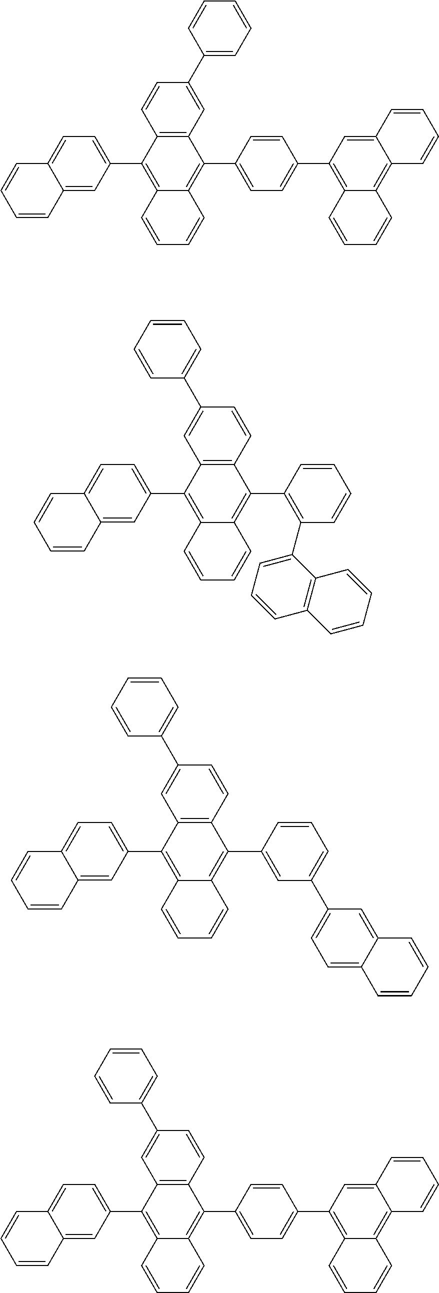

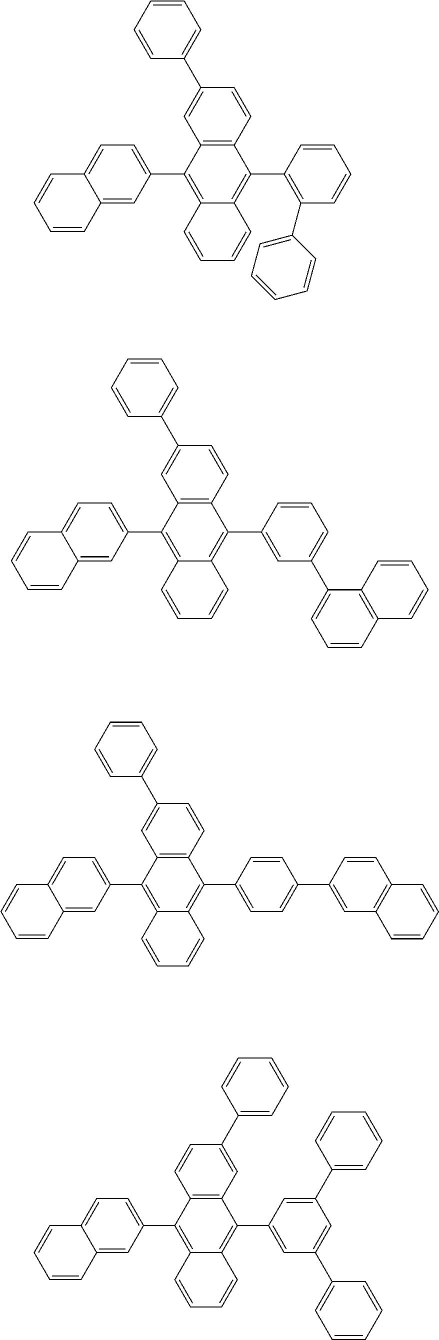

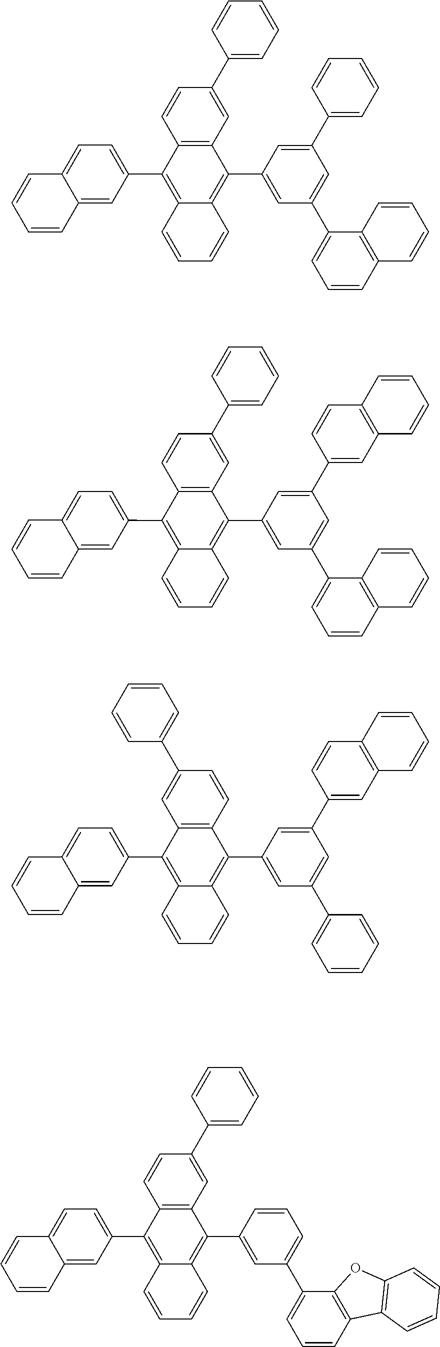

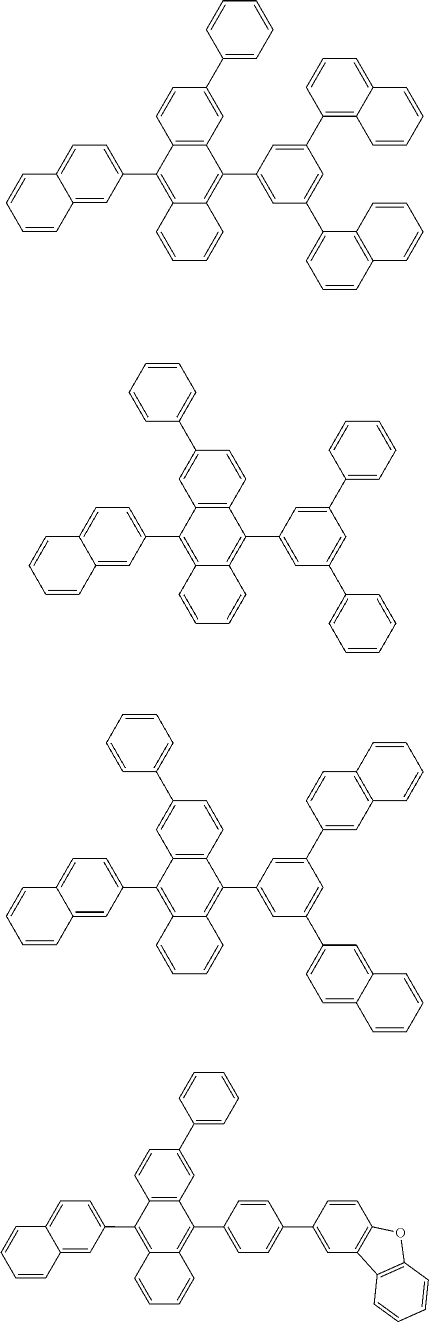

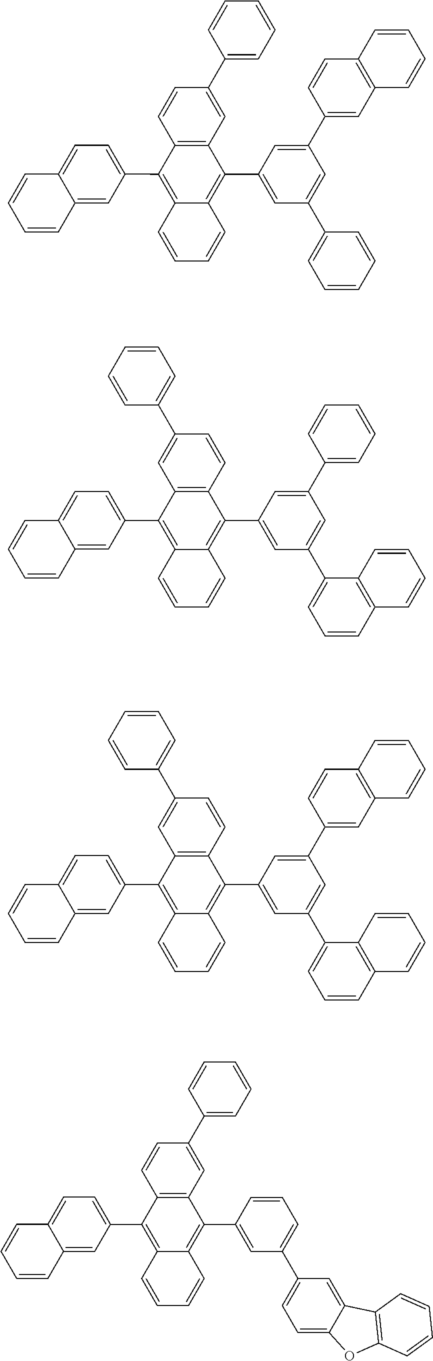

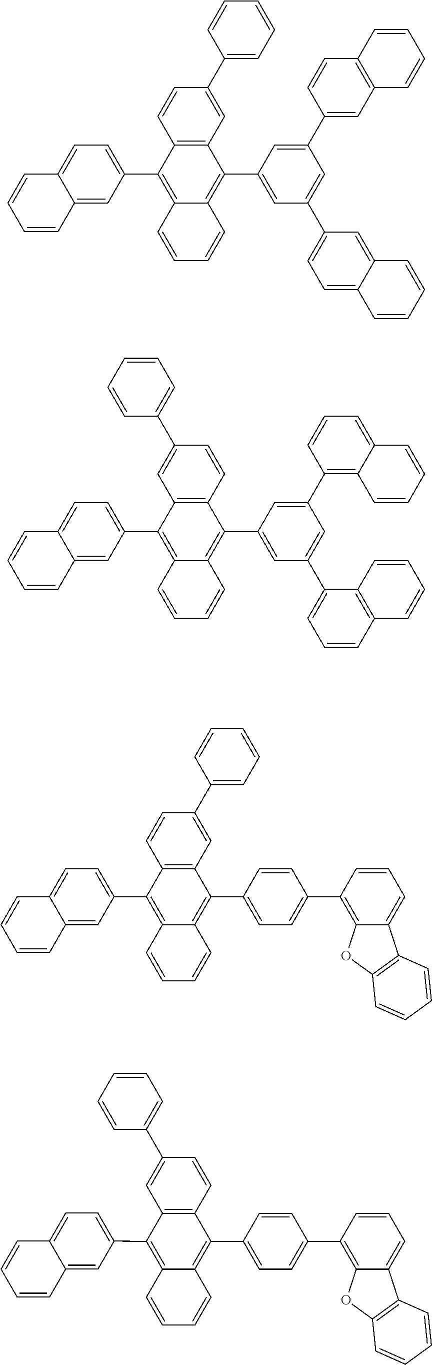

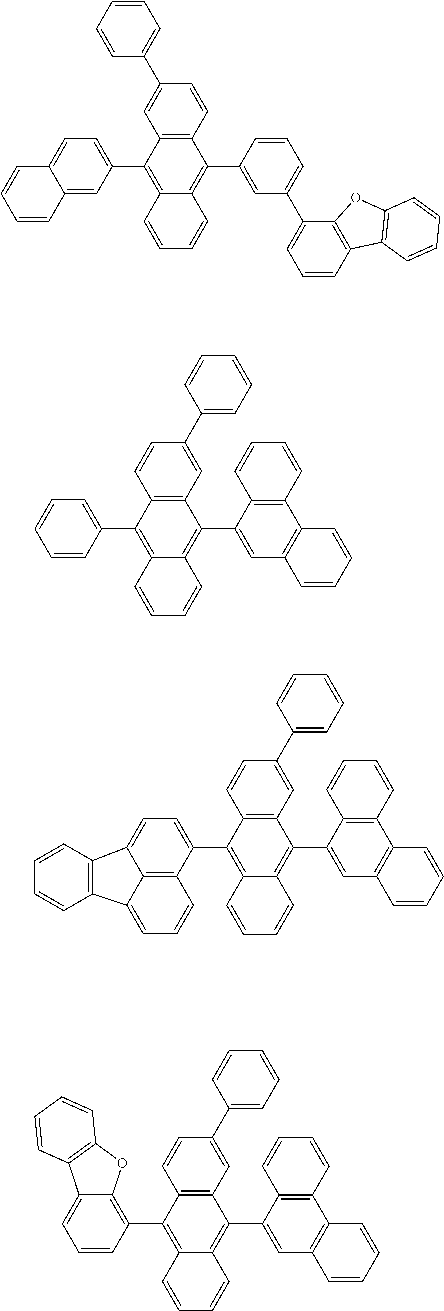

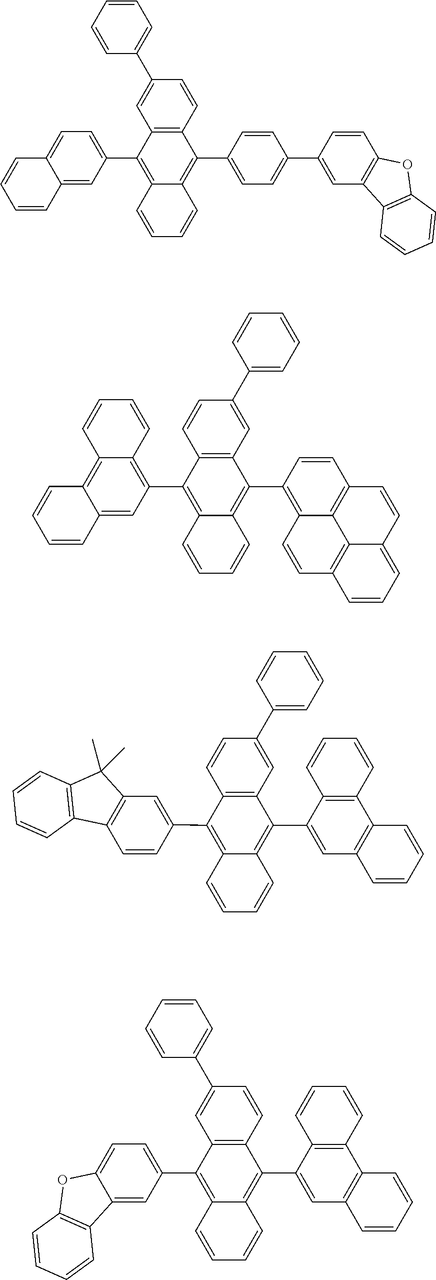

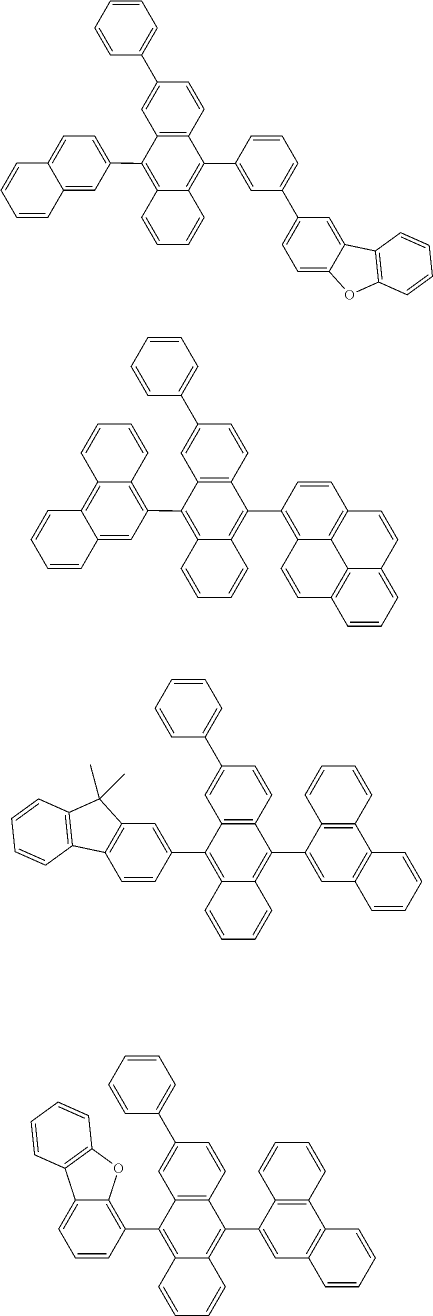

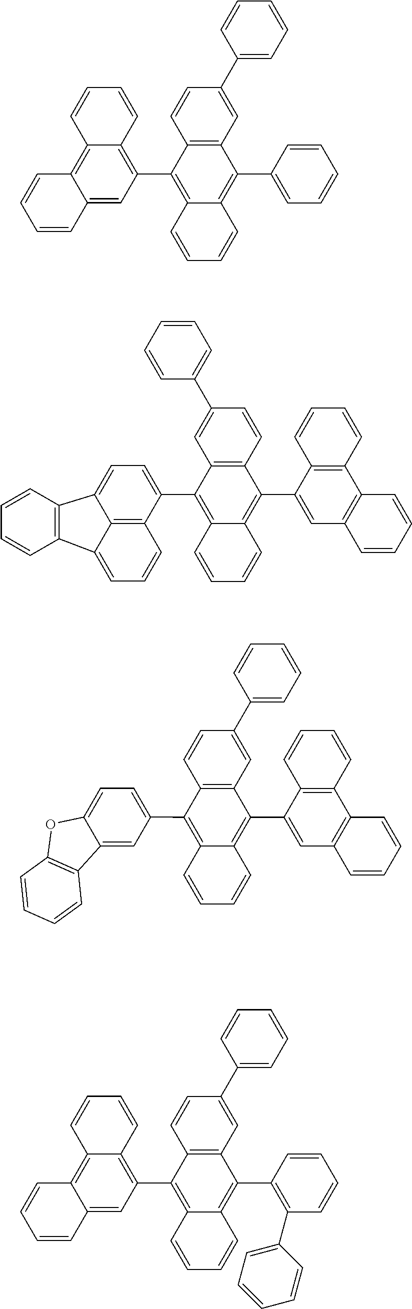

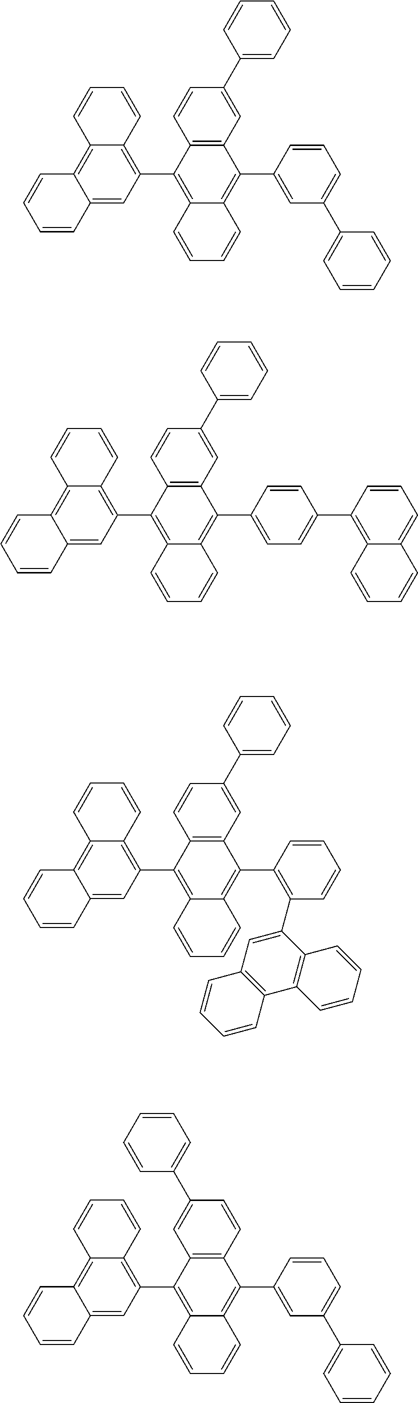

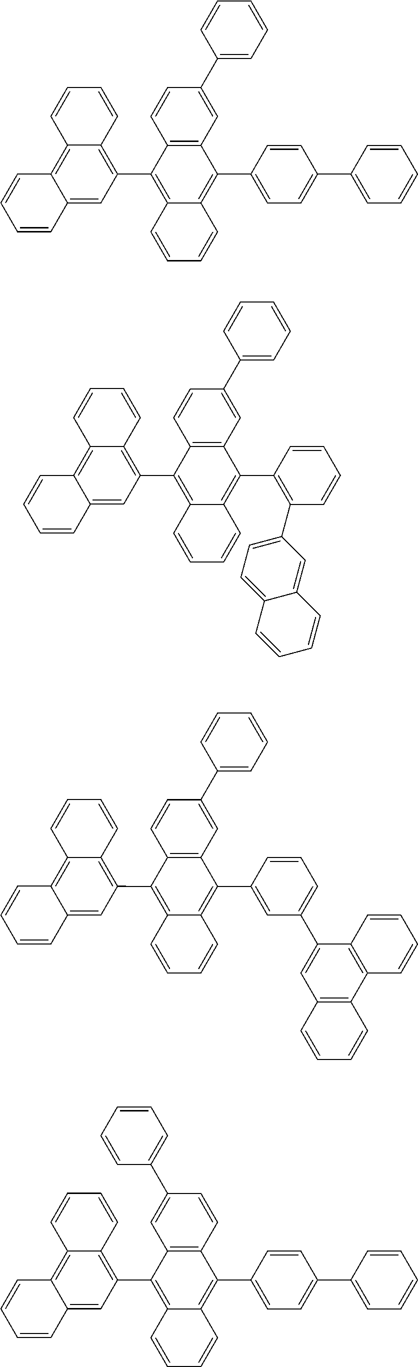

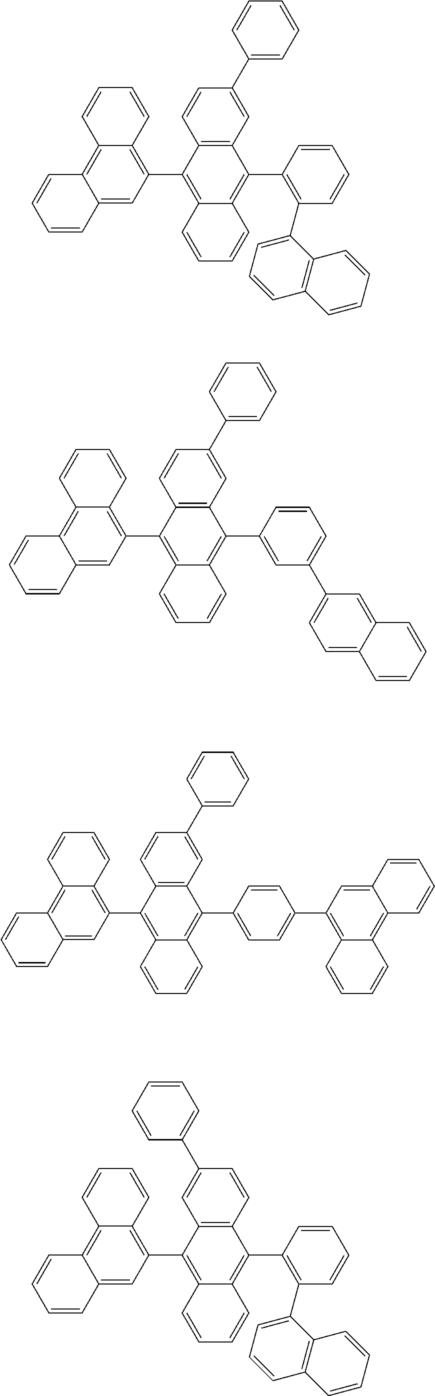

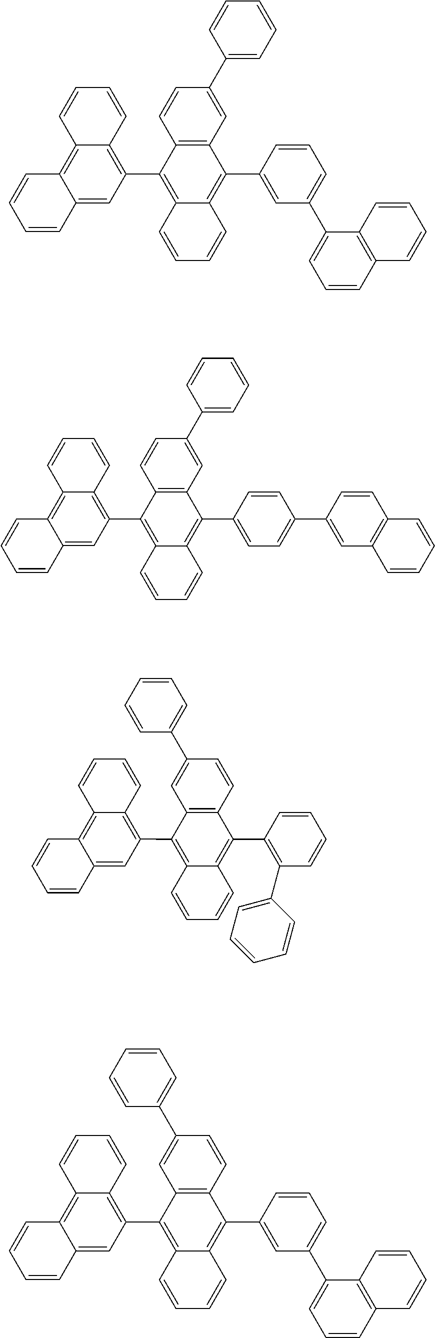

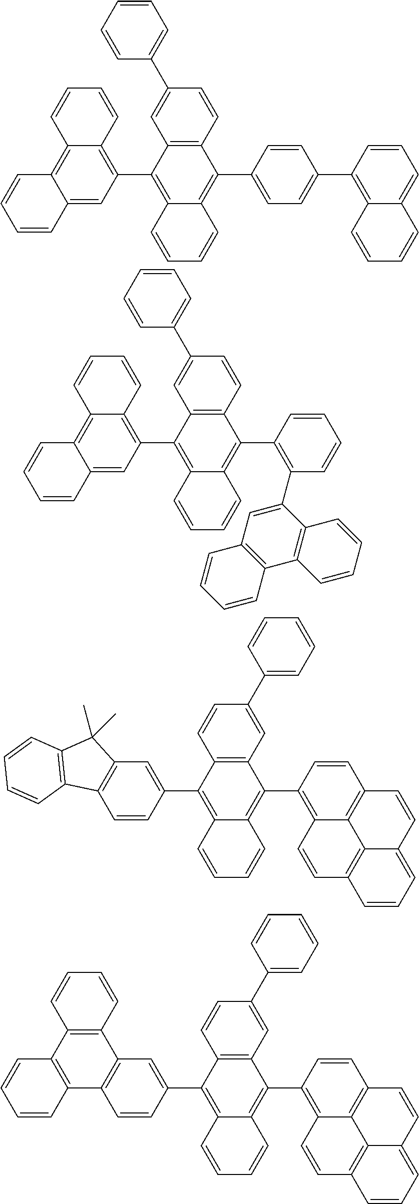

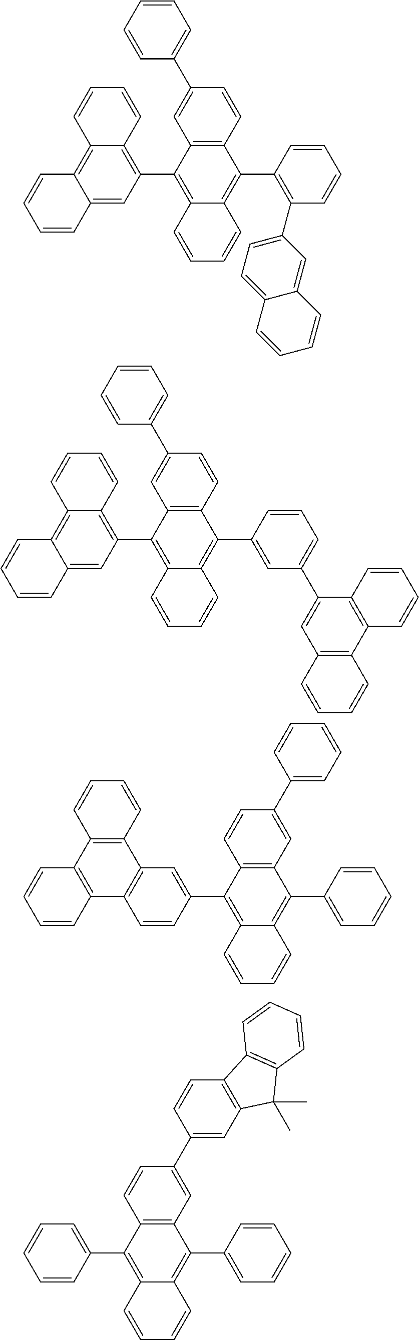

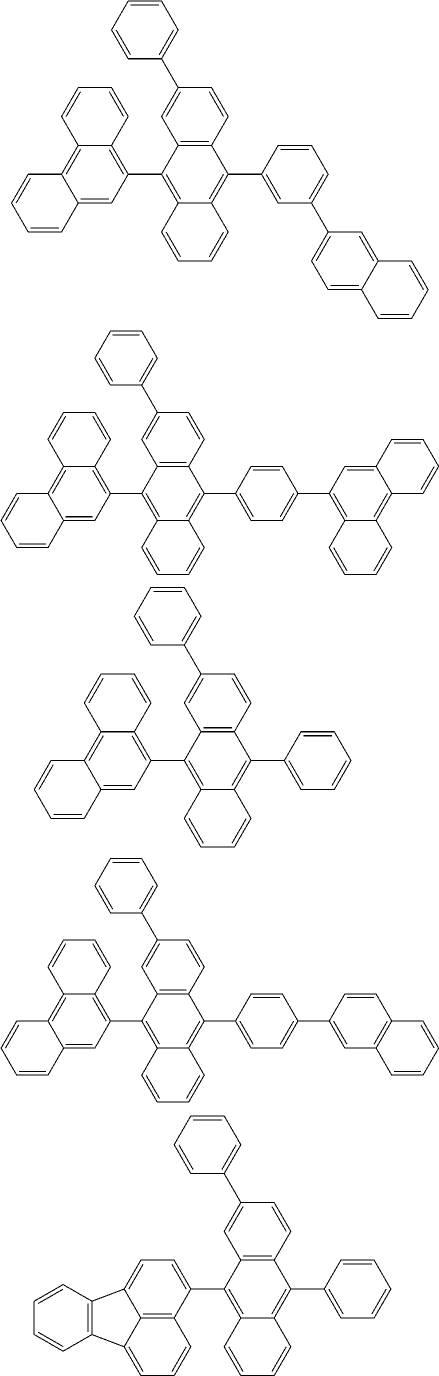

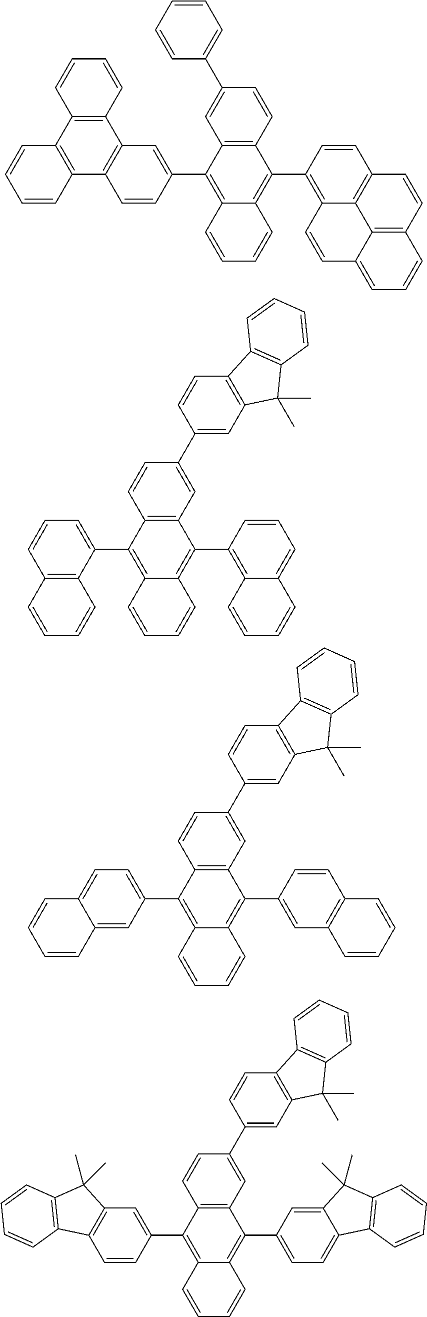

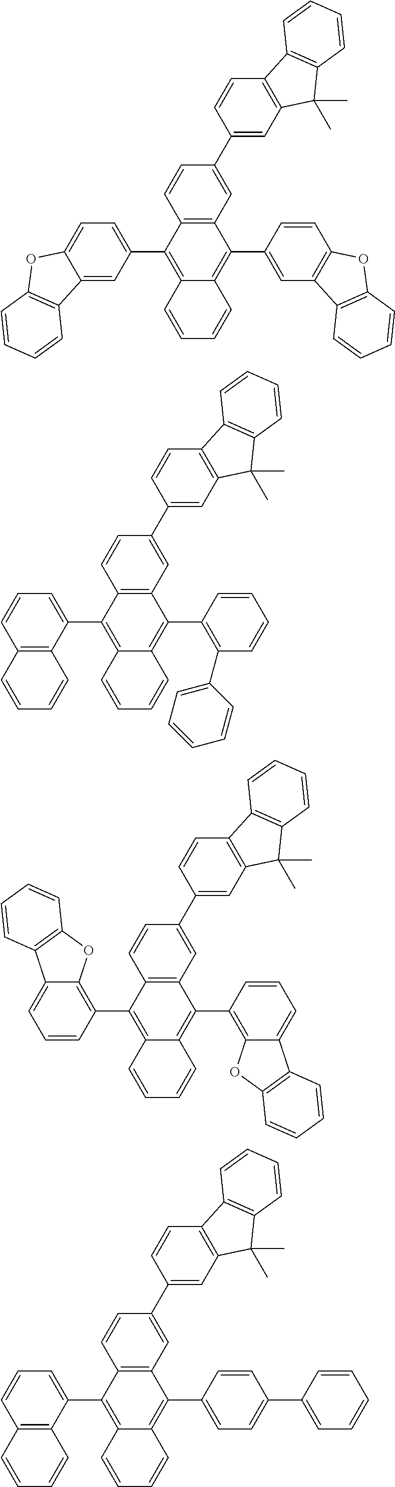

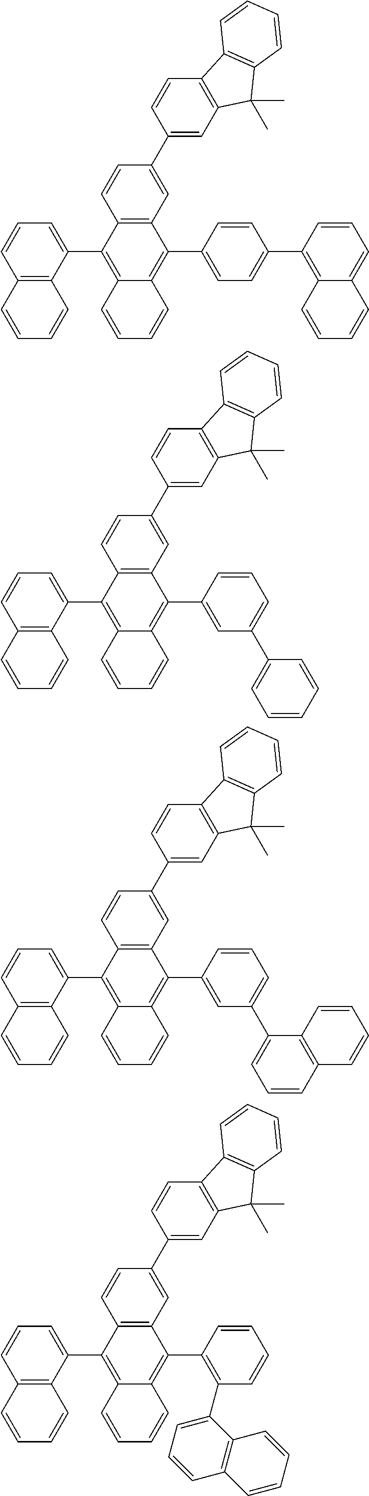

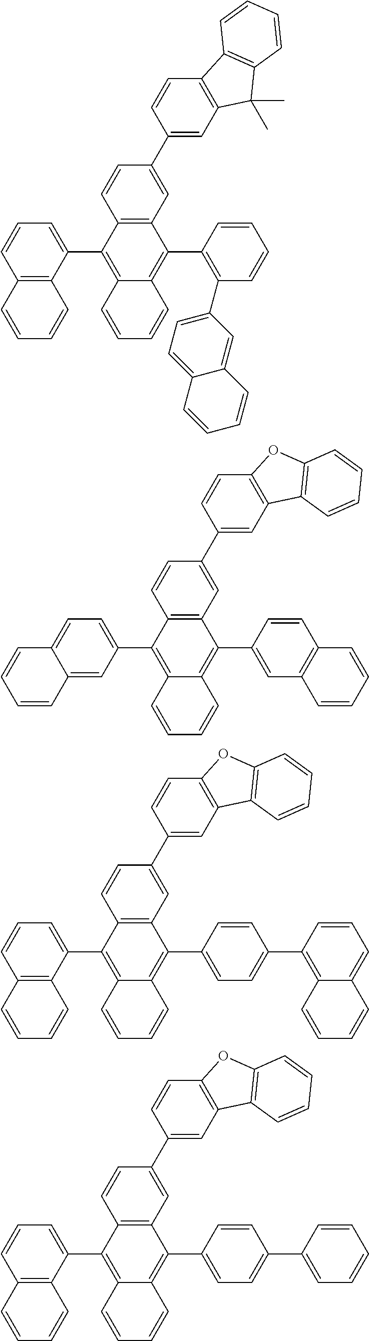

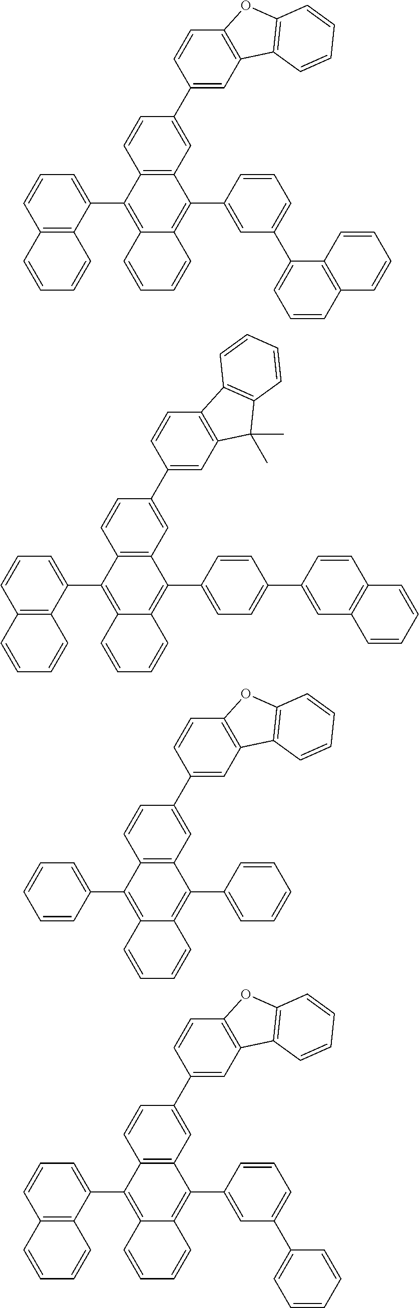

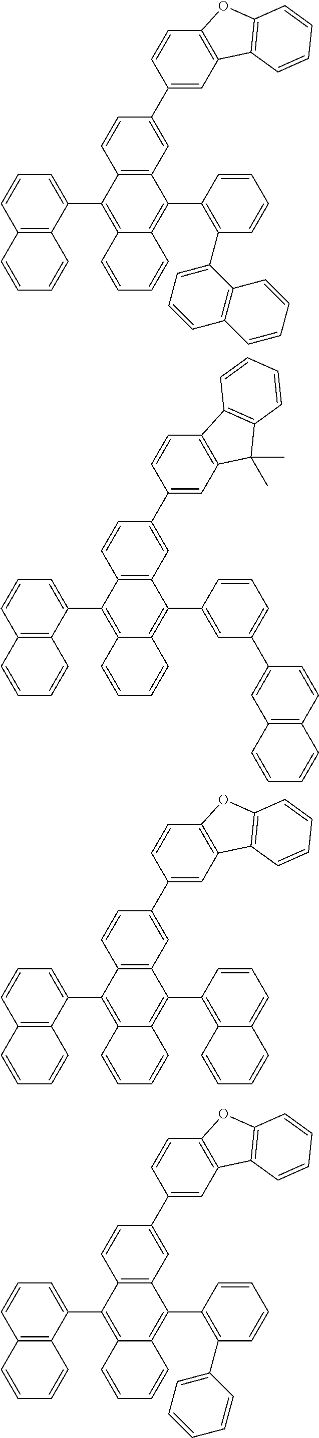

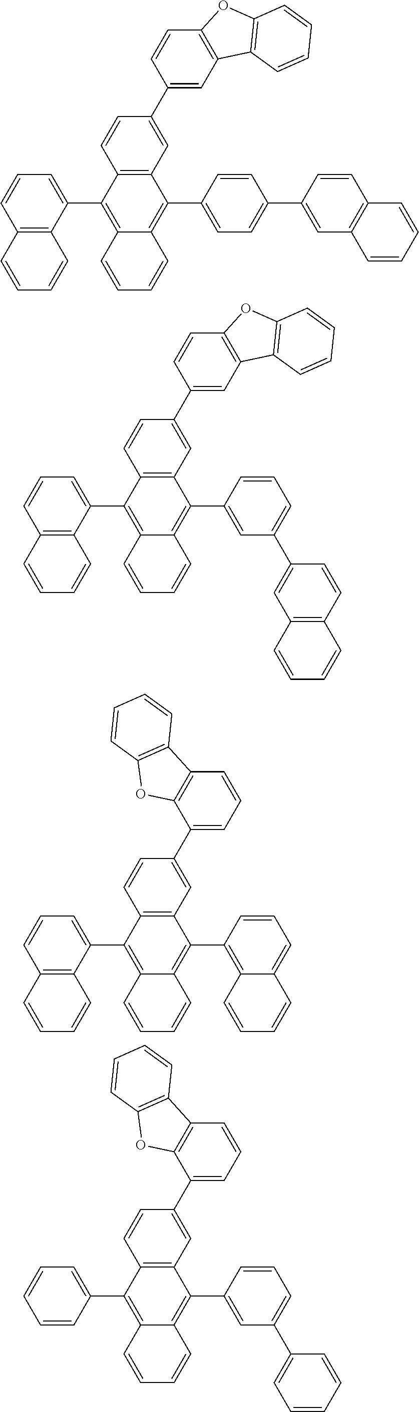

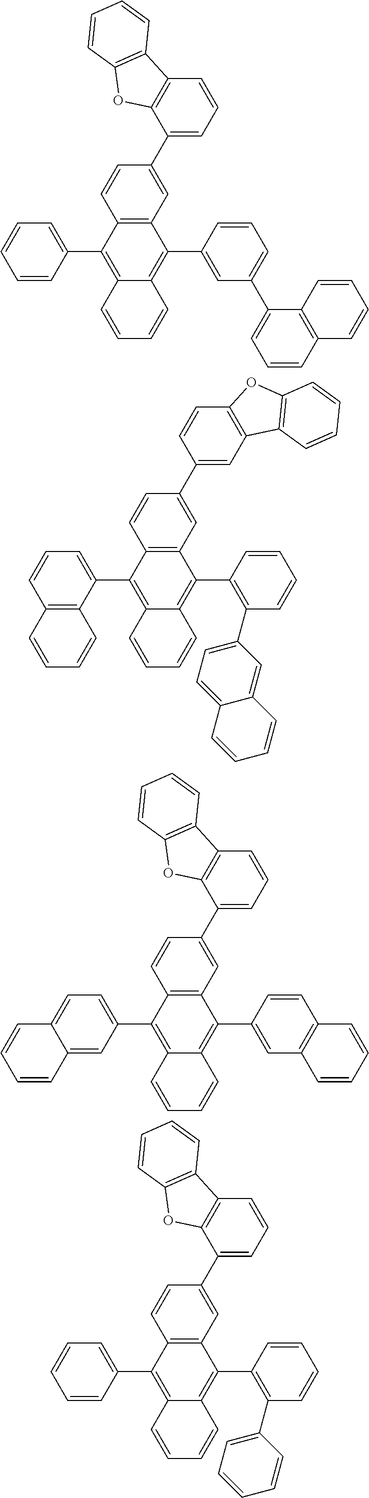

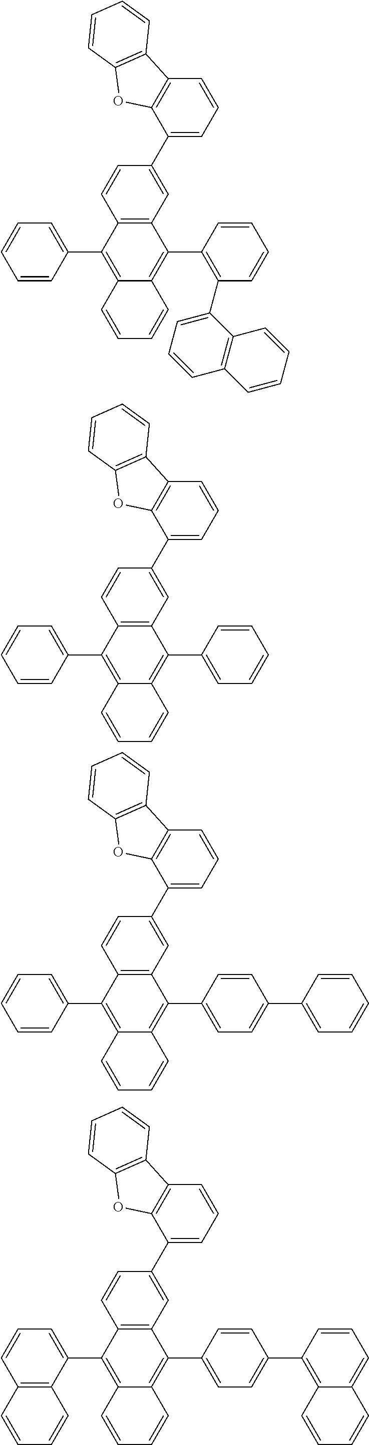

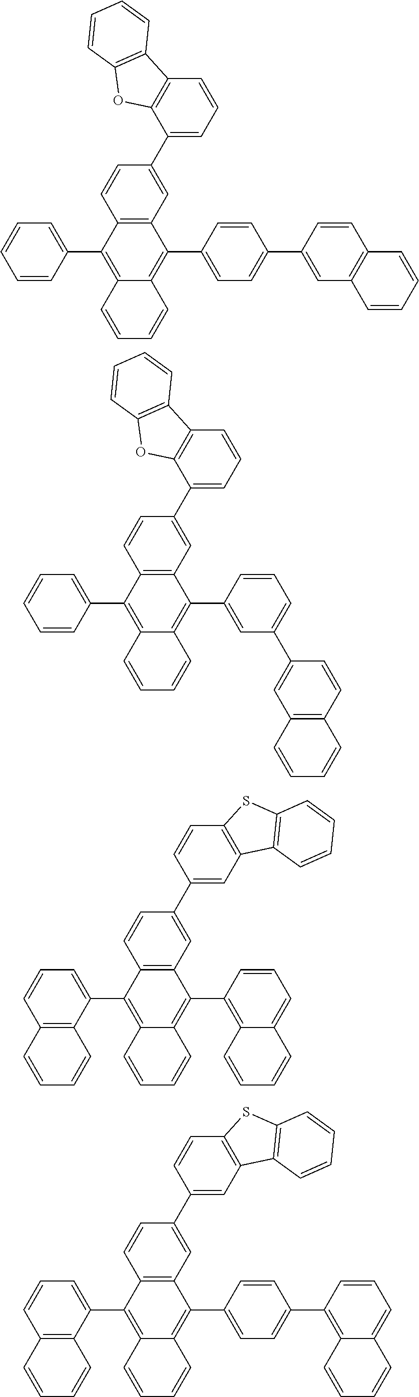

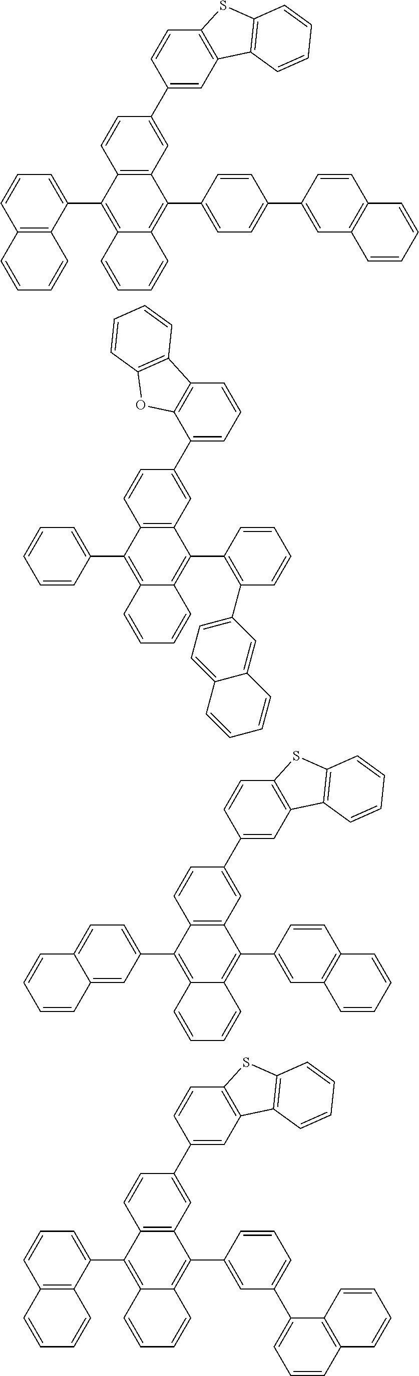

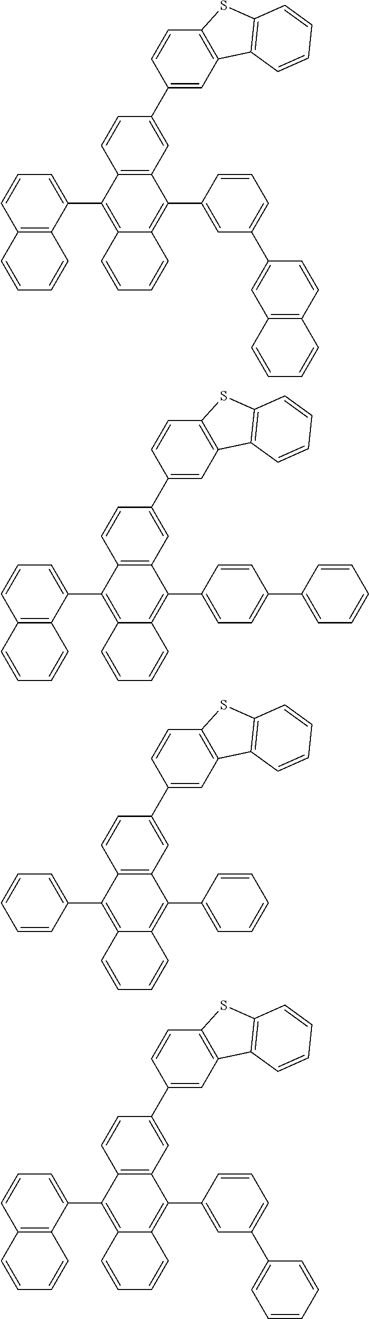

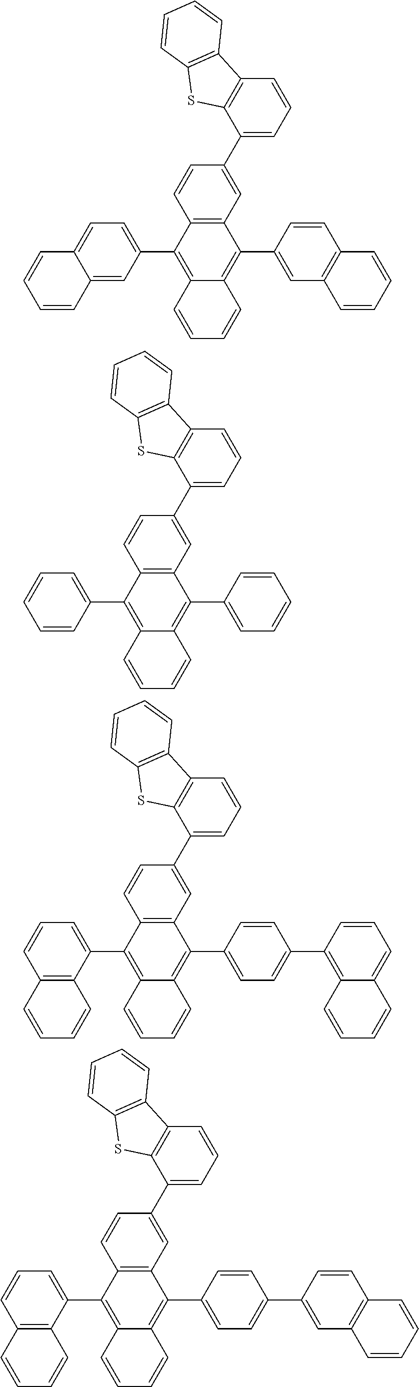

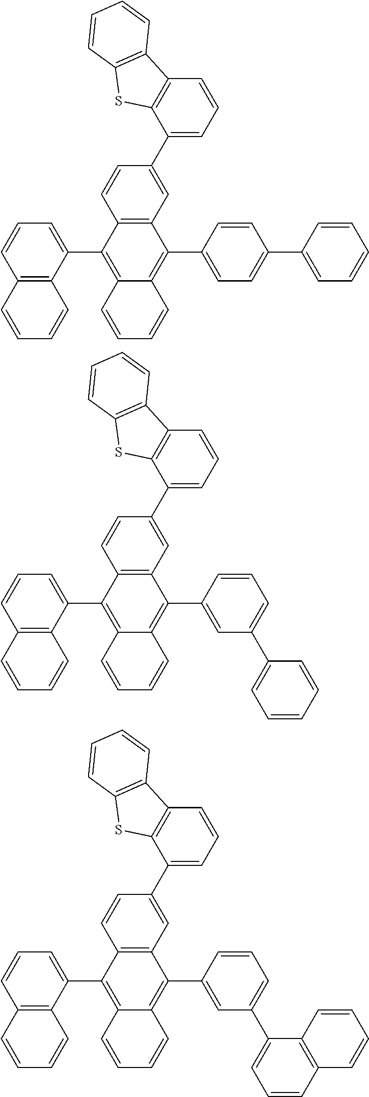

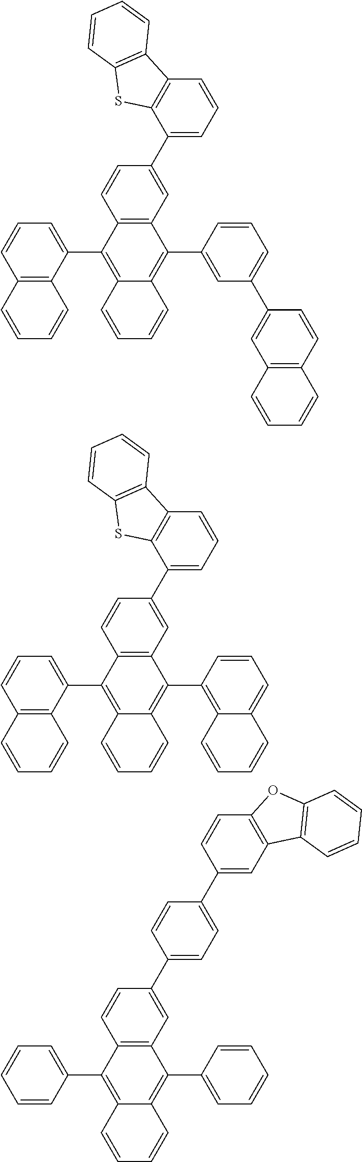

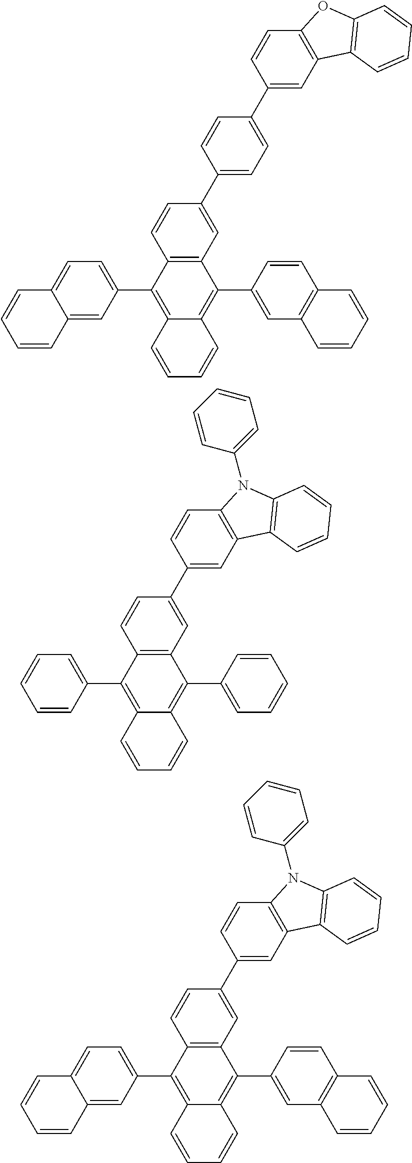

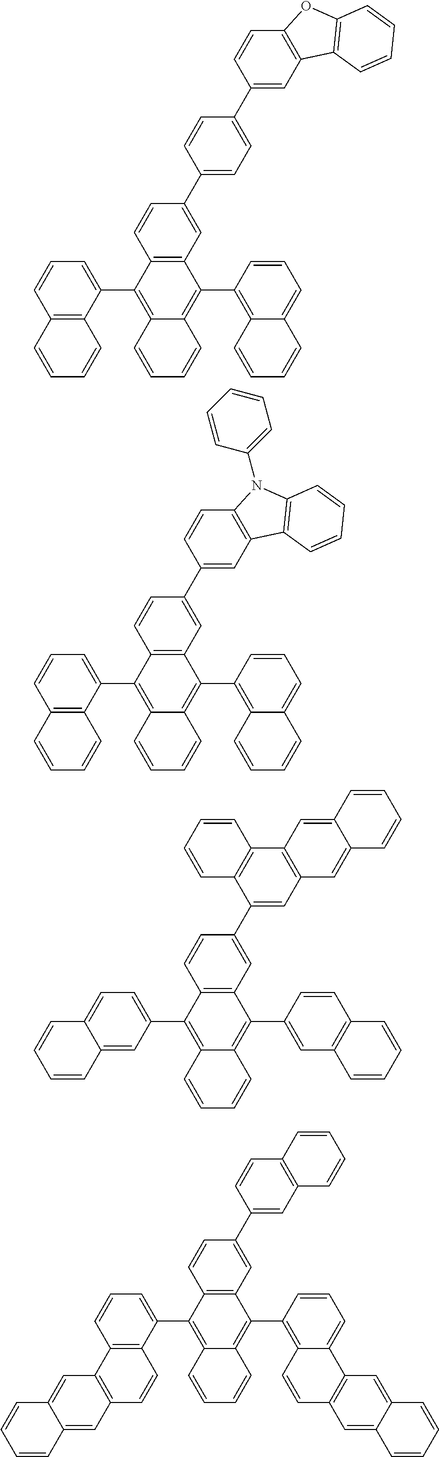

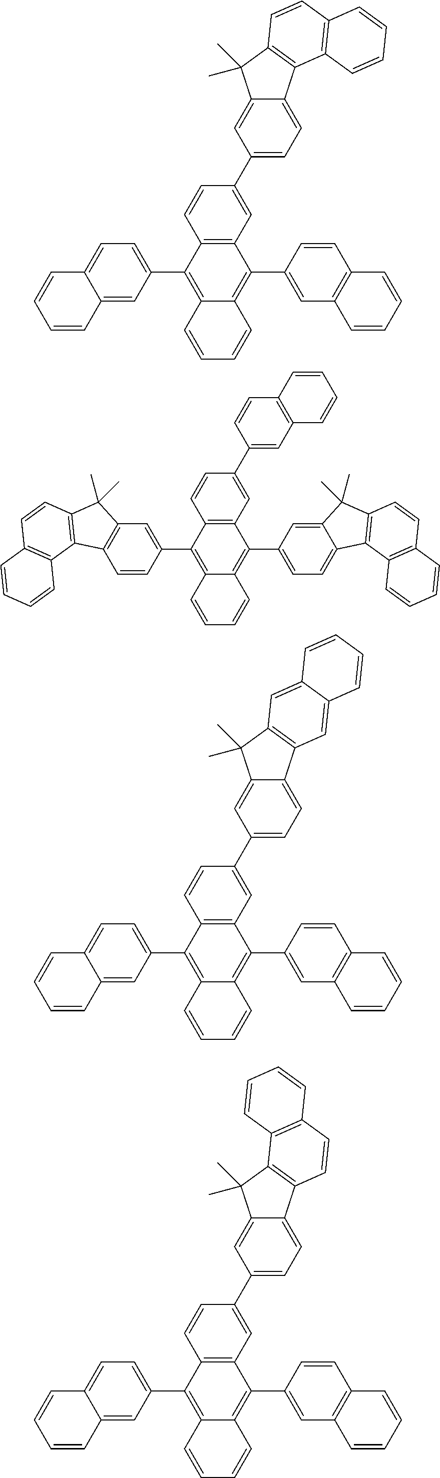

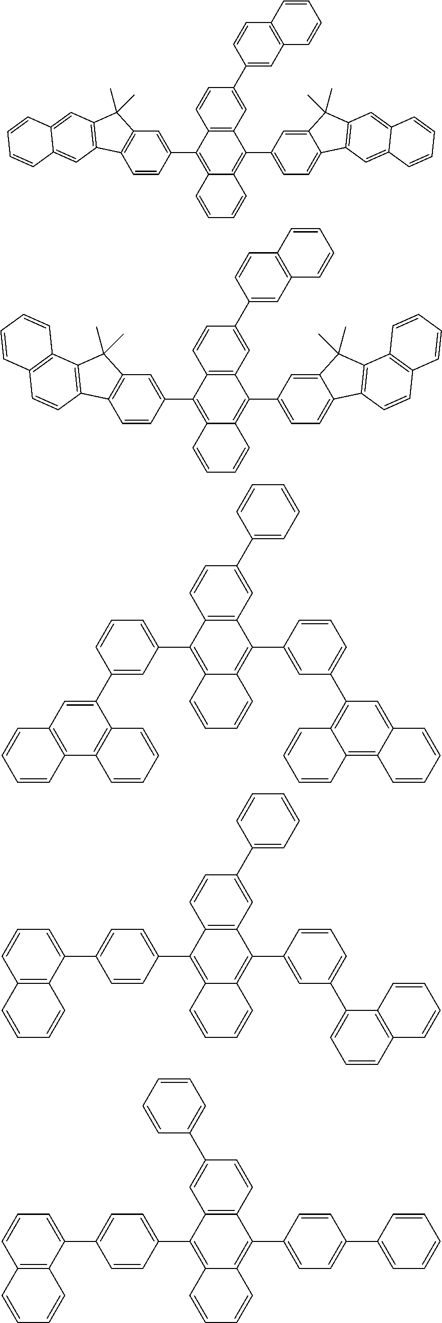

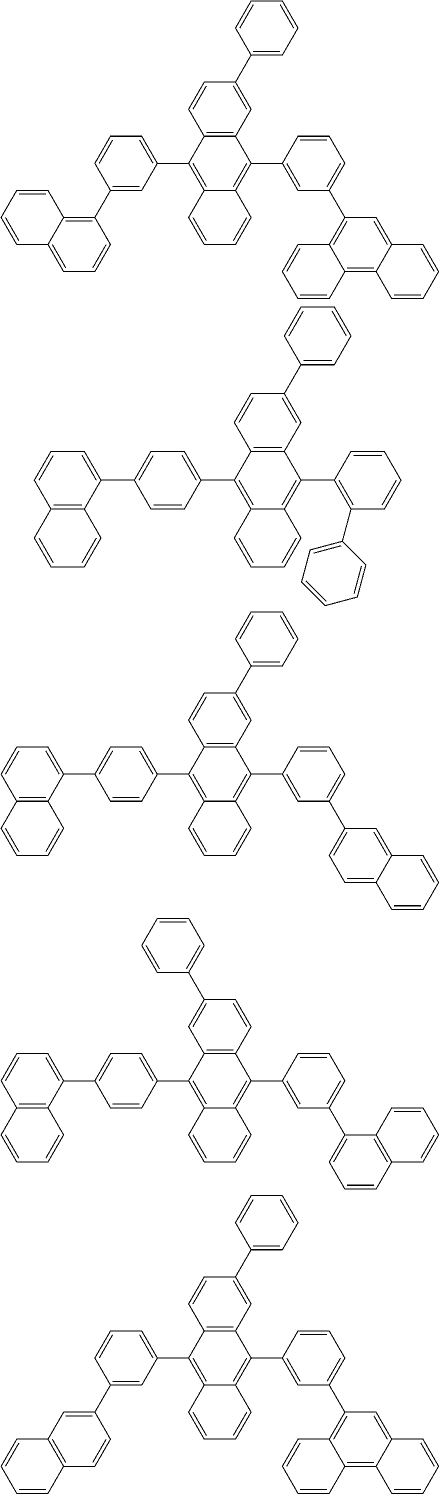

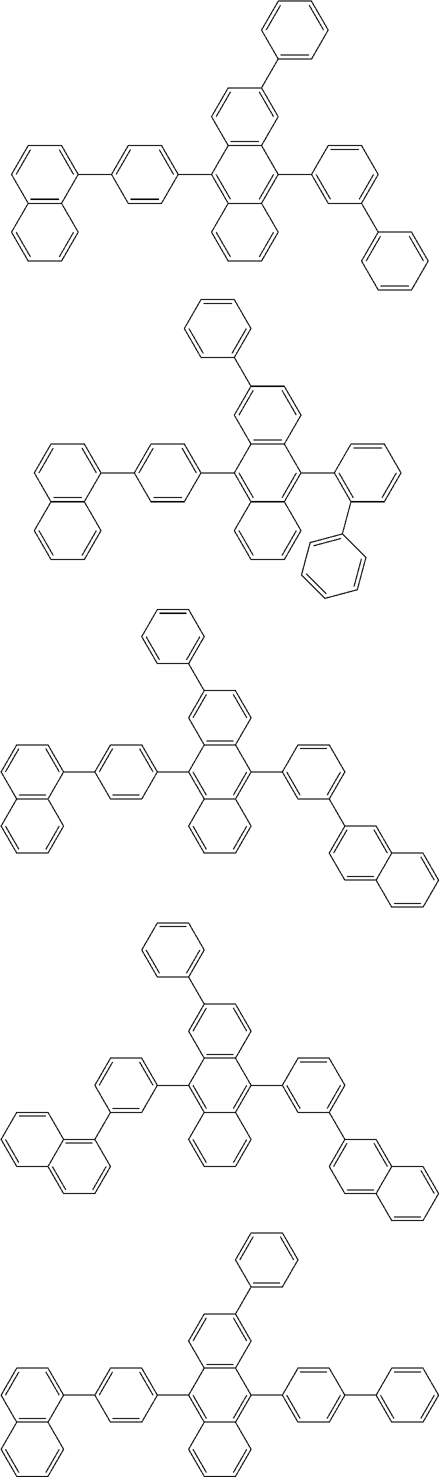

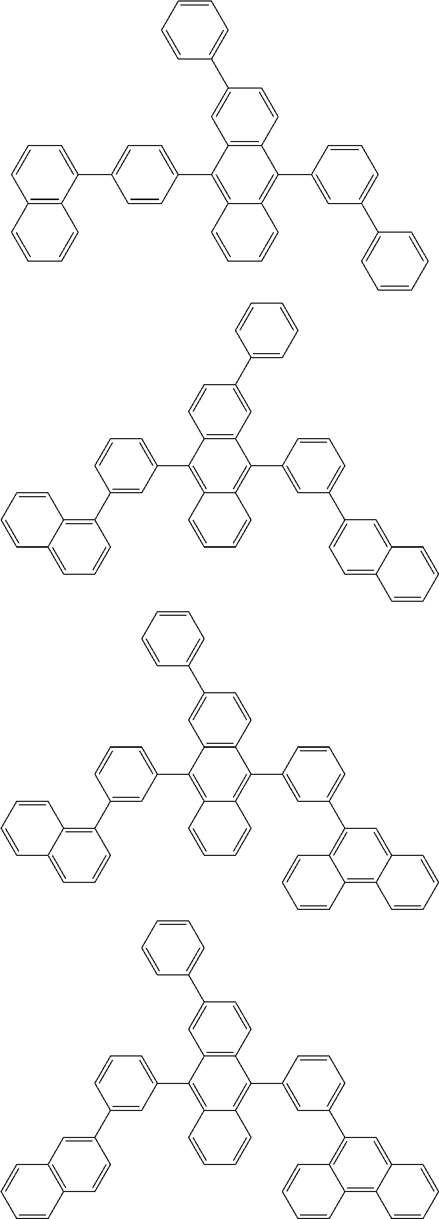

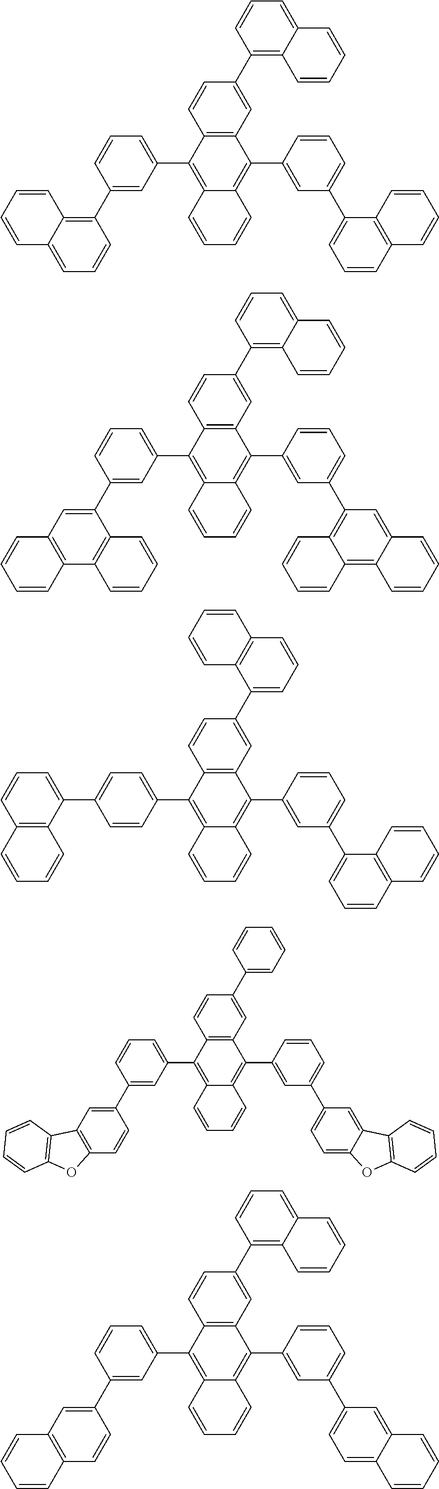

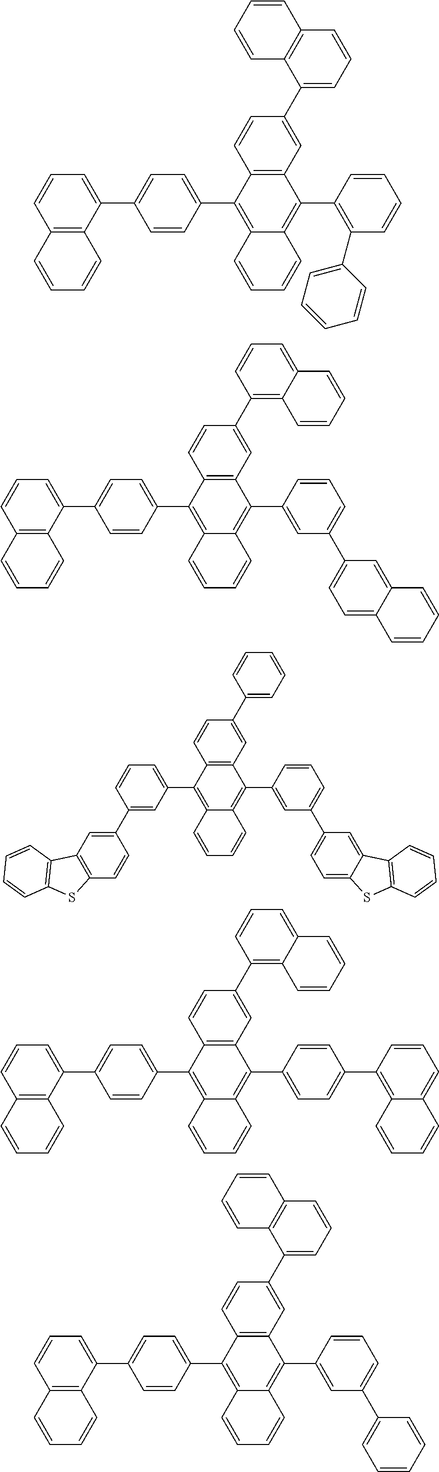

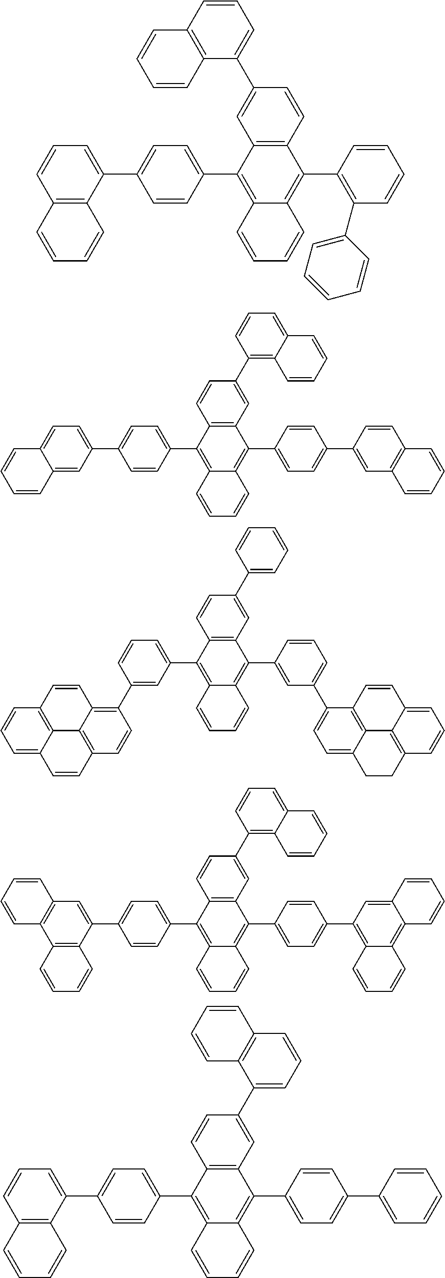

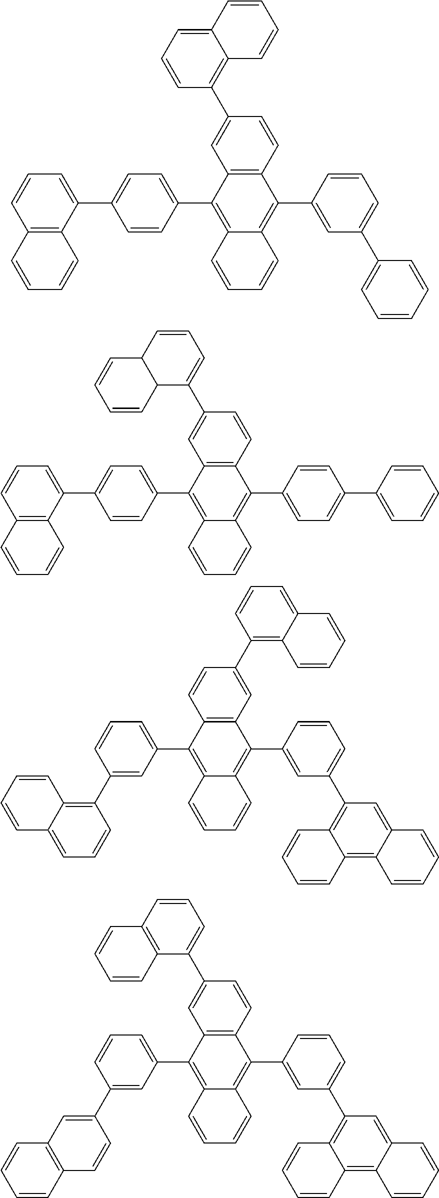

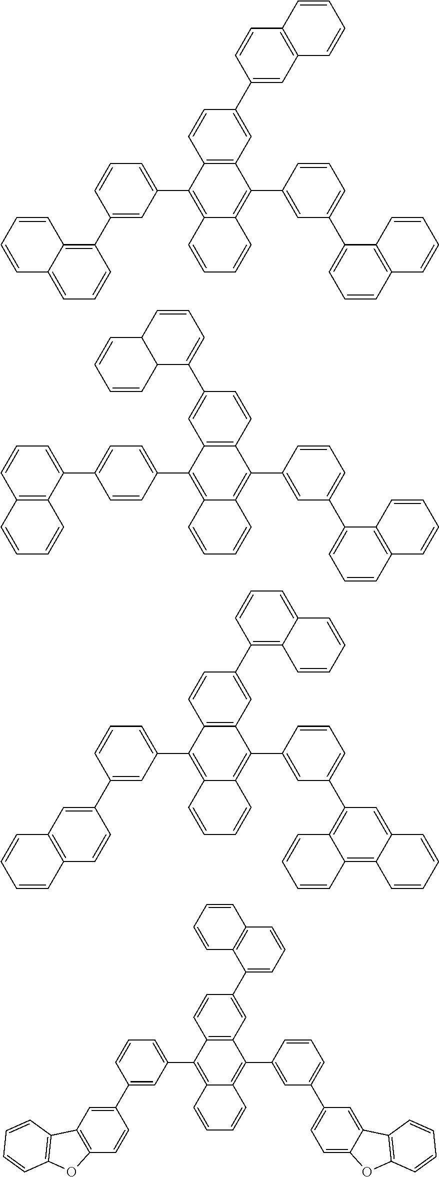

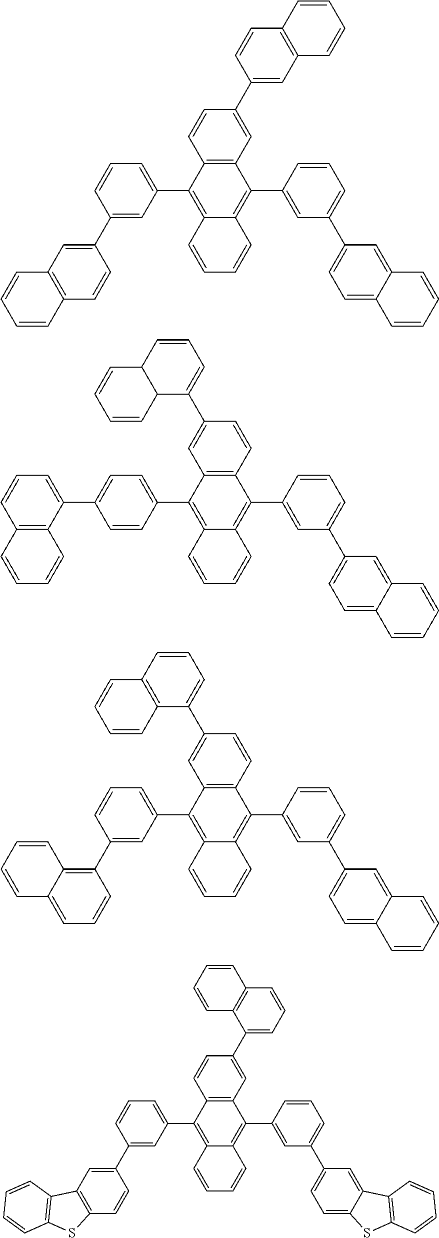

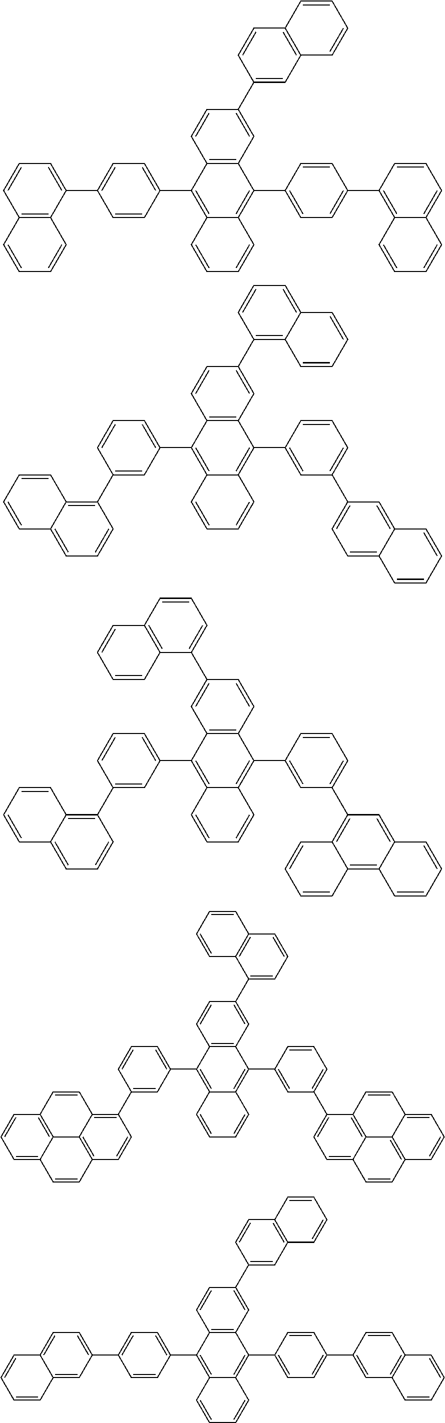

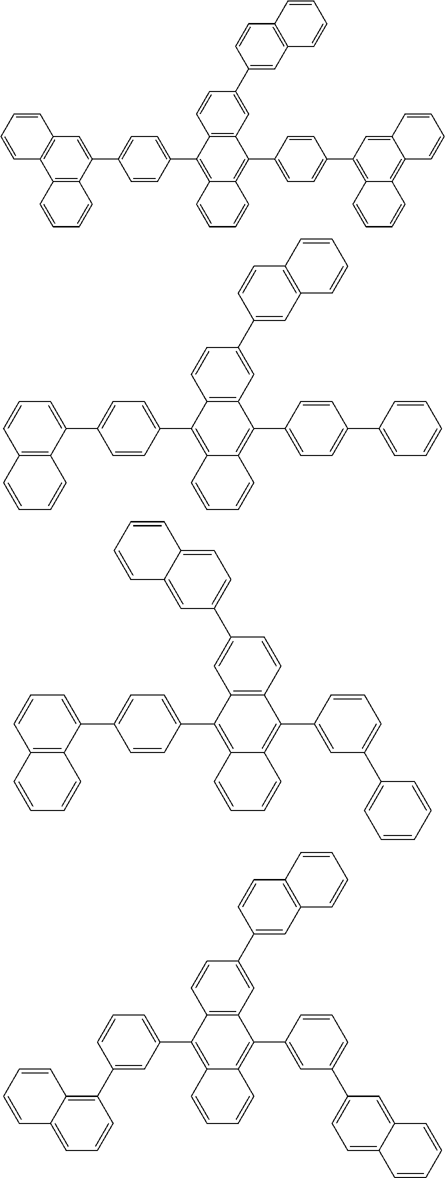

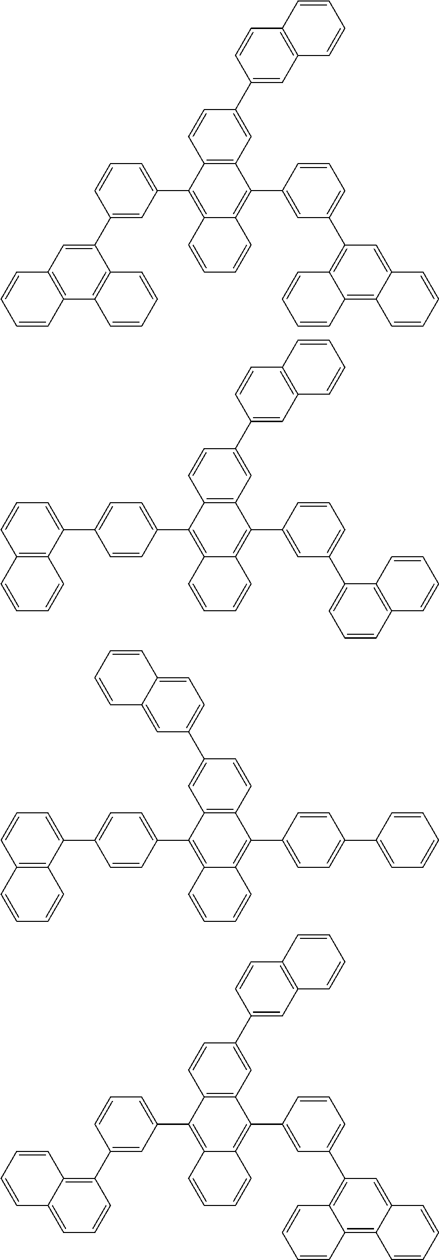

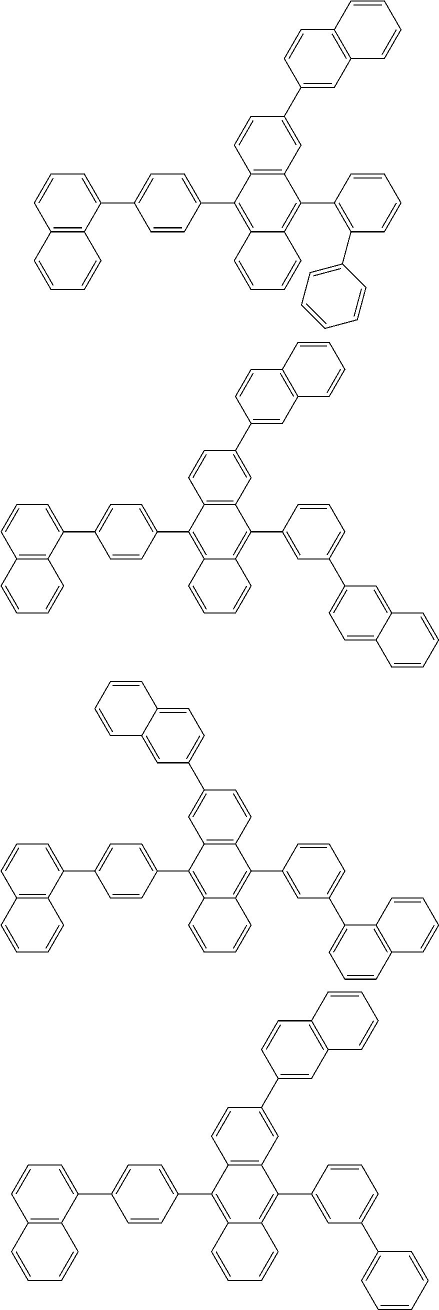

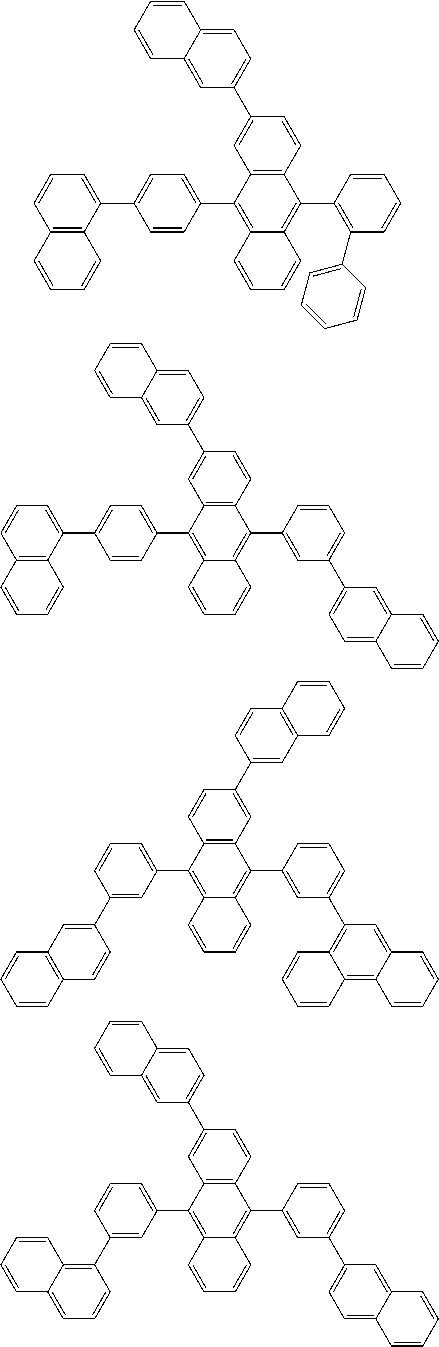

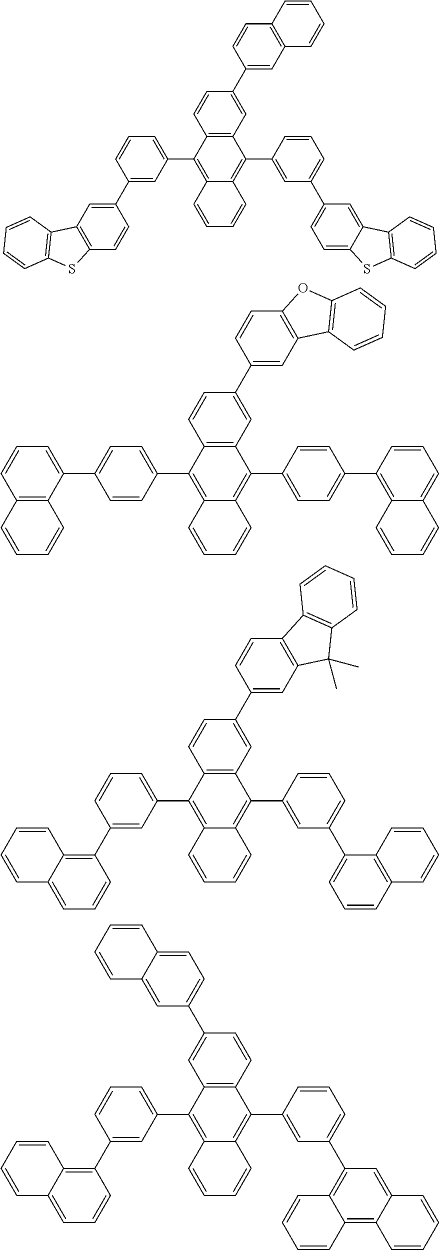

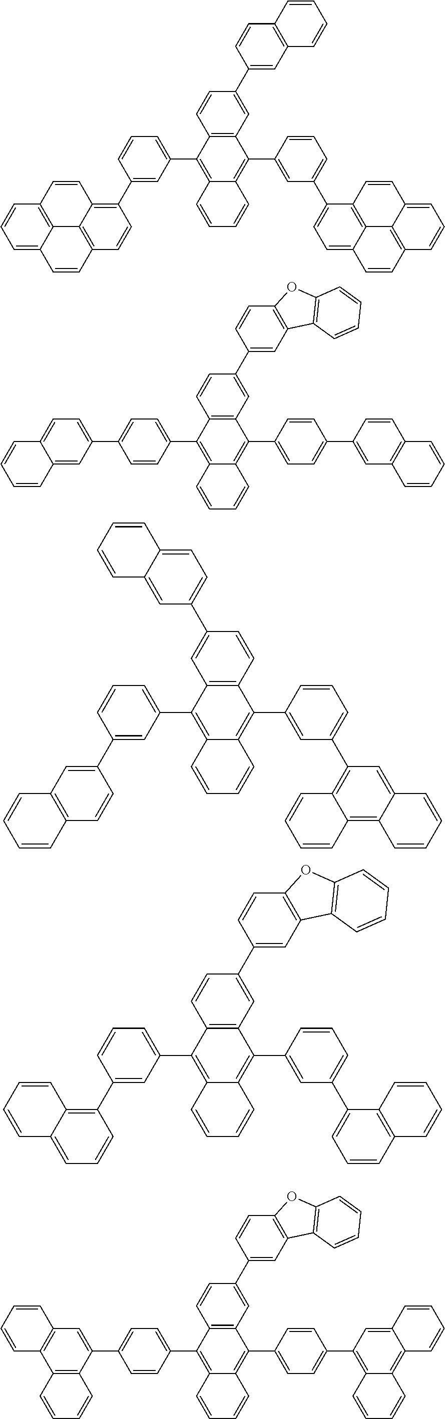

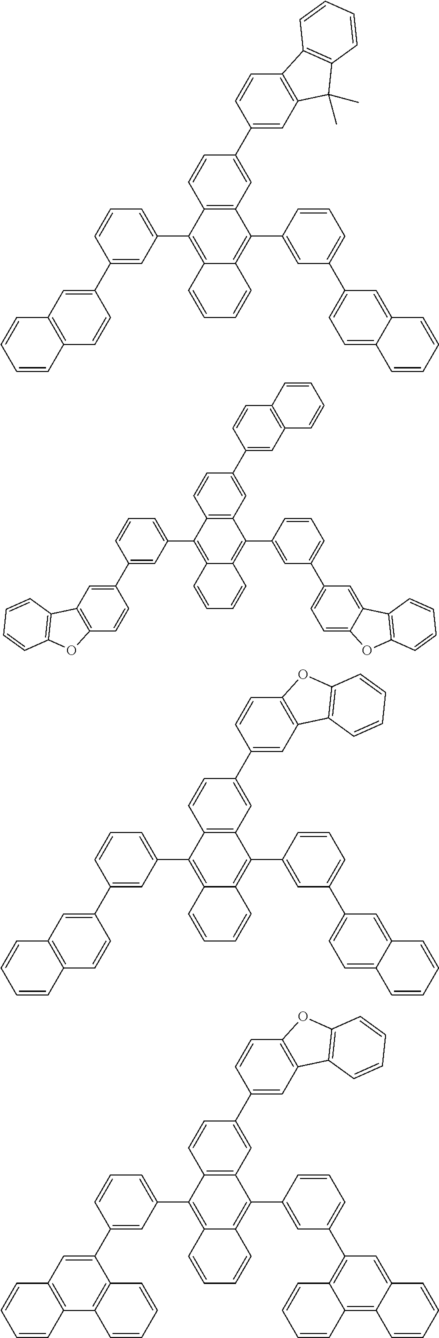

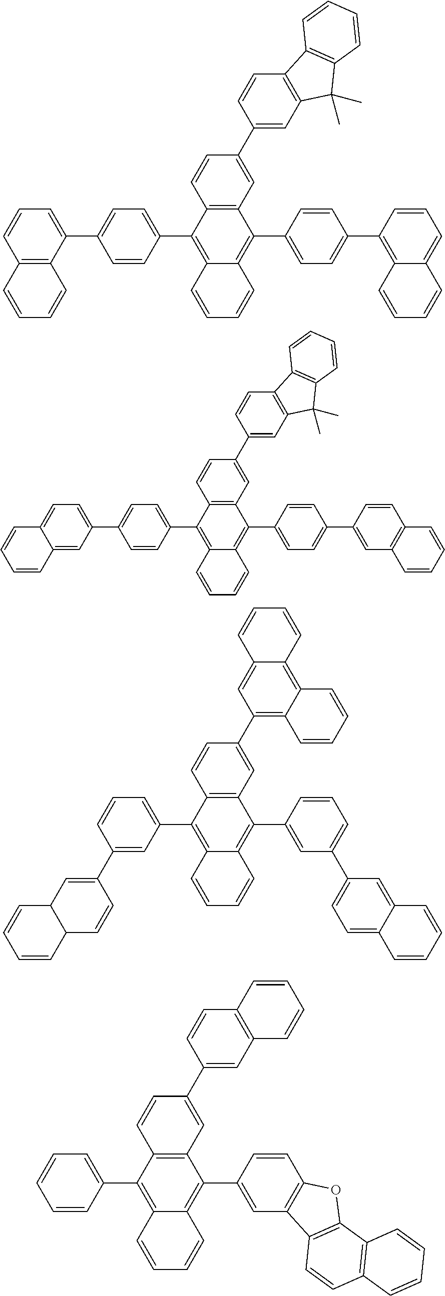

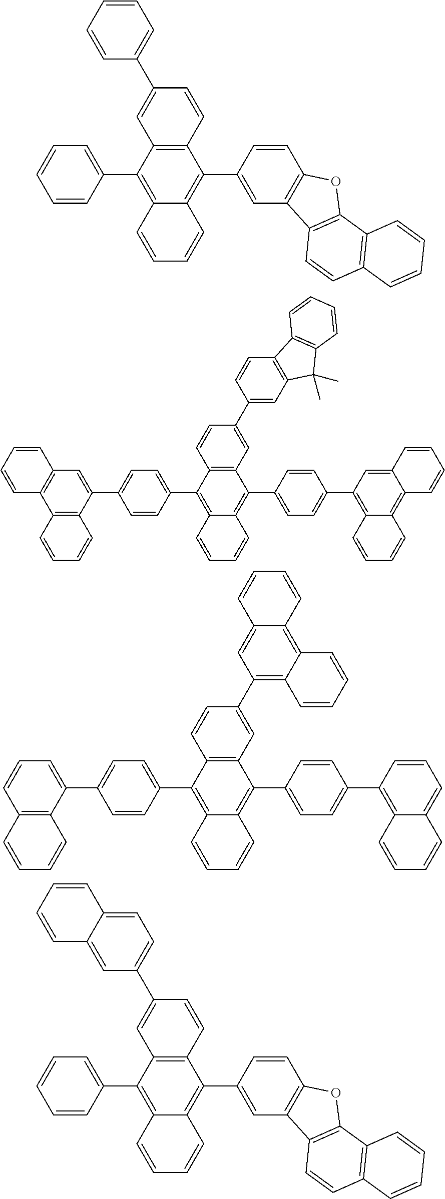

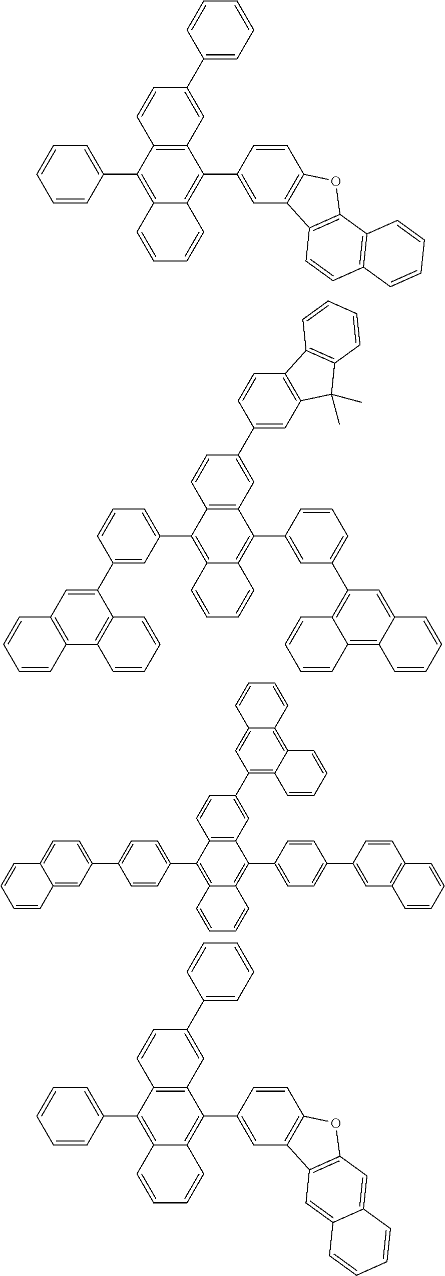

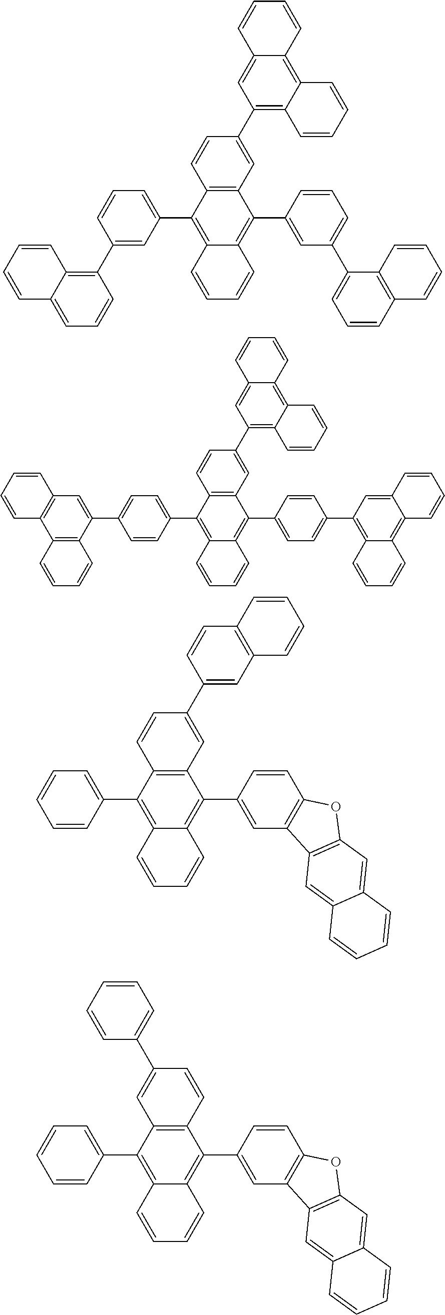

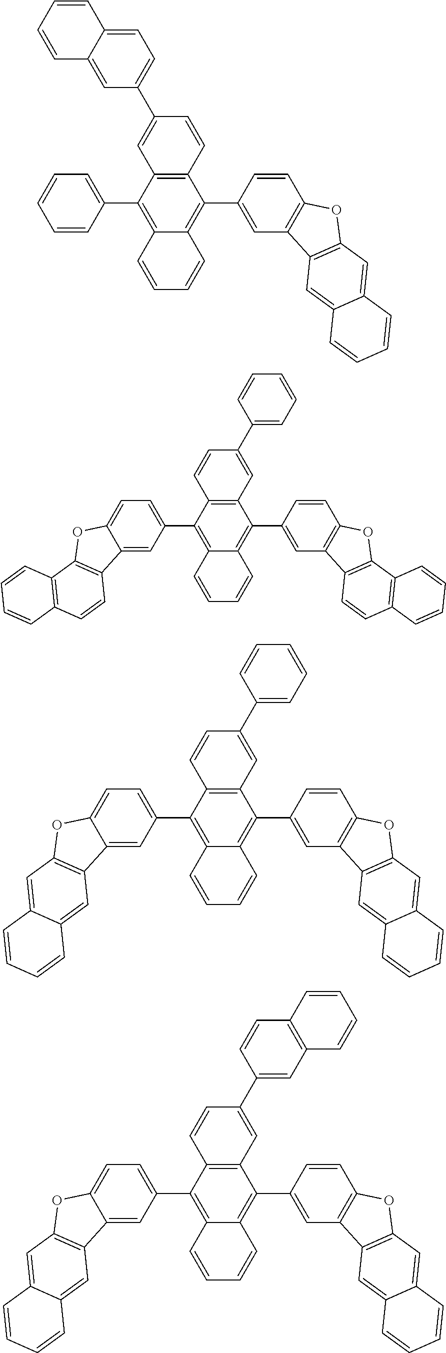 [SMILES: c1ccc(-c2c3ccccc3c(-c3ccc4oc5cc6ccccc6cc5c4c3)c3ccc(-c4ccc5ccccc5c4)cc23)cc1 ; c1ccc(-c2ccc3c(-c4ccc5oc6c7ccccc7ccc6c5c4)c4ccccc4c(-c4ccc5oc6c7ccccc7ccc6c5c4)c3c2)cc1 ; c1ccc(-c2ccc3c(-c4ccc5oc6cc7ccccc7cc6c5c4)c4ccccc4c(-c4ccc5oc6cc7ccccc7cc6c5c4)c3c2)cc1 ; c1ccc2cc(-c3ccc4c(-c5ccc6oc7cc8ccccc8cc7c6c5)c5ccccc5c(-c5ccc6oc7cc8ccccc8cc7c6c5)c4c3)ccc2c1]

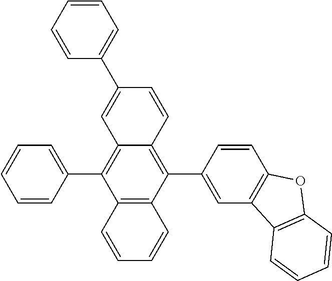 [SMILES: c1ccc(-c2ccc3c(-c4ccc5oc6ccccc6c5c4)c4ccccc4c(-c4ccccc4)c3c2)cc1]

### [Compound Represented by the Formula (11)]

Next, a compound represented by the formula (11) will be described.

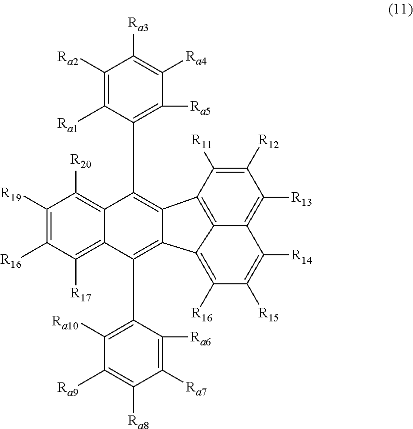

In the formula (11),

any one or more sets among one or more sets of adjacent two or more of R₁₁ to R₂₀, one or more sets of adjacent two or more of Rₐ₁ to Rₐ₅, and one or more sets of adjacent two or more of Rₐ₆ to Rₐ₁₀ form a substituted or unsubstituted, saturated or unsaturated ring including 3 to 30 ring atoms by bonding with each other;

R₁₁ to R₂₀, Rₐ₁ to Rₐ₅ and Rₐ₆ to Rₐ₁₀ which do not form the ring are independently,

a hydrogen atom,

a substituted or unsubstituted alkyl group including 1 to 30 carbon atoms,

a substituted or unsubstituted cycloalkyl group including 3 to 30 ring carbon atoms,

a substituted or unsubstituted alkoxy group including 1 to 30 carbon atoms,

a substituted or unsubstituted alkylthio group including 1 to 30 carbon atoms,

a substituted or unsubstituted amino group,

a substituted or unsubstituted aryl group including 6 to 30 ring carbon atoms,

a substituted or unsubstituted heterocyclic group including 5 to 30 ring atoms.

a substituted or unsubstituted alkenyl group including 2 to 30 carbon atoms,

a substituted or unsubstituted aryloxy group including 6 to 30 ring carbon atoms,

a substituted or unsubstituted arylthio group including 6 to 30 ring carbon atoms,

a substituted or unsubstituted phosphanyl group,

a substituted or unsubstituted phosphoryl group,

a substituted or unsubstituted silyl group,

a substituted or unsubstituted arylcarbonyl group including 6 to 30 ring carbon atoms,

a cyano group, a nitro group, a carboxyl group, or

a halogen atom.

At least one set of adjacent two or more of R₁₁ to R₁₆, R₁₇ to R₂₀, Rₐ₁ to Rₐ₅, or Rₐ₆ to Rₐ₁₀ form a ring by bonding with each other.

Specific examples in which “one or more sets of adjacent two or more of R₁₁ to R₂₀, one or more sets of adjacent two or more of Rₐ₁ to Rₐ₅, and one or more sets of adjacent two or more of Rₐ₆ to Rₐ₁₀” form a substituted or unsubstituted, saturated or unsaturated ring including 3 to 30 ring atoms by bonding with each other are explained.

Specific examples in which adjacent two or more form a ring by bonding with each other include, for example, the following partial structure by taking R₁₇ to R₂₀ in the formula (11) as an example. In the following partial structure, adjacent three of R₁₈ and R₁₉ and R₂₀ form a ring by bonding with each other.

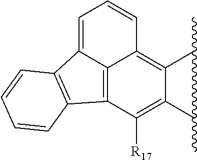

Further, Specific examples in which “one or more sets of adjacent two or more” form a ring by bonding each other include, for example, the following partial structure by taking R₁₁ to R₁₆ in the formula (11) as an example. In the following partial structure, two sets of R₁₂ and R₁₃, and R₁₄ and R₁₅ form two separate rings by bonding with each other.

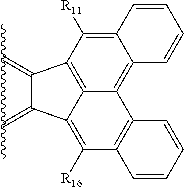

In one embodiment, R₁₂ and R₁₃ in the formula (11) form a substituted or unsubstituted, saturated or unsaturated ring including 3 to 30 ring atoms by bonding with each other.

In one embodiment, the compound represented by the formula (11) is a compound represented by the following formula (11-1).

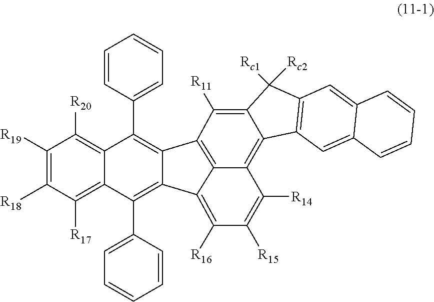

In the formula (11-1), R₁₁, R₁₄ to R₂₀ are as defined in the formula (11).

R_{c1} and R_{c2} are independently,

a hydrogen atom,

an unsubstituted alkyl group including 1 to 50 carbon atoms,

an unsubstituted alkenyl group including 2 to 50 carbon atoms,

an unsubstituted alkynyl group including 2 to 50 carbon atoms,

an unsubstituted cycloalkyl group including 3 to 50 ring carbon atoms,

—Si(R₉₀₁)(R₉₀₂)(R₉₀₃),

—O—(R₉₀₄),

—S—(R₉₀₅),

a halogen atom, a cyano group, a nitro group,

an unsubstituted aryl group including 6 to 50 ring carbon atoms, or

an unsubstituted monovalent heterocyclic group including 5 to 50 ring atoms;

R₉₀₁ to R₉₀₇ are independently,

a hydrogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms;

when two or more of each of R₉₀₁ to R₉₀₇ are present, the two or more of each of R₉₀₁ to R₉₀₇ may be the same as or different to each other.

In one embodiment, two or more among R₁₈ to R₂₀ in the formula (11) form a substituted or unsubstituted, saturated or unsaturated ring including 3 to 30 ring atoms by bonding with each other.

In one embodiment, the compound represented by the formula (11) is a compound represented by the following formula (11-2).

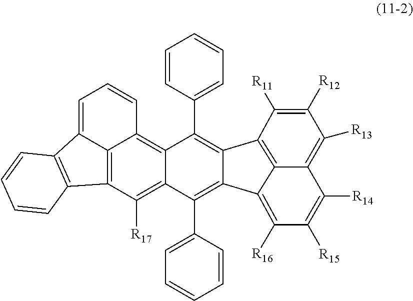

In the formula (11-2), R₁₁ to R₁₇ are as defined in the formula (11).

In one embodiment, R₁₁ to R₂₀, Rₐ₁ to Rₐ₅ and Rₐ₆ to Rₐ₁₀ in the formula (11), which do not form a ring are independently,

a hydrogen atom,

an unsubstituted aryl group including 6 to 50 ring carbon atoms, or

an unsubstituted monovalent heterocyclic group including 5 to 50 ring atoms.

In one embodiment, the organic layer includes an emitting layer, and

the emitting layer contains the compound represented by the formula (1) and the compound represented by the formula (11). In this case, the compound represented by the formula (1) functions as a host material of the emitting layer, and the compound represented by the formula (11) functions as a dopant material of the emitting layer.

Specific examples of the compound represented by the formula (11) will be described below, but these are merely examples, and the compound represented by the formula (11) is not limited to the following specific examples.

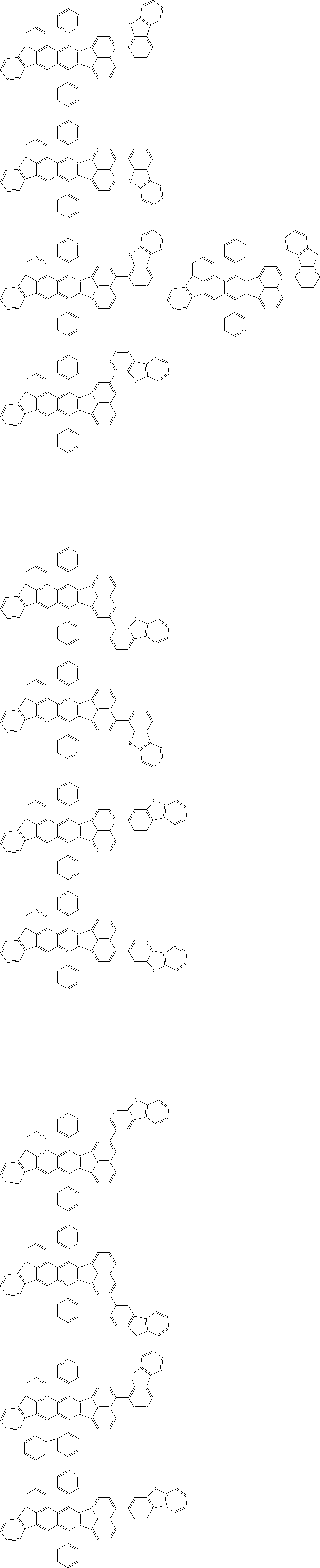

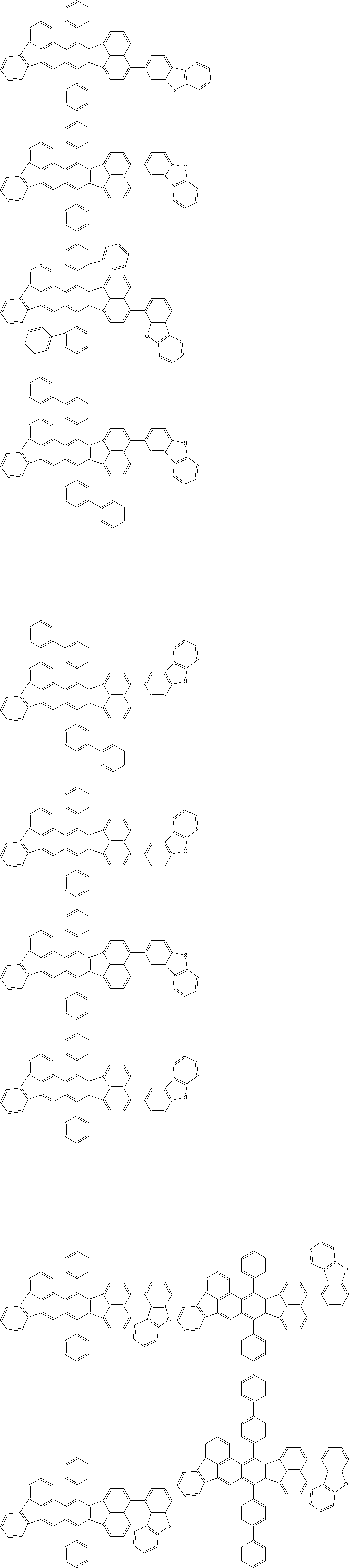

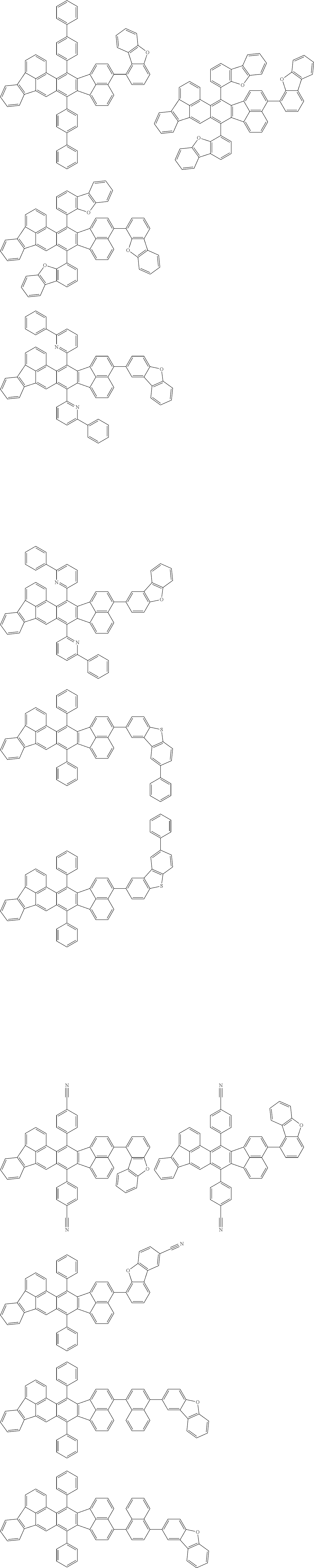

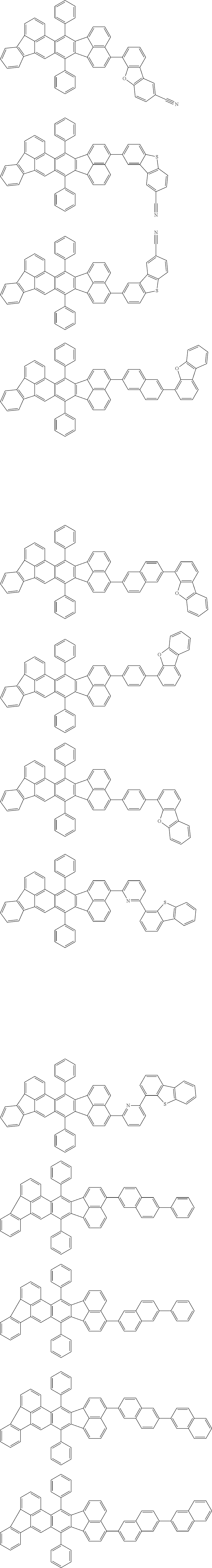

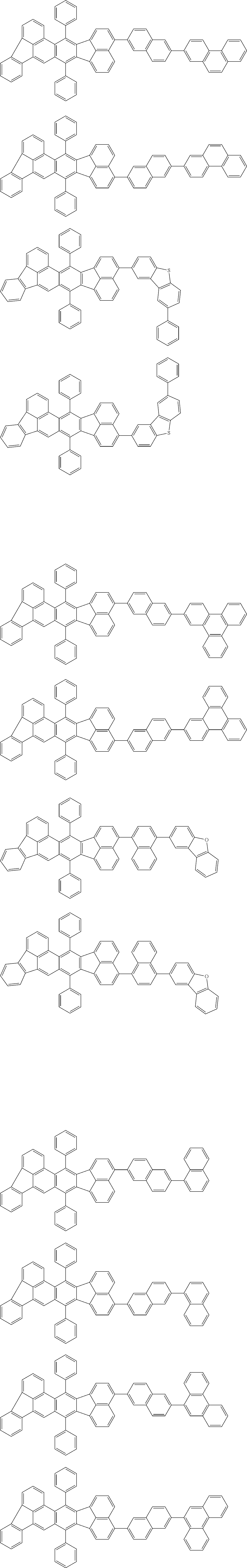

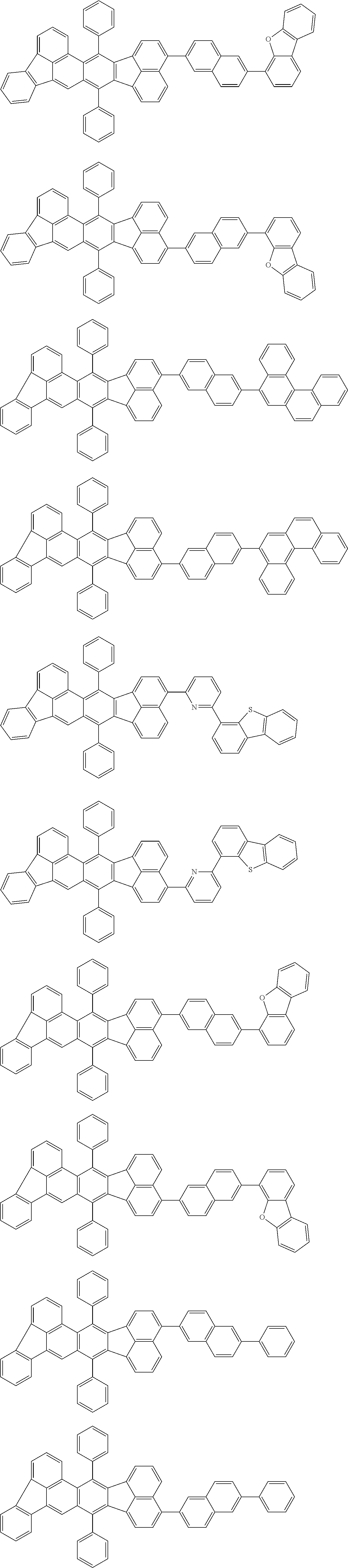

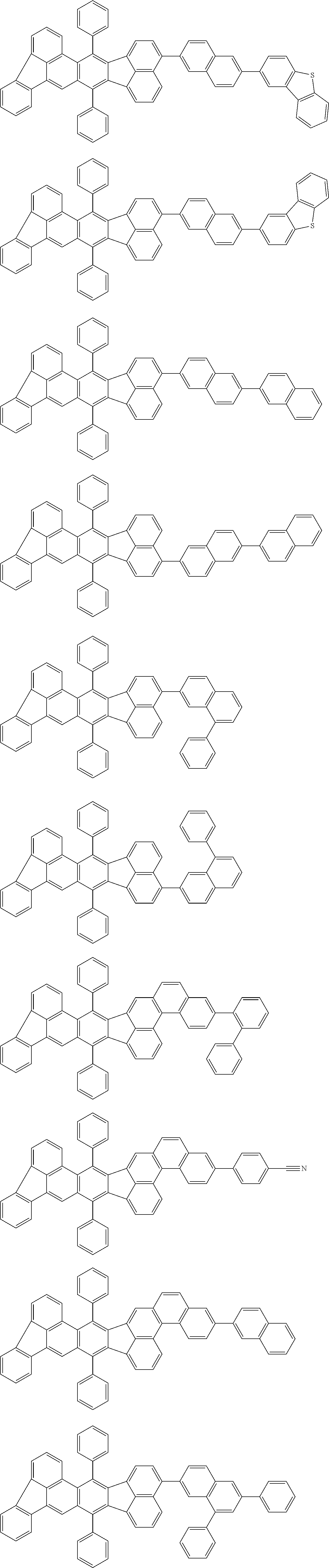

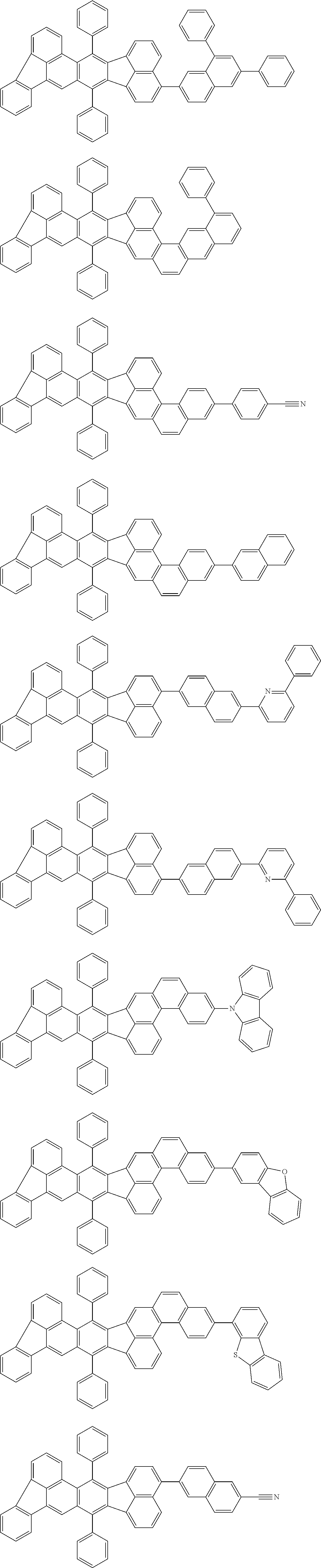

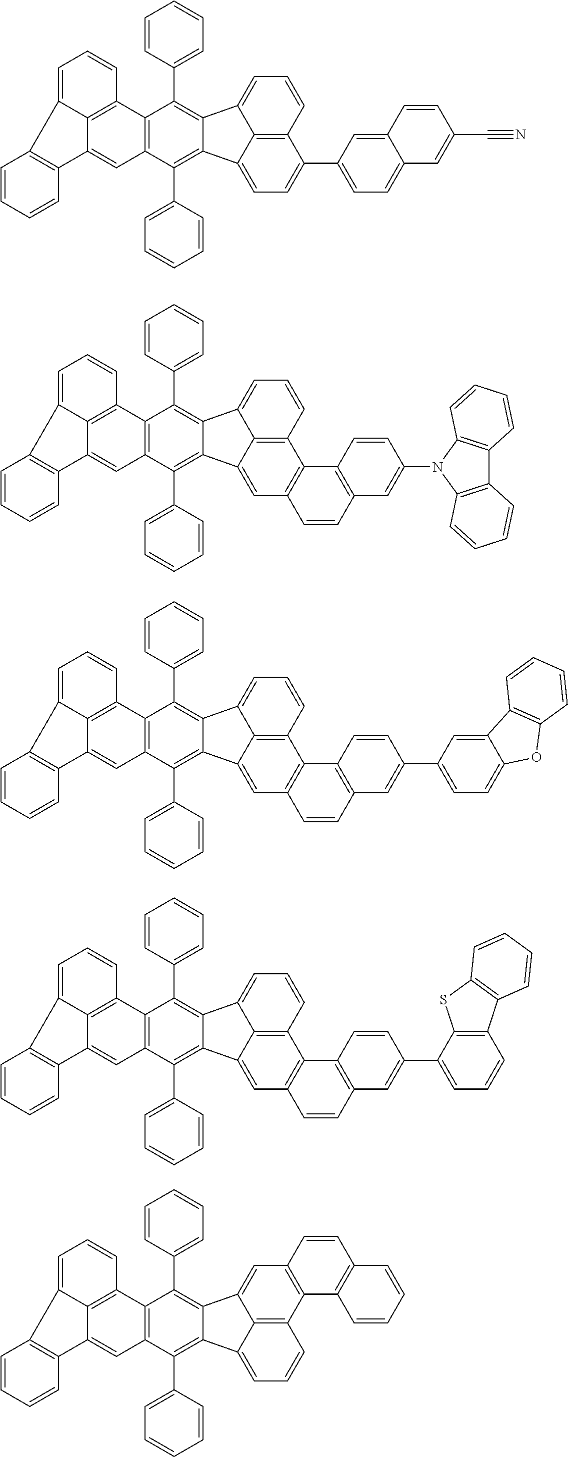

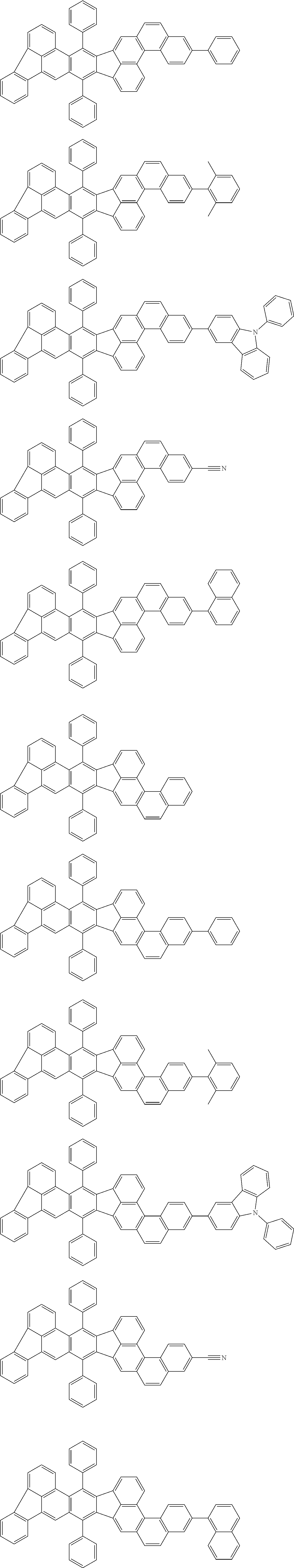

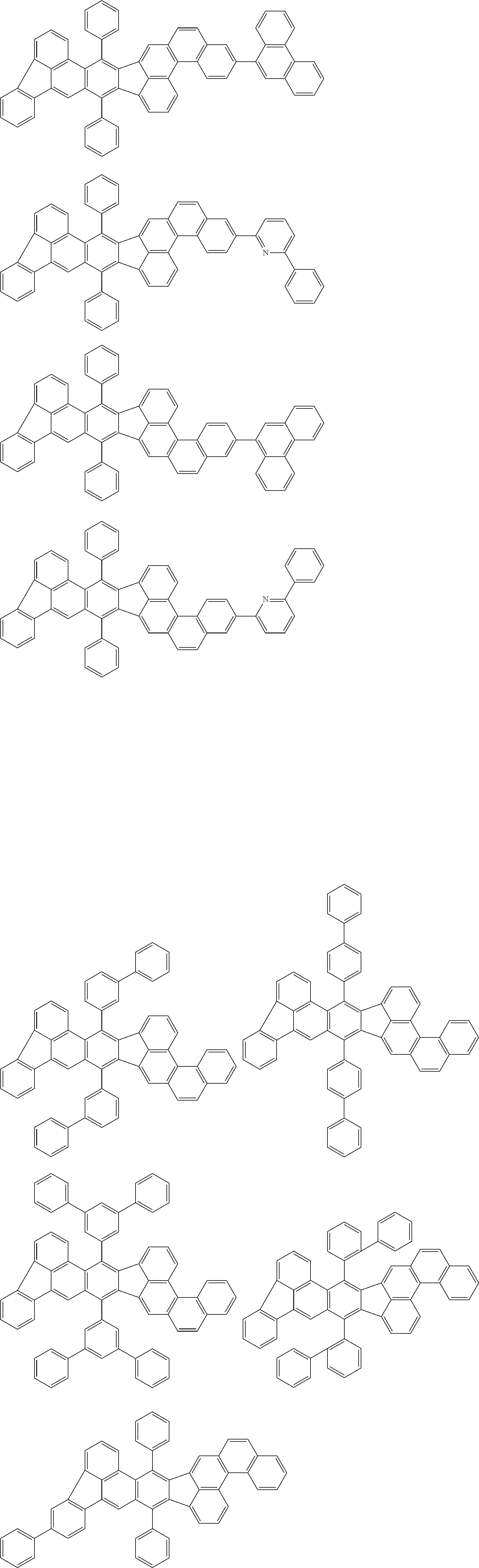

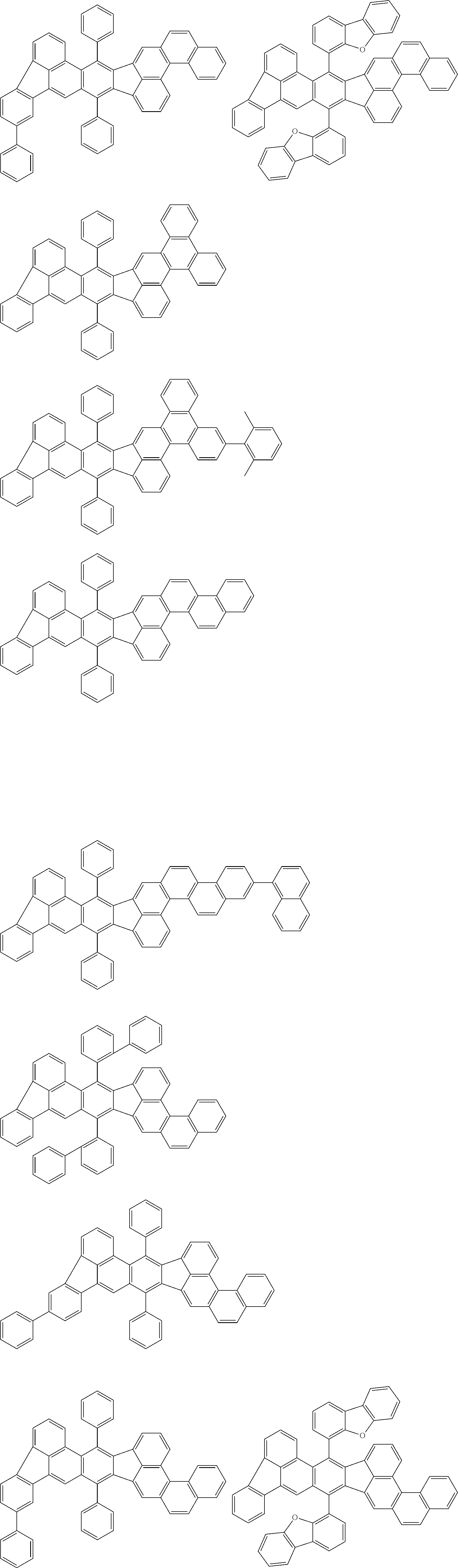

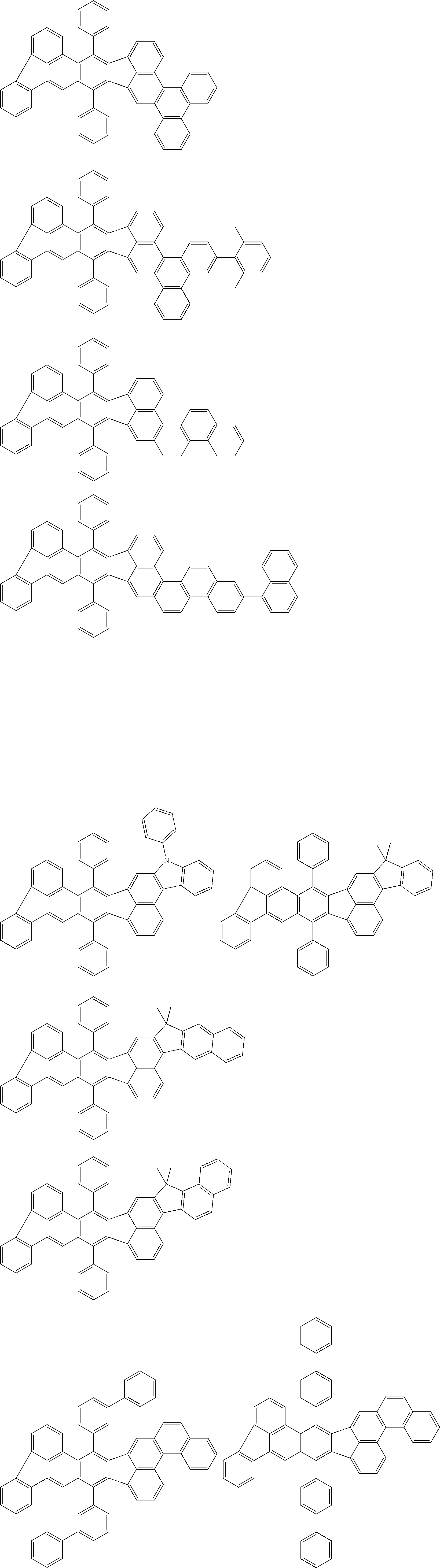

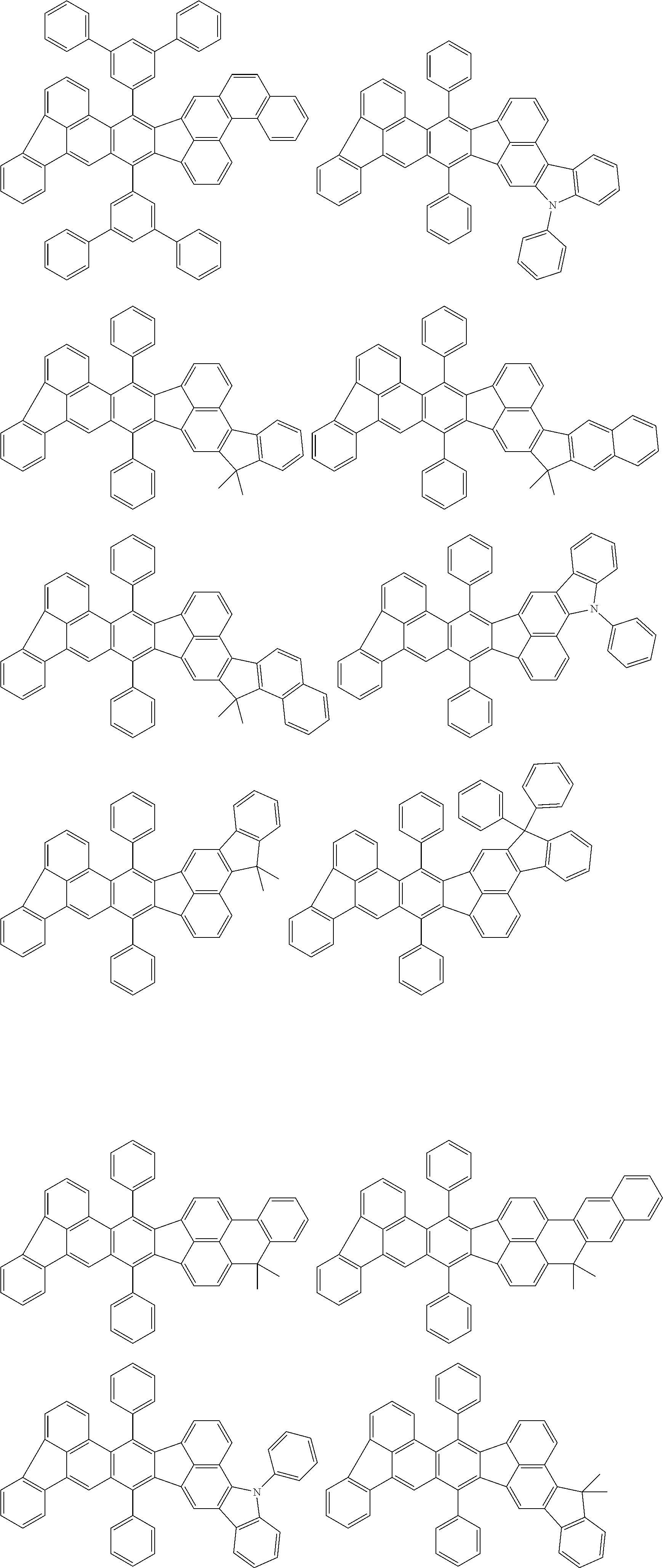

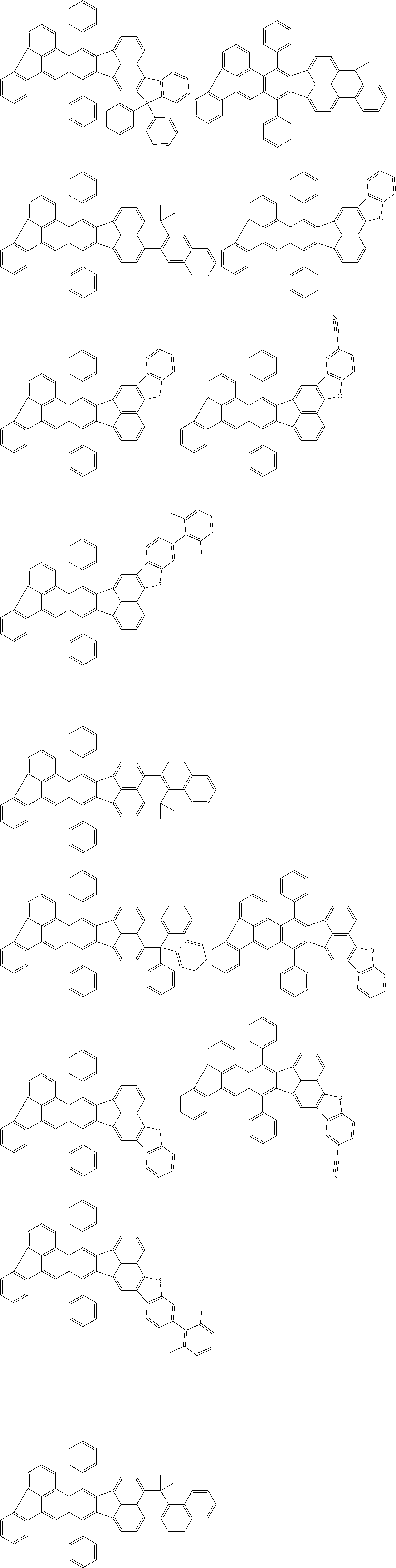

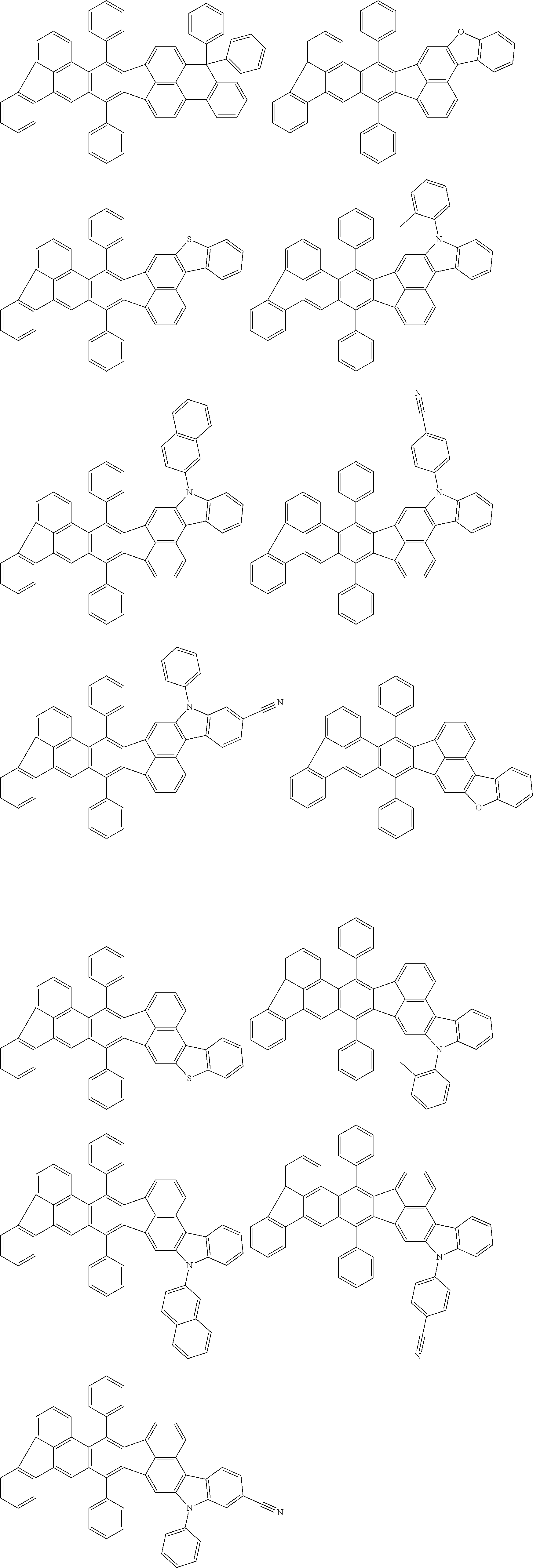

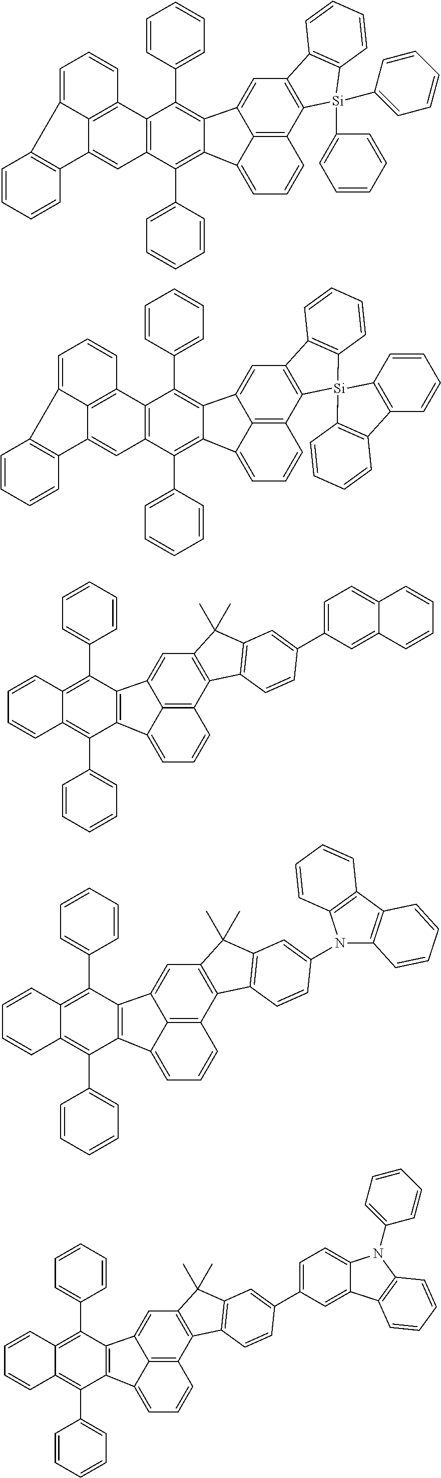

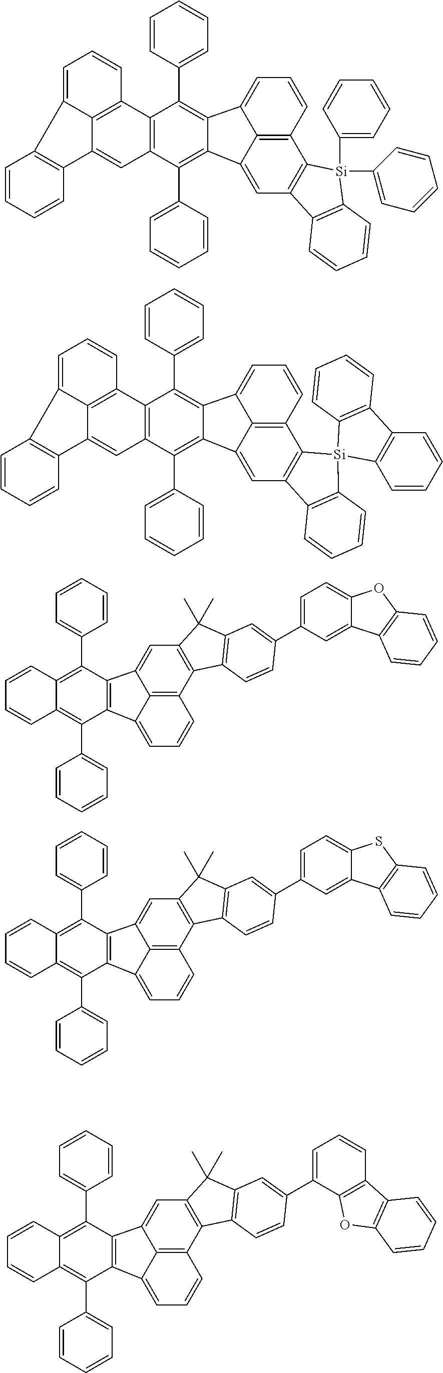

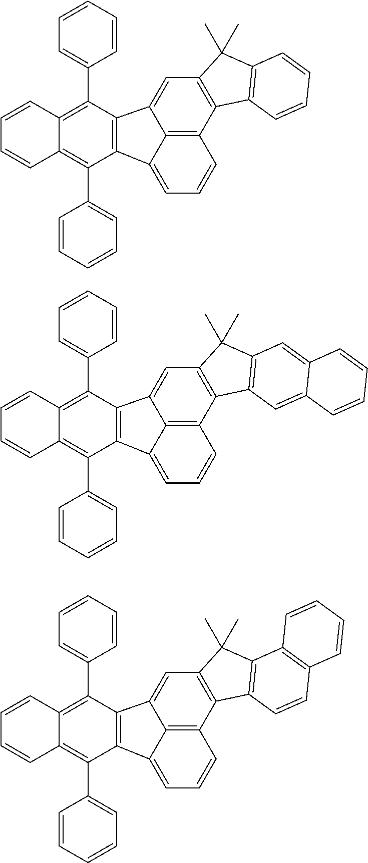

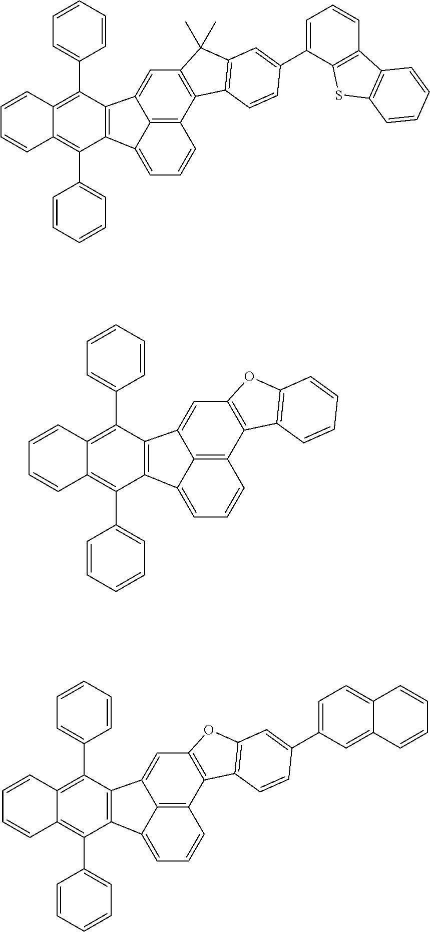 [SMILES: CC1(C)c2cc(-c3cccc4c3sc3ccccc34)ccc2-c2c1cc1c3c(cccc23)-c2c-1c(-c1ccccc1)c1ccccc1c2-c1ccccc1 ; c1ccc(-c2c3c(c(-c4ccccc4)c4ccccc24)-c2cc4oc5ccccc5c4c4cccc-3c24)cc1 ; c1ccc(-c2c3c(c(-c4ccccc4)c4ccccc24)-c2cc4oc5cc(-c6ccc7ccccc7c6)ccc5c4c4cccc-3c24)cc1]

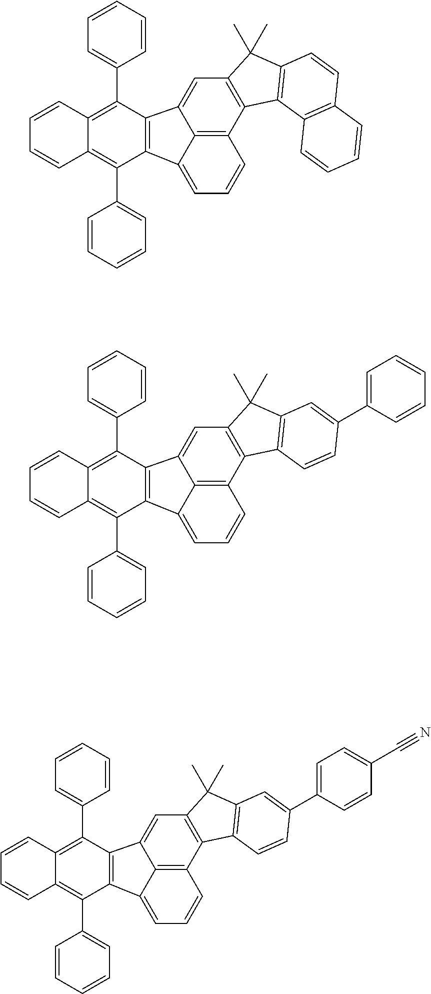

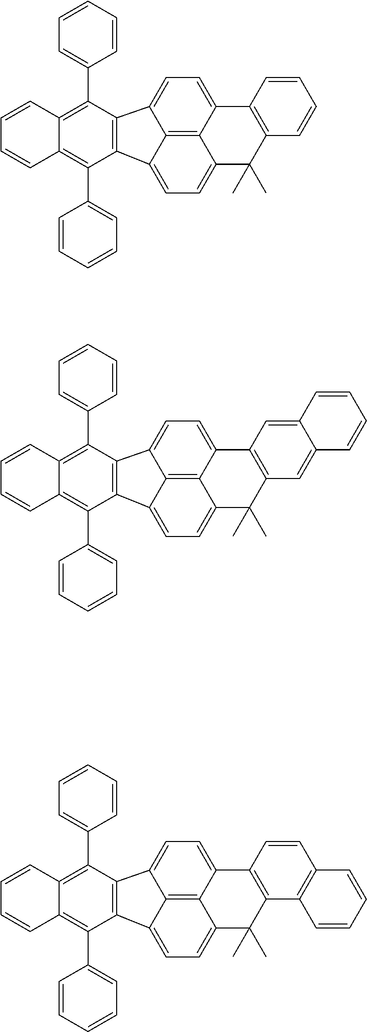

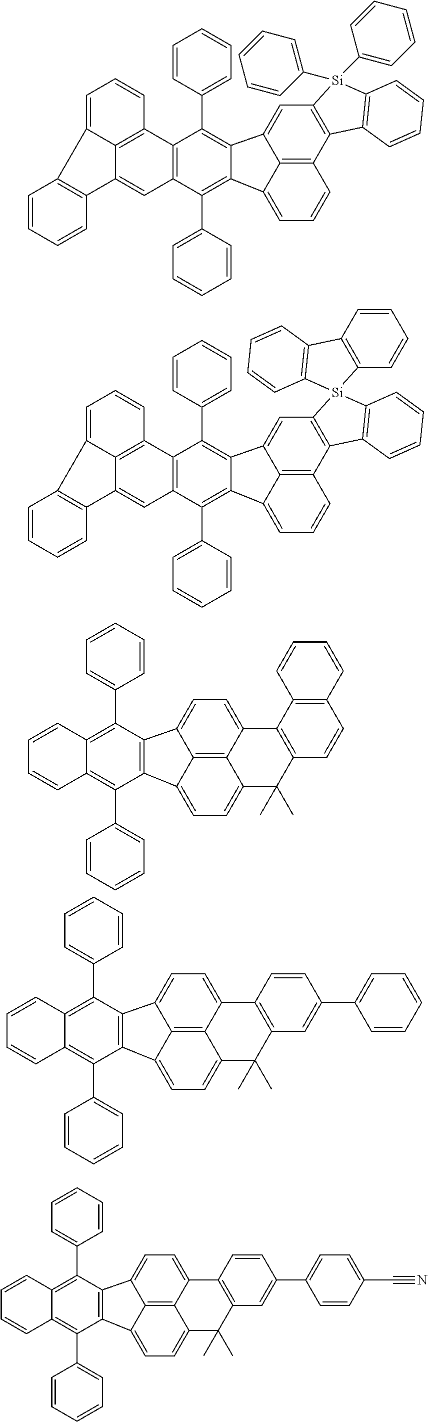

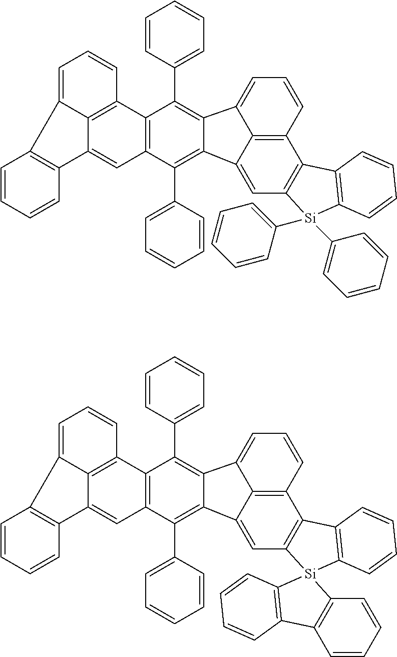 [SMILES: c1ccc(-c2c3cc4c5ccccc5c5cccc(c3c(-c3ccccc3)c3c6cccc7c8c(cc(c23)c76)[Si](c2ccccc2)(c2ccccc2)c2ccccc2-8)c54)cc1 ; c1ccc(-c2c3cc4c5ccccc5c5cccc(c3c(-c3ccccc3)c3c6cccc7c8c(cc(c23)c76)[Si]2(c3ccccc3-c3ccccc32)c2ccccc2-8)c54)cc1]

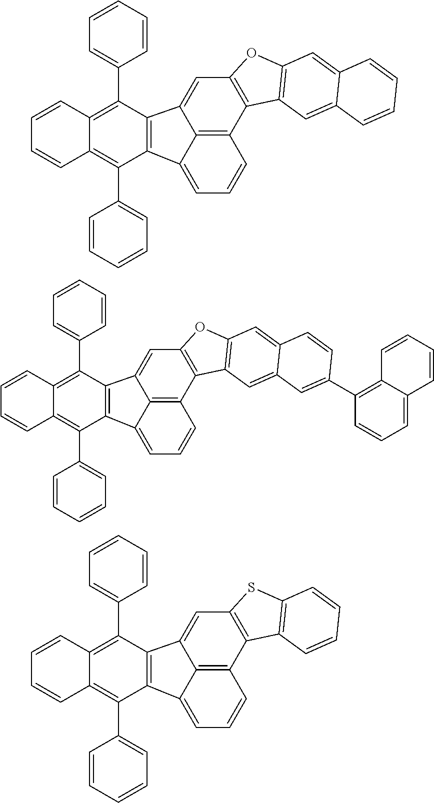

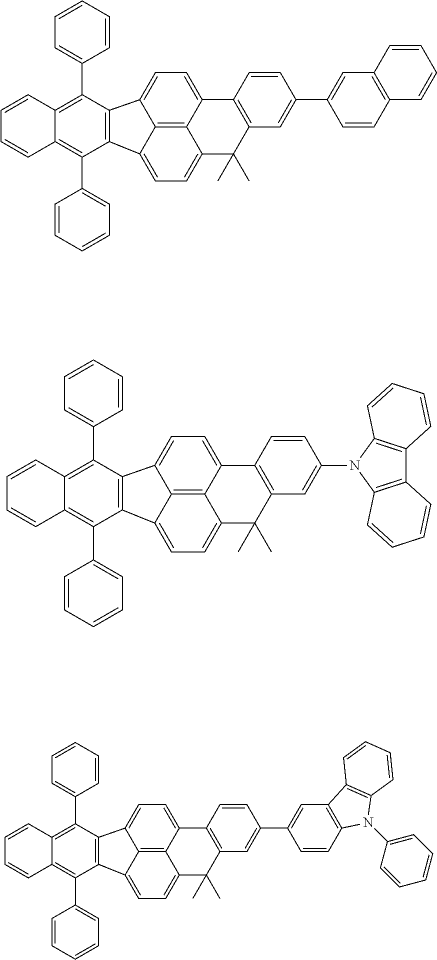

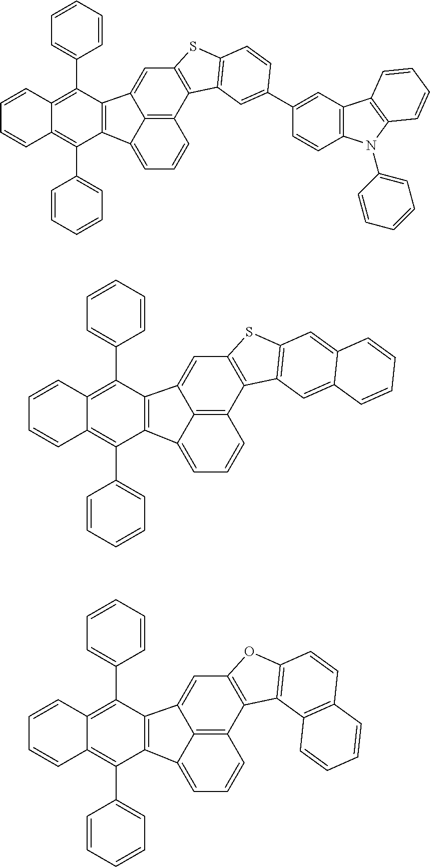 [SMILES: c1ccc(-c2c3c(c(-c4ccccc4)c4ccccc24)-c2cc4sc5ccc(-c6ccc7c(c6)c6ccccc6n7-c6ccccc6)cc5c4c4cccc-3c24)cc1 ; c1ccc(-c2c3c(c(-c4ccccc4)c4ccccc24)-c2cc4sc5cc6ccccc6cc5c4c4cccc-3c24)cc1 ; c1ccc(-c2c3c(c(-c4ccccc4)c4ccccc24)-c2cc4oc5ccc6ccccc6c5c4c4cccc-3c24)cc1]

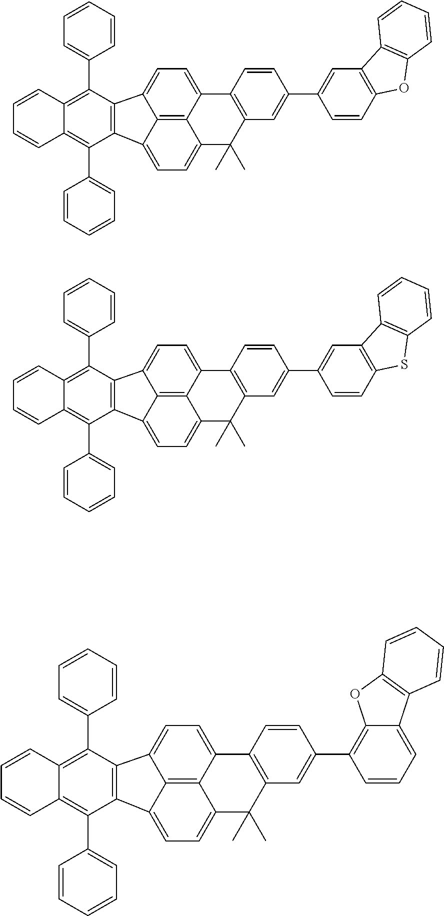 [SMILES: CC1(C)c2cc(-c3ccc4oc5ccccc5c4c3)ccc2-c2ccc3c4c(ccc1c24)-c1c-3c(-c2ccccc2)c2ccccc2c1-c1ccccc1 ; CC1(C)c2cc(-c3ccc4sc5ccccc5c4c3)ccc2-c2ccc3c4c(ccc1c24)-c1c-3c(-c2ccccc2)c2ccccc2c1-c1ccccc1 ; CC1(C)c2cc(-c3cccc4c3oc3ccccc34)ccc2-c2ccc3c4c(ccc1c24)-c1c-3c(-c2ccccc2)c2ccccc2c1-c1ccccc1]

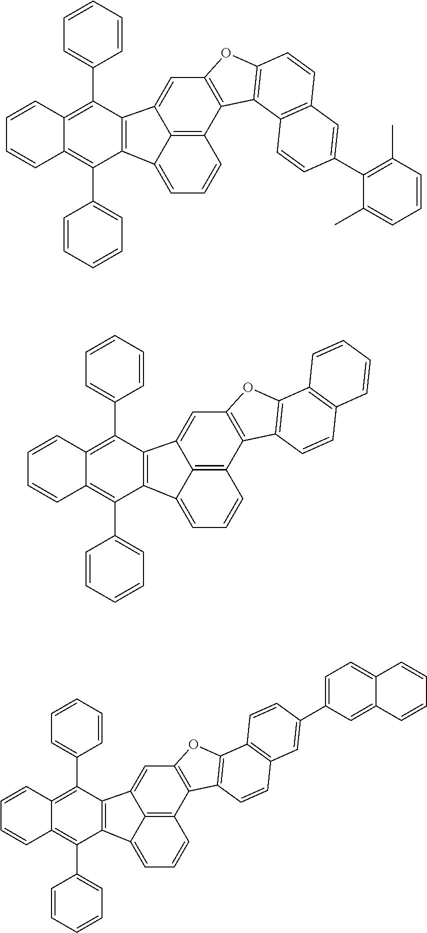 [SMILES: Cc1cccc(C)c1-c1ccc2c(ccc3oc4cc5c6c(cccc6c4c32)-c2c-5c(-c3ccccc3)c3ccccc3c2-c2ccccc2)c1 ; c1ccc(-c2c3c(c(-c4ccccc4)c4ccccc24)-c2cc4oc5c6ccccc6ccc5c4c4cccc-3c24)cc1 ; c1ccc(-c2c3c(c(-c4ccccc4)c4ccccc24)-c2cc4oc5c6ccc(-c7ccc8ccccc8c7)cc6ccc5c4c4cccc-3c24)cc1]

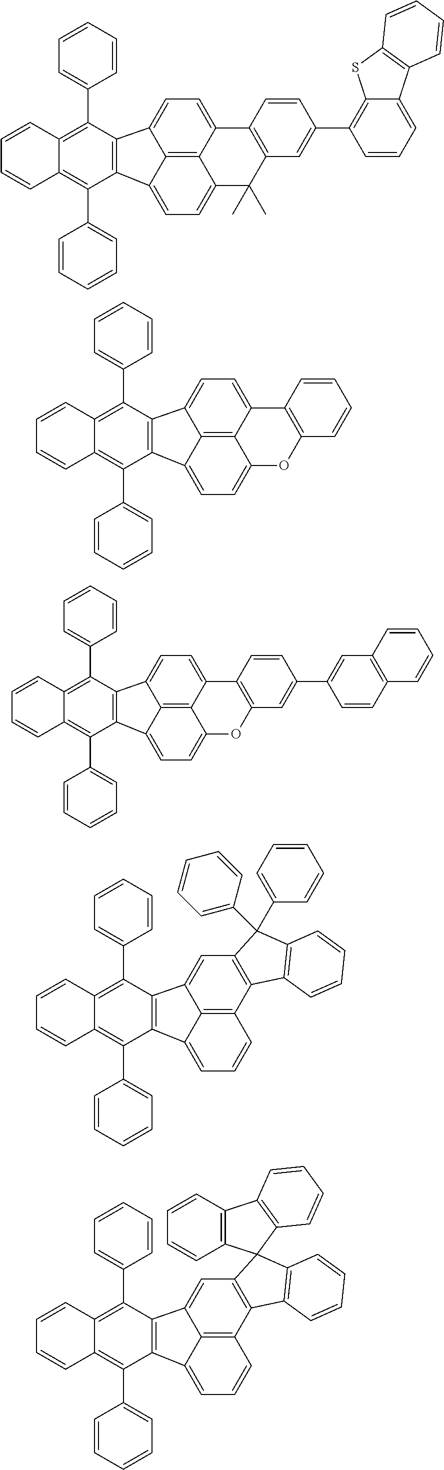

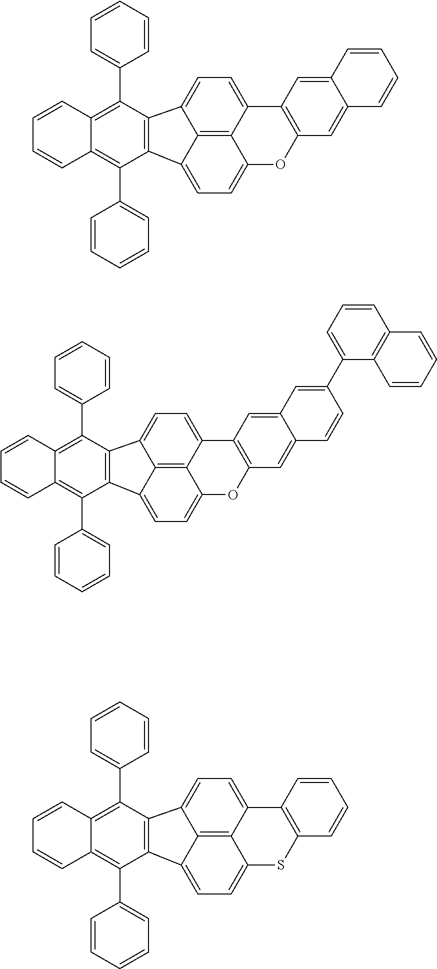 [SMILES: c1ccc(-c2c3ccccc3c(-c3ccccc3)c3c4ccc5c6cc7ccccc7cc6oc6ccc(c23)c4c65)cc1 ; c1ccc(-c2c3ccccc3c(-c3ccccc3)c3c4ccc5c6cc7cc(-c8cccc9ccccc89)ccc7cc6oc6ccc(c23)c4c65)cc1 ; c1ccc(-c2c3ccccc3c(-c3ccccc3)c3c4ccc5c6ccccc6sc6ccc(c23)c4c65)cc1]

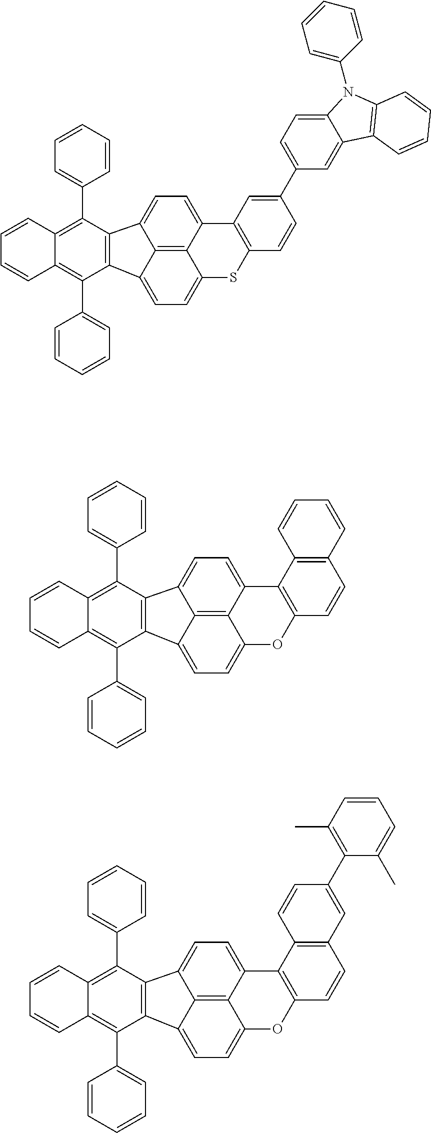 [SMILES: c1ccc(-c2c3ccccc3c(-c3ccccc3)c3c4ccc5c6cc(-c7ccc8c(c7)c7ccccc7n8-c7ccccc7)ccc6sc6ccc(c23)c4c65)cc1 ; c1ccc(-c2c3ccccc3c(-c3ccccc3)c3c4ccc5c6c(ccc7ccccc76)oc6ccc(c23)c4c65)cc1 ; Cc1cccc(C)c1-c1ccc2c(ccc3oc4ccc5c6c(-c7ccccc7)c7ccccc7c(-c7ccccc7)c6c6ccc(c32)c4c56)c1]

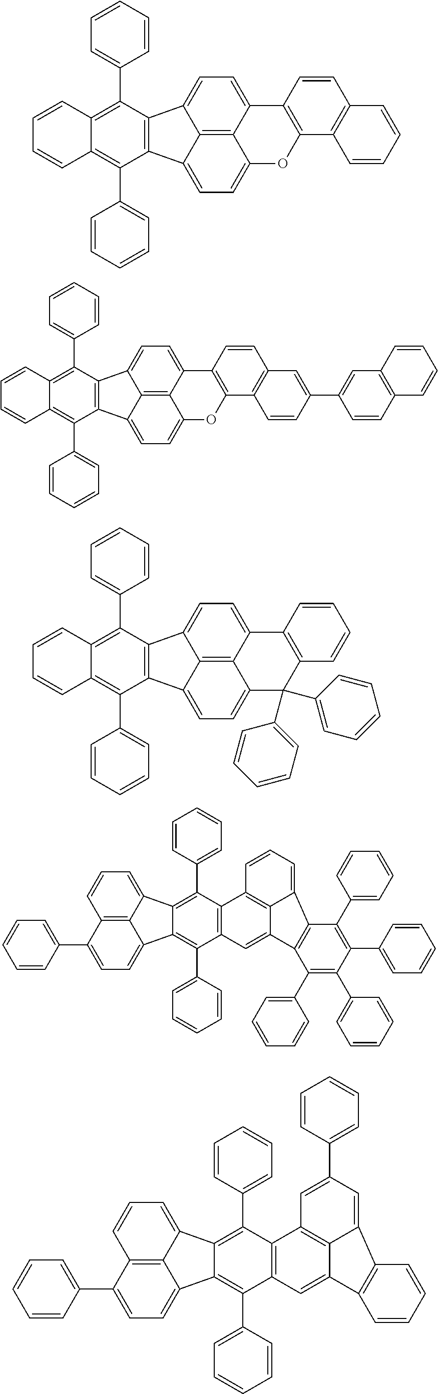

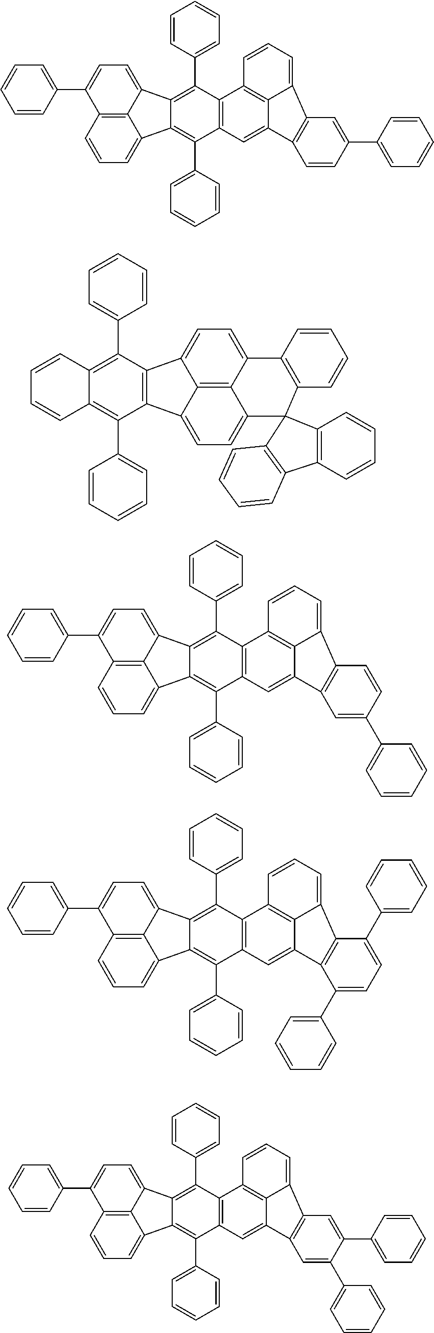

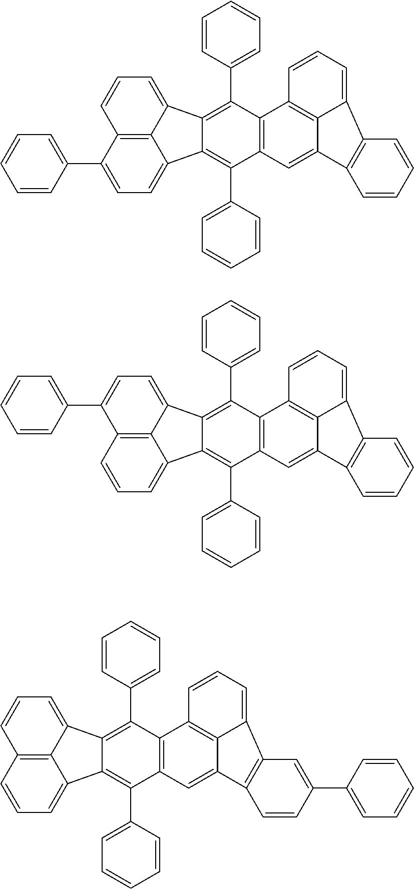

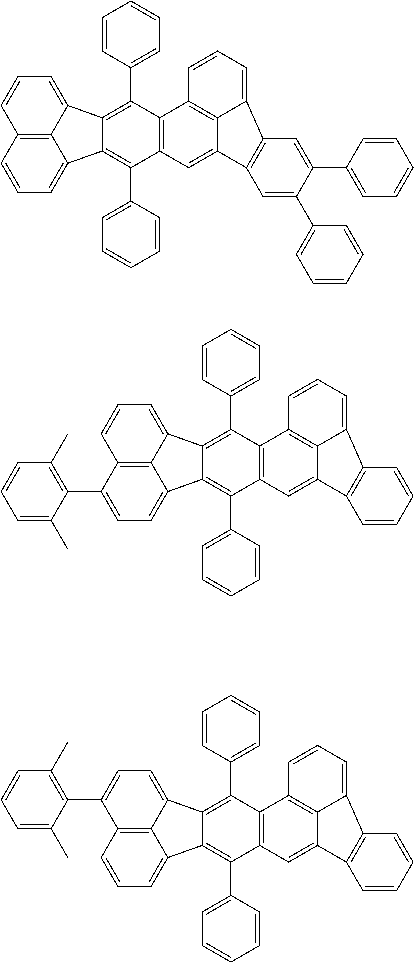

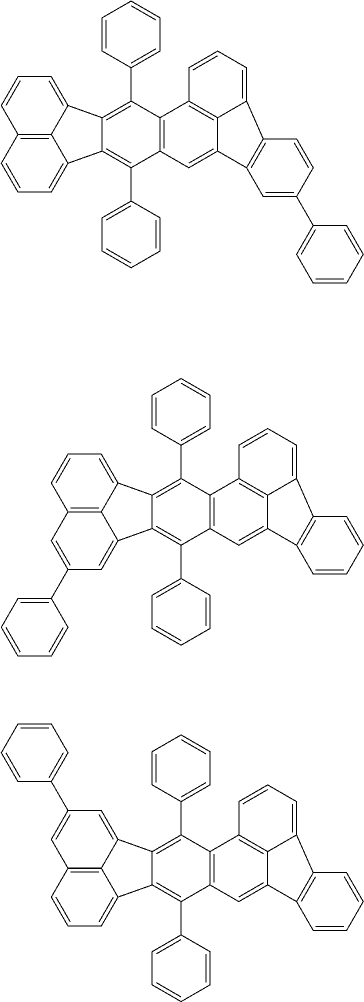

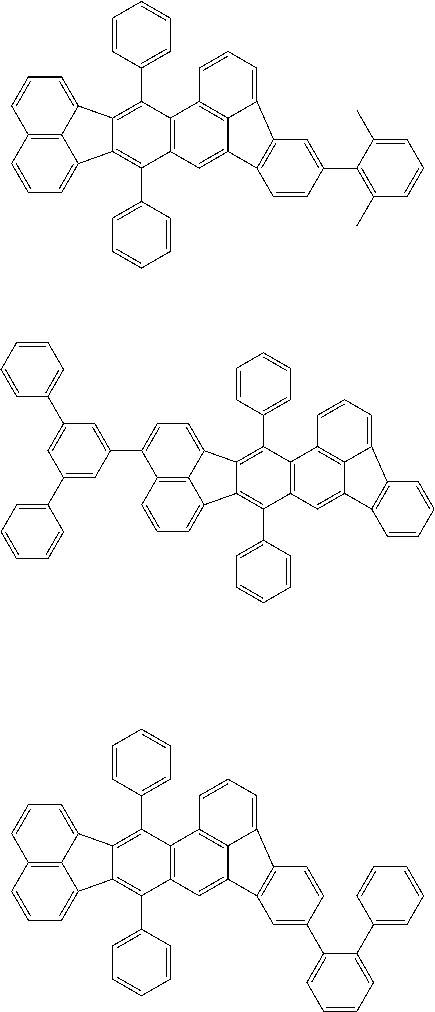

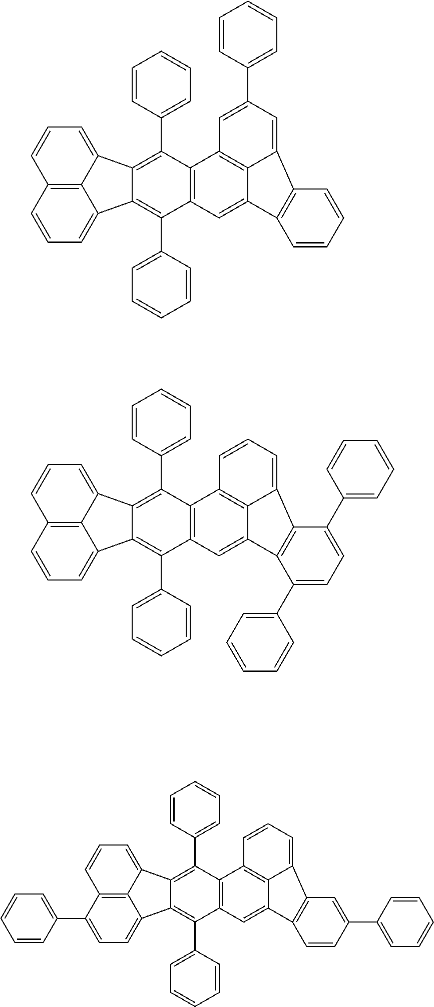

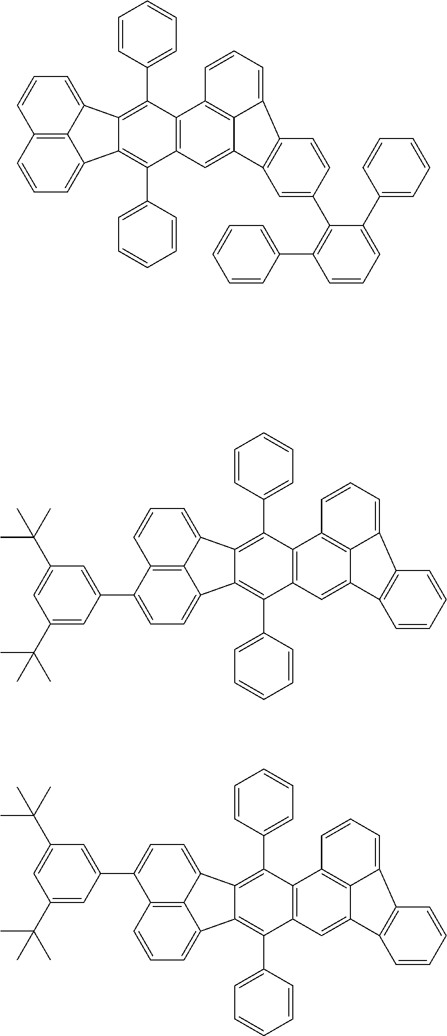

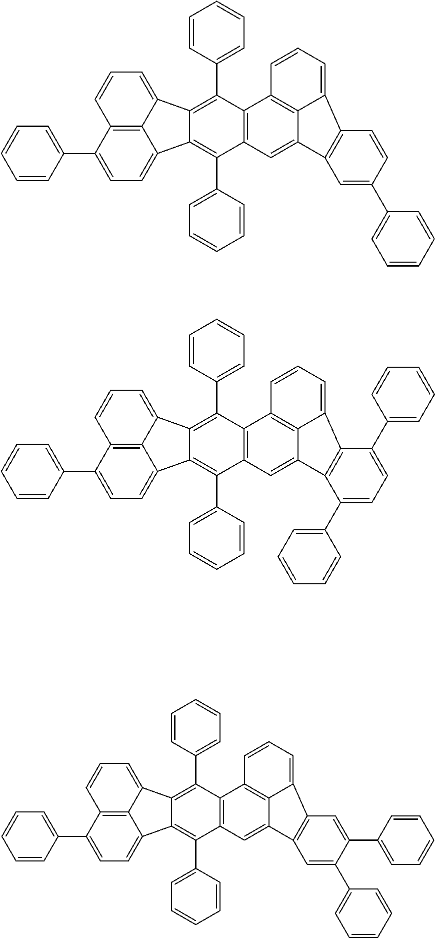

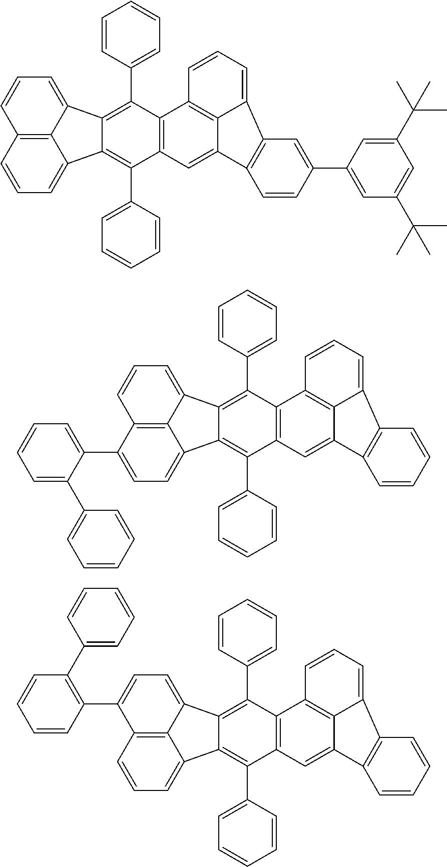

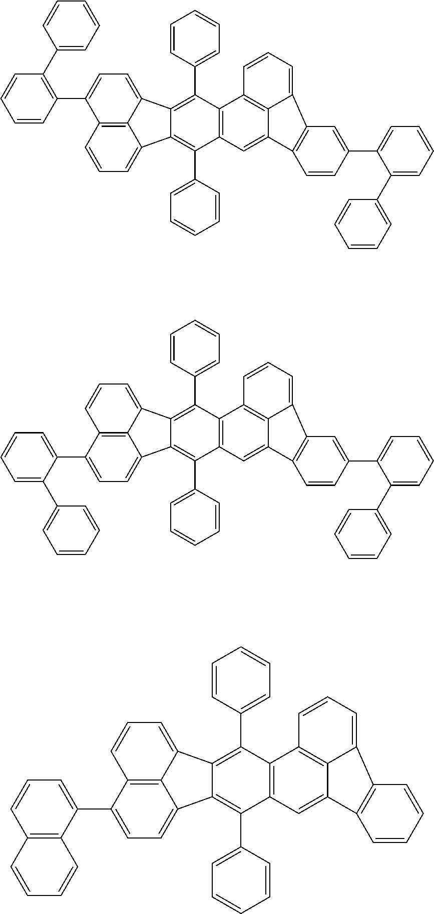

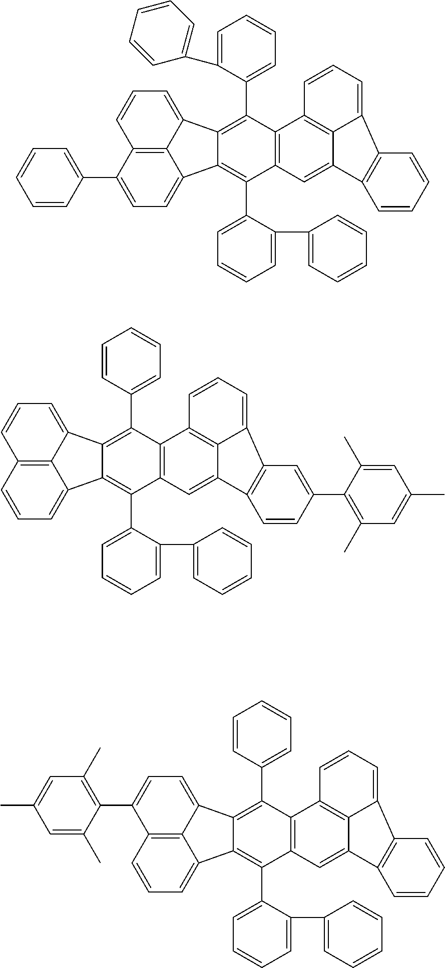

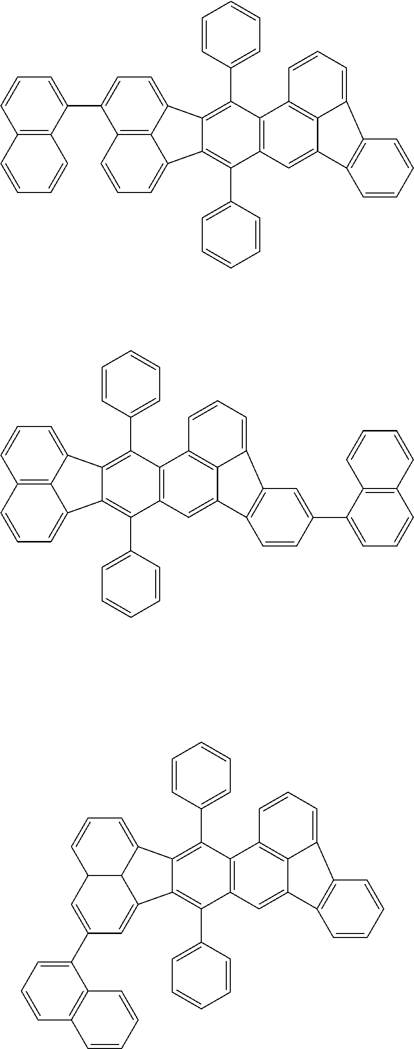

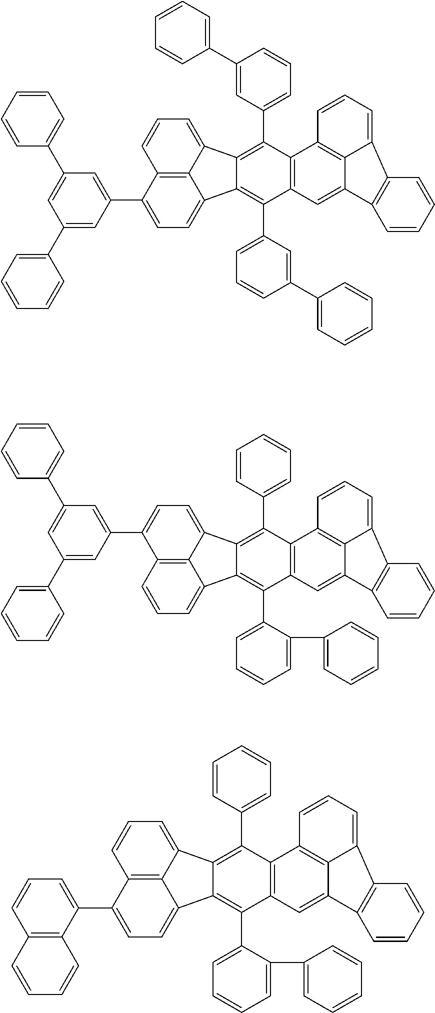

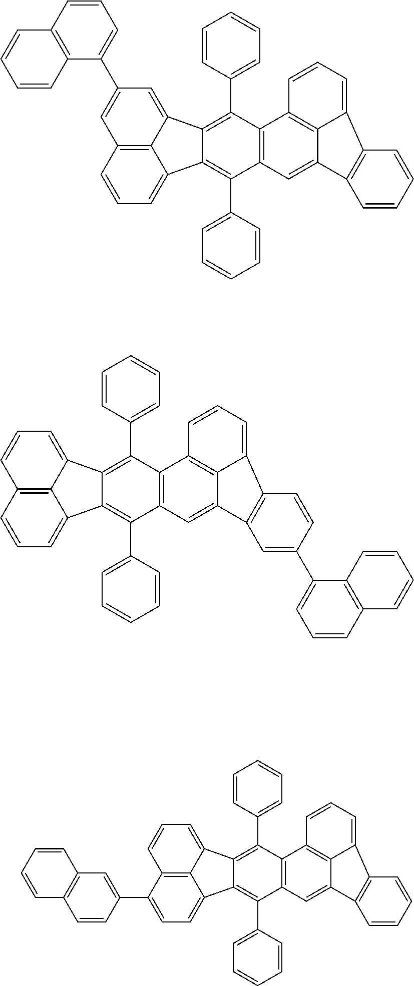

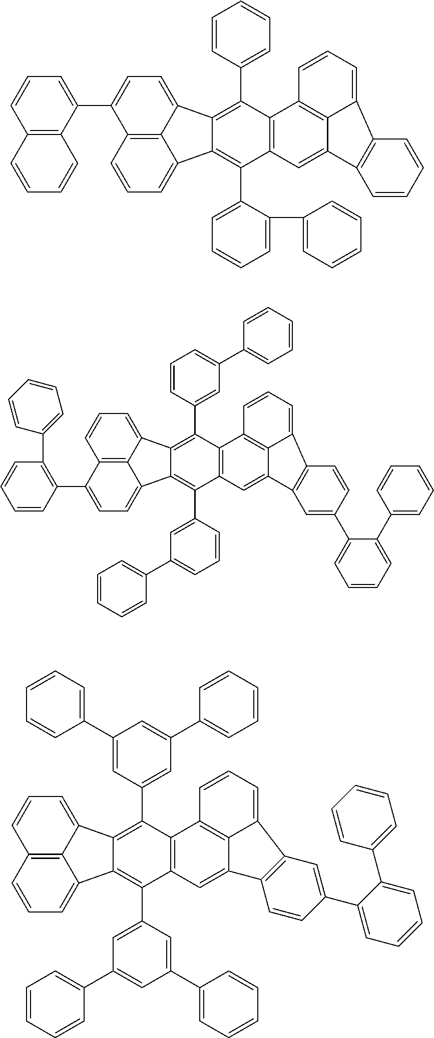

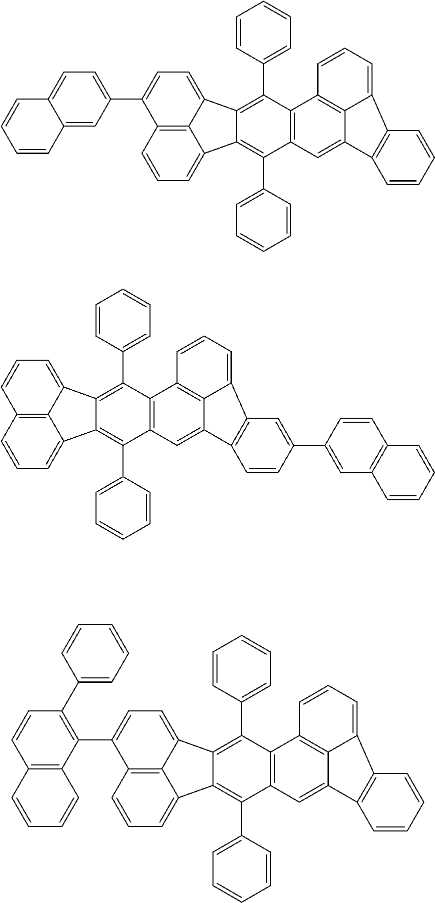

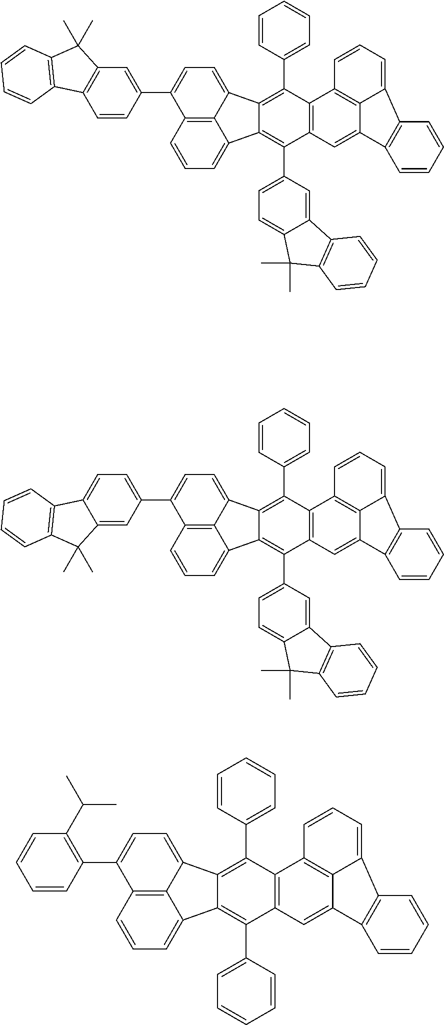

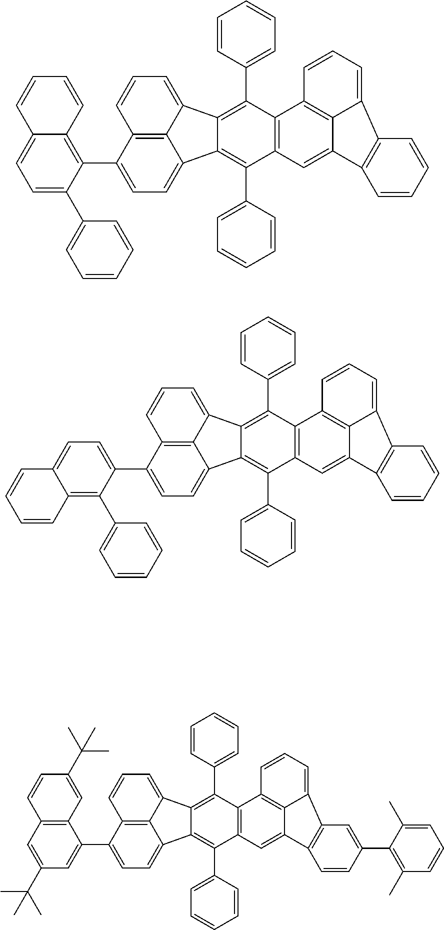

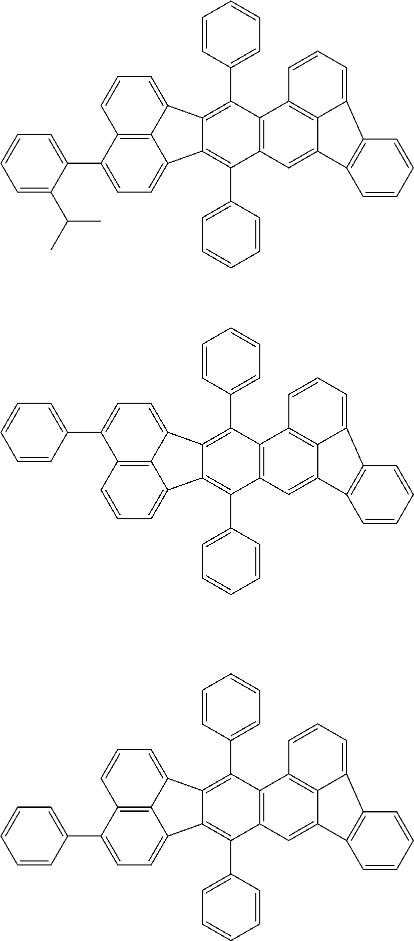

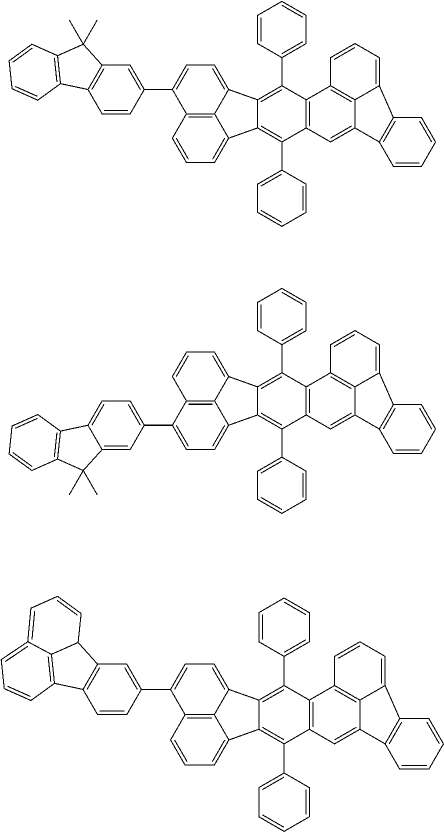

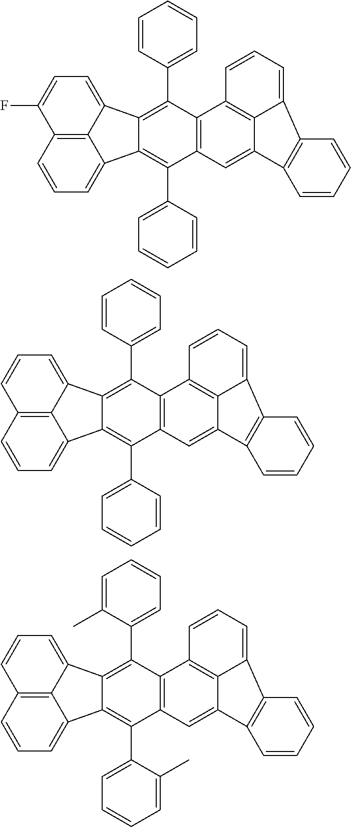

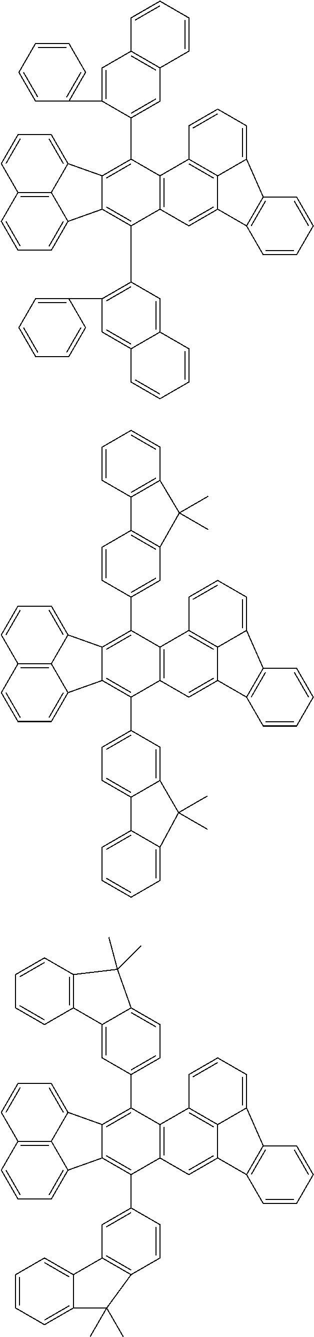

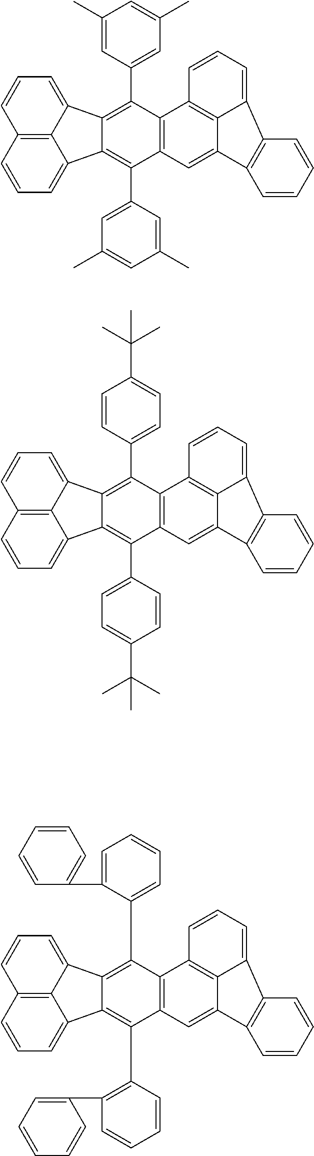

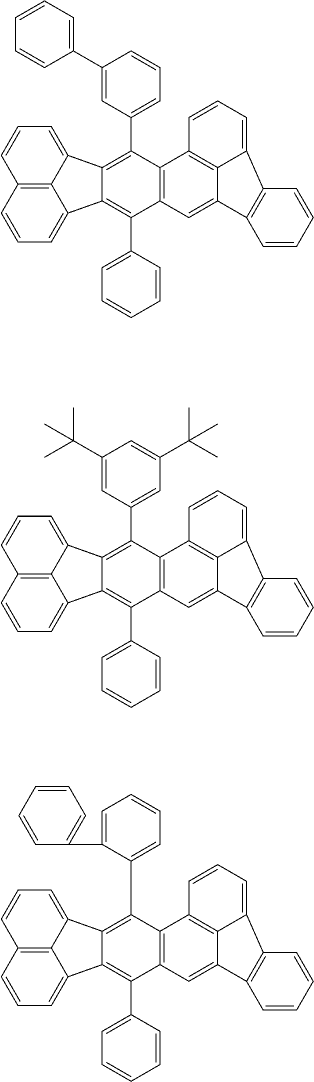

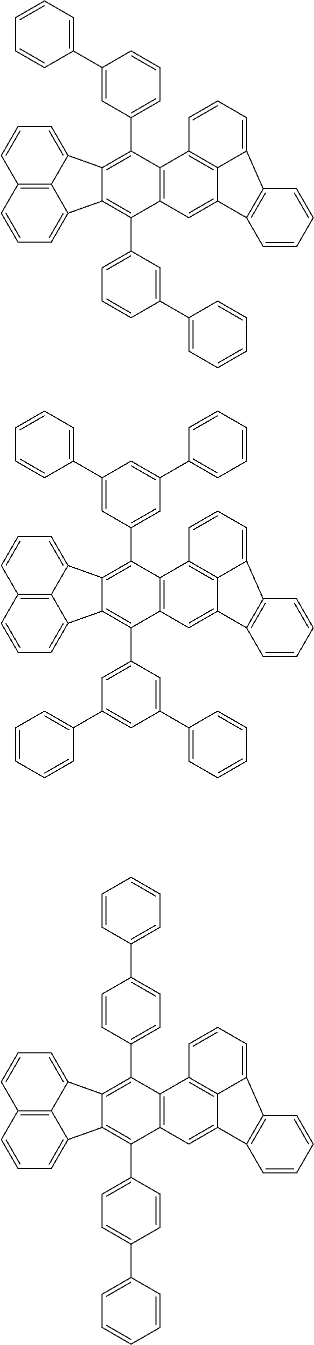

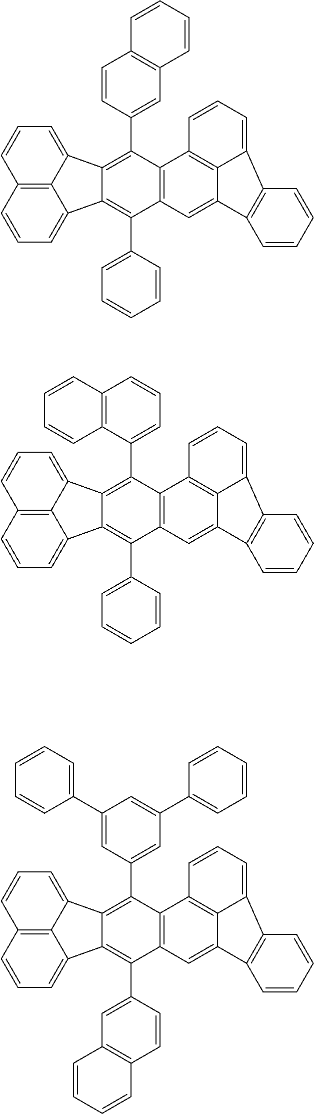

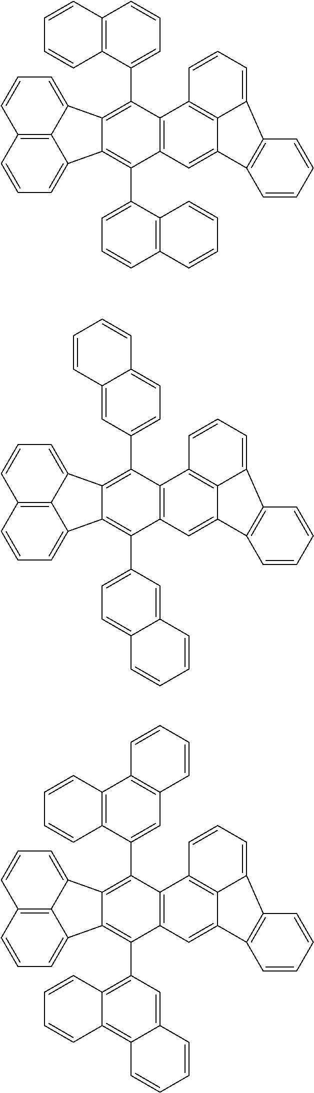

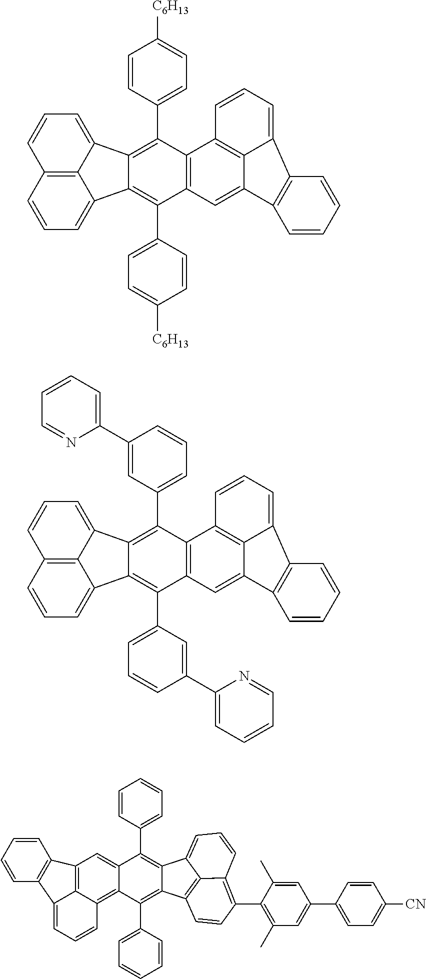

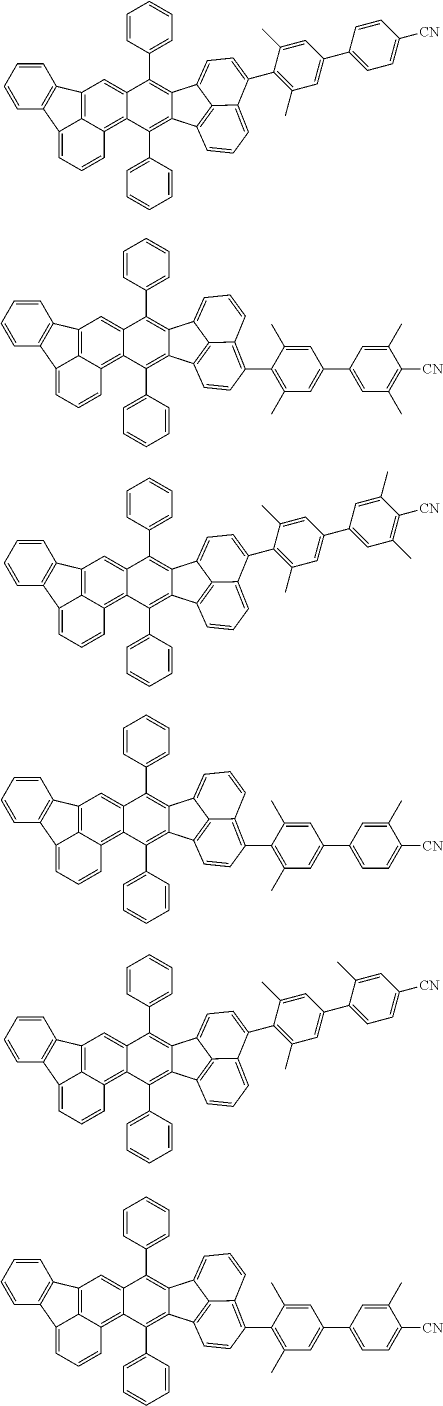

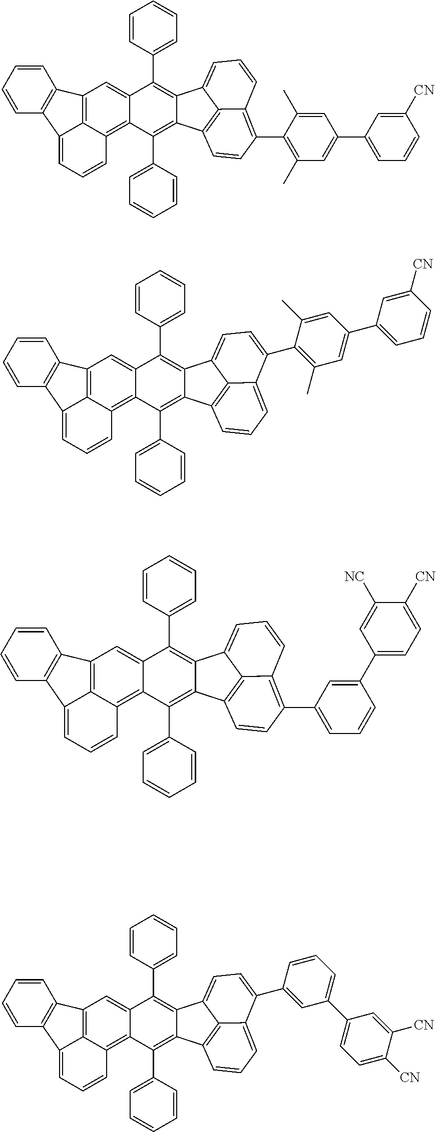 [SMILES: Cc1cc(-c2cccc(C#N)c2)cc(C)c1-c1ccc2c3c(-c4ccccc4)c4c(cc5c6ccccc6c6cccc4c65)c(-c4ccccc4)c3c3cccc1c32 ; Cc1cc(-c2cccc(C#N)c2)cc(C)c1-c1ccc2c3c(-c4ccccc4)c4cc5c6ccccc6c6cccc(c4c(-c4ccccc4)c3c3cccc1c23)c65 ; N#Cc1ccc(-c2cccc(-c3ccc4c5c(-c6ccccc6)c6c(cc7c8ccccc8c8cccc6c87)c(-c6ccccc6)c5c5cccc3c54)c2)cc1C#N ; N#Cc1ccc(-c2cccc(-c3ccc4c5c(-c6ccccc6)c6cc7c8ccccc8c8cccc(c6c(-c6ccccc6)c5c5cccc3c45)c87)c2)cc1C#N]

### [Compound A]

Next, a compound A having a Stokes shift of 20 nm or less and an emission peak wavelength of 440 nm to 465 nm will be described.

The compound A is not particularly limited as long as the Stokes shift and the emission peak wavelength are within the above range, and may be a compound having any chemical structure.

Generally, the Stokes shift of a molecule in a state in which rotational motion and interatomic vibration are suppressed due to a rigid structure in the molecule tends to be small. By designing such highly rigid structures, a compound having a Stokes shift of 20 nm or less can be obtained.

In one embodiment, the organic layer includes an emitting layer, and

the emitting layer contains the compound represented by the formula (1), and the compound A.

In this case, the compound represented by the formula (1) functions as a host material of the emitting layer, and the compound A functions as a dopant material of the emitting layer.

In one embodiment, the compound A is one or more selected from the group consisting of a compound represented by the following formula (A-1) and a compound represented by the following formula (A-2).

### [Compound Represented by the Formula (A-1)]

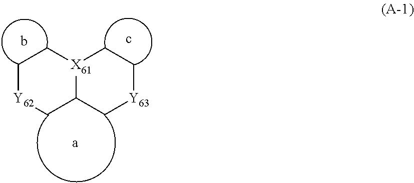

In the formula (A-1),

ring a, ring b and ring c are independently,

a substituted or unsubstituted aromatic hydrocarbon ring including 6 to 50 ring carbon atoms, or

a substituted or unsubstituted heterocyclic ring including 5 to 50 ring atoms.

X₆₁ is B or N.

Y₆₂ and Y₆₃ are independently NR_{d}, O, S, or a single bond;

provided that when X₆₁ is B, Y₆₂ and Y₆₃ are independently NR_{d}, O or S.

When X₆₁ is N, Y₆₂ and Y₆₃ are single bonds.

R_{d} forms a substituted or unsubstituted heterocyclic ring by bonding with the ring a, the ring b, or the ring c, or does not form a substituted or unsubstituted heterocyclic ring.

R_{d}'s which do not form the substituted or unsubstituted heterocyclic ring are independently,

a substituted or unsubstituted alkyl group including 1 to 50 carbon atoms,

a substituted or unsubstituted alkenyl group including 2 to 50 carbon atoms,

a substituted or unsubstituted alkynyl group including 2 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms.

In one embodiment, the compound represented by the formula (A-1) is a compound represented by the following formula (A-1-1):

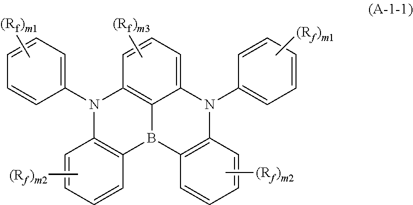

In the formula (A-1-1),

each R_{f} is a substituent.

each m1 is an integer of 0 to 5.

each m2 is an integer of 0 to 4.

m3 is an integer of 0 to 3.

When two or more of each of m1 to m3 are present, two or more R_{f}'s may be the same as or different to each other.

In one embodiment, R_{f}'s are independently,

a substituted or unsubstituted alkyl group including 1 to 50 carbon atoms,

a substituted or unsubstituted alkenyl group including 2 to 50 carbon atoms,

a substituted or unsubstituted alkynyl group including 2 to 50 carbon atoms,

a substituted or unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a halogen atom, a cyano group, a nitro group,

a substituted or unsubstituted aryl group including 6 to 50 ring carbon atoms, or

a substituted or unsubstituted monovalent heterocyclic group including 5 to 50 ring atoms.

R₉₀₁ to R₉₀₇ are independently,

a hydrogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms.

when two or more of each of R₉₀₁ to R₉₀₇ are present, the two or more of each of R₉₀₁ to R₉₀₇ may be the same as or different to each other.

Specific examples of the compound represented by the formula (A-1) will be described below, but these are merely examples, and the compound represented by the formula (A-1) is not limited to the following specific examples.

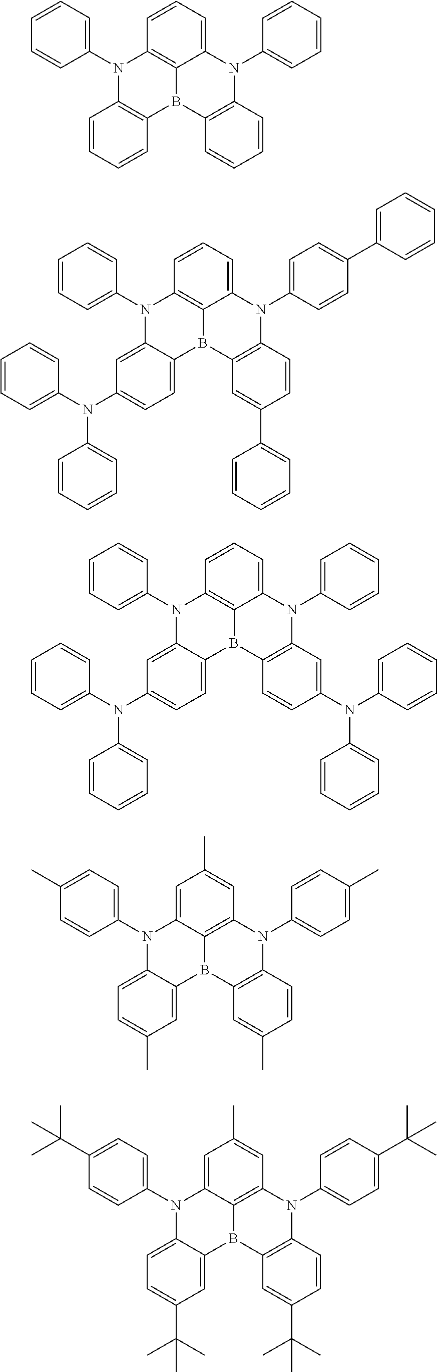

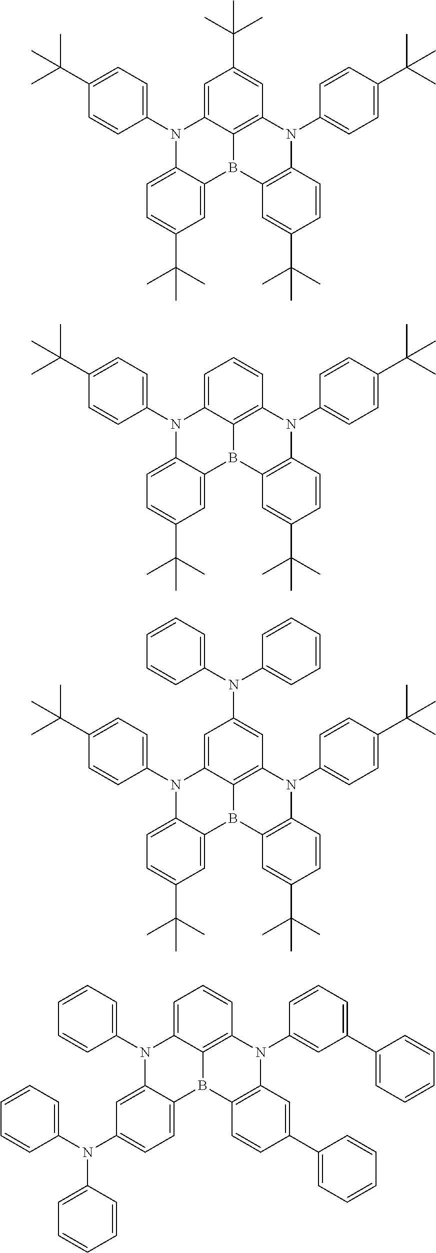

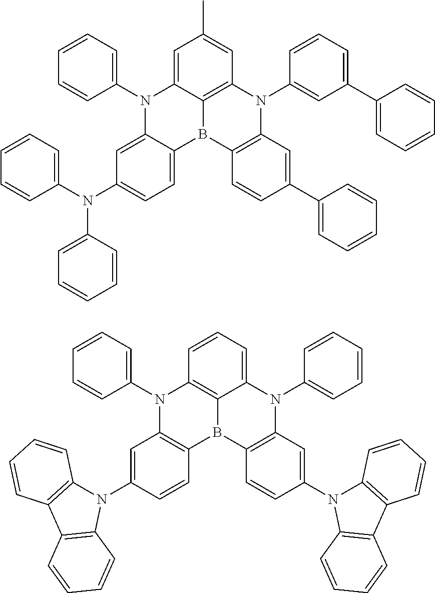

### [Compound Represented by the Formula (A-2)]

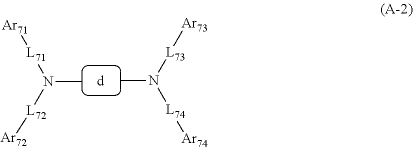

In the formula (A-2),

ring d is a substituted or unsubstituted aromatic hydrocarbon ring including 10 to 50 ring carbon atoms, or

a substituted or unsubstituted heterocyclic ring including 12 to 50 ring atoms.

L₇₁ to L₇₄ are independently,

a single bond,

a substituted or unsubstituted arylene group including 6 to 50 ring carbon atoms, or

a substituted or unsubstituted divalent heterocyclic group including 5 to 50 ring atoms.

Ar₇₁ to Ar₇₄ are independently,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms;

provided that when the ring d is a substituted or unsubstituted aromatic hydrocarbon ring including 10 to 50 ring carbon atoms, two or more among Ar₇₁ to Ar₇₄ are independently an aryl group including 6 to 50 ring carbon atoms, that is substituted by an alkyl group including 1 to 50 carbon atoms, or a monovalent heterocyclic group including 5 to 50 ring atoms, that is substituted by an alkyl group including 1 to 50 carbon atoms.

In one embodiment, the compound represented by the formula (A-2) is a compound represented by the following formula (A-2-1).

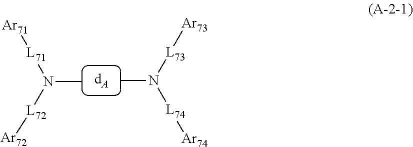

In the formula (A-2-1), L₇₁ to L₇₄ and Ar₇₁ to Ar₇₄ are as defined in the formula (A-2), and

ring d_{A} is a substituted or unsubstituted aromatic hydrocarbon ring including 10 to 50 ring carbon atoms.

In one embodiment, the ring d_{A} is a substituted or unsubstituted pyrene ring.

In one embodiment, a substituent of the ring d_{A} is,

a substituted or unsubstituted alkyl group including 1 to 50 carbon atoms,

a substituted or unsubstituted cycloalkyl group including 3 to 50 ring carbon atoms,

—Si(R₉₀₁)(R₉₀₂)(R₉₀₃),

a halogen atom, a cyano group, or a nitro group.

R₉₀₁ to R₉₀₃ are independently,

a hydrogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms.

When two or more of each of R₉₀₁ to R₉₀₃ are present, the two or more of each of R₉₀₁ to R₉₀₃ may be the same as or different to each other.

In the other embodiment of the formula (A-2), the compound represented by the formula (A-2) is a compound represented by the following formula (A-2-2):

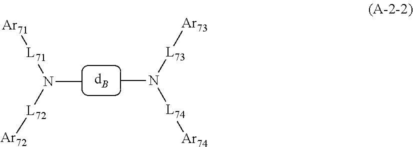

In the formula (A-2-2), L₇₁ to L₇₄ and Ar₇₁ to Ar₇₄ are as defined in the formula (A-2), and ring d_{B} is a substituted or unsubstituted heterocyclic ring including 12 to 50 ring atoms.

In one embodiment, the ring d_{B} is selected from a substituted or unsubstituted heterocyclic ring having the following structures:

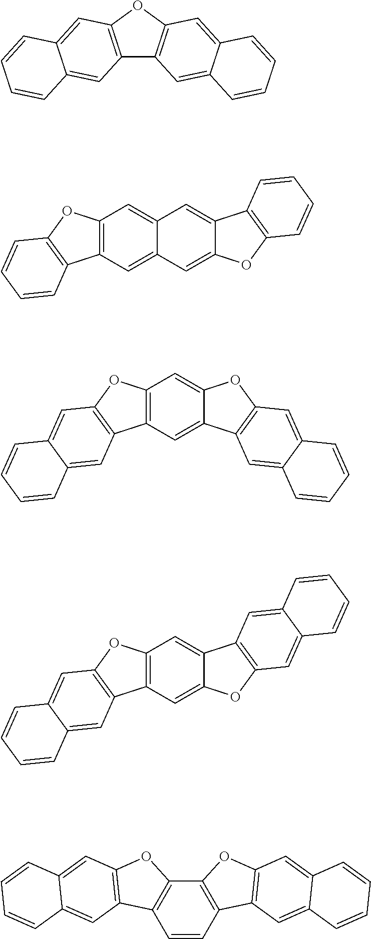 [SMILES: c1ccc2cc3c(cc2c1)oc1cc2ccccc2cc13 ; c1ccc2c(c1)oc1cc3cc4c(cc3cc12)oc1ccccc14 ; c1ccc2cc3c(cc2c1)oc1cc2oc4cc5ccccc5cc4c2cc13 ; c1ccc2cc3c(cc2c1)oc1cc2c(cc13)oc1cc3ccccc3cc12 ; c1ccc2cc3c(cc2c1)oc1c3ccc2c3cc4ccccc4cc3oc21]

In one embodiment, a substituent of the ring d_{B} is,

a substituted or unsubstituted alkyl group including 1 to 50 carbon atoms,

a substituted or unsubstituted cycloalkyl group including 3 to 50 ring carbon atoms,

—Si(R₉₀₁)(R₉₀₂)(R₉₀₃),

a halogen atom, a cyano group, or a nitro group.

R₉₀₁ to R₉₀₃ are independently,

a hydrogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms.

When two or more of each of R₉₀₁ to R₉₀₃ are present, the two or more of each of R₉₀₁ to R₉₀₃ may be the same as or different to each other.

Specific examples of the compound represented by the formula (A-2) will be described below, but these are merely examples, and the compound represented by the formula (A-2) is not limited to the following specific examples.

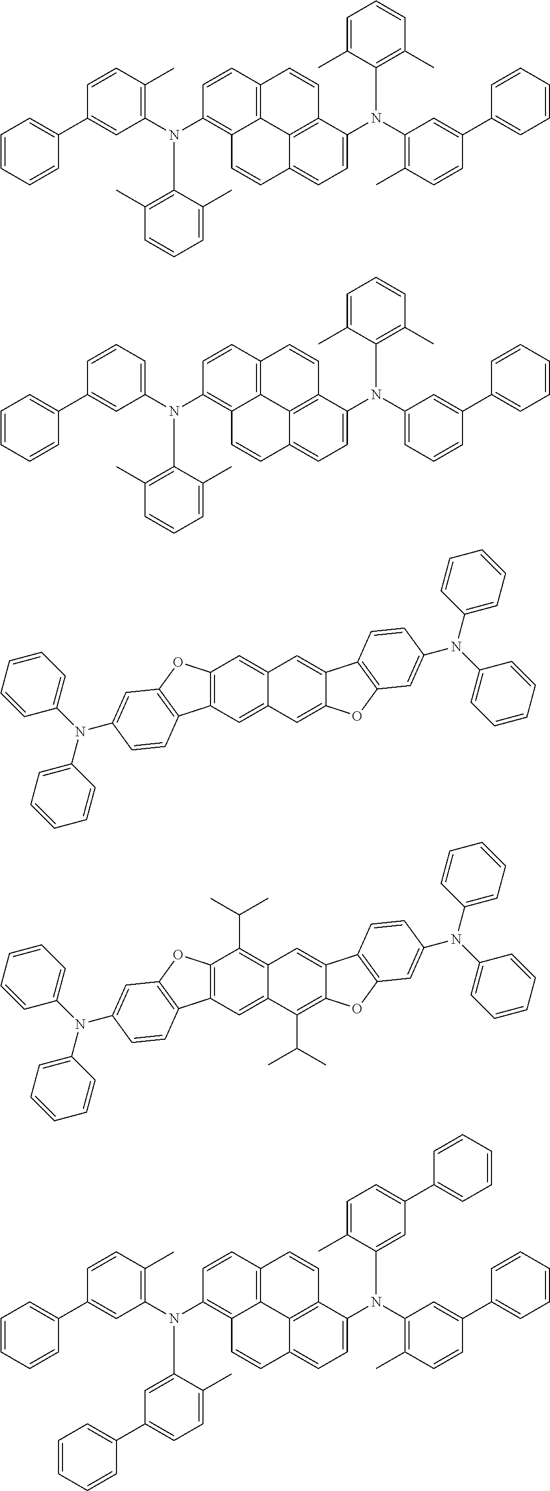 [SMILES: Cc1ccc(-c2ccccc2)cc1N(c1c(C)cccc1C)c1ccc2ccc3c(N(c4cc(-c5ccccc5)ccc4C)c4c(C)cccc4C)ccc4ccc1c2c43 ; Cc1cccc(C)c1N(c1cccc(-c2ccccc2)c1)c1ccc2ccc3c(N(c4cccc(-c5ccccc5)c4)c4c(C)cccc4C)ccc4ccc1c2c43 ; c1ccc(N(c2ccccc2)c2ccc3c(c2)oc2cc4cc5c(cc4cc23)oc2cc(N(c3ccccc3)c3ccccc3)ccc25)cc1 ; CC(C)c1c2cc3c(oc4cc(N(c5ccccc5)c5ccccc5)ccc43)c(C(C)C)c2cc2c1oc1cc(N(c3ccccc3)c3ccccc3)ccc12 ; Cc1ccc(-c2ccccc2)cc1N(c1cc(-c2ccccc2)ccc1C)c1ccc2ccc3c(N(c4cc(-c5ccccc5)ccc4C)c4cc(-c5ccccc5)ccc4C)ccc4ccc1c2c43]

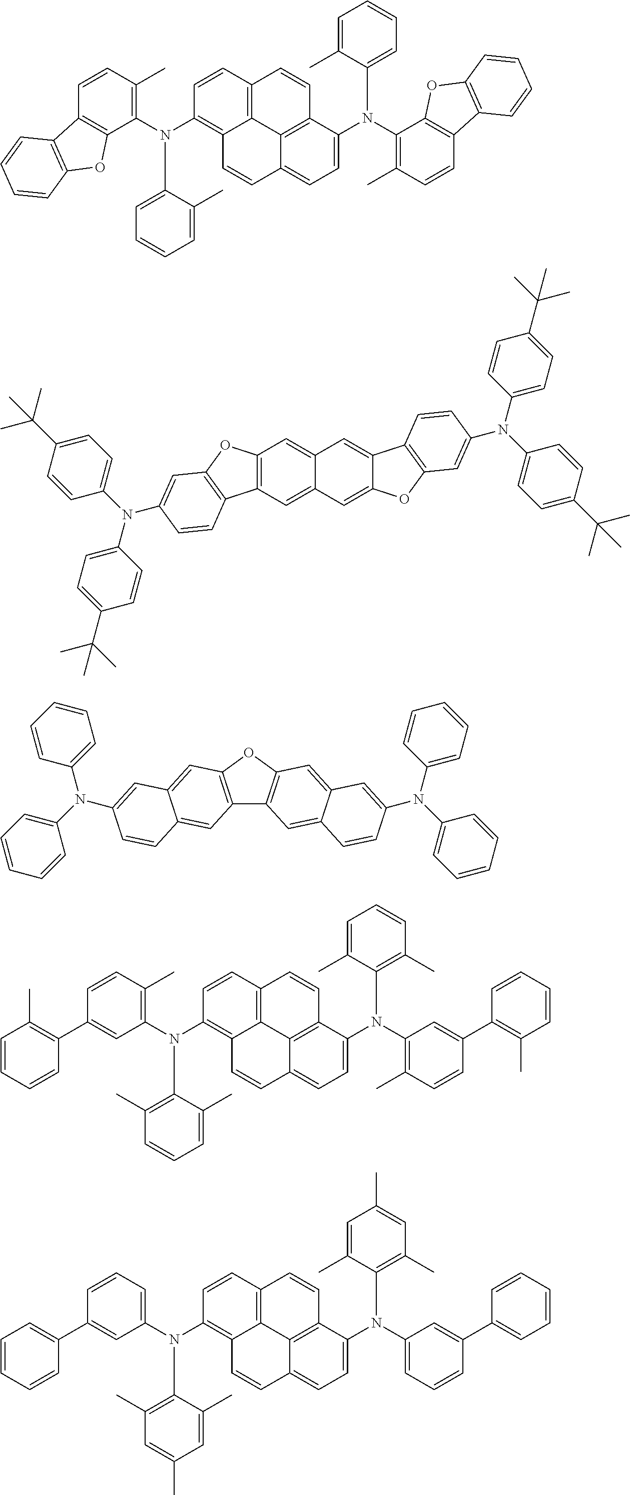

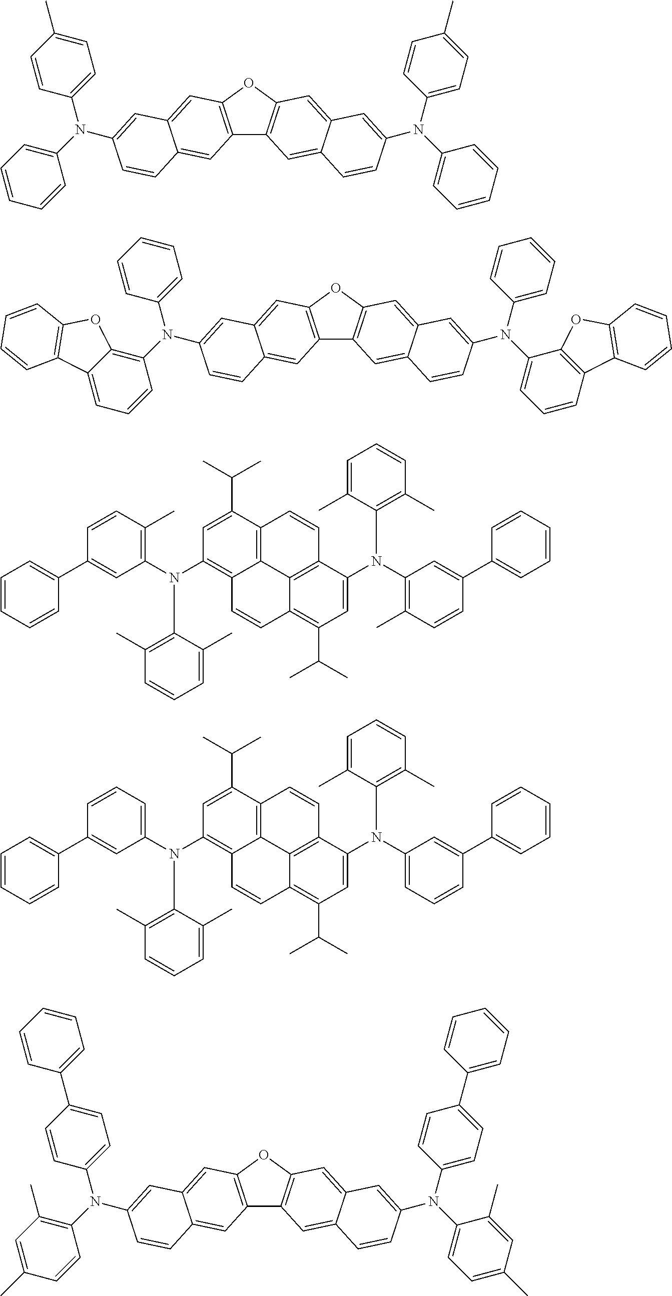

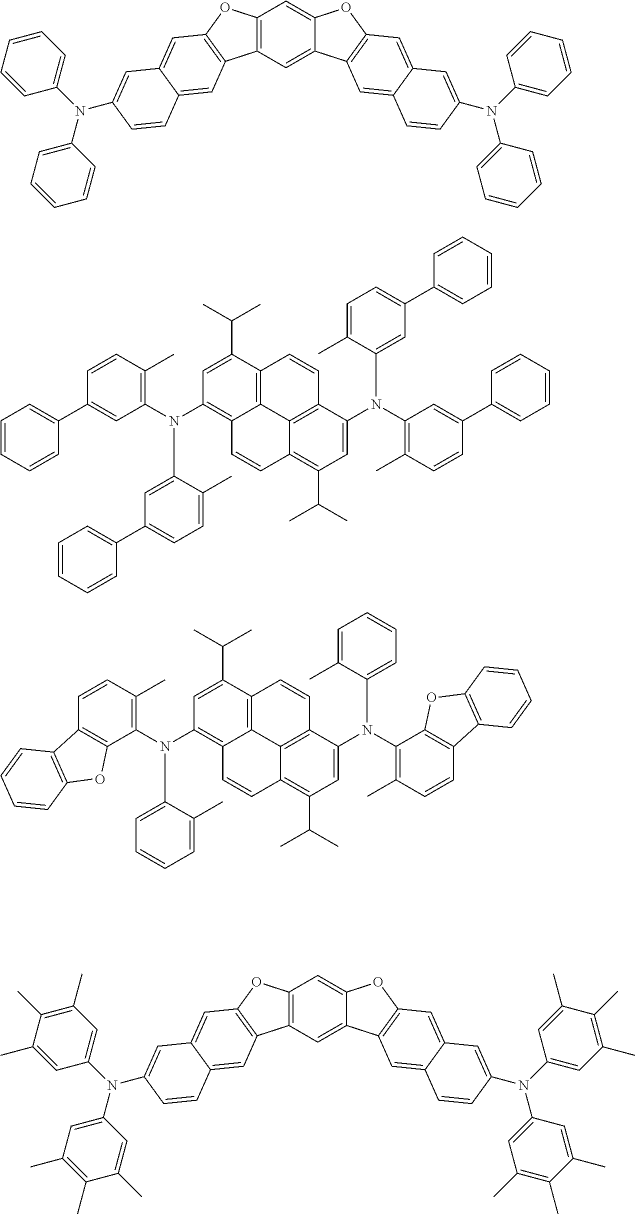

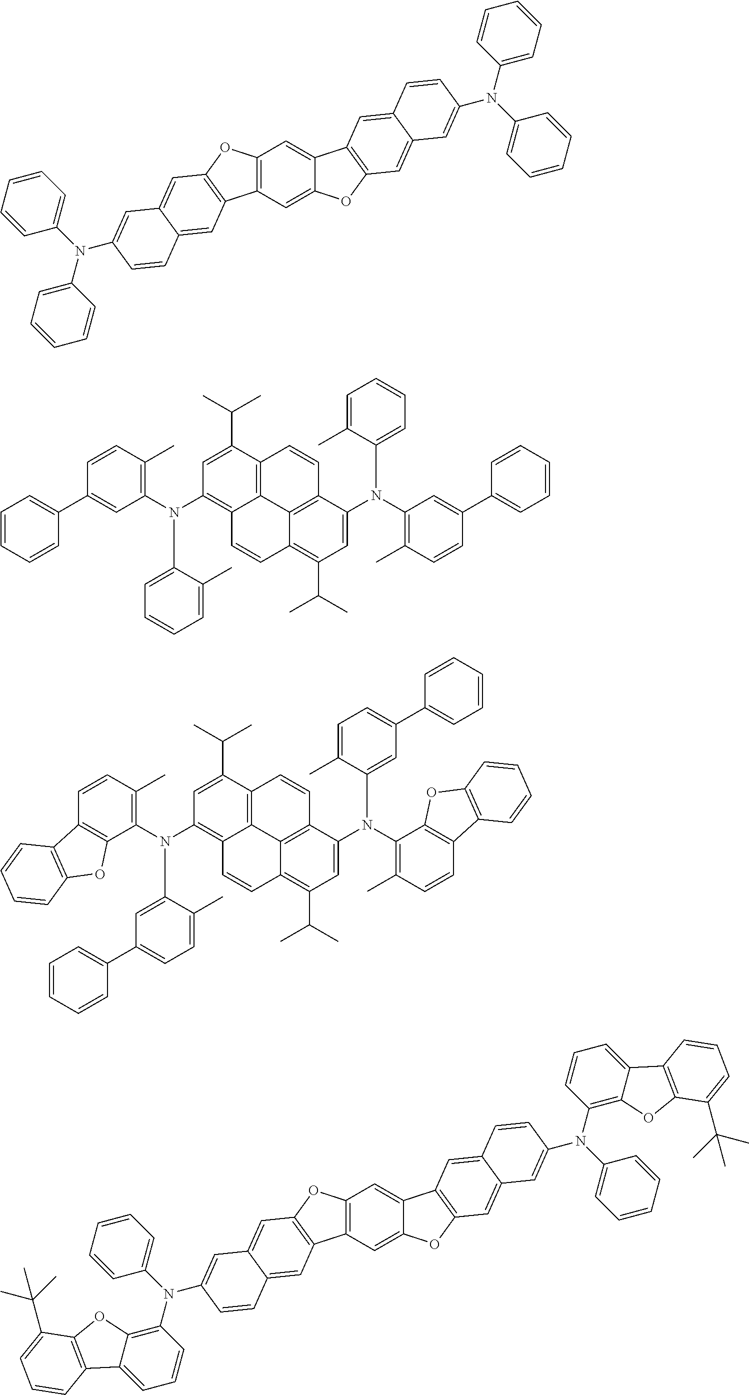 [SMILES: c1ccc(N(c2ccccc2)c2ccc3cc4c(cc3c2)oc2cc3c(cc24)oc2cc4cc(N(c5ccccc5)c5ccccc5)ccc4cc23)cc1 ; Cc1ccccc1N(c1cc(-c2ccccc2)ccc1C)c1cc(C(C)C)c2ccc3c(N(c4ccccc4C)c4cc(-c5ccccc5)ccc4C)cc(C(C)C)c4ccc1c2c43 ; Cc1ccc(-c2ccccc2)cc1N(c1cc(C(C)C)c2ccc3c(N(c4cc(-c5ccccc5)ccc4C)c4c(C)ccc5c4oc4ccccc45)cc(C(C)C)c4ccc1c2c43)c1c(C)ccc2c1oc1ccccc12 ; CC(C)(C)c1cccc2c1oc1c(N(c3ccccc3)c3ccc4cc5c(cc4c3)oc3cc4c(cc35)oc3cc5cc(N(c6ccccc6)c6cccc7c6oc6c(C(C)(C)C)cccc67)ccc5cc34)cccc12]

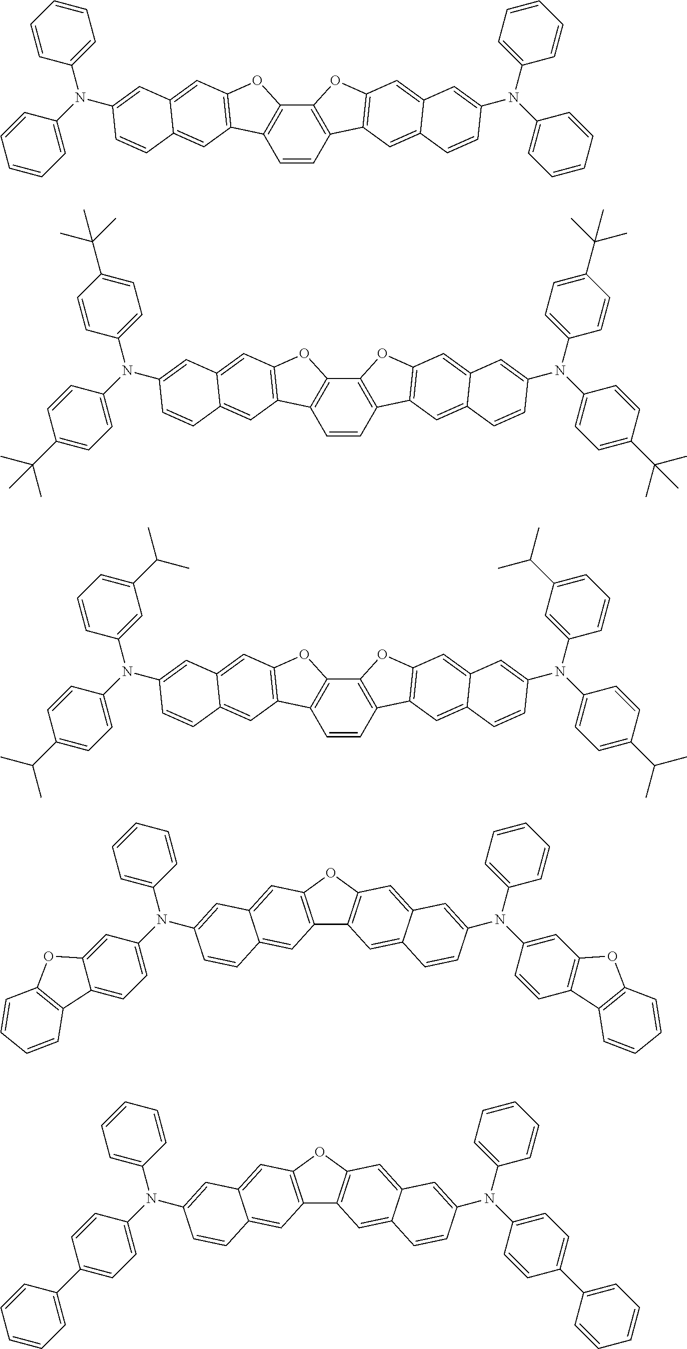

The organic EL device of the second aspect of the invention is characterized in that,

the organic layer further includes a hole-blocking layer directly in contact with the emitting layer, and

the hole-blocking layer contains either or both of a compound represented by the following formula (21) and a compound represented by the following formula (31).

Here, a “hole-blocking layer” is a layer provided between an emitting layer and an electron-transporting layer forthe purpose of preventing holes from leaking from the emitting layer to the electron-transporting layer, and also functions as an electron-transporting layer for transporting electrons injected from a cathode to the emitting layer.

### [Compound Represented by the Formula (21)]

Next, a compound represented by the formula (21) will be described.

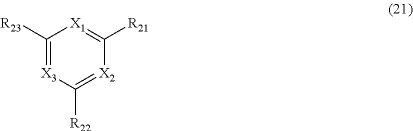

In the formula (21),

X₁ to X₃ are independently, N or CR_{b}; provided that one or more of X₁ to X₃ is N.

R_{b} is

a hydrogen atom,

a halogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms.

When two R_{b}'s are present, the two R_{b}'s may be the same as or different to each other.

R_{b} does not form a ring by binding with adjacent R₂₁ to R₂₃.

R₂₁ to R₂₃ are independently,

-(L₂)ₘ-(Ar₂)ₙ,

a hydrogen atom,

a substituted or unsubstituted alkyl group including 1 to 50 carbon atoms,

a substituted or unsubstituted alkenyl group including 2 to 50 carbon atoms,

a substituted or unsubstituted alkynyl group including 2 to 50 carbon atoms,

a substituted or unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a halogen atom, a cyano group, a nitro group,

a substituted or unsubstituted aryl group including 6 to 50 ring carbon atoms, or

a substituted or unsubstituted monovalent heterocyclic group including 5 to 50 ring atoms.

R₉₀₁ to R₉₀₇ are independently,

a hydrogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms.

When two or more of each of R₉₀₁ to R₉₀₇ are present, the two or more of each of R₉₀₁ to R₉₀₇ may be the same as or different to each other.

L₂ is

a substituted or unsubstituted arylene group including 6 to 50 ring carbon atoms, or

a substituted or unsubstituted divalent heterocyclic group including 5 to 50 ring atoms.

m is an integer of 0 to 2.

When m is 0, L₂ is a single bond.

When m is 2, two L₂'s may be the same as or different to each other.

Ar₂ is

a substituted or unsubstituted aryl group including 6 to 50 ring carbon atoms, or

a substituted or unsubstituted monovalent heterocyclic group including 5 to 50 ring atoms.

n is an integer of 1 or 2, and when n is 2, two Ar₂'s may be the same as or different to each other; provided that when n is 2, m is 1 or more.

In one embodiment, two among X₁ to X₃ in the formula (21) are N. That is, the central skeleton is a pyrimidine ring.

In one embodiment, R₂₁ to R₂₃ in the formula (21) are independently,

a substituted or unsubstituted aryl group including 6 to 50 ring carbon atoms.

In one embodiment, the compound represented by the formula (21) is a compound represented by the following formula (21-1):

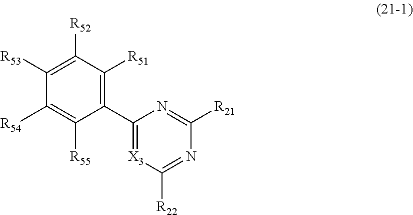

In the formula (21-1), R₂₁, R₂₂ and X₃ are as defined in the formula (21).

R₅₁ to R₅₅ are independently,

a hydrogen atom,

an unsubstituted alkyl group including 1 to 50 carbon atoms,

an unsubstituted alkenyl group including 2 to 50 carbon atoms,

an unsubstituted alkynyl group including 2 to 50 carbon atoms,

an unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a halogen atom, a cyano group, a nitro group,

an unsubstituted aryl group including 6 to 50 ring carbon atoms, or

an unsubstituted monovalent heterocyclic group including 5 to 50 ring atoms.

One or more sets of adjacent two or more among R₅₁ to R₅₅ form a substituted or unsubstituted, saturated or unsaturated ring including 3 to 30 ring atoms by bonding with each other, or do not form a ring.

R₉₀₁ to R₉₀₇ are independently,

a hydrogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms.

When two or more of each of R₉₀₁ to R₉₀₇ are present, the two or more of each of R₉₀₁ to R₉₀₇ may be the same as or different to each other.

In one embodiment, the compound represented by the formula (21-1) is a compound represented by the following formula (21-2):

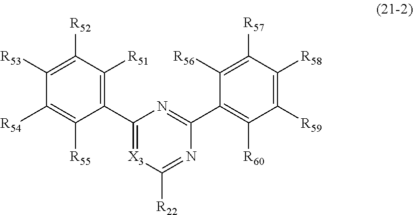

In the formula (21-2), R₂₂, X₃ and R₅₁ to R₅₅ are as defined in the formula (21-1).

R₅₆ to R₆₀ are independently,

a hydrogen atom,

an unsubstituted alkyl group including 1 to 50 carbon atoms,

an unsubstituted alkenyl group including 2 to 50 carbon atoms,

an unsubstituted alkynyl group including 2 to 50 carbon atoms,

an unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a halogen atom, a cyano group, a nitro group,

an unsubstituted aryl group including 6 to 50 ring carbon atoms, or

an unsubstituted monovalent heterocyclic group including 5 to 50 ring atoms.

R₉₀₁ to R₉₀₇ are independently,

a hydrogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms.

When two or more of each of R₉₀₁ to R₉₀₇ are present, the two or more of each of R₉₀₁ to R₉₀₇ may be the same as or different to each other.

In one embodiment, the compound represented by the formula (21-2) is a compound represented by the following formula (21-3):

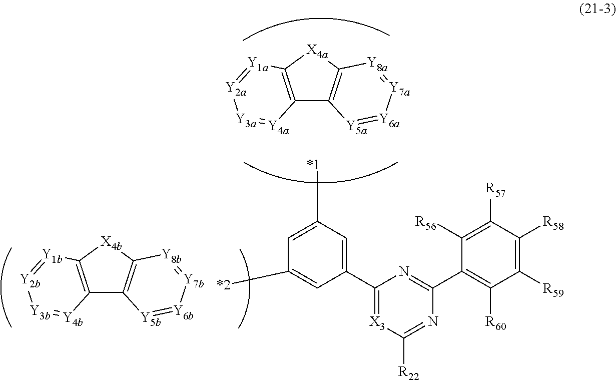

In the formula (21-3), R₂₂, X₃ and R₅₆ to R₆₀ are as defined in the formula (21-2).

Y₁ₐ to Y₈ₐ are independently, a CR₆₁ₐ or N.

Y_{1b} to Y_{8b} are independently, a CR_{61b} or N.

X₄a is O, S or NR₆₁ₐ.

X_{4b} is O, S or NR_{61b}.

R₆₁ₐ and R_{61b} are independently,

a hydrogen atom,

an unsubstituted alkyl group including 1 to 50 carbon atoms,

an unsubstituted alkenyl group including 2 to 50 carbon atoms,

an unsubstituted alkynyl group including 2 to 50 carbon atoms,

an unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a halogen atom, a cyano group, a nitro group,

an unsubstituted aryl group including 6 to 50 ring carbon atoms, or

an unsubstituted monovalent heterocyclic group including 5 to 50 ring atoms.

R₉₀₁ to R₉₀₇ are independently,

a hydrogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms.

When two or more of each of R₉₀₁ to R₉₀₇ are present, the two or more of each of R₉₀₁ to R₉₀₇ may be the same or different.

When a plurality of R₆₁ₐ's are present, the plurality of R₆₁ₐ'S may be the same or different.

When a plurality of R_{61b}'s are present, the plurality of R_{61b}'s may be the same or different.

One or more sets of two or more R₆₁ₐ's substituting adjacent atoms form a substituted or unsubstituted, saturated or unsaturated ring including 3 to 30 ring atoms by bonding with each other, or do not form a ring;

one or more sets of two or more R_{61b}'s substituting adjacent atoms form a substituted or unsubstituted, saturated or unsaturated ring including 3 to 30 ring atoms by bonding with each other, or do not form a ring;

provided that one of R₆₁ₐ's is a single bond which is bonded with *1, or one of the atoms constituting the ring formed by bonding one or more sets of two or more R₆₁ₐ's substituting adjacent atoms with each other is bonded with *1 via a single bond; and

one of R_{61b}'s is a single bond which is bonded with *2, or one of the atoms constituting the ring formed by bonding one or more sets of two or more R_{61b}'s substituting adjacent atoms with each other is bonded with *2 via a single bond.

Specific examples of the group composed of X₄ₐ and Y₁ₐ to Y₁ₐ when “one of R₆₁ₐ's is a single bond bonding with benzene ring or R_{61c} is a single bond bonding with *1” include:

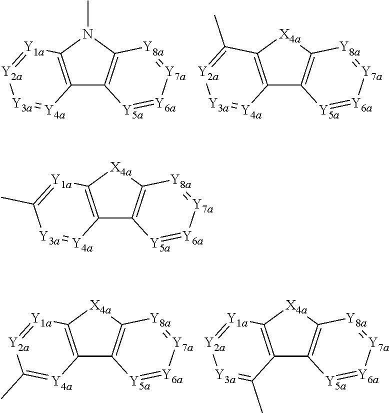

Further, specific examples of the group composed of X₄ₐ and Y₁ₐ to Y₈ₐ when “one of the atoms constituting the ring formed by bonding one or more sets of two or more of R₆₁ₐ's substituting adjacent atoms with each other is bonded with a carbon atom of the benzene ring via a single bond” include:

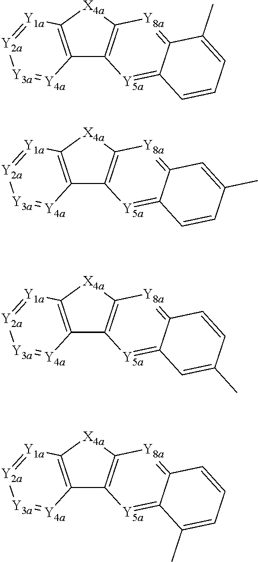

In one embodiment, the compound represented by the formula (21) is a compound represented by the following formula (21-4):

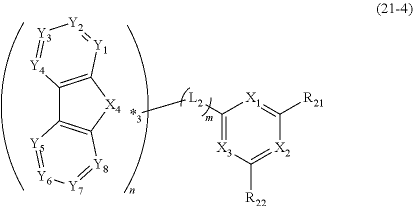

In the formula (21-4), X₁ to X₃, R₂₁, R₂₂, L₂, m and n are as defined in the formula (21).

Y₁ to Y₈ are independently, CR₆₁ₑ or N.

X₄ is O, S or NR₆₁ₑ.

R₆₁ₑ's are independently,

a hydrogen atom,

an unsubstituted alkyl group including 1 to 50 carbon atoms,

an unsubstituted alkenyl group including 2 to 50 carbon atoms,

an unsubstituted alkynyl group including 2 to 50 carbon atoms,

an unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a halogen atom, a cyano group, a nitro group,

an unsubstituted aryl group including 6 to 50 ring carbon atoms, or

an unsubstituted monovalent heterocyclic group including 5 to 50 ring atoms.

R₉₀₁ to R₉₀₇ are independently,

a hydrogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms.

When two or more of each of R₉₀₁ to R₉₀₇ are present, the two or more of each of R₉₀₁ to R₉₀₇ may be the same or different.

When a plurality of R₆₁ₑ's are present, the plurality of R₆₁ₑ's may be the same as or different to each other.

One or more sets of two or more R₆₁ₑ's substituting adjacent atoms form a substituted or unsubstituted, saturated or unsaturated ring including 3 to 30 ring atoms by bonding with each other, or do not form a ring;

provided that one of R₆₁ₑ's is a single bond which is bonded with *3, or one of the atoms constituting the ring formed by bonding one or more sets of two or more R₆₁ₑ's substituting adjacent atoms with each other is bonded with *3 via a single bond.

Specific examples of the compound represented by the formula (21) will be described below, but these are merely examples, and the compound represented by the formula (21) is not limited to the following specific examples.

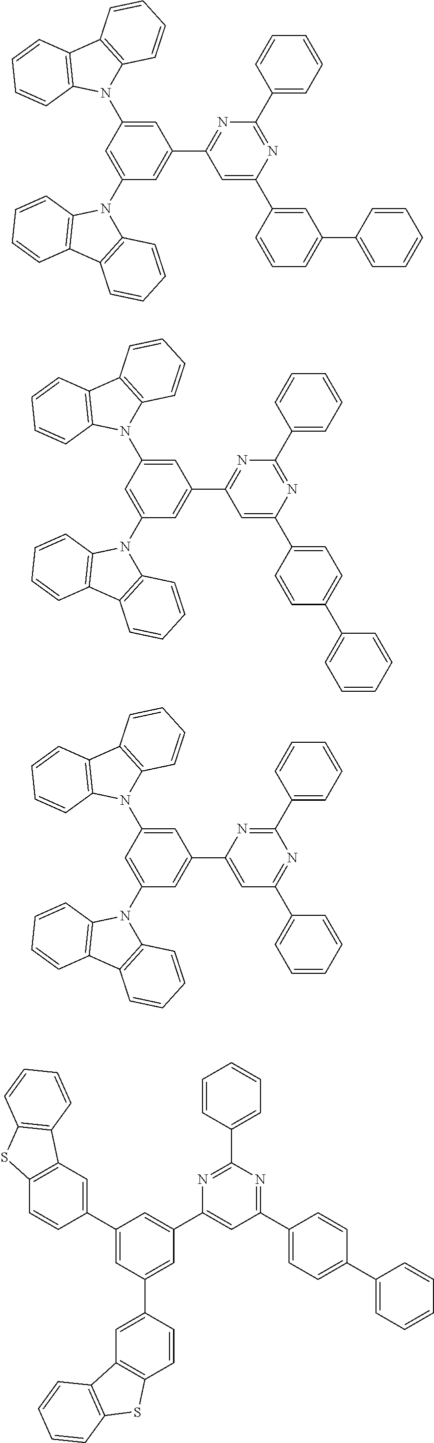

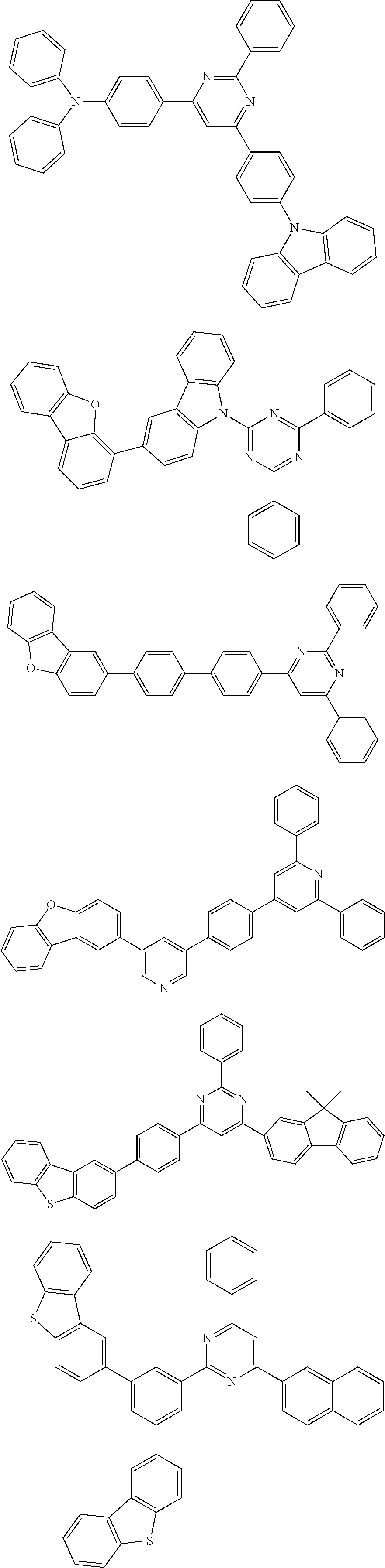

[Compound represented by the formula (31)]

Next, a compound represented by the formula (31) will be described.

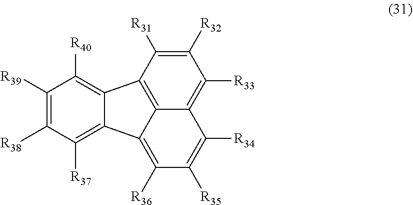

In the formula (31),

one or more among R₃₁ to R₄₀ are -(L₃)ₚ-Ar₃.

When two or more -(L₃)ₚ-Ar₃ are present, the two or more -(L₃)ₚ-Ar₃ may be the same as or different to each other.

L₃ is

a substituted or unsubstituted arylene group including 6 to 50 ring carbon atoms, or

a substituted or unsubstituted divalent heterocyclic group including 5 to 50 ring atoms.

p is an integer of 0 to 3.

When p is 0, L₃ is a single bond.

When p is 2 or more, the plurality of L₃'s may be the same as or different to each other.

Ar₃ is

a substituted or unsubstituted aryl group including 6 to 50 ring carbon atoms, or

a substituted or unsubstituted monovalent heterocyclic group including 5 to 50 ring atoms.

One or more sets of adjacent two or more among R₃₁ to Roe which are not -(L₃)ₚ-Ar₃ and R₃₇ to R₄₀ which are not -(L₃)ₚ-Ar₃ form a substituted or unsubstituted, saturated or unsaturated ring including 3 to 30 ring atoms by bonding with each other, or do not form a ring.

R₃₁ to R₄₀ which are not involved in the ring formation are independently,

a hydrogen atom,

a substituted or unsubstituted alkyl group including 1 to 50 carbon atoms,

a substituted or unsubstituted alkenyl group including 2 to 50 carbon atoms,

a substituted or unsubstituted alkynyl group including 2 to 50 carbon atoms,

a substituted or unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a halogen atom, a cyano group, a nitro group,

a substituted or unsubstituted aryl group including 6 to 50 ring carbon atoms, or

a substituted or unsubstituted monovalent heterocyclic group including 5 to 50 ring atoms.

R₉₀₁ to R₉₀₇ are independently,

a hydrogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms.

When two or more of each of R₉₀₁ to R₉₀₇ are present, the two or more of each of R₉₀₁ to R₉₀₇ may be the same or different.

In one embodiment, p in the formula (31) is preferably 0 or 1.

In one embodiment, the compound represented by the formula (31) is a compound represented by the following formula (31-1):

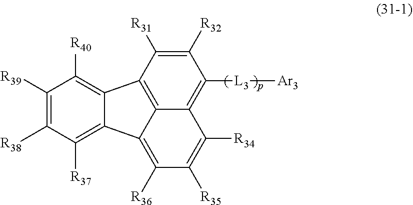

In the formula (31-1), L₃, p, Ar₃, R₃₁, R₃₂, and R₃₄ to R₄₀ are as defined in the formula (31).

In one embodiment, the compound represented by the formula (31) is a compound represented by the following formula (31-1H):

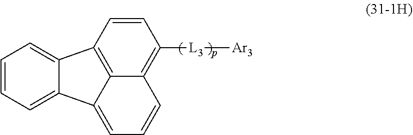

In the formula (31-1H), L₃, p and Ar₃ are as defined in the formula (31).

Specific examples of the compound represented by the formula (31) will be described below, but these are merely examples, and the compound represented by the formula (31) is not limited to the following specific examples.

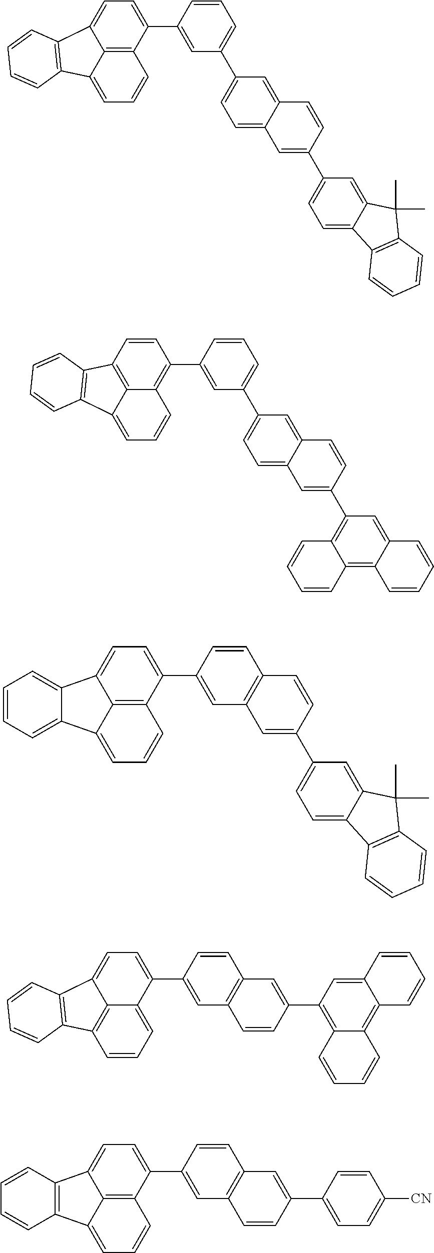

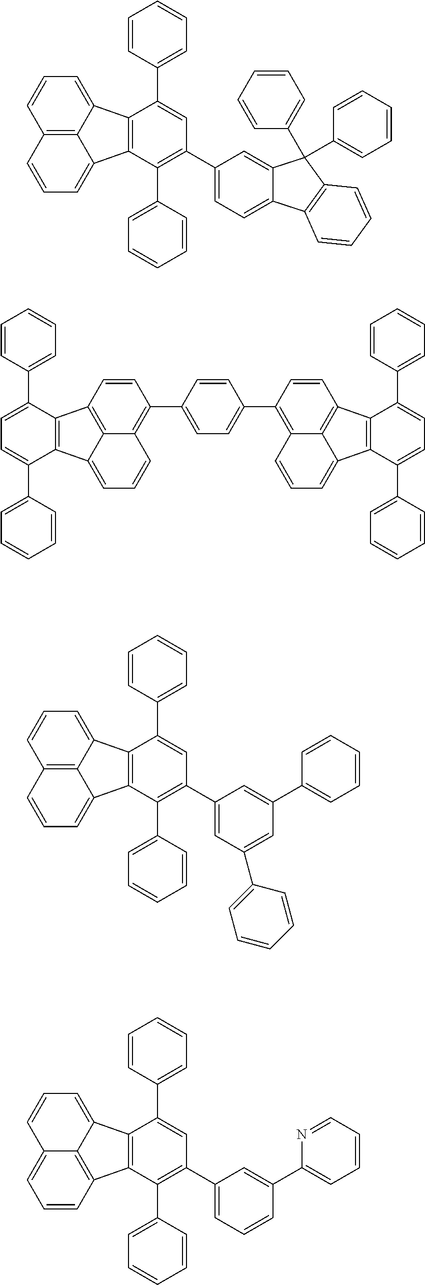

The organic EL device of the third aspect of the invention is characterized that, the organic layer further includes an electron-blocking layer directly in contact with an emitting layer, and

the electron-blocking layer contains either or both of a compound represented by the following formula (41) and a compound represented by the following formula (51).

Here, an “electron-blocking layer” is a layer provided between an emitting layer and an hole-transporting layer for the purpose of preventing electrons from leaking from the emitting layer to the hole-transporting layer, and also functions as a hole-transporting layer for transporting holes injected from an anode to an emitting layer.

### [Compound Represented by the Formula (41)]

Hereinafter, a compound represented by the formula (41) will be described.

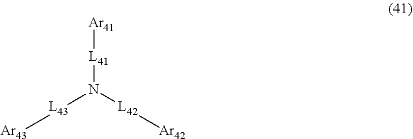

In the formula (41),

L₄₁ to L₄₃ are independently,

a single bond,

a substituted or unsubstituted arylene group including 6 to 50 ring carbon atoms, or

a substituted or unsubstituted divalent heterocyclic group including 5 to 50 ring atoms.

Ar₄ to Ar₄ are independently,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms.

In one embodiment, the compound represented by the formula (41) is a compound represented by the following formula (41-1):

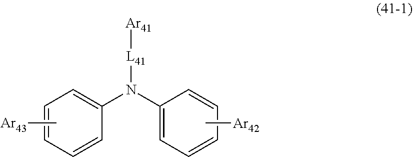

In the formula (41-1), Ar₄ to Ar₄₁ and L₄₁ are as defined in the formula (41). Ar₄₂ and Ar₄₃ are each bonded with any carbon atoms constituting the substituted phenyl group on which they substitute.

In one embodiment, the compound represented by the formula (41) is a compound represented by the following formula (41-2):

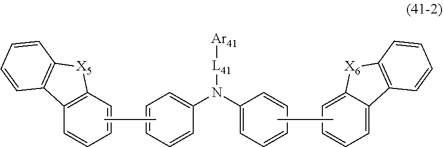

In the formula (41-2), Ar₄₁ and L₄₁ are as defined in the formula (41).

X₅ and X₆ are independently, O, S or N (R₉₀₆).

R₉₀₆ is,

a hydrogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms.

When two R₉₀₆'s are present, the two R₉₀₆'s may be the same or different.

Either of carbon atoms constituting one benzene ring of the monovalent heterocyclic group including X₅ or X₆ and either of carbon atoms constituting the phenyl group substituted with a central nitrogen atom are bonded.

In one embodiment, the compound represented by the formula (41) is a compound represented by the following formula (41-3):

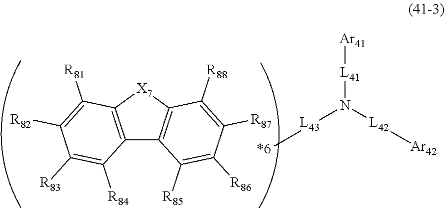

In the formula (41-3), Ar₄₁, Ar₄₂ and L₄₁ to L₄₃ are as defined in the formula (41).

X₇ is O, S or NR₈₉.

R₈₁ to R₈₉ are independently,

a hydrogen atom,

an unsubstituted alkyl group including 1 to 50 carbon atoms,

an unsubstituted alkenyl group including 2 to 50 carbon atoms,

an unsubstituted alkynyl group including 2 to 50 carbon atoms,

an unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a halogen atom, a cyano group, a nitro group,

an unsubstituted aryl group including 6 to 50 ring carbon atoms, or

an unsubstituted monovalent heterocyclic group including 5 to 50 ring atoms.

R₉₀₁ to R₉₀₇ are independently,

a hydrogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms.

When two or more of each of R₉₀₁ to R₉₀₇ are present, the two or more of each of R₉₀₁ to R₉₀₇ may be the same or different.

One or more sets of two or more R₈₁ to R₈₉ substituting adjacent atoms form a substituted or unsubstituted, saturated or unsaturated ring including 3 to 30 ring atoms by bonding with each other, or do not form a ring;

provided that one of R₈₁ to R₈₉ is a single bond which is bonded with *6, or one of the atoms constituting the ring formed by bonding one or more sets of two or more among R₈₁ to R₈₉ substituting adjacent atoms with each other is bonded with *6 via a single bond.

Specific examples of the compound represented by the formula (41) will be described below, but these are merely examples, and the compound represented by the formula (41) is not limited to the following specific examples.

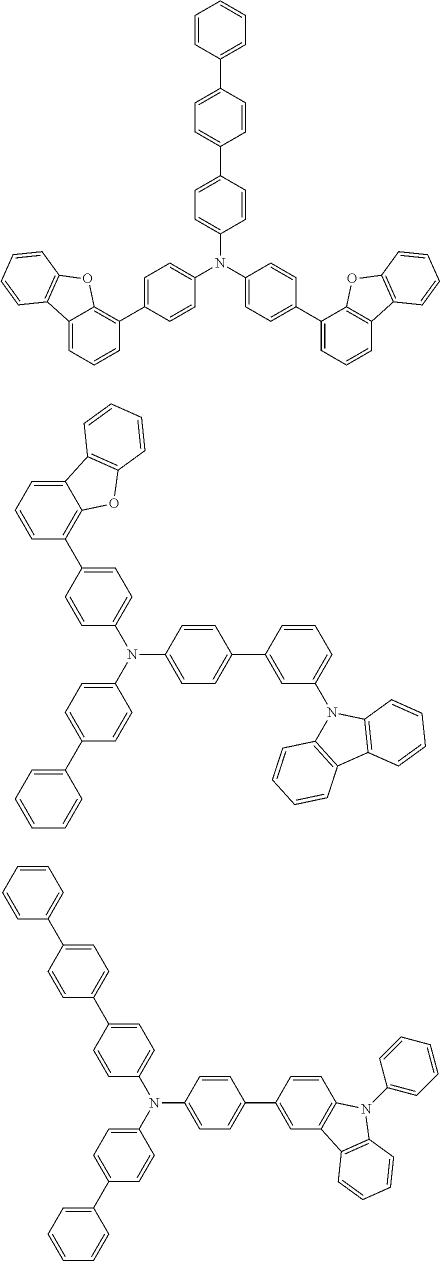

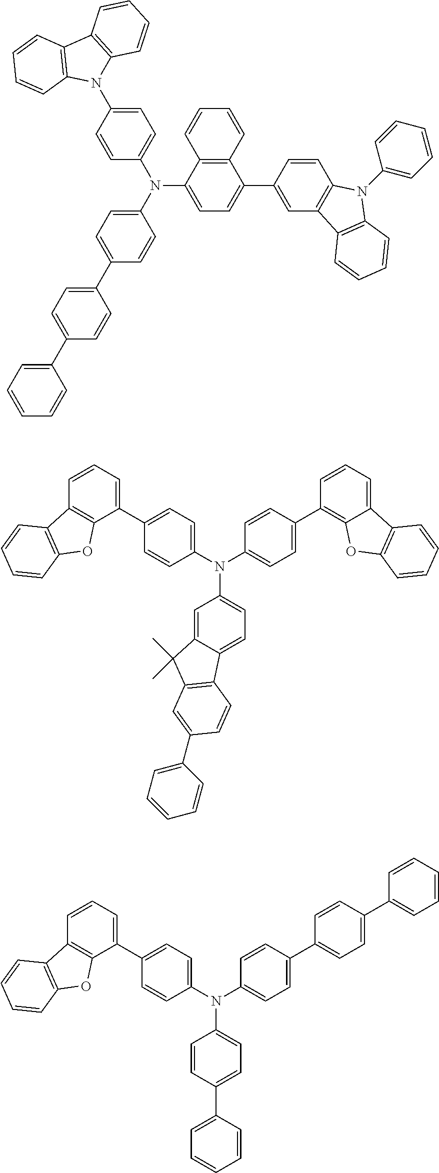 [SMILES: c1ccc(-c2ccc(-c3ccc(N(c4ccc(-n5c6ccccc6c6ccccc65)cc4)c4ccc(-c5ccc6c(c5)c5ccccc5n6-c5ccccc5)c5ccccc45)cc3)cc2)cc1 ; CC1(C)c2cc(-c3ccccc3)ccc2-c2ccc(N(c3ccc(-c4cccc5c4oc4ccccc45)cc3)c3ccc(-c4cccc5c4oc4ccccc45)cc3)cc21 ; c1ccc(-c2ccc(-c3ccc(N(c4ccc(-c5ccccc5)cc4)c4ccc(-c5cccc6c5oc5ccccc56)cc4)cc3)cc2)cc1]

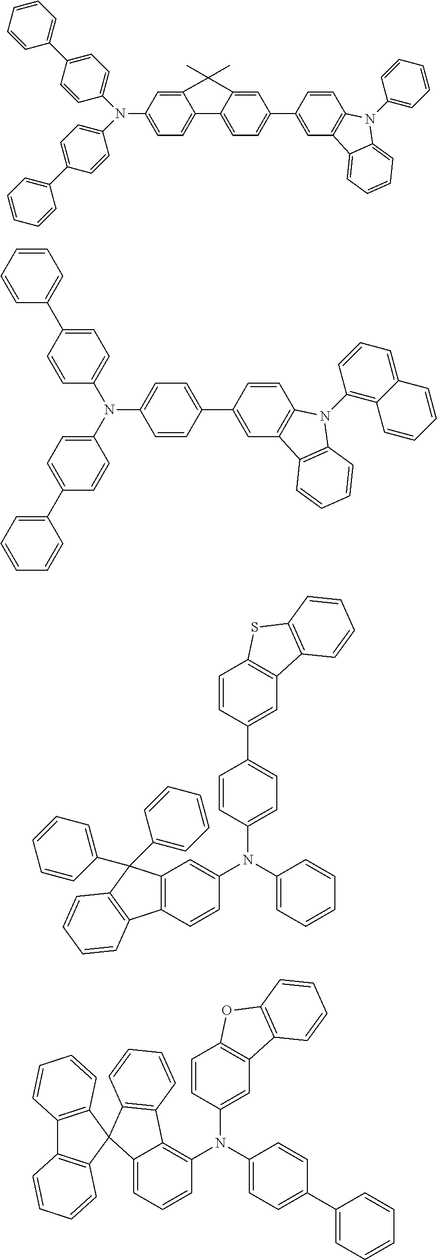

### [Compound Represented by the Formula (51)]

Next, a compound represented by the formula (51) will be described.

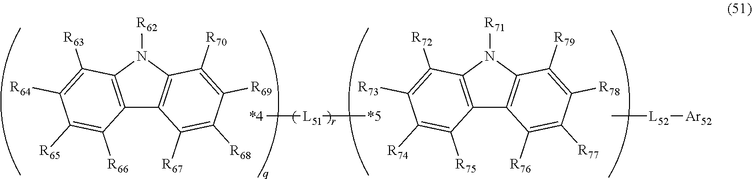

In the formula (51),

R₆₂ to R₇₉ are independently,

a hydrogen atom,

an unsubstituted alkyl group including 1 to 50 carbon atoms,

an unsubstituted alkenyl group including 2 to 50 carbon atoms,

an unsubstituted alkynyl group including 2 to 50 carbon atoms,

an unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a halogen atom, a cyano group, a nitro group,

an unsubstituted aryl group including 6 to 50 ring carbon atoms, or

an unsubstituted monovalent heterocyclic group including 5 to 50 ring atoms.

R₉₀₁ to R₉₀₇ are independently,

a hydrogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms.

When two or more of each of R₉₀₁ to R₉₀₇ are present, the two or more of each of R₉₀₁ to R₉₀₇ may be the same or different.

One or more sets of two or more R₆₂ to R₇₀ substituting adjacent atoms form a substituted or unsubstituted, saturated or unsaturated ring including 3 to 30 ring atoms by bonding with each other, or do not form a ring;

one or more sets of two or more R₇₁ to R₇₉ substituting adjacent atoms form a substituted or unsubstituted, saturated or unsaturated ring including 3 to 30 ring atoms by bonding with each other, or do not form a ring;

provided that one of R₆₂ to R₇₀ is a single bond which is bonded with *4, or one of the atoms constituting the ring formed by bonding one or more sets of two or more among R₆₂ to R₇₀ substituting adjacent atoms with each other is bonded with *4 via a single bond; and

one of R₇₁ to R₇₉ is a single bond which is bonded with *5, or one of the atoms constituting the ring formed by bonding one or more sets of two or more among R₇₁ to R₇₉ substituting adjacent atoms with each other is bonded with *5 via a single bond.

Also, one of R₇₁ to R₇₉ which is not bonded with *5 is a single bond which is bonded with L₅₂, or another one of the atoms constituting the ring formed by bonding one or more sets of two or more among R₇₁ to R₇₉ substituting adjacent atoms with each other is bonded with L₅₂ via a single bond;

L₅₁'s are independently,

a substituted or unsubstituted arylene group including 6 to 50 ring carbon atoms, or

a substituted or unsubstituted divalent heterocyclic group including 5 to 50 ring atoms.

q is an integer of 0 to 3.

When q is 2 or more, two or more of each of R₆₂ to R₇₀ may be the same as or different to each other provided that when q is 0, L₅₁'s terminated by a hydrogen atom.

r is an integer of 0 to 2.

When r is 0, L₅₁ is a single bond.

When r is 2, two L₅₁'s may be the same as or different to each other; provided that when q is 2 or more, r is 1 or 2.

L₅₂ is

a single bond,

a substituted or unsubstituted arylene group including 6 to 50 ring carbon atoms, or

a substituted or unsubstituted divalent heterocyclic group including 5 to 50 ring atoms.

Ar₅₂ is

a substituted or unsubstituted aryl group including 6 to 50 ring carbon atoms, or

a substituted or unsubstituted monovalent heterocyclic group including 5 to 50 ring atoms.

In one embodiment, the compound represented by the formula (51) is a compound represented by the following formula (51-1).

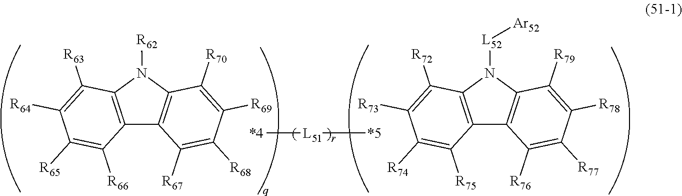

In the formula (51-1), R₆₂ to R₇₀, R₇₂ to R₇₉, *4, *5, q, L₅₁, r, L₅₂ and Ar₅₂ are as defined in the formula (51).

In one embodiment, q in the formula (51) is 1.

In one embodiment, the compound represented by the formula (51) is a compound represented by the following formula (51-2).

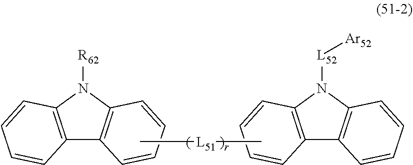

In the formula (51-2), R₆₂, L₅₁, r, L₅₂ and Ar₅₂ are as defined in the formula (51).

In one embodiment, the compound represented by the formula (51) is selected from the group consisting of a compound represented by the following formula (51-3a), a compound represented by the following formula (51-3b), and a compound represented by the following formula (51-3c).

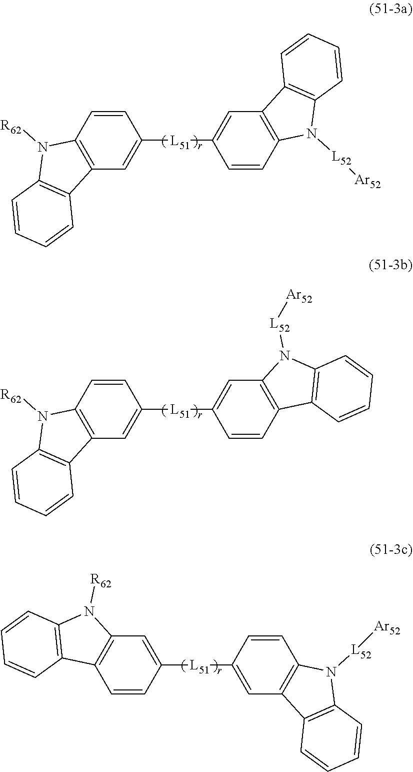

In the formulas (51-3a) to (51-3c), R₆₂, L₅₁, r, L₅₂ and Ar₅₂ are as defined in the formula (51).

Specific examples of the compound represented by the formula (51) will be described below, but these are merely examples, and the compound represented by the formula (51) is not limited to the following specific examples.

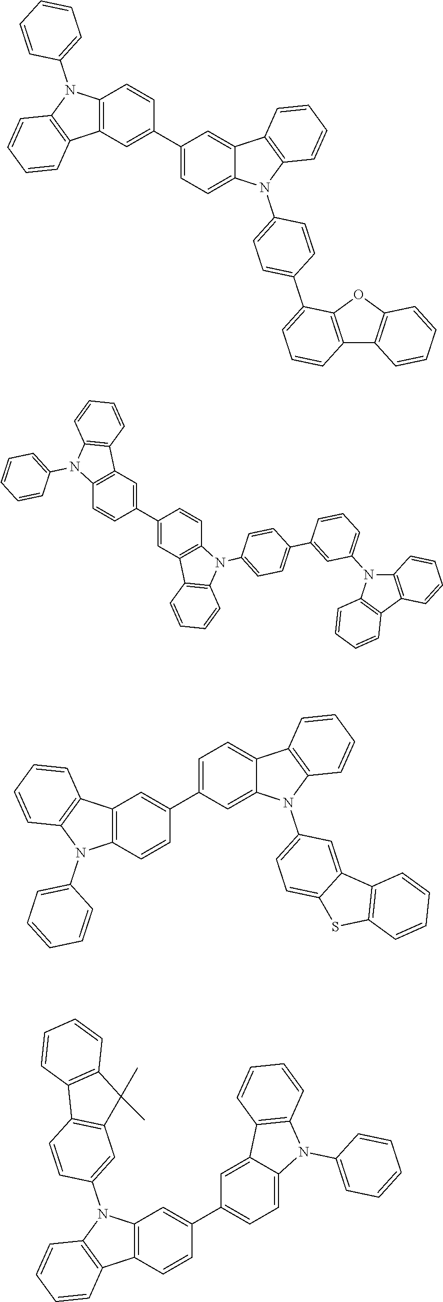 [SMILES: c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccccc4n5-c4ccc(-c5cccc(-n6c7ccccc7c7ccccc76)c5)cc4)ccc32)cc1 ; c1ccc(-n2c3ccccc3c3cc(-c4ccc5c6ccccc6n(-c6ccc7sc8ccccc8c7c6)c5c4)ccc32)cc1 ; CC1(C)c2ccccc2-c2ccc(-n3c4ccccc4c4ccc(-c5ccc6c(c5)c5ccccc5n6-c5ccccc5)cc43)cc21]

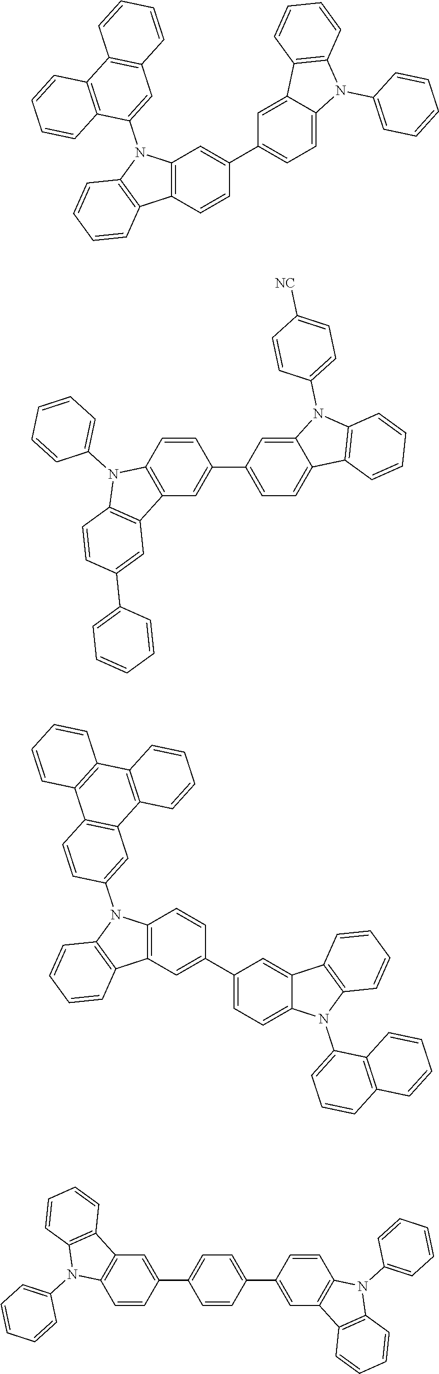 [SMILES: c1ccc(-n2c3ccccc3c3cc(-c4ccc5c6ccccc6n(-c6cc7ccccc7c7ccccc67)c5c4)ccc32)cc1 ; N#Cc1ccc(-n2c3ccccc3c3ccc(-c4ccc5c(c4)c4cc(-c6ccccc6)ccc4n5-c4ccccc4)cc32)cc1 ; c1ccc2c(-n3c4ccccc4c4cc(-c5ccc6c(c5)c5ccccc5n6-c5ccc6c7ccccc7c7ccccc7c6c5)ccc43)cccc2c1 ; c1ccc(-n2c3ccccc3c3cc(-c4ccc(-c5ccc6c(c5)c5ccccc5n6-c5ccccc5)cc4)ccc32)cc1]

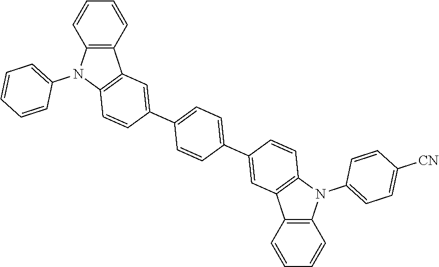 [SMILES: N#Cc1ccc(-n2c3ccccc3c3cc(-c4ccc(-c5ccc6c(c5)c5ccccc5n6-c5ccccc5)cc4)ccc32)cc1]

In one embodiment, in the compound represented by any of the formulas (1), (11), (21), (31), (41) and (51), the substituent in the case of “substituted or unsubstituted” is a group selected from the group consisting of:

an unsubstituted alkyl group including 1 to 50 carbon atoms,

an unsubstituted alkenyl group including 2 to 50 carbon atoms,

an unsubstituted alkynyl group including 2 to 50 carbon atoms,

an unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇)

(where,

R₉₀₁ to R₉₀₇ are independently,

a hydrogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms;

when two or more of each of R₉₀₁ to R₉₀₇ are present, the two or more of each of R₉₀₁ to R₉₀₇ may be the same or different.),

a halogen atom, a cyano group, a nitro group,

an unsubstituted aryl group including 6 to 50 ring carbon atoms, and

an unsubstituted monovalent heterocyclic group including 5 to 50 ring atoms.

In one embodiment, in the compound represented by any of the formulas (1), (11), (21), (31), (41) and (51), the substituent in the case of “substituted or unsubstituted” is a group selected from the group consisting of:

an alkyl group including 1 to 50 carbon atoms,

an aryl group including 6 to 50 ring carbon atoms, and

a monovalent heterocyclic group including 5 to 50 ring atoms.

In one embodiment, in the compound represented by any of the formulas (1), (11), (21), (31), (41) and (51), the substituent in the case of “substituted or unsubstituted” is a group selected from the group consisting of:

an alkyl group including 1 to 18 carbon atoms,

an aryl group including 6 to 18 ring carbon atoms, and

a monovalent heterocyclic group including 5 to 18 ring atoms.

Specific examples of the above groups are as described in the section of [Definitions] of this specification.

As described above, known materials and device configurations may be applied to the organic EL device of the first aspect of the invention, as long as the device includes a cathode, an anode, and an organic layer between the cathode and the anode, wherein the organic layer contains the compound represented by the formula (1) and the compound represented by the formula (11), and the effect of the invention is not impaired.

As described above, known materials and device configurations may be applied to the organic EL device of the second aspect of the invention, as long as the device includes a cathode, an anode, and an organic layer between the cathode and the anode, wherein the organic layer includes an emitting layer and a hole-blocking layer directly in contact with the emitting layer, the emitting layer contains the compound represented by the formula (1) and the compound represented by the formula (11); the hole-blocking layer contains the compound represented by the formula (21) and/or the compound represented by the formula (31); and the effect of the invention is not impaired.

As described above, known materials and device configurations may be applied to the organic EL device of the third aspect of the invention, as long as the device includes a cathode, an anode, and an organic layer between the cathode and the anode, wherein the organic layer includes an emitting layer and an electron-blocking layer directly in contact with the emitting layer, the emitting layer contains the compound represented by the formula (1) and the compound represented by the formula (11); the electron-blocking layer contains the compound represented by the formula (41); and the effect of the invention is not impaired.

As described above, known materials and device configurations may be applied to the organic EL device of the fourth aspect of the invention, as long as the device includes a cathode, an anode, and an organic layer between the cathode and the anode, wherein the organic layer includes an emitting layer, a hole-blocking layer directly in contact with the emitting layer and an electron-blocking layer directly in contact with the emitting layer, and the emitting layer contains the compound represented by the formula (1) and the compound represented by the formula (11); the hole-blocking layer contains the compound represented by the formula (21) and/or the compound represented by the formula (31); the electron-blocking layer contains the compound represented by the formula (41); and the effect of the invention is not impaired.

As described above, known materials and device configurations may be applied to the organic EL device of the fifth aspect of the invention, as long as the device includes a cathode, an anode, and an organic layer between the cathode and the anode, wherein the organic layer includes two or more emitting layers, and one or more of the two or more emitting layers contains the compound represented by the formula (1) and the compound represented by the formula (11); and the effect of the invention is not impaired.

As described above, known materials and device configurations may be applied to the organic EL device of the other fifth aspect of the invention, as long as the device includes a cathode, an anode, and an organic layer between the cathode and the anode, wherein the organic layer includes two or more emitting layers, and one or more of the two or more emitting layers contains the compound represented by the formula (1) and a compound A having a Stokes shift of 20 nm or less and an emission peak wavelength of 440 nm to 465 nm; and the effect of the invention is not impaired.

Hereinafter, parts which can be used in one embodiment of the organic EL device of the first to fifth aspects of the invention, materials for forming respective layers, other than the compound represented by any of the formula (1), (11), (21), (31), (41) and (51), and the like, will be described later.

### (Substrate)

A substrate is used as a support of an emitting device. As the substrate, glass, quartz, plastics or the like can be used, for example. Further, a flexible substrate may be used. The “flexible substrate” means a bendable (flexible) substrate, and specific examples thereof include a plastic substrate formed of polycarbonate, polyvinyl chloride, and the like.

### (Anode)

For the anode formed on the substrate, metals, alloys, electrically conductive compounds, mixtures thereof, and the like, which have a large work function (specifically 4.0 eV or more) are preferably used. Specific examples thereof include indium oxide-tin oxide (ITO: Indium Tin Oxide), silicon or silicon oxide-containing indium oxide-tin oxide, indium oxide-zinc oxide, tungsten oxide, zinc oxide-containing indium oxide, and graphene. In addition thereto, specific examples thereof include gold (Au), platinum (Pt), a nitride of a metallic material (for example, titanium nitride), and the like.

### (Hole-Injecting Layer)

The hole-injecting layer is a layer containing a substance having high hole-injecting property. As such a substance having high hole-injecting property, molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, an aromatic amine compound, or a polymer compound (oligomers, dendrimers, polymers, etc.) can be used.

### (Hole-Transporting Layer)

The hole-transporting layer is a layer containing a substance having high hole-transporting property. For the hole-transporting layer, an aromatic amine compound, a carbazole derivative, an anthracene derivative, and the like can be used. A polymer compound such as poly(N-vinylcarbazole) (abbreviation: PVK) and poly(4-vinyltriphenylamine) (abbreviation: PVTPA) can also be used. However, a substance other than the above-described substances may be used as long as the substance has higher hole-transporting property in comparison with an electron-transporting property. It should be noted that the layer containing the material having high hole-transporting properties may be formed into not only a monolayer, but also a layer in which two or more layers formed of the above-described materials are stacked.

### (Guest Material for Emitting Layer)

The emitting layer is a layer containing a substance having a high emitting property, and various materials can be used for forming it. For example, as the substance having a high emitting property, a fluorescent compound which emits fluorescence or a phosphorescent compound which emits phosphorescence can be used. The fluorescent compound is a compound which can emit from a singlet excited state, and the phosphorescent compound is a compound which can emit from a triplet excited state.

As a blue fluorescent emitting material which can be used for an emitting layer, pyrene derivatives, styrylamine derivatives, chrysene derivatives, fluoranthene derivatives, fluorene derivatives, diamine derivatives, triarylamine derivatives, and the like can be used. As a green fluorescent emitting material which can be used for an emitting layer, aromatic amine derivatives and the like can be used. Asa red fluorescent emitting material which can be used for an emitting layer, tetracene derivatives, diamine derivatives and the like can be used.

As a blue phosphorescent emitting material which can be used for an emitting layer, metal complexes such as iridium complexes, osmium complexes, platinum complexes and the like are used. As a green phosphorescent emitting material which can be used for an emitting layer, iridium complexes and the like are used. As a red phosphorescent emitting material which can be used for an emitting layer, metal complexes such as iridium complexes, platinum complexes, terbium complexes, europium complexes and the like are used.

### (Host Material for Emitting Layer)

The emitting layer may have a constitution in which the substance having a high emitting property (guest material) is dispersed in another substance (host material). As a substance for dispersing the substance having a high emitting property, a variety of substances can be used, and it is preferable to use a substance having a higher lowest unoccupied orbital level (LUMO level) and a lower highest occupied orbital level (HOMO level) than the substance having a high emitting property.

As a substance for dispersing the substance having a high emitting property (host material), 1) metal complexes such as aluminum complexes, beryllium complexes, zinc complexes, and the like; 2) heterocyclic compounds such as oxadiazole derivatives, benzimidazole derivatives, phenanthroline derivatives, and the like; 3) fused aromatic compounds such as carbazole derivatives, anthracene derivatives, phenanthrene derivatives, pyrene derivatives, chrysene derivatives, or the like; and 3) aromatic amine compounds such as triarylamine derivatives, aromatic amine derivatives, and the like are used.

### (Electron-Transporting Layer)

An electron-transporting layer is a layer that contains a substance having a high electron-transporting property. For the electron-transporting layer, 1) metal complexes such as aluminum complexes, beryllium complexes, zinc complexes, and the like; 2) heteroaromatic complexes such as imidazole derivatives, benzimidazole derivatives, azine derivatives, carbazole derivatives, phenanthroline derivatives, and the like; and 3) polymer compounds can be used.

### (Electron-Injecting Layer)

An electron-injecting layer is a layer which contains a substance having a high electron-injecting property. For the electron-injecting layer, metal complex compounds such as lithium (Li), ytterbium (Yb), lithium fluoride (LiF), cesium fluoride (CsF), calcium fluoride (CaF₂), 8-hydroxyquinolinolato-lithium (Liq); alkali metals such as lithium oxide (LiOₓ); alkaline earth metals; and a compound thereof can be used.

### (Intermediate Layer)

In a tandem-type organic EL device, an intermediate layer is provided.

### (Cathode)

For the cathode, metals, alloys, electrically conductive compounds, mixtures thereof, and the like, which have a small work function (specifically, 3.8 eV or less) are preferably used. Specific examples of such a cathode material include elements belonging to Group 1 or Group 2 of the Periodic Table of the Elements, i.e., alkali metals such as lithium (Li) and cesium (Cs), alkaline earth metals such as magnesium (Mg), calcium (Ca) and strontium (Sr), and alloys containing these metals (e.g., MgAg and AlLi); and rare earth metals such as europium (Eu) and ytterbium (Yb), and alloys containing these metals.

In the organic EL device of the first aspect, the methods for forming the respective layers are not particularly limited. A conventionally-known method for forming each layer according to a vacuum deposition process, a spin coating process or the like can be used. Each layer such as an emitting layer can be formed by a known method such as a vacuum deposition process, a molecular beam deposition process (MBE process), or an application process such as a dipping process, a spin coating process, a casting process, a bar coating process and a roll coating process, which uses a solution prepared by dissolving the material in a solvent.

In the organic EL device of the first aspect of the invention, the thickness of each layer is not particularly limited, but is generally preferable that the thickness be in the range of several nm to 1 μm in order to suppress defects such as pinholes, to suppress applied voltages to be low, and to improve luminous efficiency.

### [Electronic Appliance]

The electronic appliance which is the fifth aspect of the invention is characterized in including the organic electroluminescence device of the first to fifth aspects described above.

Specific examples of the electronic appliance include a display component such as an organic EL panel module, and the like; a display device such as a television, a cellular phone, a personal computer, and the like; and an emitting device such as a light, a vehicular lamp, and the like.

### EXAMPLES

Next, the invention will be explained in more detail referring to the following Examples and Comparative Examples, but the invention should not be construed as limiting the scope of the invention by these Examples.

Compounds used in Examples 1 to 7 and Comparative Examples 1 to 2 are as follows.

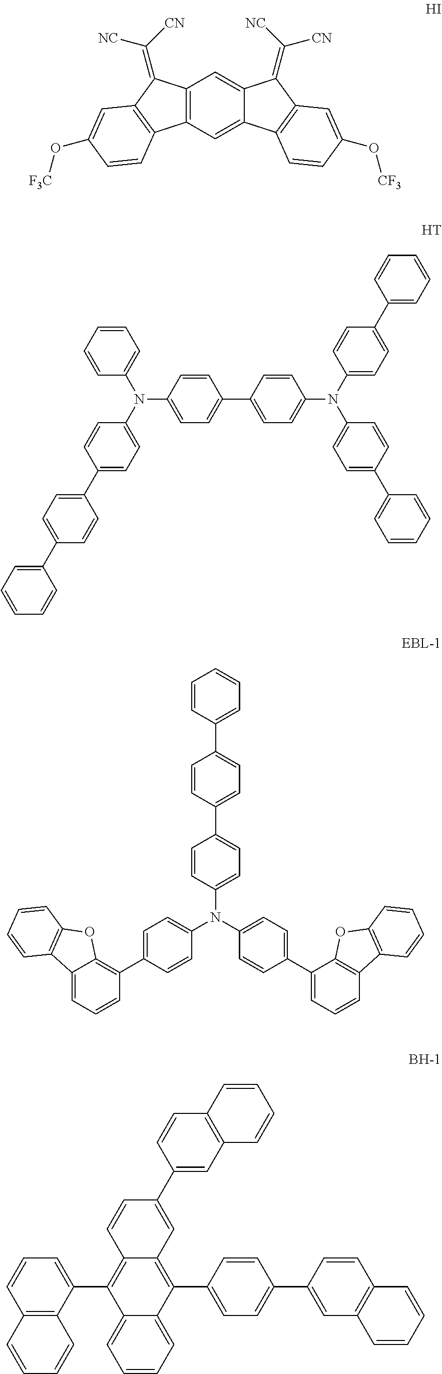

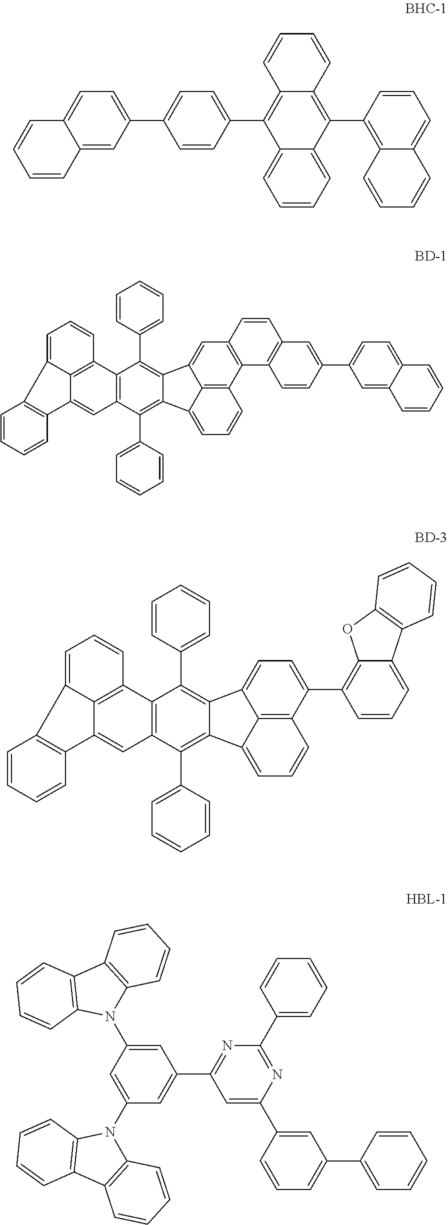

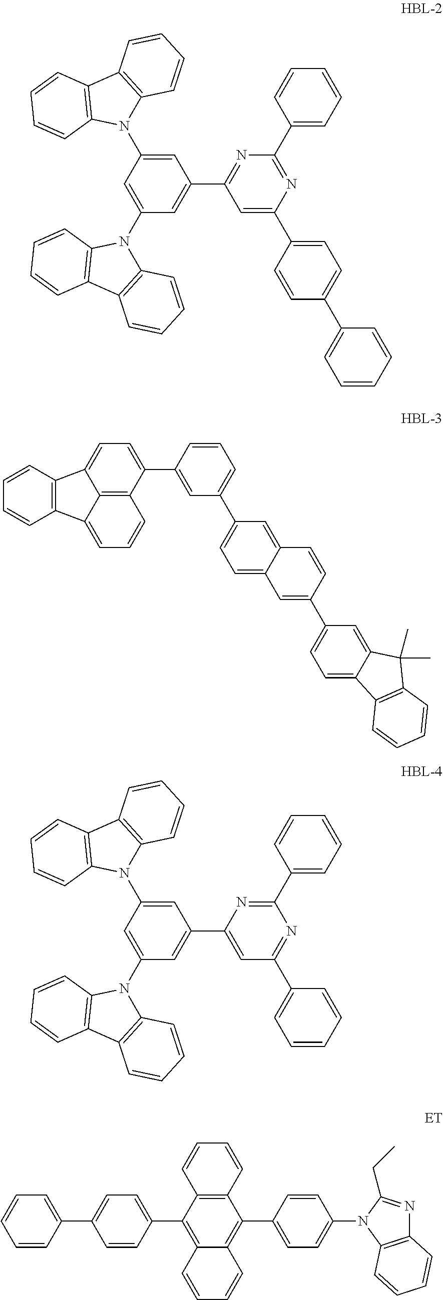

### Example 1

### (Fabrication of Organic EL Device)

A 25 mm×75 mm×1.1 mm-thick glass substrate with an ITO transparent electrode (anode) (manufactured by GEOMATEC Co., Ltd.) was subjected to ultrasonic cleaning in isopropyl alcohol for 5 minutes, and then subjected to UV-ozone cleaning for 30 minutes. The thickness of the ITO film was 130 nm.

The glass substrate with the transparent electrode after being cleaned was mounted onto a substrate holder in a vacuum vapor deposition apparatus. First, a compound HI was deposited on a surface on the side on which the transparent electrode was formed so as to cover the transparent electrode to form an HI film having a thickness of 5 nm. This HI film functions as a hole-injecting layer.

Subsequent to the formation of the HI film, a compound HT was deposited thereon to form an HT film having a thickness of 80 nm on the HI film. The HT film functions as a hole-transporting layer (first hole-transporting layer).

Subsequent to the formation of the HT film, a compound EBL-1 was deposited thereon to form an EBL-1 film having a thickness of 10 nm on the HT film. The EBL-1 film functions as an electron-blocking layer (second hole-transporting layer).

A compound BH-1 (host material) and a compound BD-1 (dopant material) were co-deposited on the EBL-1 film such that the proportion of the compound BD-1 became 2 mass %, and a BH-1:BD-1 film having a thickness of 25 nm was formed. This BH-1:BD-1 film functions as an emitting layer.

A compound ET was deposited on the emitting layer to form an ET film having a thickness of 15 nm. The ET film functions as an electron-transporting layer. LiF was deposited on the ET film to forma LiF film having a thickness of 1 nm. Metal Al was deposited on the LiF film to form a metal cathode having a thickness of 80 nm to obtain an organic EL device.

The layer configuration of the obtained organic EL device is as follows:

ITO(130)/HI(5)/HT(80)/EBL-1(10)/BH-1:BD-1(25: 2 mass %)/ET(15)/LiF(1)/Al(80)

Numerical values in parentheses indicate film thickness (unit: nm).

### (Evaluation of Organic EL Device)

Voltage was applied to the organic EL device to be 50 mA/cm² in current density, and the lifetime LT95 (hr), that means the time until the luminance decreases 95% of the initial luminance (LT95@50 mA/cm²) was measured. The results are shown in Table 1.

### Example 2

### (Fabrication of Organic EL Device)

A25 mm×75 mm×1.1 mm-thick glass substrate with an ITO transparent electrode (anode) (manufactured by GEOMATEC Co., Ltd.) was subjected to ultrasonic cleaning in isopropyl alcohol for 5 minutes, and then subjected to UV-ozone cleaning for 30 minutes. The thickness of the ITO film was 130 nm.

The glass substrate with the transparent electrode after being cleaned was mounted onto a substrate holder in a vacuum vapor deposition apparatus. First, a compound HI was deposited on a surface on the side on which the transparent electrode was formed so as to cover the transparent electrode to form an HI film having a thickness of 5 nm. This HI film functions as a hole-injecting layer.

Subsequent to the formation of the HI film, a compound HT was deposited thereon to form an HT film having a thickness of 80 nm on the HI film. The HT film functions as a hole-transporting layer (first hole-transporting layer).

Subsequent to the formation of the HT film, a compound EBL-1 was deposited thereon to form an EBL-1 film having a thickness of 10 nm on the HT film. The EBL-1 film functions as an electron-blocking layer (second hole-transporting layer).

A compound BH-1 (host material) and a compound BD-1 (dopant material) were co-deposited on the EBL-1 film such that the proportion of the compound BD-1 became 2 mass %, and a BH-1:BD-1 film having a thickness of 25 nm was formed. This BH-1:BD-1 film functions as an emitting layer.

A compound HBL-1 was deposited on the emitting layer to form an HBL-1 film having a thickness of 10 nm. This HBL-1 film functions as a hole-blocking layer (first electron-transporting layer). Subsequent to the formation of the HBL-1 film, a compound ET was deposited thereon to form an ET film having a thickness of 15 nm on the HBL-1 film. The ET film functions as an electron-transporting layer (second electron-transporting layer). LiF was deposited on the ET film to form a LiF film having a thickness of 1 nm. Metal Al was deposited on the LiF film to form a metal cathode having a thickness of 80 nm to obtain an organic EL device.

The layer configuration of the obtained organic EL device is as follows:

ITO(130)/HI(5)/HT(80)/EBL-1(10)/BH-1:BD-1(25:2 mass %)/HBL-1(10)/ET(15)/LiF(1)/Al(80)

Numerical values in parentheses indicate film thickness (unit: nm).

### Example 3 and Comparative Example 1

The organic EL devices were fabricated and evaluated in the same manner as in Example 2 except that the compounds shown in Table 1 were used as the host materials and the dopant materials of the emitting layer, and the materials for the hole-blocking layer. The results are shown in Table 1.

TABLE 1

| | | | Hole-blocking | |
|---|---|---|---|---|
| | Host | Dopant | layer | |
| | material | material | material | LT95 [h] |
| Example 1 | Compound | Compound | — | 354 |
| | BH-1 | BD-1 | | |
| Example 2 | Compound | Compound | Compound | 399 |
| | BH-1 | BD-1 | HBL-1 | |
| Example 3 | Compound | Compound | Compound | 399 |
| | BH-1 | BD-1 | HBL-4 | |
| Comp. Ex. 1 | Compound | Compound | Compound | 90 |
| | BHC-1 | BD-1 | HBL-1 | |

From the results in Table 1, it can be seen that the device lifetime of Example 1 using a tri-substituted anthracene compound (BH-1) is greatly improved as compared with Comparative Example 1 using a di-substituted anthracene compound (BHC-1).

Further, it can be seen that the use of the compound HBL-1 or HBL-4 as the hole-blocking layer in Examples 2 and 3 further improves the device lifetime compared to Example 1 in which no hole-blocking layer is provided. On the other hand, in Comparative Example 1, even if a hole-blocking layer using the same compound HBL-1 as in Example 1 is provided, it is understood that the device lifetime is very poor.

### Example 4

The organic EL device was fabricated and evaluated in the same manner as in Example 1 except that the host material and the dopant material shown in the following Table 2 were used. The results are shown in Table 2.

The layer configuration of the obtained organic EL device is as follows:

ITO(130)/HI(5)/HT(80)/EBL-1(10)/BH-1:BD-3(25:2 mass %)/ET(15)/LiF(1)/Al(80)

Numerical values in parentheses indicate film thickness (unit: nm).

### Examples 5 to 7 and Comparative Example 2

The organic EL devices were fabricated and evaluated in the same manner as in Example 2 except that the host material, the dopant material, and the material for the hole-blocking layer shown in the following Table 2 were used. The results are shown in Table 2.

The layer configuration of the obtained organic EL device is as follows:

ITO(130)/HI(5)/HT(80)/EBL-1(10)/BH-1:BD-3(25:2 mass %)/HBL-1 to HBL-3(10)/ET(15)/LiF(1)/Al(80)

Numerical values in parentheses indicate film thickness (unit: nm).

TABLE 2

| | | | Hole-blocking | |
|---|---|---|---|---|
| | Host | Dopant | layer | |
| | material | material | material | LT95 [h] |
| Example 4 | Compound | Compound | — | 222 |
| | BH-1 | BD-3 | | |
| Example 5 | Compound | Compound | Compound | 305 |
| | BH-1 | BD-3 | HBL-1 | |
| Example 6 | Compound | Compound | Compound | 289 |
| | BH-1 | BD-3 | HBL-2 | |
| Example 7 | Compound | Compound | Compound | 250 |
| | BH-1 | BD-3 | HBL-3 | |
| Comp. Ex. 2 | Compound | Compound | Compound | 145 |
| | BHC-1 | BD-3 | HBL-1 | |

From the results in Table 2, it can be seen that the device lifetime of Examples 4 to 7 using a tri-substituted anthracene compound (BH-1) is greatly improved as compared with Comparative Example 2 using a di-substituted anthracene compound BHC-1.

Further, it can be seen that the use of the compound HBL-1 or HBL-3 as the hole-blocking layer in Examples 5 to 7 further improves the device lifetime compared to Example 4 in which no hole-blocking layer is provided. On the other hand, in Comparative Example 2, even though a hole-blocking layer using the same compound HBL-1 as in Example 5 was provided, it is understood that the device lifetime is very inferior.

### <Compound>

The compounds represented by the formula (1) used for fabricating the organic EL device of Example 8 and subsequent Examples are as follows. The following 3BH-1 is the same compound as BH-1 used in Example 1 to 7.

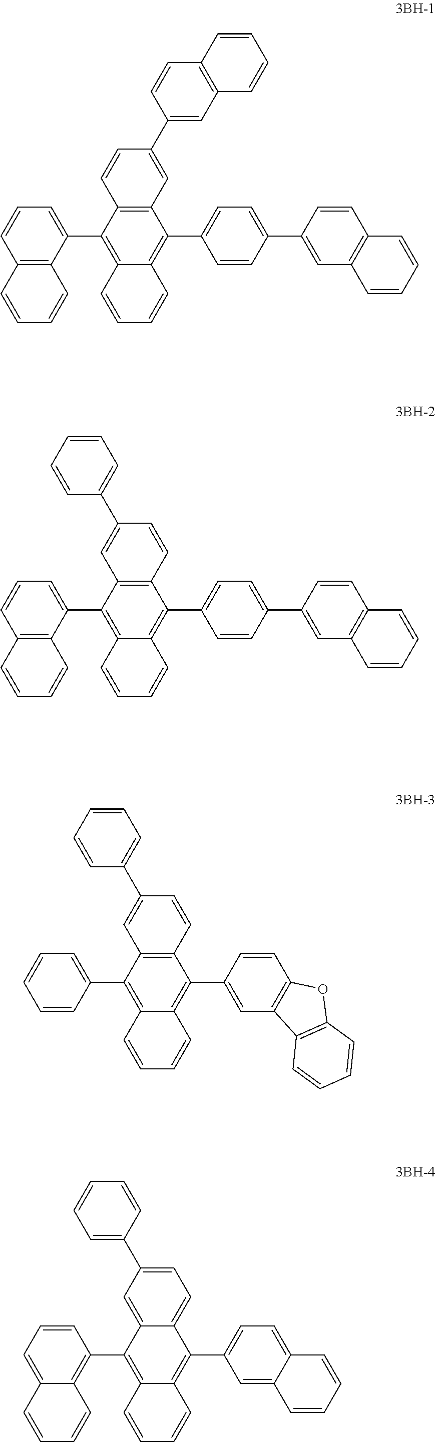

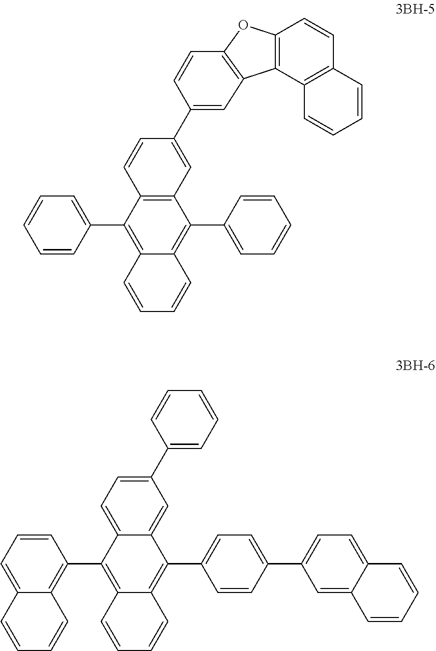

The compounds represented by the formula (11) used for fabricating the organic EL device of Example 8 and subsequent Examples are as follows:

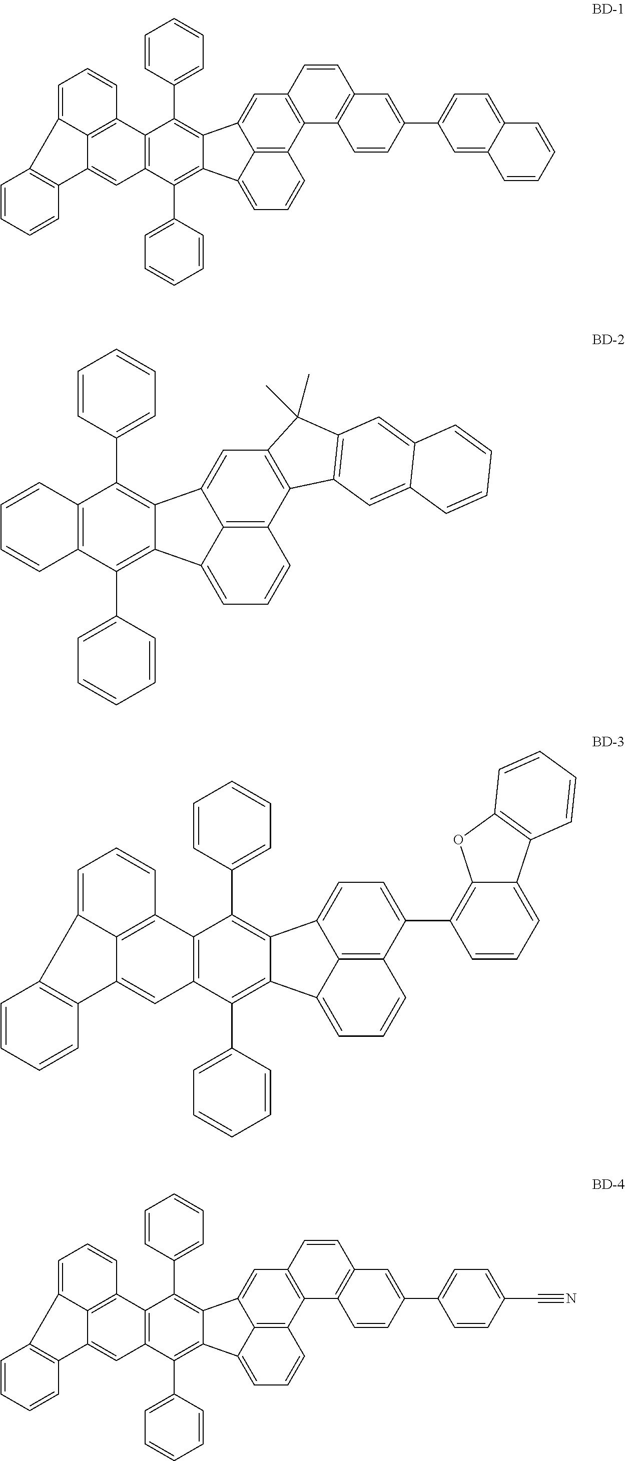

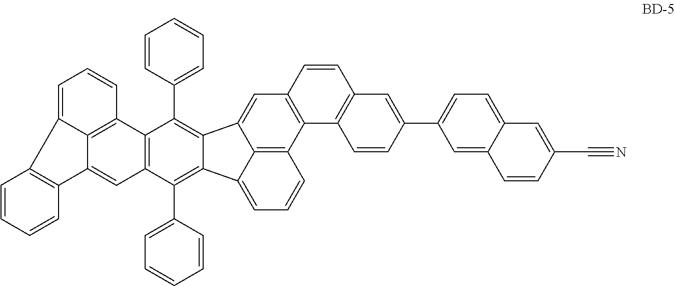 [SMILES: N#Cc1ccc2cc(-c3ccc4c(ccc5cc6c7c(-c8ccccc8)c8c(cc9c%10ccccc%10c%10cccc8c%109)c(-c8ccccc8)c7c7cccc(c54)c76)c3)ccc2c1]

The compounds represented by the formula (A-1) used for fabricating the organic EL device of Example 8 and subsequent Examples are as follows:

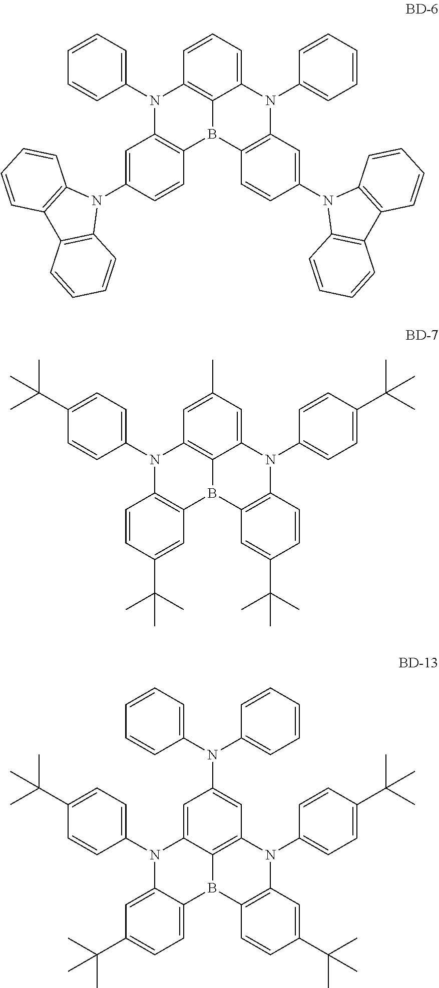

The compounds represented by the formula (A-2) used for fabricating the organic EL device of Example 8 and subsequent Examples are as follows:

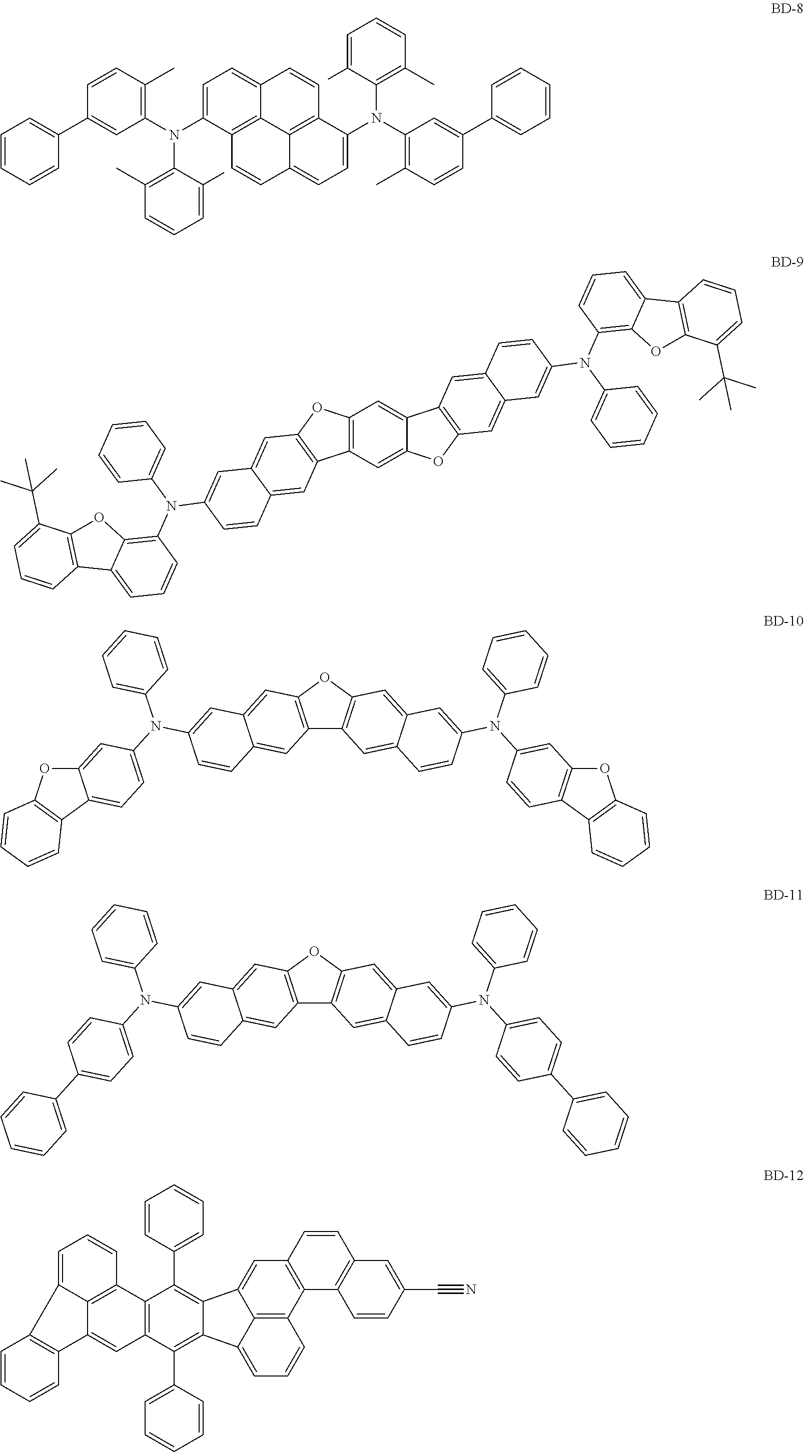

The compounds used as the host material in fabricating the organic EL device of Comparative Example 3 and subsequent Comparative Examples areas follows. The following Ref. 2BH-1 is the same compound as BHC-1 used in Comparative Examples 1 and 2.

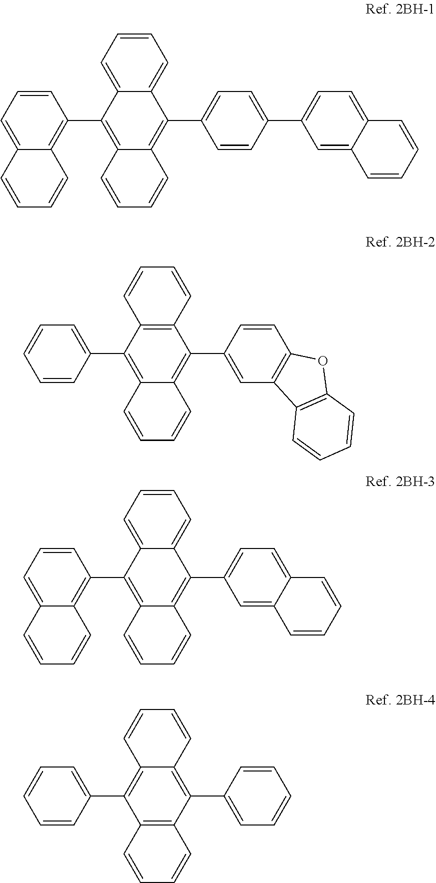

The compound used as the dopant material in fabricating the organic EL device of Comparative Example 3 and subsequent Comparative Examples is as follows:

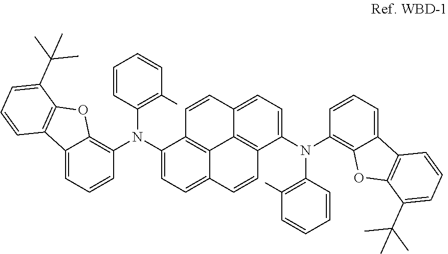 [SMILES: Cc1ccccc1N(c1ccc2ccc3c(N(c4ccccc4C)c4cccc5c4oc4c(C(C)(C)C)cccc45)ccc4ccc1c2c43)c1cccc2c1oc1c(C(C)(C)C)cccc12]

The structure of the other compounds used for fabricating the organic EL device of Example 8 and subsequent Examples, and Comparative Example 3 and subsequent Comparative Examples are as follows:

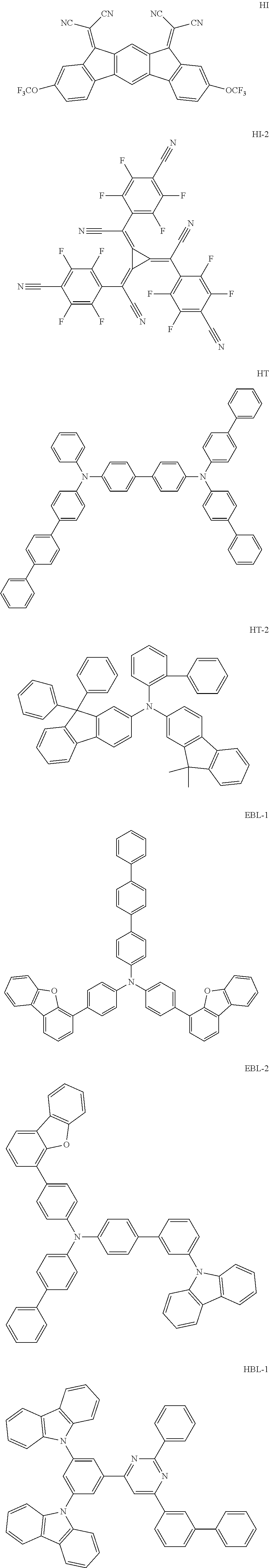

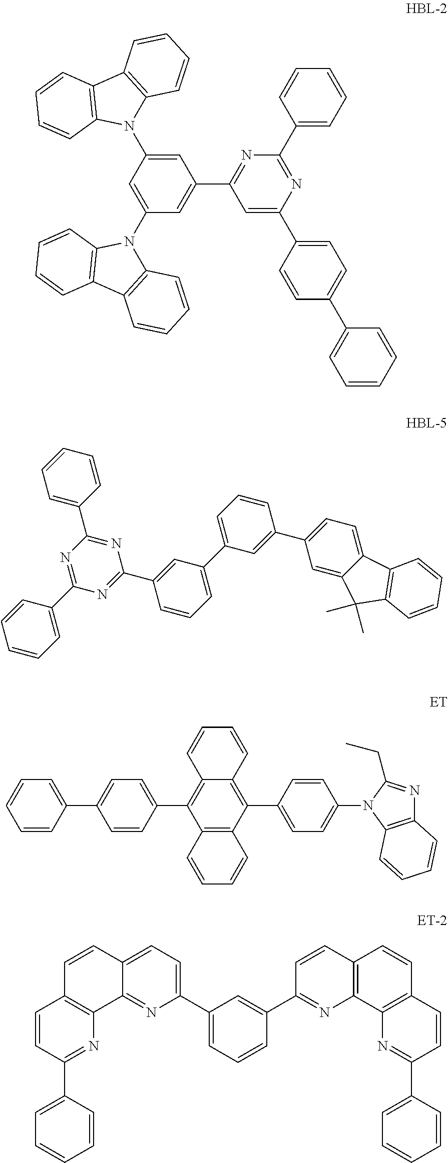 [SMILES: c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; CC1(C)c2ccccc2-c2ccc(-c3cccc(-c4cccc(-c5nc(-c6ccccc6)nc(-c6ccccc6)n5)c4)c3)cc21 ; CCc1nc2ccccc2n1-c1ccc(-c2c3ccccc3c(-c3ccc(-c4ccccc4)cc3)c3ccccc23)cc1 ; c1ccc(-c2ccc3ccc4ccc(-c5cccc(-c6ccc7ccc8ccc(-c9ccccc9)nc8c7n6)c5)nc4c3n2)cc1]

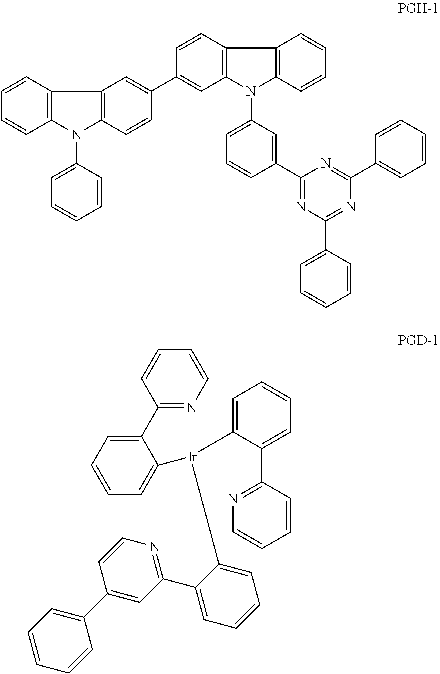

### <Fabrication of Organic EL Device>

The organic EL devices were fabricated and evaluated as follows.

### Example 8

A 25 mm×75 mm×1.1 mm-thick glass substrate with an ITO transparent electrode (anode) (manufactured by GEOMATEC Co., Ltd.) was subjected to ultrasonic cleaning in isopropyl alcohol for 5 minutes, and then subjected to UV-ozone cleaning for 30 minutes. The thickness of the ITO film was 130 nm.

The glass substrate with the transparent electrode after being cleaned was mounted onto a substrate holder in a vacuum vapor deposition apparatus. First, a compound HI was deposited on a surface on the side on which the transparent electrode was formed so as to cover the transparent electrode to form an HI film having a thickness of 5 nm. This HI film functions as a hole-injecting layer.

Subsequent to the formation of the HI film, a compound HT was deposited thereon to form an HT film having a thickness of 80 nm on the HI film. The HT film functions as a hole-transporting layer (first hole-transporting layer).

Subsequent to the formation of the HT film, a compound EBL-1 was deposited thereon to form an EBL-1 film having a thickness of 10 nm on the HT film. The EBL-1 film functions as an electron-blocking layer (second hole-transporting layer).

A compound 3BH-2 (host material) and a compound BD-1 (dopant material) were co-deposited on the EBL-1 film such that the proportion of the compound BD-1 became 2 mass %, and a 3BH-2:BD-1 film having a thickness of 25 nm was formed. This 3BH-2:BD-1 film functions as an emitting layer.

A compound HBL-1 was deposited on the emitting layer to form an HBL-1 film having a thickness of 10 nm. This HBL-1 film functions as a hole-blocking layer (first electron-transporting layer). Subsequent to the formation of the HBL-1 film, a compound ET was deposited thereon to form an ET film having a thickness of 15 nm on the HBL-1 film. The ET film functions as an electron-transporting layer (second electron-transporting layer). LiF was deposited on the ET film to form a LiF film having a thickness of 1 nm. Metal Al was deposited on the LiF film to form a metal cathode having a thickness of 80 nm to obtain an organic EL device.

The layer configuration of the obtained organic EL device is as follows:

ITO(130)/HI(5)/HT(80)/EBL-1(10)/3BH-2:BD-1(25:2 MASS %)/HBL-1(10)/ET(15)/LIF(1)/Al(80)

Numerical values in parentheses indicate film thickness (unit: nm).

### Examples 9 to 14

The organic EL device of Examples 9 to 14 were fabricated in the same manner as in Example 8 except that the dopant material of the emitting layer of Example 8 was replaced with the dopant material described in Table 3.

### Comparative Example 3

The organic EL device of Comparative Example 3 was fabricated in the same manner as in Example 8 except that the dopant material of the emitting layer of Example 8 was replaced with the dopant material described in Table 3, and the dopant material was co-deposited to be in the proportion of 4 mass %.

### <Evaluation of Organic EL Device>

The organic EL devices fabricated in Examples 8 to 14 and Comparative Example 3 were evaluated as follows. The results are shown in Table 3. The Stokes shift values of the dopant materials used in Examples 8 to 14 and that of the dopant material used in Comparative Example 3 are also shown in Table 3.

External Quantum Efficiency EQE (%)

Voltage was applied to the organic EL device to be 10 mA/cm² in current density, thereby measuring an EL emission spectrum by using Spectroradiometer CS-1000 (manufactured by Konica Minolta, Inc.). External quantum efficiency (EQE %) was calculated from the obtained spectral radiance spectrum.

Stokes Shift (SS) (Nm) of the Dopant Materials

The dopant material was dissolved in toluene at a concentration of 10⁻⁵ mol/L or more and 10⁻⁴ mol/L or less to prepare a measurement sample. A measurement sample in a quartz cell was irradiated with continuous light in the UV-visible region at room temperature (300 K), and the absorption spectrum (vertical axis: absorbance, horizontal axis: wavelength) was measured. The absorption spectrum was measured using a spectrophotometer U-3900/3900H type manufactured by Hitachi High-Tech Science Corporation. The dopant material was dissolved in toluene at a concentration of 10⁻⁶ mol/L or more and 10⁻⁵ mol/L or less to prepare a measurement sample. A measurement sample in a quartz cell was irradiated with excitation light at room temperature (300 K), and the fluorescence spectrum (vertical axis: fluorescence intensity, horizontal axis: wavelength) was measured. The fluorescence spectrum was measured using a fluorescent spectrophotometer F-7000 type manufactured by Hitachi High-Tech Science Corporation.

From these absorption spectra and fluorescence spectra, the difference between the absorption maximum wavelength and the fluorescence maximum wavelength was calculated to obtain a Stokes shift (SS).

Emission Peak Wavelength a (Nm)

Voltage was applied to the organic EL device to be 10 mA/cm² in current density, thereby measuring an EL emission spectrum by using Spectroradiometer CS-1000 (manufactured by Konica Minolta, Inc.). The emission peak wavelength was obtained from the obtained spectral radiance spectrum.

TABLE 3

| | Host | Dopant | EQE | SS | λ |
|---|---|---|---|---|---|
| | material | material | [%] | [nm] | [nm] |
| Example 8 | 3BH-2 | BD-1 | 8.6 | 9 | 464 |
| Example 9 | 3BH-2 | BD-2 | 6.8 | 8 | 465 |
| Example 10 | 3BH-2 | BD-3 | 8.7 | 11 | 461 |
| Example 11 | 3BH-2 | BD-4 | 7.1 | 8 | 464 |
| Example 12 | 3BH-2 | BD-5 | 7.3 | 8 | 464 |
| Example 13 | 3BH-2 | BD-6 | 7.3 | 14 | 464 |
| Example 14 | 3BH-2 | BD-7 | 6.7 | 14 | 462 |
| Comp. Ex. 3 | 3BH-2 | Ref. WBD-1 | 6.4 | 25 | 457 |

From the results in Table 3, it can be seen that Examples 8 to 14 in which the tri-substituted anthracene compound 3BH-2 represented by the formula (1) was used in combination with compounds BD-1 to BD-7, which are a compound A that emits blue light with a small Stokes shift (SS), have a high device efficiency (external quantum efficiency), compared to Comparative Example 3 in which the tri-substituted anthracene compound 3BH-2 is used in combination with a compound Ref. WBD-1 which emits blue light with a large Stokes shift (SS).

### <Fabrication and Evaluation of Organic EL Device>

The organic EL devices of Examples 15 to 20 and Comparative Example 4 were fabricated and evaluated as follows.

### Examples 15, 16 and 18

The organic EL devices of Examples 15, 16 and 18 were fabricated in the same manner as in Example 8 except that the materials of the emittin g layer of Example 8 were replaced with the host material and the dopant material described in Table 4.

### Examples 17, 19 and 20 and Comparative Example 4

The organic EL devices of Examples 17, 19 and 20 and Comparative Example 4 were fabricated in the same manner as in Example 8 except that the materials of the emitting layer of Example 8 were replaced with the host material and the dopant material described in Table 4, and the dopant material was co-deposited to be in the proportion of 4 mass %.

TABLE 4

| | Host | Dopant | EQE | SS | λ |
|---|---|---|---|---|---|
| | material | material | [%] | [nm] | [nm] |
| Example 15 | 3BH-1 | BD-3 | 7.4 | 11 | 462 |
| Example 16 | 3BH-1 | BD-6 | 6.2 | 14 | 465 |
| Example 17 | 3BH-1 | BD-8 | 5.4 | 14 | 461 |
| Example 18 | 3BH-1 | BD-9 | 6.5 | 15 | 460 |
| Example 19 | 3BH-1 | BD-10 | 5.5 | 20 | 460 |
| Example 20 | 3BH-1 | BD-11 | 5.5 | 20 | 461 |
| Comp. Ex. 4 | 3BH-1 | Ref. WBD-1 | 5.1 | 25 | 458 |

From the results in Table 4, it can be seen that Examples 15 to 20 in which the tri-substituted anthracene compound 3BH-1 represented by the formula (1) is used in combination with compounds BD-3, BD-6 and BD-8 to BD-11, which are a compound A that emits blue light with a small Stokes shift (SS), has a high device efficiency (external quantum efficiency), compared to Comparative Example 4 in which the tri-substituted anthracene compound 3BH-1 is used in combination with a compound Ref. WBD-1 which emits blue light with a large Stokes shift (SS).

From the results of the Tables 3 and 4, it can be seen that the tri-substituted anthracene compounds 3BH-2 and 3BH-1 represented by the formula (1) can be applied to an organic EL device of blue fluorescence because energy transfer is more likely to occur and device efficiency (external quantum efficiency) is improved when they are used in combination with a compound A that emits blue light with a small Stokes shift (SS) than the case where they are used in combination with a compound that emits blue light with a large Stokes shift (SS).

### <Fabrication of Organic EL Device>

The organic EL devices were fabricated as follows.

### Examples 21 and 22, and Comparative Examples 5 and 6

The organic EL devices of Examples 21 and 22, and Comparative Examples 5 and 6 were fabricated in the same manner as in Example 8 except that the the host material and the dopant material of the emitting layer in Example 8 were replaced with the host material and the dopant material described in Tables 5 and 6.

### <Evaluation of Organic EL Device>

The organic EL devices fabricated in Examples 21 and 22, and Comparative Examples 5 and 6 were evaluated as follows. The results are shown in Table 5 and Table 6.

Driving Voltage (V) Initial characteristics of the obtained organic EL devices were measured by driving at a constant current of 10 mA/cm² of DC (direct current) at room temperature.

Device Lifetime (LT90)

Voltage was applied to the organic EL device to be 50 mA/cm² in current density, and the time until the luminance decreases 90% of the initial luminance are measured.

The Stokes shift (SS) and the emission peak wavelength A were measured by the method described in Example 4.

TABLE 5

| | Host | Dopant | Driving | LT90 | SS | λ |
|---|---|---|---|---|---|---|
| | material | material | voltage [V] | [h] | [nm] | [nm] |
| Example 21 | 3BH-1 | BD-6 | 3.2 | 81 | 14 | 465 |
| Comp. Ex. 5 | Ref. 2BH-1 | BD-6 | 3.7 | 71 | 14 | 464 |

TABLE 6

| | Host | Dopant | Driving | LT90 | SS | λ |
|---|---|---|---|---|---|---|
| | material | material | voltage [V] | [h] | [nm] | [nm] |
| Example 22 | 3BH-3 | BD-6 | 3.3 | 83 | 14 | 463 |
| Comp. Ex. 6 | Ref. 2BH-2 | BD-6 | 3.7 | 33 | 14 | 463 |

From the results in Tables 5 and 6, it can be seen that the combination of the tri-substituted anthracene compound 3BH-1 or 3BH-3 represented by the formula (1) and the compound BD-6, which is a compound A that emits blue light with a small Stokes shift (SS), results in a blue fluorescent device that can be driven at low voltage and has a long lifetime, compared to the combination of the bi-substituted compound Ref. 2BH-1 or Ref. 2BH-2 and the compound BD-6.

### <Fabrication of Organic EL Device>

The organic EL devices were fabricated and evaluated as follows.

### Examples 23 to 25, and Comparative Examples 7 and 8

The organic EL devices of Examples 23 to 25, and Comparative Examples 7 to 8 were fabricated in the same manner as in Example 8 except that the host material and the dopant material of the emitting layer in Example 8 were replaced with the host material and the dopant material described in Tables 7 or 8, and the dopant material was co-deposited to be in the proportion of 4 mass %; and evaluated in the same manner as in Example 21.

TABLE 7

| | Host | Dopant | Driving | LT90 | SS | λ |
|---|---|---|---|---|---|---|
| | material | material | voltage [V] | [h] | [nm] | [nm] |
| Example 23 | 3BH-2 | BD-8 | 3.4 | 210 | 14 | 459 |
| Example 24 | 3BH-1 | BD-8 | 3.3 | 360 | 14 | 460 |
| Comp. Ex. 7 | Ref. 2BH-1 | BD-8 | 3.7 | 144 | 14 | 458 |

TABLE 8

| | Host | Dopant | Driving | LT90 | SS | λ |
|---|---|---|---|---|---|---|
| | material | material | voltage [V] | [h] | [nm] | [nm] |
| Example 25 | 3BH-4 | BD-8 | 3.4 | 144 | 14 | 459 |
| Comp. Ex 8 | Ref. 2BH-3 | BD-8 | 3.8 | 108 | 14 | 458 |

From the results in Tables 7 and 8, it can be seen that the combination of the tri-substituted anthracene compound 3B-1, 3BH-2 or 3BH-4 represented by the formula (1) and the compound BD-8, which is a compound A that emits blue light with a small Stokes shift (SS), results in a blue fluorescent device that can be driven at low voltage and has a long lifetime, compared to the combination of the bi-substituted compound Ref. 2BH-1 or Ref. 2BH-3 and the compound BD-8.

### <Fabrication of Organic EL Device>

The organic EL devices were fabricated and evaluated as follows.

### Examples 26 and 27, and Comparative Example 9

The organic EL devices of Examples 26 and 27, and Comparative Example 9 were fabricated in the same manner as in Example 8 except that the materials of the emitting layer in Example 8 were replaced with the dopant material described in Table 9, and the dopant material was co-deposited to be in the proportion of 4 mass %; and evaluated in the same manner as in Example 21.

TABLE 9

| | Host | Dopant | Driving | LT90 | SS | λ |
|---|---|---|---|---|---|---|
| | material | material | voltage [V] | [h] | [nm] | [nm] |
| Example 26 | 3BH-2 | BD-11 | 3.3 | 109 | 20 | 459 |
| Example 27 | 3BH-1 | BD-11 | 3.2 | 190 | 20 | 461 |
| Comp. Ex. 9 | Ref. 2BH-1 | BD-11 | 3.6 | 90 | 20 | 458 |

From the results in Table 9, it can be seen that the combination of the tri-substituted anthracene compound 3BH-1 or 3BH-2 represented by the formula (1) and the compound BD-11, which is a compound A that emits blue light with a small Stokes shift (SS), results in a blue fluorescent device that can be driven at low voltage and has a long lifetime, compared to the combination of the bi-substituted compound Ref. 2BH-1 and the compound BD-11.

### Examples 28 and 30

The organic EL devices were fabricated in the same manner as in Example 1 except that the host material and the dopant material shown in the following Table 10 were used and evaluated in the same manner as in Example 1. The results are shown in Table 10.

### Examples 29 and 31, and Comparative Example 10

The organic EL devices were fabricated in the same manner as in Example 2 except that the host material, the dopant material, and the material for the hole-blocking layer shown in the following Table 10 were used, and evaluated in the same manner as in Example 1. The results are shown in Table 10.

TABLE 9

| | Host | Dopant | Driving | LT90 | SS | λ |
|---|---|---|---|---|---|---|
| | material | material | voltage [V] | [h] | [nm] | [nm] |
| Example 26 | 3BH-2 | BD-11 | 3.3 | 109 | 20 | 459 |
| Example 27 | 3BH-1 | BD-11 | 3.2 | 190 | 20 | 461 |
| Comp. Ex. 9 | Ref. 2BH-1 | BD-11 | 3.6 | 90 | 20 | 458 |

From the results in Table 10, it can be seen that the device lifetime of Examples 28 to 31 using the tri-substituted anthracene compound (3BH-1 or 3BH-2) is greatly improved as compared with Comparative Example 10 using the di-substituted anthracene compound Ref. 2BH-1.

Further, it can be seen that the use of the compound HBL-5 as the hole-blocking layer in Examples 29 and 31 further improves the device lifetime compared to Examples 28 and 30 in which no hole-blocking layer was provided. On the other hand, in Comparative Example 10, even though the hole-blocking layer using the same compound HBL-5 as in Examples 29 and 31 is provided, it is understood that the device lifetime is very inferior.

### Examples 32 and 34

The organic EL devices were fabricated in the same manner as in Example 1 except that the host material and the dopant material shown in the following Table 11 were used, and the lifetime (LT90) was evaluated in the same manner as in Example 21. The results are shown in Table 11.

### Examples 33 and 35, and Comparative Example 11

The organic EL devices were fabricated in the same manner as in Example 2 except that the host material, the dopant material and the material of the hole-blocking layer shown in the following Table 11 were used, and the lifetime (LT90) was evaluated in the same manner as in Example 21. The results are shown in Table 11.

TABLE 11

| | Host | Dopant | Hole-blocking | LT90 |
|---|---|---|---|---|
| | material | material | layer material | [h] |
| Example 32 | 3BH-2 | BD-5 | — | 266 |
| Example 33 | 3BH-2 | BD-5 | HBL-5 | 408 |
| Example 34 | 3BH-1 | BD-5 | — | 345 |
| Example 35 | 3BH-1 | BD-5 | HBL-5 | 414 |
| Comp. Ex. 11 | Ref. 2BH-1 | BD-5 | HBL-5 | 86 |

From the results in Table 11, it can be seen that the device lifetime of Examples 32 to 35 using the tri-substituted anthracene compound (3BH-1 and 3BH-2) was greatly improved as compared with Comparative Example 11 using the di-substituted anthracene compound 2BH-1.

Further, it can be seen that the use of the compound HBL-5 in the hole-blocking layer in Examples 33 and 35 further improves the device lifetime compared to Examples 32 and 34 in which no hole-blocking layer was provided. On the other hand, in Comparative Example 11, even though the hole-blocking layer using the same compound HBL-5 as in Examples 33 and 35 was provided, it is understood that the device lifetime is very inferior.

### Example 36 and Comparative Example 12

The organic EL devices were fabricated in the same manner as in Example 8 except that the host material and the dopant material shown in the following Table 12 were used, and evaluated in the same manner as in Example 21. The results are shown in Table 12.

TABLE 12

| | Host | Dopant | Driving | LT90 | SS | λ |
|---|---|---|---|---|---|---|
| | material | material | voltage [V] | [h] | [nm] | [nm] |
| Example 36 | 3BH-5 | BD-3 | 3.38 | 374 | 11 | 463 |
| Comp. Ex. 12 | Ref. 2BH-4 | BD-3 | 4.08 | 42 | 11 | 463 |

From the results in Table 12, it can be seen that the combination of the tri-substituted anthracene compound 3BH-5 represented by the formula (1) and the compound BD-3, which is a compound A that emits blue light with a small Stokes shift (SS), results in a blue fluorescent device that can be driven at low voltage and has a long lifetime, compared to the combination of the bi-substituted compound Ref. 2BH-4 and the compound BD-3.

### Example 37

### <Fabrication of Tandem-Type Organic EL Device>

A 25 mm×75 mm×1.1 mm-thick glass substrate with an ITO transparent electrode (anode) (manufactured by GEOMATEC Co., Ltd.) was subjected to ultrasonic cleaning in isopropyl alcohol for 5 minutes, and then subjected to UV-ozone cleaning for 1 minute. The thickness of the ITO film was 130 nm.

### Formation of First Emitting Unit

The glass substrate with the transparent electrode line after being cleaned was mounted onto a substrate holder in a vacuum vapor deposition apparatus, and a compound HT-2 and a compound HI-2 were co-deposited on a surface on the side on which the transparent electrode line was formed so as to cover the transparent electrode to form a hole-injecting layer having a thickness of 10 nm. The concentration of the compound HT-2 was 97 mass %, and the concentration of the compound HI-2 was 3 mass % in the hole-injecting layer.

Next, the compound HT-2 was vapor-deposited on the hole-injecting layer to form a first hole-transporting layer having a thickness of 70 nm.

Next, a compound EBL-2 was vapor-deposited on the first hole-transporting layer to form a second hole-transporting layer having a thickness of 10 nm.

Next, a compound 3BH-2 and a compound BD-7 were co-deposited on the second hole-transporting layer to form a blue fluorescent emitting layer as a first emitting layer having a thickness of 25 nm. The concentration of the compound 3BH-2 was 98 mass %, and the concentration of the compound BD-7 was 2 mass % in the blue fluorescence emitting layer.

Next, a compound HBL-2 was vapor-deposited on the blue fluorescent emitting layer to form an electron-transporting layer having a thickness of 10 nm.

Formation of First Charge-Generating Layer

Next, a compound ET-2 and lithium (Li) were co-deposited on the electron-transporting layer to forma first N layer having a thickness of 10 nm. The concentration of the compound ET-2 was 96 mass %, and the concentration of Li was 4 mass % in the first N layer.

Next, the compound HT-2 and the compound HI-2 were co-deposited on the first N layer to form a first Player having a thickness of 10 nm. The concentration of the compound HT-2 was 90 mass %, and the concentration of compound HI-2 was 10 mass % in the first P layer.

Formation of Second Emitting Unit

Next, the compound EBL-2 was vapor-deposited on the first P layer to form a first hole-transporting layer having a thickness of 10 nm.

Next, a compound PGH-1 and a compound PGD-1 were co-deposited on the first hole-transporting layer to form a yellow phosphorescent emitting layer as a second emitting layer having a thickness of 48 nm. The concentration of the compound PGH-1 was 80 mass %, and the concentration of compound PGD-1 was 20 mass % in the yellow phosphorescent emitting layer.

Next, a compound ET was vapor-deposited on the yellow phosphorescent emitting layer to form an electron-transporting layer having a thickness of 10 nm.

Formation of Second Charge-Generating Layer

Next, the compound ET-2 and lithium (Li) were co-deposited on the electron-transporting layer to form a second N layer having a thickness of 35 nm. The concentration of the compound ET-2 was 96 mass %, and the concentration of Li was 4 mass % in the second N layer.

Next, the compound HT-2 and a compound HI-2 were co-deposited on the second N layer to forma second Player having a thickness of 10 nm. The concentration of the compound HT-2 was 90 mass %, and the concentration of compound HI-2 was 10 mass % in the second P layer.

Formation of Third Emitting Unit

Next, the compound HT-2 was vapor-deposited on the second P layer to form a first hole-transporting layer having a thickness of 70 nm.

Next, the compound EBL-2 was vapor-deposited on the first hole-transporting layer to form a second hole-transporting layer having a thickness of 10 nm.

Next, the compound 3BH-2 and the compound BD-7 were co-deposited on the second hole-transporting layer to form a blue fluorescent emitting layer as a third emitting layer having a thickness of 25 nm. The concentration of the compound 3BH-2 was 98 mass %, and the concentration of the compound BD-7 was 2 mass % in the blue fluorescence emitting layer.

Next, the compound HBL-2 was vapor-deposited on the blue fluorescent emitting layer to form a first electron-transporting layer having a thickness of 10 nm.

Next, the compound ET was vapor-deposited on the first electron-transporting layer to form a second electron-transporting layer having a thickness of 10 nm.

Next, lithium fluoride (LiF) was vapor-deposited on the second electron-transporting layer to form an electron-injecting layer having a thickness of 1 nm.

Then, metal aluminum (AI) was vapor-deposited on the electron-injecting layer to form a metal Al cathode having a thickness of 80 nm.

As described above, a bottom-emission type organic EL device was fabricated.

The layer configuration of the obtained organic EL device is as follows:

ITO(130)/HT-2:HI-2(10, 97%:3%)/HT-2(70)/EBL-2(10)/3BH-2:BD-7(25, 98%:2%)/HBL-2(10)/ET-2:Li(10, 96%:4%)/HT-2:HI-2(10, 90%:10%)/EBL-2(10)/PGH-1:PGD-1(48, 80%:20%)/ET(10)/ET-2:Li(35, 96%:4%)/HT-2:HI-2(10, 90%:10%)/HT-2(70)/EBL-2(10)/3BH-2:BD-7(25, 98%:2%)/HBL-2(10)/ET(10)/LiF(1)/Al(80)

Numerical values in parentheses indicate film thickness (unit: nm).

Similarly, in the parentheses, numerical values expressed in percentage indicates that, for example, for HT-2: HI-2 (10, 97%:3%), the ratio (mass %) of a compound HT-2 and a compound HI-2 in the hole-injecting layer is HT-1:H-2=97 mass %: 3 mass %.

### <Evaluation of Organic EL Device>

Driving Voltage (V)

Initial characteristics of the obtained organic EL devices were measured by driving at a constant current of 10 mA/cm² of DC (direct current) at room temperature.

The Stokes shift (SS) and the emission peak wavelength A were measured by the method described in Example 8.

### Examples 38 to 40, and Comparative Examples 13 and 14

The organic EL devices were fabricated in the same manner as in Example 37 except that the host material and the dopant material shown in the following Table 13 were used and evaluated in the same manner as in Example 37.

TABLE 13

| | Host | Dopant | Driving | SS |
|---|---|---|---|---|
| | material | material | voltage [V] | [nm] |
| Example 37 | 3BH-2 | BD-7 | 11.2 | 14 |
| Example 38 | 3BH-2 | BD-13 | 11.2 | 14 |
| Example 39 | 3BH-6 | BD-7 | 11.2 | 14 |
| Example 40 | 3BH-6 | BD-13 | 11.1 | 14 |
| Comp. Ex. 13 | Ref. 2BH-1 | BD-7 | 12.1 | 14 |
| Comp. Ex. 14 | Ref. 2BH-1 | BD-13 | 12.0 | 14 |

The combination of the tri-substituted anthracene compound 3BH-2 or 3BH-6 represented by the formula (1) and the compound BD-7 or BD-13, which is a compound A that emits blue light with a small Stokes shift (SS), results in a blue fluorescent device that can be driven at low voltage, compared to the combination of the bi-substituted compound Ref. 2BH-1 and the compound BD-7 or BD-13.

The organic EL devices were fabricated and evaluated as follows.

### Examples 41 to 43 and Comparative Example 15

The organic EL devices of Examples 41 to 43, and Comparative Example 15 were fabricated and evaluated in the same manner as in Example 8 except that the materials of the emitting layer in Example 8 were replaced with the host material and the dopant material described in Table 14. The results are shown in Table 14.

TABLE 14

| | Host | Dopant | EQE | SS | λ |
|---|---|---|---|---|---|
| | material | material | [%] | [nm] | [nm] |
| Example 41 | 3BH-6 | BD-1 | 8.6 | 9 | 464 |
| Example 42 | 3BH-6 | BD-8 | 7.2 | 14 | 461 |
| Example 43 | 3BH-6 | BD-9 | 9.1 | 15 | 460 |
| Comp. Ex. 15 | 3BH-6 | ref. WBD-1 | 6.4 | 25 | 458 |

From the results in Table 14, it can be seen that Examples 41 to 43 in which the tri-substituted anthracene compound 3BH-6 represented by the formula (1) i was used in combination with compounds BD-1, BD-8 and BD-9, which are a compound A that emits blue light with a small Stokes shift (SS), have a high device efficiency (external quantum efficiency), compared to Comparative Example 15 in which the tri-substituted anthracene compound 3BH-6 was used in combination with the compound Ref. WBD-1 which emits blue light with a large Stokes shift (SS).

From the results of the Table 14, it can be seen that the tri-substituted anthracene compound 3BH-6 represented by the formula (1) can be applied to an organic EL device of blue fluorescence because energy transfer is more likely to occur and device efficiency (external quantum efficiency) is improved when they are combined with a compound A that emits blue light with a small Stokes shift (SS) than when they are combined with a compound that emits blue light with a large Stokes shift (SS).

### Examples 44 to 46, and Comparative Examples 16 to 18

The organic EL devices of Examples 44 to 46 and Comparative Examples 16 to 18 were fabricated in the same manner as in Example 8 except that the materials of the emitting layer in Example 8 were replaced with the host material and the dopant material described in Tables 15 to 17; and evaluated in the same manner as in Example 21. The results are shown in Tables 15 to 17.

TABLE 15

| | Host | Dopant | Driving | LT90 | SS | λ |
|---|---|---|---|---|---|---|
| | material | material | voltage [V] | [h] | [nm] | [nm] |
| Example 44 | 3BH-6 | BD-1 | 3.5 | 760 | 9 | 464 |
| Comp. Ex. 16 | Ref. 2BH-1 | BD-1 | 3.9 | 352 | 9 | 464 |

TABLE 16

| | Host | Dopant | Driving | LT90 | SS | λ |
|---|---|---|---|---|---|---|
| | material | material | voltage [V] | [h] | [nm] | [nm] |
| Example 45 | 3BH-6 | BD-8 | 3.4 | 210 | 14 | 461 |
| Comp. Ex. 17 | Ref. 2BH-1 | BD-8 | 3.7 | 144 | 14 | 461 |

TABLE 17

| | Host | Dopant | Driving | LT90 | SS | λ |
|---|---|---|---|---|---|---|
| | material | material | voltage [V] | [h] | [nm] | [nm] |
| Example 46 | 3BH-6 | BD-9 | 3.4 | 311 | 15 | 460 |
| Comp. Ex. 18 | Ref. 2BH-1 | BD-9 | 3.7 | 198 | 15 | 460 |

From the results in Tables 15 to 17, it can be seen that the combination of the tri-substituted anthracene compound 3BH-6 represented by the formula (1) and the compound BD-1, BD-8 or BD-9, which is a compound A that emits blue light with a small Stokes shift (SS), results in a blue fluorescent device that can be driven at low voltage and has a long lifetime, compared to the combination of the bi-substituted compound Ref. 2BH-1 and the compound BD-1, BD-8 or BD-9.

Although only some exemplary embodiments and/or examples of this invention have been described in detail above, those skilled in the art will readily appreciate that many modifications are possible in the exemplary embodiments and/or examples without materially departing from the novel teachings and advantages of this invention. Accordingly, all such modifications are intended to be included within the scope of this invention.

The documents described in the specification are incorporated herein by reference in its entirety.

1. An organic electroluminescence device comprising: a cathode; an anode; and an organic layer between the cathode and the anode,
wherein the organic layer comprises a compound represented by the following formula (1) and a compound represented by the following formula (11). 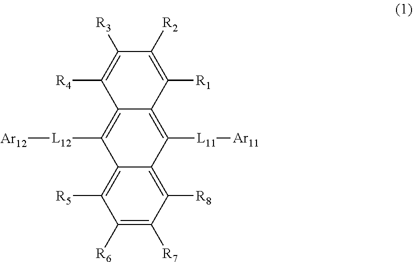
wherein in the formula (1),
one or more among R₁ to R₈ are -L₁₃-Ar₁₃;
L₁₁ to L₁₃ are independently,
a single bond,
a substituted or unsubstituted arylene group including 6 to 50 ring carbon atoms, or
a substituted or unsubstituted divalent heterocyclic group including 5 to 50 ring atoms;
when two or more L₁₃'s are present, the two or more L₁₃'s may be the same as or different to each other;
Ar₁₁ to Ar₁₃ are independently,
a substituted or unsubstituted aryl group including 6 to 50 ring carbon atoms, or
a substituted or unsubstituted monovalent heterocyclic group including 5 to 50 ring atoms;
when two or more Ar₁₃'s are present, the two or more Ar₁₃'s may be the same as or different to each other;
R₁ to R₈ which are not -L₁₃-Ar₁₃ are independently,
a hydrogen atom,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
a substituted or unsubstituted aryl group including 6 to 50 ring carbon atoms, or
a substituted or unsubstituted monovalent heterocyclic group including 5 to 50 ring atoms;
R₉₀₁ to R₉₀₇ are independently,
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and
when two or more of each of R₉₀₁ to R₉₀₇ are present, the two or more of each of R₉₀₁ to R₉₀₇ are the same or different: 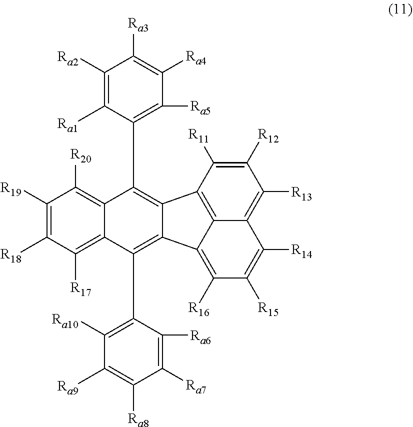
wherein in the formula (11),
any one or more sets among one or more sets of adjacent two or more of R₁₁ to R₂₀, one or more sets of adjacent two or more of Rₐ₁ to Rₐ₅, and one or more sets of adjacent two or more of Rₐ₆ to Rₐ₁₀, form a substituted or unsubstituted, saturated or unsaturated ring including 3 to 30 ring atoms by bonding with each other;
R₁₁ to R₂₀, Rₐ₁ to Rₐ₅ and Rₐ₆ to Rₐ₁₀ which do not form the ring are independently,
a hydrogen atom,
a substituted or unsubstituted alkyl group including 1 to 30 carbon atoms,
a substituted or unsubstituted cycloalkyl group including 3 to 30 ring carbon atoms,
a substituted or unsubstituted alkoxy group including 1 to 30 carbon atoms,
a substituted or unsubstituted alkylthio group including 1 to 30 carbon atoms,
a substituted or unsubstituted amino group,
a substituted or unsubstituted aryl group including 6 to 30 ring carbon atoms,
a substituted or unsubstituted heterocyclic group including 5 to 30 ring atoms.
a substituted or unsubstituted alkenyl group including 2 to 30 carbon atoms,
a substituted or unsubstituted aryloxy group including 6 to 30 ring carbon atoms,
a substituted or unsubstituted arylthio group including 6 to 30 ring carbon atoms,
a substituted or unsubstituted phosphanyl group,
a substituted or unsubstituted phosphoryl group,
a substituted or unsubstituted silyl group,
a substituted or unsubstituted arylcarbonyl group including 6 to 30 ring carbon atoms,
a cyano group, a nitro group, a carboxyl group, or
a halogen atom.

2. An organic electroluminescence device comprising: a cathode; an anode; and an organic layer between the cathode and the anode,
wherein the organic layer comprises a compound represented by the following formula (1), and
a compound A having a Stokes shift of 20 nm or less and an emission peak wavelength of 440 nm to 465 nm: 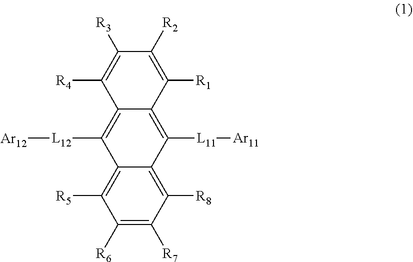
wherein in the formula (1),
one or more among R₁ to R₈ are -L₁₃-Ar₁₃;
L₁₁ to L₁₃ are independently,
a single bond,
a substituted or unsubstituted arylene group including 6 to 50 ring carbon atoms, or
a substituted or unsubstituted divalent heterocyclic group including 5 to 50 ring atoms;
when two or more L₁₃'s are present, the two or more L₁₃'s may be the same as or different to each other;
Ar₁₁ to Ar₁₃ are independently,
a substituted or unsubstituted aryl group including 6 to 50 ring carbon atoms, or
a substituted or unsubstituted monovalent heterocyclic group including 5 to 50 ring atoms;
when two or more Ar₁₃'s are present, the two or more Ar₁₃'s may be the same as or different to each other;
R₁ to R₈ which are not -L₁₃-Ar₁₃ are independently,
a hydrogen atom,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
a substituted or unsubstituted aryl group including 6 to 50 ring carbon atoms, or
a substituted or unsubstituted monovalent heterocyclic group including 5 to 50 ring atoms;
R₉₀₁ to R₉₀₇ are independently,
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and
when two or more of each of R₉₀₁ to R₉₀₇ are present, the two or more of each of R₉₀₁ to R₉₀₇ are the same or different.

3. The organic electroluminescence device according to claim 1, wherein L₁₁ to L₁₃ in the formula (1) are independently, a single bond, or
a substituted or unsubstituted arylene group including 6 to 50 ring carbon atoms.

4. The organic electroluminescence device according to claim 1, wherein L₁₁ to L₁₃ in the formula (1) are independently, a single bond, or
a group selected from the group consisting of:
a substituted or unsubstituted phenylene group,
a substituted or unsubstituted biphenylene group,
a substituted or unsubstituted terphenylene group,
a substituted or unsubstituted quaterphenylene group, and
a substituted or unsubstituted naphthylene group.

5. The organic electroluminescence device according to claim 1, wherein Ar₁₁ to Ar₁₃ in the formula (1) are independently a substituted or unsubstituted aryl group including 6 to 30 ring carbon atoms.

6. The organic electroluminescence device according to claim 1, wherein Ar₁₁ to Ar₁₃ in the formula (1) are independently selected from the group consisting of:
a substituted or unsubstituted phenyl group,
a substituted or unsubstituted naphthyl group,
a substituted or unsubstituted fluorenyl group,
a substituted or unsubstituted 9,9′-spirobifluorenyl group,
a substituted or unsubstituted benzofluorenyl group,
a substituted or unsubstituted phenanthryl group, and
a substituted or unsubstituted benzophenanthryl group.

7. The organic electroluminescence device according to claim 1, wherein one or more among Ar₁₁ to Ar₁₃ in the formula (1) are independently a substituted or unsubstituted monovalent heterocyclic group including 5 to 30 ring atoms.

8. The organic electroluminescence device according to claim 1, wherein the compound represented by the formula (1) is a compound represented by the following formula (1-1): 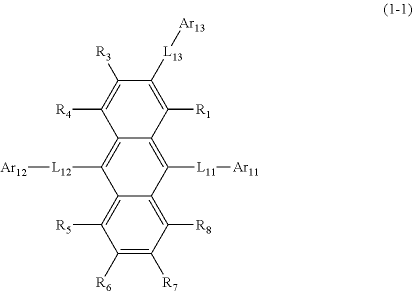
wherein in the formula (1-1), L₁₁ to L₁₃, Ar₁₁ to Ar₁₃, R₁, R₃, R₄ and R₅ to R₈ are as defined in the formula (1).

9. The organic electroluminescence device according to claim 1, wherein the compound represented by the formula (1) is a compound represented by the following formula (1-1H): 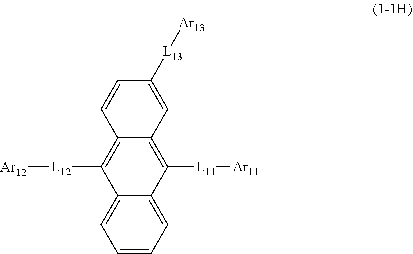
wherein in the formula (1-1H), L₁₁ to L₁₃ and Ar₁₁ to Ar₁₃ are as defined in the formula (1).

10. The organic electroluminescence device according to claim 1, wherein the group represented by -L₁₃-Ar₁₃ in the formula (1) is selected from the group consisting of:
a substituted or unsubstituted phenyl group,
a substituted or unsubstituted naphthyl group,
a substituted or unsubstituted biphenyl group,
a substituted or unsubstituted phenanthrenyl group,
a substituted or unsubstituted benzophenanthrenyl group,
a substituted or unsubstituted fluorenyl group,
a substituted or unsubstituted benzofluorenyl group,
a substituted or unsubstituted dibenzofuranyl group,
a substituted or unsubstituted naphthobenzofuranyl group,
a substituted or unsubstituted dibenzothiophenyl group, and
a substituted or unsubstituted carbazolyl group.

11. The organic electroluminescence device according to claim 1, wherein the compound represented by the formula (1) is selected from the group consisting of a compound represented by the following formula (1-2), a compound represented by the following formula (1-3), and a compound represented by the following formula (1-4): 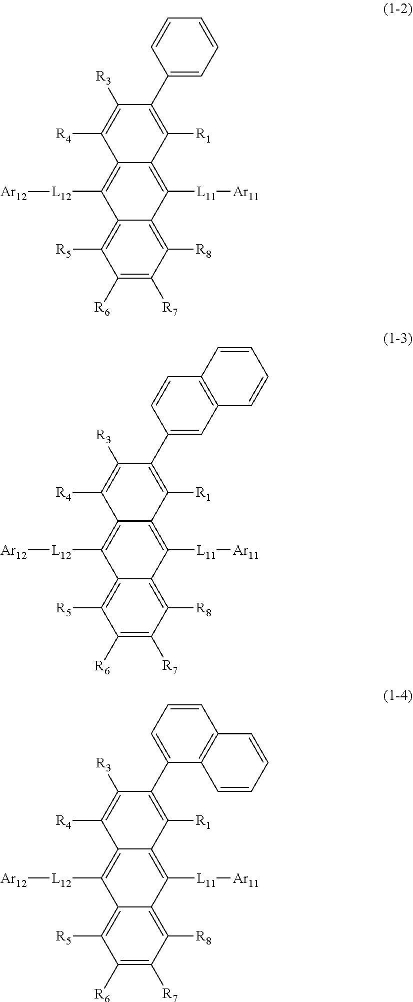
wherein in the formulas (1-2) to (1-4), L₁₁, L₁₂, Ar₁₁, Ar₁₂, R₁, R₃, R₄ and R₅ to R₈ are as defined in the formula (1).

12. The organic electroluminescence device according to claim 1, wherein R₁ to R₈ which are not -L₁₃-Ar₁₃ in the formula (1) are hydrogen atoms.

13. The organic electroluminescence device according to claim 1, wherein R₁₂ and R₁₃ in the formula (11) form a substituted or unsubstituted, saturated or unsaturated ring including 3 to 30 ring atoms by bonding with each other.

14. The organic electroluminescence device according to claim 1, wherein the compound represented by the formula (11) is a compound represented by the following formula (11-1): 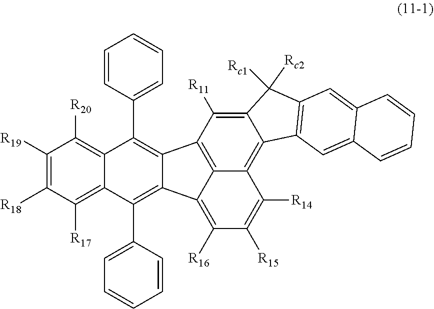
wherein in the formula (11-1), R₁₁, and R₁₄ to R₂₀ are as defined in the formula (11);
R_{c1} and R_{c2} are independently,
a hydrogen atom,
an unsubstituted alkyl group including 1 to 50 carbon atoms,
an unsubstituted alkenyl group including 2 to 50 carbon atoms,
an unsubstituted alkynyl group including 2 to 50 carbon atoms,
an unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
an unsubstituted aryl group including 6 to 50 ring carbon atoms, or
an unsubstituted monovalent heterocyclic group including 5 to 50 ring atoms;
R₉₀₁ to R₉₀₇ are independently,
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and
when two or more of each of R₉₀₁ to R₉₀₇ are present, the two or more of each of R₉₀₁ to R₉₀₇ are the same or different.

15. The organic electroluminescence device according to claim 1, wherein two or more among R₁₈ to R₂₀ in the formula (11) form a substituted or unsubstituted, saturated or unsaturated ring including 3 to 30 ring atoms by bonding with each other.

16. The organic electroluminescence device according to claim 1, wherein the compound represented by the formula (11) is a compound represented by the following formula (11-2): 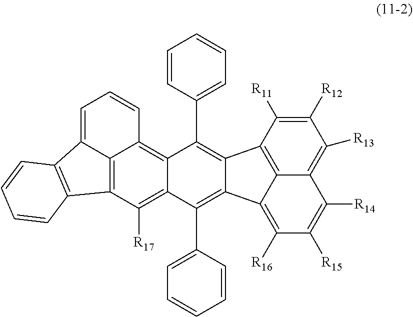
wherein in the formula (11-2), R₁₁ to R₁₇ are as defined in the formula (11).

17. The organic electroluminescence device according to claim 1, wherein R₁₁ to R₂₀, Rₐ₁ to Rₐ₅ and Rₐ₆ to Rₐ₁₀ in the formula (11), which do not form a ring are independently,
a hydrogen atom,
an unsubstituted aryl group including 6 to 50 ring carbon atoms, or
an unsubstituted monovalent heterocyclic group including 5 to 50 ring atoms.

18. The organic electroluminescence device according to claim 1, wherein the organic layer comprises an emitting layer, and
the emitting layer comprises the compound represented by the formula (1) and the compound represented by the formula (11).

19. The organic electroluminescence device according to claim 2, wherein the organic layer comprises an emitting layer, and
the emitting layer comprises the compound represented by the formula (1) and the compound A.

20. The organic electroluminescence device according to claim 2, wherein the compound A is one or more selected from the group consisting of a compound represented by the following formula (A-1) and a compound represented by the following formula (A-2): 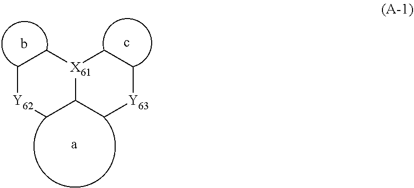
wherein in the formula (A-1),
ring a, ring b and ring c are independently,
a substituted or unsubstituted aromatic hydrocarbon ring including 6 to 50 ring carbon atoms, or
a substituted or unsubstituted heterocyclic ring including 5 to 50 ring atoms;
X₆₁ is B or N;
Y₆₂ and Y₆₃ are independently NR_{d}, O, S, or a single bond;
provided that when X₆₁ is B, Y₆₂ and Y₆₃ are independently NR_{d}, O or S; when X₆₁ is N, Y₆₂ and Y₆₃ are single bonds;
R_{d} forms a substituted or unsubstituted heterocyclic ring by bonding with the ring a, the ring b, or the ring c, or does not form a substituted or unsubstituted heterocyclic ring; and
R_{d}'s which do not form the substituted or unsubstituted heterocyclic ring are independently,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms: 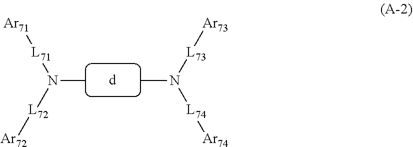
wherein in the formula (A-2),
ring d is a substituted or unsubstituted aromatic hydrocarbon ring including 6 to 50 ring carbon atoms, or a substituted or unsubstituted heterocyclic ring including 5 to 50 ring atoms;
L₇₁ to L₇₄ are independently,
a single bond,
a substituted or unsubstituted arylene group including 6 to 50 ring carbon atoms, or
a substituted or unsubstituted divalent heterocyclic group including 5 to 50 ring atoms;
Ar₇₁ to Ar₇₄ are independently,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms;
provided that when the ring d is a substituted or unsubstituted aromatic hydrocarbon ring including 10 to 50 ring carbon atoms, two or more among Ar₇₁ to Ar₇₄ are independently an aryl group including 6 to 50 ring carbon atoms, that is substituted by an alkyl group including 1 to 50 carbon atoms, or a monovalent heterocyclic group including 5 to 50 ring atoms, that is substituted by an alkyl group including 1 to 50 carbon atoms.

21. The organic electroluminescence device according to claim 18, wherein the organic layer further comprises a hole-blocking layer directly in contact with the emitting layer,
wherein the hole-blocking layer comprises either or both of a compound represented by the following formula (21) and a compound represented by the following formula (31): 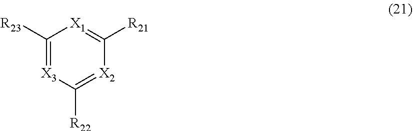
wherein in the formula (21),
X₁ to X₃ are independently N or CR_{b}; provided that one or more among X₁ to X₃ are N;
R_{b} is
a hydrogen atom,
a halogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms;
when two R_{b}'s are present, the two R_{b}'s may be the same as or different to each other;
R_{b} does not form a ring by bonding with adjacent R₂₁ to R₂₃;
R₂₁ to R₂₃ are independently,
-(L₂)ₘ-(Ar₂)ₙ,
a hydrogen atom,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
a substituted or unsubstituted aryl group including 6 to 50 ring carbon atoms, or
a substituted or unsubstituted monovalent heterocyclic group including 5 to 50 ring atoms;
R₉₀₁ to R₉₀₇ are independently,
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; when two or more of each of R₉₀₁ to R₉₀₇ are present, the two or more of each of R₉₀₁ to R₉₀₇ are the same or different;
L₂ is
a substituted or unsubstituted arylene group including 6 to 50 ring carbon atoms, or
a substituted or unsubstituted divalent heterocyclic group including 5 to 50 ring atoms;
m is an integer of 0 to 2; when m is 0, L₂ is a single bond; when m is 2, two L₂'s may be the same or different;
Ar₂ is
a substituted or unsubstituted aryl group including 6 to 50 ring carbon atoms, or
a substituted or unsubstituted monovalent heterocyclic group including 5 to 50 ring atoms;
n is an integer of 1 or 2, and when n is 2, two Ar₂'s may be the same as or different to each other; provided that when n is 2, m is 1 or more: 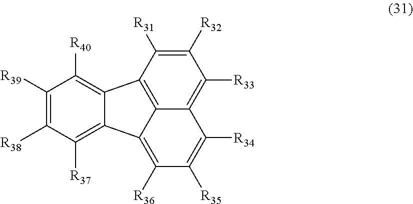
wherein in the formula (31),
one or more among R₃₁ to R₄₀ are -(L₃)ₚ-Ar₃; when two or more -(L₃)ₚ-Ar₃'s are present, the two or more -(L₃)ₚ-Ar₃'s may be the same as or different to each other;
L₃ is
a substituted or unsubstituted arylene group including 6 to 50 ring carbon atoms, or
a substituted or unsubstituted divalent heterocyclic group including 5 to 50 ring atoms;
p is an integer of 0 to 3; when p is 0, L₃ is a single bond; when p is 2 or more, a plurality of L₃'s may be the same as or different to each other;
Ar₃ is
a substituted or unsubstituted aryl group including 6 to 50 ring carbon atoms, or
a substituted or unsubstituted monovalent heterocyclic group including 5 to 50 ring atoms;
one or more sets of adjacent two or more among R₃₁ to R₃₆ which are not -(L₃)ₚ-Ar₃ and R₃₇ to R₄₀ which are not -(L₃)ₚ-Ar₃ form a substituted or unsubstituted, saturated or unsaturated ring including 3 to 30 ring atoms by bonding with each other, or do not form a ring;
R₃₁ to R₄₀ which are not -(L₃)ₚ-Ar₃ and which are not involved in the ring formation are independently,
a hydrogen atom,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
a substituted or unsubstituted aryl group including 6 to 50 ring carbon atoms, or
a substituted or unsubstituted monovalent heterocyclic group including 5 to 50 ring atoms;
R₉₀₁ to R₉₀₇ are independently,
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and
when two or more of each of R₉₀₁ to R₉₀₇ are present, the two or more of each of R₉₀₁ to R₉₀₇ are the same or different.

22. The organic electroluminescence device according to claim 21, wherein two among X₁ to X₃ in the formula (21) are N.

23. The organic electroluminescence device according to claim 21, wherein R₂₁ to R₂₃ in the formula (21) are independently,
a substituted or unsubstituted aryl group including 6 to 50 ring carbon atoms.

24. The organic electroluminescence device according to claim 21, wherein the compound represented by the formula (21) is a compound represented by the following formula (21-1): 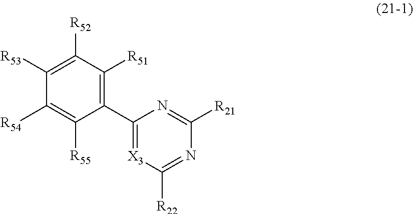
wherein in the formula (21-1), R₂₁, R₂₂ and X₃ are as defined in the formula (21);
R₅₁ to R₅₅ are independently,
a hydrogen atom,
an unsubstituted alkyl group including 1 to 50 carbon atoms,
an unsubstituted alkenyl group including 2 to 50 carbon atoms,
an unsubstituted alkynyl group including 2 to 50 carbon atoms,
an unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
an unsubstituted aryl group including 6 to 50 ring carbon atoms, or
an unsubstituted monovalent heterocyclic group including 5 to 50 ring atoms;
one or more sets of adjacent two or more among R₅₁ to R₅₅ form a substituted or unsubstituted, saturated or unsaturated ring including 3 to 30 ring atoms by bonding with each other, or do not form a ring;
R₉₀₁ to R₉₀₇ are independently,
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and
when two or more of each of R₉₀₁ to R₉₀₇ are present, the two or more of each of R₉₀₁ to R₉₀₇ are the same or different.

25. The organic electroluminescence device according to claim 24, wherein the compound represented by the formula (21-1) is a compound represented by the following formula (21-2): 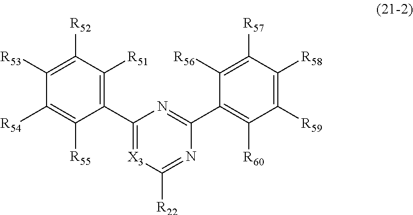
wherein in the formula (21-2), R₂₂, X₃ and R₅₁ to R₅₅ are as defined in the formula (21-1);
R₅₆ to R₆₀ are independently,
a hydrogen atom,
an unsubstituted alkyl group including 1 to 50 carbon atoms,
an unsubstituted alkenyl group including 2 to 50 carbon atoms,
an unsubstituted alkynyl group including 2 to 50 carbon atoms,
an unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
an unsubstituted aryl group including 6 to 50 ring carbon atoms, or
an unsubstituted monovalent heterocyclic group including 5 to 50 ring atoms;
R₉₀₁ to R₉₀₇ are independently,
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and
when two or more of each of R₉₀₁ to R₉₀₇ are present, the two or more of each of R₉₀₁ to R₉₀₇ are the same or different.

26. The organic electroluminescence device according to claim 25, wherein the compound represented by the formula (21-2) is a compound represented by the following formula (21-3): 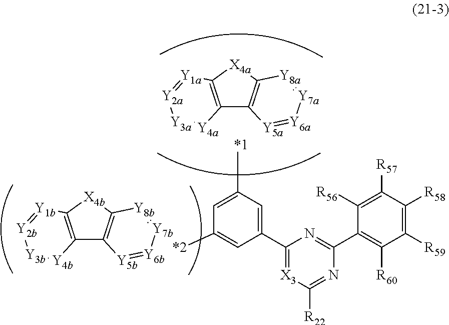
wherein in the formula (21-3), R₂₂, X₃ and R₅₆ to R₆₀ are as defined in the formula (21-2);
Y₁ₐ to Y₈ₐ are independently CR₆₁ₐ or N;
Y_{1b} to Y_{8b} are independently CR_{61b} or N;
X₄ₐ is O, S or NR₆₁ₐ;
X_{4b} is O, S or NR_{61b};
R₆₁ₐ and R_{61b} are independently,
a hydrogen atom,
an unsubstituted alkyl group including 1 to 50 carbon atoms,
an unsubstituted alkenyl group including 2 to 50 carbon atoms,
an unsubstituted alkynyl group including 2 to 50 carbon atoms,
an unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
an unsubstituted aryl group including 6 to 50 ring carbon atoms, or
an unsubstituted monovalent heterocyclic group including 5 to 50 ring atoms;
R₉₀₁ to R₉₀₇ are independently,
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms;
when two or more of each of R₉₀₁ to R₉₀₇ are present, the two or more of each of R₉₀₁ to R₉₀₇ are the same or different;
when a plurality of R₆₁ₐ's are present, the plurality of R₆₁ₐ's may be the same as or different to each other;
when a plurality of R_{61b}'s are present, the plurality of R_{61b}'s may be the same as or different to each other;
one or more sets of two or more R₆₁ₐ's substituting adjacent atoms form a substituted or unsubstituted, saturated or unsaturated ring including 3 to 30 ring atoms by bonding with each other, or do not form a ring;
one or more sets of two or more R_{61b}'s substituting adjacent atoms form a substituted or unsubstituted, saturated or unsaturated ring including 3 to 30 ring atoms by bonding with each other, or do not form a ring;
provided that one of R₆₁ₐ's is a single bond which is bonded with *1, or one of the atoms constituting the ring formed by bonding one or more sets of R₆₁ₐ's substituting adjacent atoms with each other is bonded with *1 via a single bond; and
one of R_{61b}'s is a single bond which is bonded with *2, or one of the atoms constituting the ring formed by bonding one or more sets of two or more R_{61b}'s substituting adjacent atoms with each other is bonded with *2 via a single bond.

27. The organic electroluminescence device according to claim 21, wherein the compound represented by the formula (21) is a compound represented by the following formula (21-4): 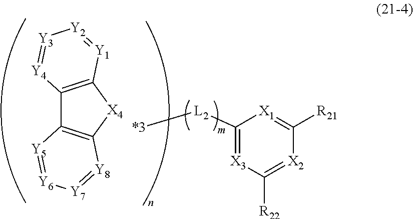
wherein in the formula (21-4), X₁ to X₃, R₂₁, R₂₂, L₂, m, and n are as defined in the formula (21);
Y₁ to Y₈ are independently CR₆₁ₑ or N;
X₄ is O, S or NR₆₁ₑ;
R₆₁ₑ's are independently,
a hydrogen atom,
an unsubstituted alkyl group including 1 to 50 carbon atoms,
an unsubstituted alkenyl group including 2 to 50 carbon atoms,
an unsubstituted alkynyl group including 2 to 50 carbon atoms,
an unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
an unsubstituted aryl group including 6 to 50 ring carbon atoms, or
an unsubstituted monovalent heterocyclic group including 5 to 50 ring atoms;
R₉₀₁ to R₉₀₇ are independently,
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and
when two or more of each of R₉₀₁ to R₉₀₇ are present, the two or more of each of R₉₀₁ to R₉₀₇ are the same or different;
when a plurality of R₆₁ₑ's are present, the plurality of R₆₁ₑ's may be the same as or different to each other;
one or more sets of two or more R₆₁ₑ's substituting adjacent atoms form a substituted or unsubstituted, saturated or unsaturated ring including 3 to 30 ring atoms by bonding with each other, or do not form a ring;
provided that one of R₆₁ₑ's is a single bond which is bonded with *3, or one of the atoms constituting the ring formed by bonding one or more sets of two or more R₆₁ₑ's substituting adjacent atoms with each other is bonded with *3 via a single bond.

28. The organic electroluminescence device according to claim 21, wherein the compound represented by the formula (31) is a compound represented by the following formula (31-1): 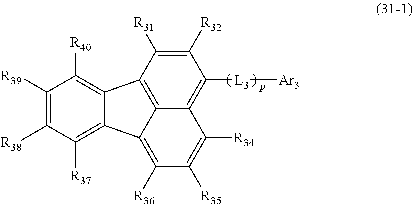
wherein in the formula (31-1), L₃, p, Ar₃, R₃₁, R₃₂, and R₃₄ to R₄₀ are as defined in the formula (31).

29. The organic electroluminescence device according to claim 21, wherein the compound represented by the formula (31) is a compound represented by the following formula (31-1H): 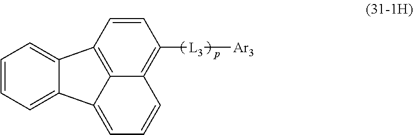
wherein in the formula (31-1H), L₃, p and Ar₃ are as defined in the formula (31).

30. The organic electroluminescence device according to claim 18, wherein the organic layer further comprises an electron-blocking layer directly in contact with an emitting layer,
wherein the electron-blocking layer comprises either or both of a compound represented by the following formula (41) and a compound represented by the following formula (51): 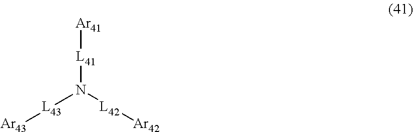
wherein in the formula (41),
L₄₁ to L₄₃ are independently,
a single bond,
a substituted or unsubstituted arylene group including 6 to 50 ring carbon atoms, or
a substituted or unsubstituted divalent heterocyclic group including 5 to 50 ring atoms;
Ar₄₁ to Ar₄₃ are independently,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms: 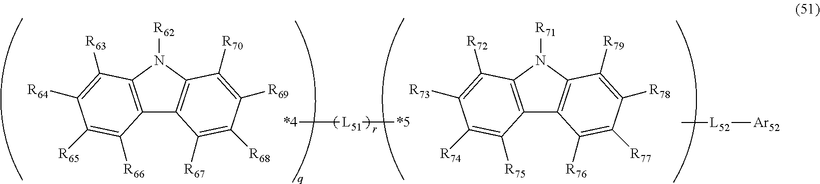
wherein in the formula (51),
R₆₂ to R₇₉ are independently,
a hydrogen atom,
an unsubstituted alkyl group including 1 to 50 carbon atoms,
an unsubstituted alkenyl group including 2 to 50 carbon atoms,
an unsubstituted alkynyl group including 2 to 50 carbon atoms,
an unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
an unsubstituted aryl group including 6 to 50 ring carbon atoms, or
an unsubstituted monovalent heterocyclic group including 5 to 50 ring atoms;
R₉₀₁ to R₉₀₇ are independently,
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and
when two or more of each of R₉₀₁ to R₉₀₇ are present, the two or more of each of R₉₀₁ to R₉₀₇ are the same or different;
one or more sets of two or more among R₆₂ to R₇₀ substituting adjacent atoms form a substituted or unsubstituted, saturated or unsaturated ring including 3 to 30 ring atoms by bonding with each other, or do not form a ring;
one or more sets of two or more among R₇₁ to R₇₉ substituting adjacent atoms form a substituted or unsubstituted, saturated or unsaturated ring including 3 to 30 ring atoms by bonding with each other, or do not form a ring;
provided that one of R₆₂ to R₇₀ is a single bond which is bonded with *4, or one of the atoms constituting the ring formed by bonding one or more sets of two or more among R₆₂ to R₇₀ substituting adjacent atoms with each other is bonded with *4 via a single bond;
one of R₇₁ to R₇₉ is a single bond which is bonded with *5, or one of the atoms constituting the ring formed by bonding one or more sets of two or more among R₇₁ to R₇₉ substituting adjacent atoms with each other is bonded with *5 via a single bond; and one of R₇₁ to R₇₉ which is not bonded with *5 is a single bond which is bonded with L₅₂, or another one of the atoms constituting the ring formed by bonding one or more sets of two or more among R₇₁ to R₇₉ substituting adjacent atoms with each other is bonded with L₅₂ via a single bond;
L₅₁'s are independently,
a substituted or unsubstituted arylene group including 6 to 50 ring carbon atoms, or
a substituted or unsubstituted divalent heterocyclic group including 5 to 50 ring atoms;
q is an integer of 0 to 3; when q is 2 or more, two or more of each of R₆₂ to R₇₀ may be the same as or different to each other; provided that when q is 0, L₅₁ is terminated by a hydrogen atom;
r is an integer of 0 to 2; when r is 0, L₅₁ is a single bond; when r is 2, two L₅₁'s may be the same as or different to each other; provided that when q is 2 or more, r is 1 or 2;
L₅₂ is
a single bond,
a substituted or unsubstituted arylene group including 6 to 50 ring carbon atoms, or
a substituted or unsubstituted divalent heterocyclic group including 5 to 50 ring atoms;
Ar₅₂ is
a substituted or unsubstituted aryl group including 6 to 50 ring carbon atoms, or
a substituted or unsubstituted monovalent heterocyclic group including 5 to 50 ring atoms.

31. The organic electroluminescence device according to claim 30, wherein the compound represented by the formula (41) is a compound represented by the following formula (41-1): 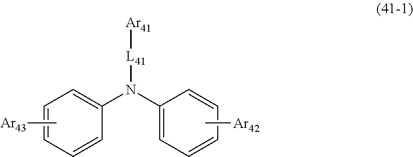
wherein in the formula (41-1), Ar₄₁ to Ar₄₃ and L₄₁ are as defined in the formula (41).

32. The organic electroluminescence device according to claim 30, wherein the compound represented by the formula (41) is a compound represented by the following formula (41-2): 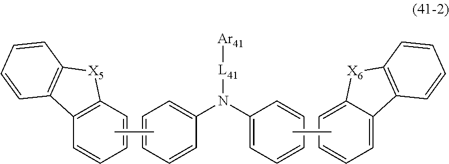
wherein in the formula (41-2), Ar₄₁ and L₄₁ are as defined in the formula (41);
X₅ and X₆ are independently O, S or N(R₉₀₆);
R₉₀₆ is
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; when two R₉₀₆'s are present, the two R₉₀₆ may be the same or different.

33. The organic electroluminescence device according to claim 30, wherein the compound represented by the formula (41) is a compound represented by the following formula (41-3): 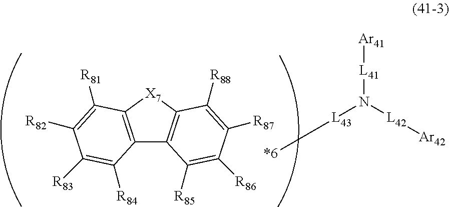
wherein in the formula (41-3), Ar₄₁, Ar₄₂ and L₄₁ to L₄₃ are as defined in the formula (41);
X₇ is O, S or NR₈₉;
R₈₁ to R₈₉ are independently,
a hydrogen atom,
an unsubstituted alkyl group including 1 to 50 carbon atoms,
an unsubstituted alkenyl group including 2 to 50 carbon atoms,
an unsubstituted alkynyl group including 2 to 50 carbon atoms,
an unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
an unsubstituted aryl group including 6 to 50 ring carbon atoms, or
an unsubstituted monovalent heterocyclic group including 5 to 50 ring atoms;
R₉₀₁ to R₉₀₇ are independently,
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms. when two or more of each of R₉₀₁ to R₉₀₇ are present, the two or more of each of R₉₀₁ to R₉₀₇ are the same or different; and
one or more sets of two or more among R₈₁ to R₈₉ substituting adjacent atoms form a substituted or unsubstituted, saturated or unsaturated ring including 3 to 30 ring atoms by bonding with each other, or do not form a ring;
provided that one of R₈₁ to R₈₉ is a single bond which is bonded with *6, or one of the atoms constituting the ring formed by bonding one or more sets of two or more among R₈₁ to R₈₉ substituting adjacent atoms with each other is bonded with *6 via a single bond.

34. The organic electroluminescence device according to claim 30, wherein the compound represented by the formula (51) is a compound represented by the following formula (51-1): 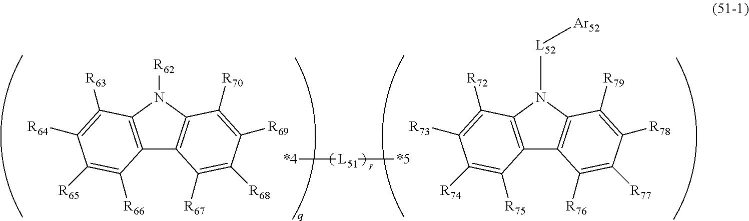
wherein in the formula (51-1), R₆₂ to R₇₀, R₇₂ to R₇₉, *4, *5, q, L₅₁, r, L₅₂ and Ar₅₂ areas defined in the formula (51).

35. The organic electroluminescence device according to claim 30, wherein q in the formula (51) is 1.

36. The organic electroluminescence device according to claim 30, wherein the compound represented by the formula (51) is a compound represented by the following formula (51-2): 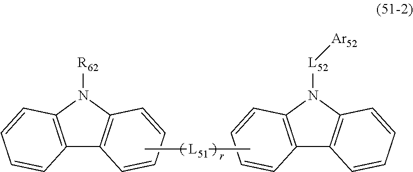
wherein in the formula (51-2), R₆₂, L₅₁, r, L₅₂ and Ar₅₂ are as defined in the formula (51).

37. The organic electroluminescence device according to claim 30, wherein the compound represented by the formula (51) is selected from the group consisting of a compound represented by the following formula (51-3a), a compound represented by the following formula (51-3b), and a compound represented by the following formula (51-3c): 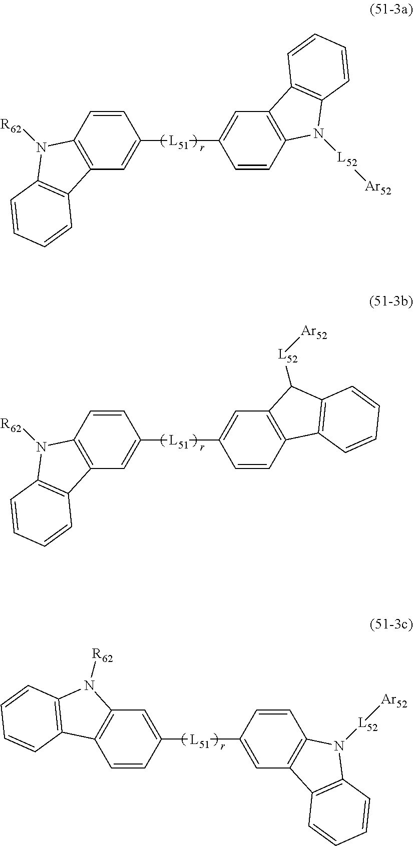
wherein in the formulas (51-3a) to (51-3c), R₆₂, L₅₁, r, L₅₂ and Ar₅₂ are as defined in the formula (51).

38. The organic electroluminescence device according to claim 1, wherein the substituent in the case of “substituted or unsubstituted” is a group selected from the group consisting of:
an unsubstituted alkyl group including 1 to 50 carbon atoms,
an unsubstituted alkenyl group including 2 to 50 carbon atoms,
an unsubstituted alkynyl group including 2 to 50 carbon atoms,
an unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇)
(where,
R₉₀₁ to R₉₀₇ are independently,
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; when two or more of each of R₉₀₁ to R₉₀₇ are present, the two or more of each of R₉₀₁ to R₉₀₇ are the same or different),
a halogen atom, a cyano group, a nitro group,
an unsubstituted aryl group including 6 to 50 ring carbon atoms, and
an unsubstituted monovalent heterocyclic group including 5 to 50 ring atoms.

39. The organic electroluminescence device according to claim 1, wherein the substituent in the case of “substituted or unsubstituted” is a group selected from the group consisting of:
an alkyl group including 1 to 50 carbon atoms,
an aryl group including 6 to 50 ring carbon atoms, and
a monovalent heterocyclic group including 5 to 50 ring atoms.

40. The organic electroluminescence device according to claim 1, wherein the substituent in the case of “substituted or unsubstituted” is a group selected from the group consisting of:
an alkyl group including 1 to 18 carbon atoms,
an aryl group including 6 to 18 ring carbon atoms, and
a monovalent heterocyclic group including 5 to 18 ring atoms.

41. An electronic appliance, comprising the organic electroluminescence device according to claim 1.
